(19) United States
(12) Patent Application Publication
(10) Pub. No.: US 2021/0164056 A1
(43) Pub. Date: Jun. 3, 2021

(54) USE OF METASTASES-SPECIFIC SIGNATURES FOR TREATMENT OF CANCER

(71) Applicant: THE UNIVERSITY OF CHICAGO, Chicago, IL (US)

(72) Inventor: Michael SPIOTTO, Chicago, IL (US)

(21) Appl. No.: 17/262,471

(22) PCT Filed: Jul. 24, 2019
(86) PCT No.: PCT/US2019/043312

§ 371 Date: Jan. 22, 2021

Related U.S. Application Data
(60) Provisional application No. 62/703,237, filed on Jul. 25, 2018.

(51) Int. Cl. C12Q 1/6886; C12Q 1/686

(52) U.S. Cl. CPC C12Q 1/6886; G01N 2800/52; C12Q 1/686

(57) ABSTRACT

The invention provides methods for treating cancer in a subject and/or predicting outcome of a lymph node metastasis (LNM) in a subject, the methods comprising obtaining a sample comprising LNM cells from the subject; measuring gene expression levels in the sample of at least ten genes; determining a LNM-specific molecular subtype comprising the gene expression levels of the at least ten genes; providing an indication as to the outcome when the LNM-specific molecular subtype indicates that the expression levels of the at least ten genes are altered in a predictive manner; and administering an aggressive cancer treatment regimen to the subject when a determination is made that the subject has LNM cells with decreased outcome.

### PRIORITY

This application claims the benefit of U.S. provisional application Ser. No. 62/703,237, filed on Jul. 25, 2018, which is incorporated by reference herein in its entirety.

### STATEMENT REGARDING FEDERALLY SPONSORED RESEARCH OR DEVELOPMENT

This invention was made with government support under DE027445 awarded by the National Institutes of Health. The government has certain rights in the invention.

### BACKGROUND OF THE INVENTION

### Field of the Invention

This disclosure relates to compositions and methods for diagnosing and treating cancer, and head and neck squamous cells cancers (HNSCCs), breast cancers, and melanomas in particular.

### Description of Related Art

Lymph node metastases (LNMs) represent an aggressive yet curable state for many solid tumors. The clinical significance of LNMs reflects, in part, an intermediate metastatic step during the sequential spread of cancers from the primary site to the lymph nodes and then to distant organs (1-3). Alternatively, LNMs may not directly lead to distant metastases but reflect the biological selection of more aggressive subpopulations that are associated with increased local and/or regional recurrence (4-6). The presence of LNMs is associated with decreased survival in gastric cancers, melanomas, and breast cancers among others (7-9). Similarly, LNMs are one of the most important prognostic features in head and neck squamous cells cancers (HNSCCs) (10), where the survival of patients with LNMs is approximately half the survival of patients without LNMs (11, 12). However, despite these worse outcomes, LNMs represent a heterogeneous disease state as evidenced by the inability of existing lymph node staging systems to accurately predict locoregional control, distant metastasis, and/or survival (13). Previous investigators have tried to stratify patients with LNMs using clinicopathological features including lymph node density, tumor and/or lymph node location, and extracapsular extension, among others (14, 15). Although several groups have sought to biologically classify LNMs in HNSCCs, and in other cancers, these efforts have primarily focused on differences between LNMs and the primary tumor rather than defining distinct subtypes within LNMs (16, 17). Given that approximately 50-60% of HNSCC patients present with LNMs (18), identifying LNM subtypes based on intrinsic molecular differences would likely provide novel biological and prognostic information.

Unsupervised integrative clustering of 72 LNMs was used to identify three distinct LNM-specific molecular subtypes associated with different rates of locoregional control and survival. The most aggressive LNM subtype displayed a lymph node-specific gene expression signature that was prognostic across multiple cancer types and was enriched for genes associated with an invasive subtype.

In many cancers, LNMs portend for worse chances for survival. However, the heterogeneous outcomes of patients with LNMs indicate a need to identify subgroups of patients at increased risk of disease recurrence. Using unsupervised clustering of RNA and miRNA profiles, three novel subtypes of LNMs were identified in Human Papillomavirus-negative Head and Neck cancers. One LNM-specific subtype had a 10-fold higher risk of locoregional recurrence in head and neck cancers and predicted for worse disease control and/or survival in breast cancers and melanomas. Identification of this aggressive subtype in patients will enable better risk stratification of patients and the selection of patients for treatment escalation. Furthermore, gene pathways selectively activated in this high risk subtype will facilitate the identification of new targets to more precisely treat patients at increased risk of disease recurrence. Finally, these results likely extend to other cancer types.

### SUMMARY OF THE INVENTION

This disclosure identified three distinct molecular subtypes of LNMs that were associated with differences in clinical outcomes after surgery and post-operative radiotherapy. These subtypes, termed Group 1, Group 2, and Group 3, displayed an immune, an invasive, and a metabolic/proliferative phenotype, respectively. The Group 2 LNM subtype was associated with significantly worse locoregional control, relapse-free survival, and overall survival. This predictive classifier was established in HNSCCs and validated in breast adenocarcinomas, and in melanomas. Since this classifier was predictive in epithelial cancers of different histologies, as well as in non-epithelial cancers, this classifier is likely predictive in all cancer types. The common biological pathways were enriched in these distinct cancer types further supporting the role of this classifier in pan-cancer analyses. Finally, the enriched pathways are commonly associated with poor prognostic features across many cancers. Thus, this signature is predictive for multiple cancer types.

In a first aspect, the invention provides a method for treating cancer in a subject, the method comprising:
(a) obtaining a sample comprising lymph node metastasis (LNM) cells from the subject;
(b) measuring gene expression levels in the sample of at least ten genes selected from the group consisting of: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3;
(c) determining a LNM-specific molecular subtype comprising the gene expression levels of the at least ten genes;
(d) providing an indication as to the outcome of locoregional control (LRC), relapse-free survival (RFS), and/or overall survival (OS) when the LNM-specific molecular subtype indicates that the expression levels of the at least ten genes are altered in a predictive manner; and
(e) administering an aggressive cancer treatment regimen to the subject when a determination is made that the subject has LNM cells with decreased LRC, decreased RFS, and/or decreased OS.

In certain embodiments of the first aspect, measuring gene expression levels of the at least ten genes comprises measurement by RNA-sequencing, miRNA sequencing, microarrays, Real-Time PCR (RT-PCR; including quantitative RT-PCR), microRNA, nanostring or other hybridization detection methods, immunohistochemistry/Western blot analyses, nuclease protection assays, and Northern blot analyses.

In certain embodiments of the first aspect, the LNM cells from the subject are obtained from a primary cancer selected from the group consisting of: human papillomavirus-negative head and neck cancer, head and neck squamous cell cancer, breast cancer, melanoma, acute lymphoblastic leukemia (ALL), acute myeloid leukemia (AML), adrenocortical carcinoma, kaposi sarcoma, aids-related lymphoma, primary CNS lymphoma, anal cancer, appendix cancer, astrocytomas, brain cancer, atypical teratoid/rhabdoid tumor, basal cell carcinoma of the skin, bile duct cancer, bladder cancer, bone cancer, bronchial tumors, Burkitt lymphoma, non-Hodgkin lymphoma, carcinoid tumor (gastrointestinal), cardiac tumors, cervical cancer, cholangiocarcinoma, chordoma, chronic lymphocytic leukemia (CLL), chronic myelogenous leukemia (CML), colorectal cancer, craniopharyngioma, cutaneous t-cell lymphoma, ductal carcinoma in situ (DCIS), endometrial cancer, esophageal cancer, esthesioneuroblastoma, extragonadal germ cell tumor, eye cancer, intraocular melanoma, retinoblastoma, fallopian tube cancer, gallbladder cancer, gastric cancer, gastrointestinal carcinoid tumor, gastrointestinal stromal tumors (GIST), germ cell tumors, gestational trophoblastic disease, hairy cell leukemia, head and neck cancer, liver cancer, Hodgkin lymphoma, hypopharyngeal cancer, islet cell tumors, pancreatic neuroendocrine tumors, Langerhans cell histiocytosis, laryngeal cancer, leukemia, lip and oral cavity cancer, lung cancer, lymphoma, Merkel cell carcinoma, mesothelioma, plasma cell neoplasms, mycosis fungoides, myelodysplastic syndromes, myelodysplastic/myeloproliferative neoplasms, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paraganglioma, parathyroid cancer, penile cancer, multiple myeloma, prostate cancer, renal cell cancer, retinoblastoma, rhabdomyosarcoma, salivary gland cancer, sarcoma, uterine sarcoma, small cell lung cancer, small intestine cancer, soft tissue sarcoma, t-cell lymphoma, testicular cancer, throat cancer, thymoma, thyroid cancer, urethral cancer, uterine cancer, vaginal cancer, and Wilms tumor.

In certain embodiments of the first aspect, the primary cancer is a head and neck squamous cell cancer.

In certain embodiments of the first aspect, the primary cancer is breast cancer.

In certain embodiments of the first aspect, the primary cancer is melanoma.

In certain embodiments of the first aspect, the aggressive cancer treatment regimen comprises surgical resection of the primary tumor and lymph node dissection followed by radiotherapy with or without chemotherapy

In certain embodiments of the first aspect, the lymph node metastasis is taken from a formalin-fixed, paraffin-embedded sample. In some embodiments, samples may be isolated from fine needle aspirates, or fresh frozen tissues.

In certain embodiments of the first aspect, the subject is a human.

In a second aspect, the invention provides a method for predicting outcome of locoregional control (LRC), relapse-free survival (RFS), and/or overall survival (OS) of a lymph node metastasis (LNM) in a subject, the method comprising:
(a) obtaining a sample comprising LNM cells from the subject;
(b) measuring gene expression levels in the sample of at least ten genes selected from the group consisting of: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3;
(c) determining a LNM-specific molecular subtype comprising the gene expression levels of the at least ten genes; and
(d) providing an indication as to the outcome of LRC, RFS, and/or OS when the LNM-specific molecular subtype indicates that the expression levels of the at least ten genes are altered in a predictive manner.

In certain embodiments of the second aspect, the LNM-specific molecular subtype for the LNM is classified as Group 1, Group 2, or Group 3. In certain embodiments, Group 1 indicates an immune subtype, Group 2 indicates a significantly worse LRC, RFS, and/or OS, and Group 3 indicates a metabolic/proliferative subtype.

In certain embodiments of the second aspect, measuring gene expression levels of the at least ten genes comprises measurement by RNAseq, miRNAseq, RT-PCR, nanostring, or other hybridization detection methods, immunohistochemistry/western blot analyses, nuclease protection assays, or Northern blot analyses.

In certain embodiments of the second aspect, the LNM cells from the subject are obtained from a primary cancer selected from the group consisting of: human papillomavirus-negative head and neck cancer, head and neck squamous cell cancer, breast cancer, melanoma, acute lymphoblastic leukemia (ALL), acute myeloid leukemia (AML), adrenocortical carcinoma, kaposi sarcoma (soft tissue sarcoma), aids-related lymphoma, primary CNS lymphoma, anal cancer, appendix cancer, astrocytomas, brain cancer, atypical teratoid/rhabdoid tumor, basal cell carcinoma of the skin, bile duct cancer, bladder cancer, bone cancer (including Ewing sarcoma and osteosarcoma and malignant fibrous histiocytoma), brain tumors, bronchial tumors, Burkitt lymphoma, non-Hodgkin lymphoma, carcinoid tumor (gastrointestinal), cardiac (heart) tumors, cervical cancer, cholangiocarcinoma, chordoma, chronic lymphocytic leukemia (CLL), chronic myelogenous leukemia (CML), colorectal cancer, craniopharyngioma, cutaneous T-cell lymphoma, ductal carcinoma in situ (DCIS), endometrial cancer (uterine cancer), esophageal cancer, esthesioneuroblastoma, extragonadal germ cell tumor, eye cancer, intraocular melanoma, retinoblastoma, fallopian tube cancer, gallbladder cancer, gastric (stomach) cancer, gastrointestinal carcinoid tumor, gastrointestinal stromal tumors (GIST), germ cell tumors, gestational trophoblastic disease, hairy cell leukemia, head and neck cancer, liver cancer, Hodgkin lymphoma, hypopharyngeal cancer, islet cell tumors, pancreatic neuroendocrine tumors, Langerhans cell histiocytosis, laryngeal cancer, leukemia, lip and oral cavity cancer, lung cancer, lymphoma, Merkel cell carcinoma, mesothelioma, plasmamesothelioma, plasma cell neoplasms, mycosis fungoides, myelodysplastic syndromes, myelodysplastic/myeloproliferative neoplasms, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paraganglioma, parathyroid cancer, penile cancer, multiple myeloma, prostate cancer, rectal cancer, renal cell (kidney) cancer, retinoblastoma, rhabdomyosarcoma, salivary gland cancer, sarcoma, uterine sarcoma, small cell lung cancer, small intestine cancer, soft tissue sarcoma, T-cell lymphoma, testicular cancer, throat cancer, thymoma, thyroid cancer, urethral cancer, uterine cancer, vaginal cancer, and Wilms tumor.

In certain embodiments of the second aspect, the primary cancer is a head and neck squamous cell cancer.

In certain embodiments of the second aspect, the primary cancer is breast cancer.

In certain embodiments of the second aspect, the primary cancer is melanoma.

In certain embodiments of the second aspect, the lymph node metastasis is taken from a formalin-fixed, paraffin-embedded sample. In some embodiments, samples may be isolated from fine needle aspirates, or fresh frozen tissues.

In certain embodiments of the second aspect, the subject is a human.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF DRAWINGS

FIGS. 1A-1H: Integrative analysis of lymph node metastasis from head and neck cancers identified 3 molecular subtypes having different rates of locoregional control and relapse-free survival. (1A) Schema of analysis. 34 patients with HNSCCs underwent surgery followed by adjuvant radiotherapy±cisplatin-based chemotherapy. 72 lymph node metastasis underwent RNAseq and miRNAseq followed by integrative clustering (iCluster) analysis to identify 3 groups: Group 1, Group 2 and Group 3. (1B-1D) Group 2 is associated with significantly worse disease control as assessed by (1B) locoregional control, (1C) local-only control, and (1D) relapse-free survival. (1E) Heat map of 2039 DEmRNAs between Group 1, Group 2 and Group 3 LNMs with FDR<0.05 and ≥1.5-fold expression. (1F-1H) Consensus clustering of DEmRNAs confirms 3 distinct groups as the optimal clustering. (1F) Consensus clustering, (1G) consensus CDF, (1H) Proportion of ambiguous clustering.

FIGS. 2A-2F: Group 2 LNM subtype was enriched in invasive gene signatures. (2A-2C) Heat map of normalized enrichment scores obtained using GSEA on the Hallmark pathway divided into pathways showing enrichment in Group 2 only (2A), Group 1 and 2 (2B) or Group 2 and 3 (2C) (for 2A-2C; *, **, and *** indicates FDR<0.25 for Group 1, Group 2, and Group 3 LNMs, respectively). (2D-2F) GESA enrichment plot for the Hallmark Epithelial Mesenchymal Transition Signature.

FIGS. 3A-3F: LNM subtypes were not present in the matched primary tumor. (3A) Integrative clustering of primary tumors identified 2 molecular subgroups that were not associated with differences in locoregional control (LRC). (3B) Heat map of Tumor Group 1 and Tumor Group 2 DEmiRNA. Consensus clustering 2039 DEG confirms 3 distinct groups. (3C) Consensus clustering confirms 2 molecular subgroups in primary HNSCC tumors. (3D) Consensus clustering of primary tumors using DEG identified in LNMs identifies 6 subgroups of primary tumors. (3E) Proportion of ambiguous clustering identifies k=6 as optimal clustering using DEGs from lymph node metastases. (3F) Clustering of primary tumors using lymph node metastases DEGs does not identify subgroups associated with improved survival.

FIGS. 4A-4D: The gene expression patterns of lymph node metastasis better predicted for patients at higher risk of locoregional failure. The prognostic performance of 4 HNSCC classifiers was tested on primary tumor (left panel) and LNMs (right panel). (4A) 172 gene signature significantly predicted LRC only in the lymph node metastases cohort. High risk and Low risk were determined as greater than or less than the average trait value, respectively. (4B) 30 gene Prognostic Index (PI) had a lower log-rank test p-value for LRC in the lymph node metastases cohort. PI-high and PI-low were determined as greater or less than the median 30 gene PI value, respectively. (4C) 10 gene radiosensitivity index (RSI) significantly predicted LRC only in the lymph node metastases cohort. Responder and Non-Responders were determined as the lesser than or greater than the 25^{th} RSI percentile, respectively. (4D) 13 gene oral cavity squamous cell carcinoma risk score significantly predicted LRC only in the lymph node metastases cohort. High risk and Low risk were determined as greater than or less than the median risk score, respectively.

FIGS. 5A-5C: A 73 gene LNM-specific classifier predicted outcomes only in LNMs across multiple cancer types. (5A) LRC of UIC HNSCC patients stratified by Group 2 or Group 1 & 3 subtypes in LNMs (left panel) and matched primary tumors (right panel). (5B) OS in a prospective breast cancer cohort stratified by Group 2 or Group 1 & 3 subtypes in LNMs (left panel) or other recurrent disease sites (right panel). (5C) Disease specific survival in a retrospective melanoma cohort stratified by Group 2 or Group 1&3 subtypes in LNMs (left panel) or other disease sites (right panel).

FIGS. 6A-6C: A 73 gene classifier identified similar Group 2-like subtype in breast cancers, melanoma, and HNSCCs. EGSEA comparing Group 2 and Group 1 & 3 subtypes from breast cancer, melanoma, and two HNSCC datasets (MDA HNSCC and UIC HNSCC) using Hallmark and KEGG. The median rank was used for all pathways. (6A) Heat map of EGSEA for Hallmark pathways. (6B) Heat map of EGSEA of KEGG supergroup pathways grouped by supergroup. (6C) Heat map of EGSEA of individual KEGG pathways. Common Hallmark and KEGG pathways were highly ranked across all Group 2-like subtypes.

FIG. 7: shows a schematic of process for patient selection.

FIGS. 8A-8F: Outcomes in HNSCC cohort. Kaplan-Meier analysis of: (8A) Locoregional control. (8B) Local-only control. (8C) Relapse-free survival. (8D) Regional control. (8E) Distant control. (8F) Overall survival.

FIGS. 9A-9C: Outcomes by molecular subtypes of LNMs. Kaplan-Meier analysis of: (9A) Regional control. (9B) Distant control. (9C) Overall survival.

FIGS. 10A-10G: DEmiRNAs in LNMs subtypes. (10A) Heat map of 24 DEmiRNAs. (10B) Proportion of ambiguous clustering plot using 24 DEmiRNAs to determine optimal clustering of lymph node metastasis. Lowest PAC value infers the optimal k (k=2). (10C-10G) Upper panels, Heat maps of the consensus matrix for the predefined cluster (10C) k=2, (10D) k=3, (10E) k=4, (10F) k=5 and (10G) k=6. Lower panels, Kaplan-Meier plots for locoregional control of the patients stratified by their consensus cluster membership. P-value determined using log-rank test.

FIGS. 11A-11H: Consensus clustering of pairwise DEGs for other predefined cluster numbers does not identify other LNMs subtypes that predict for disease recurrence. (11A, 11C, 11E & 11G) Heat map of the consensus matrix for the predefined cluster number (11A) k=2, (11C) k=4, (11E) k=5 and (11G) k=6. (11B, 11D, 11F & 11H) Kaplan-Meier plot for locoregional control of the patients stratified by the consensus cluster membership in (11B) k=2, (11D) k=4, (11F) k=5 and (11H) k=6. P value determined by the log-rank test.

FIGS. 12A-12B: Enrichment of KEGG pathways in LNMs subtypes. (12A) Heat map for the average enrichment scores of KEGG super pathways demonstrating for Group 1, Group 2, Group 3. (12B) Heat map for individual KEGG pathways enriched in Group 1 (*), Group 2 (**) or Group 3 (***).

FIGS. 13A-13D: The transcriptome of lymph node metastasis better predicts for patients at higher risk of relapse or death. The prognostic performance of 4 HNSCC classifiers was tested on primary tumor (left panel) and lymph node metastasis (right panel). (13A) 172 gene signature significantly predicted RFS in both the primary tumor in the lymph node metastases cohort. High risk and Low risk were determined as greater than or less than the average trait value, respectively. (13B) 30 gene Prognostic Index (PI) had significantly predicted RFS only in the lymph node metastases cohort. PI-high and PI-low were determined as greater or less than the median 30 gene PI value, respectively. (13C) 10 gene radiosensitivity index (RSI) significantly predicted RFS only in the lymph node metastases cohort. Responder and Non-Responders were determined as the lesser than or greater than the 25^{th} RSI percentile, respectively. (13D) 13 gene oral cavity squamous cell carcinoma risk score significantly predicted RFS in both the primary tumor and in the lymph node metastases cohort. High risk and Low risk were determined as greater than or less than the median risk score, respectively.

FIG. 14: shows a Receiver-Operator Curve for a 73 gene LNM-classifier.

### DETAILED DESCRIPTION

All publications, patents, and patent applications cited herein are hereby expressly incorporated by reference in their entirety for all purposes.

Before describing the present invention in detail, a number of terms will be defined. As used herein, the singular forms “a,” “an,” and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” as used herein represents the inherent degree of uncertainty that can be attributed to any quantitative comparison, value, measurement, or other representation. The term “substantially” is also used herein to represent the degree by which a quantitative representation can vary from a stated reference without resulting in a change in the basic function of the subject matter at issue.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “about” refers to ±10% of a given value.

In a first aspect, the present disclosure provides a method for treating cancer in a subject, the method comprising:
(a) obtaining a sample comprising lymph node metastasis (LNM) cells from the subject;
(b) measuring gene expression levels in the sample of at least ten genes selected from the group consisting of: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3;
(c) determining a LNM-specific molecular subtype comprising the gene expression levels of the at least ten genes;
(d) providing an indication as to the outcome of locoregional control (LRC), relapse-free survival (RFS), and/or overall survival (OS) when the LNM-specific molecular subtype indicates that the expression levels of the at least ten genes are altered in a predictive manner; and
(e) administering an aggressive cancer treatment regimen to the subject when a determination is made that the subject has LNM cells with decreased LRC, decreased RFS, and/or decreased OS.

In certain embodiments of the first aspect, the aggressive cancer treatment regimen comprises surgical resection of the primary tumor and lymph node dissection followed by radiotherapy with or without chemotherapy.

As defined herein, the term “LNM-specific molecular subtype” refers to a subtype determined by any combination of genes, the measured messenger RNA transcript expression levels, cDNA levels, or direct DNA expression levels, or immunohistochemistry levels of which can be used to distinguish between two biologically different tissues and/or cells and/or cellular changes. In certain embodiments, a LNM-specific molecular subtype is comprised of the gene-expression levels of at least 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 genes or less. In an embodiment, the LNM-specific molecular subtype is determined by measuring the expression of 73 genes. In certain embodiments, the genes are selected from: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3 (also see Table 6).

In certain embodiments of the first aspect, measuring gene expression levels of the at least ten genes comprises measurement by RNAseq, miRNAseq, or RT-PCR of the at least ten genes. The phrase “measuring the gene-expression levels” or “determining the gene-expression levels” as used herein refers to determining or quantifying RNA or proteins expressed by the gene or genes. The term “RNA” includes mRNA transcripts, and/or specific spliced variants of mRNA. The term “RNA product of the gene” as used herein refers to RNA transcripts transcribed from the gene and/or specific spliced variants. In some embodiments, mRNA is converted to cDNA before the gene expression levels are measured. In the case of “protein”, it refers to proteins translated from the RNA transcripts transcribed from the gene. The term “protein product of the gene” refers to proteins translated from RNA products of the gene. A number of methods can be used to detect or quantify the level of RNA products of the gene or genes within a sample, including: RNA-sequencing, miRNA sequencing, microarrays, Real-Time PCR (RT-PCR; including quantitative RT-PCR), nanostring or other hybridization detection methods, immunohistochemistry/Western blot analyses, nuclease protection assays, and Northern blot analyses. In addition, a person skilled in the art will appreciate that a number of methods can be used to determine the amount of a protein product of a gene of the invention, including immunoassays such as western blots, ELISA, and immunoprecipitation followed by SDS-PAGE and immunocytochemistry. In certain embodiments, the expression level of each gene in the gene set is determined by reverse transcribing the isolated RNA, miRNA, or mRNA into cDNA and measuring a level of fluorescence for each gene in the gene set by a nucleic acid sequence detection system following Real-Time Polymerase Chain Reaction (RT-PCR).

A person skilled in the art will appreciate that a number of detection agents can be used to determine gene expression. For example, to detect RNA products of the biomarkers, probes, primers, complementary nucleotide sequences or nucleotide sequences that hybridize to the RNA products can be used. In another example, to detect cDNA products of the biomarkers, probes, primers, complementary nucleotide sequences or nucleotide sequences that hybridize to the cDNA products can be used. To detect protein products of the biomarkers, ligands or antibodies that specifically bind to the protein products can be used.

In certain embodiments of the first aspect, the LNM cells from the subject are obtained from a primary cancer selected from the group consisting of: human papillomavirus-negative head and neck cancer, head and neck squamous cell cancer, breast cancer, melanoma, acute lymphoblastic leukemia (ALL), acute myeloid leukemia (AML), adrenocortical carcinoma, kaposi sarcoma, aids-related lymphoma, primary CNS lymphoma, anal cancer, appendix cancer, astrocytomas, brain cancer, atypical teratoid/rhabdoid tumor, basal cell carcinoma of the skin, bile duct cancer, bladder cancer, bone cancer, bronchial tumors, Burkitt lymphoma, non-Hodgkin lymphoma, carcinoid tumor (gastrointestinal), cardiac tumors, cervical cancer, cholangiocarcinoma, chordoma, chronic lymphocytic leukemia (CLL), chronic myelogenous leukemia (CML), colorectal cancer, craniopharyngioma, cutaneous T-cell lymphoma, ductal carcinoma in situ (DCIS), endometrial cancer, esophageal cancer, esthesioneuroblastoma, extragonadal germ cell tumor, eye cancer, intraocular melanoma, retinoblastoma, fallopian tube cancer, gallbladder cancer, gastric cancer, gastrointestinal carcinoid tumor, gastrointestinal stromal tumors (GIST), germ cell tumors, gestational trophoblastic disease, hairy cell leukemia, head and neck cancer, liver cancer, Hodgkin lymphoma, hypopharyngeal cancer, islet cell tumors, pancreatic neuroendocrine tumors, Langerhans cell histiocytosis, laryngeal cancer, leukemia, lip and oral cavity cancer, lung cancer, lymphoma, Merkel cell carcinoma, mesothelioma, plasma cell neoplasms, mycosis fungoides, myelodysplastic syndromes, myelodysplastic/myeloproliferative neoplasms, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paraganglioma, parathyroid cancer, penile cancer, multiple myeloma, prostate cancer, renal cell cancer, retinoblastoma, rhabdomyosarcoma, salivary gland cancer, sarcoma, uterine sarcoma, small cell lung cancer, small intestine cancer, soft tissue sarcoma, T-cell lymphoma, testicular cancer, throat cancer, thymoma, thyroid cancer, urethral cancer, uterine cancer, vaginal cancer, and Wilms tumor.

In certain embodiments of the first aspect, the lymph node metastasis is taken from a formalin-fixed, paraffin-embedded sample. In some embodiments, samples may be isolated from fine needle aspirates or fresh and/or frozen tissues.

In certain embodiments of the first aspect, the subject is a human.

In a second aspect of the invention, the invention provides a method for predicting outcome of locoregional control (LRC), relapse-free survival (RFS), and/or overall survival (OS) of a lymph node metastasis (LNM) in a subject, the method comprising:
(a) obtaining a sample comprising LNM cells from the subject;
(b) measuring gene expression levels in the sample of at least ten genes selected from the group consisting of: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3;
(c) determining a LNM-specific molecular subtype comprising the gene expression levels of the at least ten genes; and
(d) providing an indication as to the outcome of LRC, RFS, and/or OS when the LNM-specific molecular subtype indicates that the expression levels of the at least ten genes are altered in a predictive manner.

In certain embodiments of the second aspect, the LNM-specific molecular subtype for the LNM is classified as Group 1, Group 2, or Group 3. In some methods, Group 1 indicates an immune subtype, Group 2 indicates a significantly worse LRC, RFS, and/or OS, and Group 3 indicates a metabolic/proliferative subtype.

As used herein, the term “local recurrence” or “LR” refers to recurrence of cancer cells at the primary tumor site and is calculated as the time to local or regional failure. In this context, local recurrence occurs when there is a failure in local control (LC) or local-only control, such that therapeutic intervention is not successful in the eradication of cancer at the primary tumor site. Local control or local-only control refers to the successful treatment of cancer at the primary tumor site.

As used herein, the term “locoregional recurrence” or “LRR” refers to recurrence of cancer cells in tissues immediately surrounding the primary tumor site and/or lymph nodes. Locoregional recurrence occurs when there is a failure of or decreased locoregional control (LRC), such that therapeutic intervention is not completely successful in the eradication of cancer in the tissues immediately surrounding the primary tumor site and/or lymph nodes. Locoregional control refers to the successful treatment of cancer in tissues immediately surrounding the primary tumor site and/or lymph nodes. Locoregional recurrences often suggest the development of refractory cancer that is resistant to chemotherapy and radiation therapy. In addition, locoregional recurrence can be difficult to control and/or treat if: (1) the primary tumor is located or involves a vital organ or structure that limits the potential for treatment; (2) recurrence after surgery or other therapy occurs, because while likely not a result from metastasis, high rates of recurrence indicate an advanced tumor; and (3) presence of lymph node metastases indicate advanced disease.

As used herein, the terms “relapse-free survival” (RFS) and “disease-free survival” (DFS) refer to the period after a successful cancer treatment during which the patient survives without any signs or symptoms of that cancer and can be calculated as the time to any failure or death from any cause. The relapse-free survival or disease-free survival rate is often stated as a five-year rate, which is the percentage of people in a study or treatment group who are relapse-free or disease-free five years after their diagnosis or start of treatment.

As used herein, “overall survival” (OS) refers to the percentage of people in a study or treatment group who are still alive for a certain period of time after they were diagnosed with or started treatment for a disease, such as cancer, and can be calculated as the time to death from any cause. The overall survival rate is often stated as a five-year survival rate, which is the percentage of people in a study or treatment group who are alive five years after their diagnosis or the start of treatment.

In certain embodiments of the second aspect, measuring gene expression levels of the at least ten genes comprises measurement by RNAseq, miRNAseq, RT-PCR, nanostring, or other hybridization detection methods, immunohistochemistry/Western blot analyses, nuclease protection assays, or Northern blot analyses.

In certain embodiments of the second aspect, the LNM cells from the subject are obtained from a primary cancer selected from the group consisting of: human papillomavirus-negative head and neck cancer, head and neck squamous cell cancer, breast cancer, melanoma, acute lymphoblastic leukemia (ALL), acute myeloid leukemia (AML), adrenocortical carcinoma, kaposi sarcoma, aids-related lymphoma, primary CNS lymphoma, anal cancer, appendix cancer, astrocytomas, brain cancer, atypical teratoid/rhabdoid tumor, basal cell carcinoma of the skin, bile duct cancer, bladder cancer, bone cancer, bronchial tumors, Burkitt lymphoma, non-Hodgkin lymphoma, carcinoid tumor (gastrointestinal), cardiac tumors, cervical cancer, cholangiocarcinoma, chordoma, chronic lymphocytic leukemia (CLL), chronic myelogenous leukemia (CML), colorectal cancer, craniopharyngioma, cutaneous T-cell lymphoma, ductal carcinoma in situ (DCIS), endometrial cancer, esophageal cancer, esthesioneuroblastoma, extragonadal germ cell tumor, eye cancer, intraocular melanoma, retinoblastoma, fallopian tube cancer, gallbladder cancer, gastric cancer, gastrointestinal carcinoid tumor, gastrointestinal stromal tumors (GIST), germ cell tumors, gestational trophoblastic disease, hairy cell leukemia, head and neck cancer, liver cancer, Hodgkin lymphoma, hypopharyngeal cancer, islet cell tumors, pancreatic neuroendocrine tumors, Langerhans cell histiocytosis, laryngeal cancer, leukemia, lip and oral cavity cancer, lung cancer, lymphoma, Merkel cell carcinoma, mesothelioma, plasma cell neoplasms, mycosis fungoides, myelodysplastic syndromes, myelodysplastic/myeloproliferative neoplasms, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paraganglioma, parathyroid cancer, penile cancer, multiple myeloma, prostate cancer, renal cell cancer, retinoblastoma, rhabdomyosarcoma, salivary gland cancer, sarcoma, uterine sarcoma, small cell lung cancer, small intestine cancer, soft tissue sarcoma, T-cell lymphoma, testicular cancer, throat cancer, thymoma, thyroid cancer, urethral cancer, uterine cancer, vaginal cancer, and Wilms tumor.

As used herein, the terms “primary tumor” or “primary cancer” or “primary cancer tumor” can be used interchangeably and refer to the original, or first, tumor or cancer in the body of the patient. A primary tumor is a tumor growing at the anatomical site where tumor progression began and proceeded to yield a cancerous mass. Cancer cells from a primary tumor may spread (i.e., metastasize) to other parts of the body and form new, or secondary, tumors. Secondary tumors are the same type of cancer as the primary tumor.

As used herein, the term “metastasis” refers to the spread of cancer cells from its original, primary site to another, secondary part of the body. Metastasis is a complex process and depends on detachment of malignant cells from a primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. In certain embodiments, the term “metastasis” can refer to “distant metastasis” which relates to a metastasis which is remote from the primary tumor and the regional lymph node system.

In certain embodiments of the first or second aspect, the lymph node metastasis is taken from a formalin-fixed, paraffin-embedded sample. In some embodiments, samples may be isolated from fine needle aspirates, or fresh frozen tissues.

In certain embodiments of the first or second aspect, the subject is a human.

The terms “increased expression” or “decreased expression” as used herein refer to an expression level of one or more genes, or prognostic RNA transcripts, or their corresponding cDNAs, or their expression product(s) that has been found differentially expressed in LNMs compared to a control (for example, control can refer to a low risk LNMs group). The higher the expression level of a gene which predominantly has increased expression in LNMs of patients who had recurrence, the higher is the likelihood that the patient suffering from this LNM is expected to have a poor clinical outcome (i.e., higher risk of recurrence, decreased overall survival, or both). In contrast, the lower the expression level of a gene which predominantly has increased expressed in LNMs of patients who have recurrent tumors, the higher is the likelihood that the patient suffering from this LNM is expected to have a promising clinical outcome (i.e., decreased risk of recurrence, increased overall survival, or both). The lower the expression level of a gene which predominantly has decreased expression in LNMs of patients who had recurrence, the higher is the likelihood that the patient suffering from this LNM is expected to have a poor clinical outcome (i.e., higher risk of recurrence, decreased overall survival, or both). In contrast, the higher the expression level of a gene which predominantly has decreased expressed in LNMs of patients who have recurrent tumors, the higher is the likelihood that the patient suffering from this LNM is expected to have a promising clinical outcome (i.e., decreased risk of recurrence, increased overall survival or both).

As used herein, the terms “control” or “normalized sample” can be based on one or more cancer samples that are not from the patient being tested. In certain embodiments, a cancer cell or tumor may be determined to have a relative increased or decreased level of expression by comparing the expression levels of genes in the sample from the patient to the median expression levels of these genes across all cancer patients.

As used herein, the terms “treatment,” “treat,” or “treating” generally refer to a method of reducing the effects of a disease or condition or symptom of the disease or condition. Thus, in the disclosed methods, treatment can refer to about a 5%, 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is about a 5% reduction in one or more symptoms of the disease in a subject as compared to a control. Thus, the reduction can be about a 5%, 10%, 20%, 30%, 40%, 50%, 60%, 70%, 80%, 90%, 100% or any percent reduction between 5 and 100% as compared to native or control levels. It is understood that treatment does not necessarily refer to a cure or complete ablation of the disease, condition, or symptoms of the disease or condition.

As defined herein, the term “cancer treatment regimen” is determined by a medical professional or team of medical professionals and can be specific to each patient and/or cancer type, as is known in the art. Whether a treatment is aggressive or not will generally depend on the cancer-type, the age of the patient, etc. For example, in breast cancer adjuvant chemotherapy is a common aggressive treatment given to complement the less aggressive standards of surgery and hormonal therapy. Those skilled in the art are familiar with various other aggressive and less aggressive cancer treatment regimens for each type of cancer. A cancer treatment regimen is defined by the National Comprehensive Cancer Network (NCCN), and has been defined in the NCCN Guidelines® as including one or more of: 1) imaging (CT scan, PET/CT, MRI, chest X-ray), 2) discussion and/or offering of tumor resection if the tumor(s) is determined to be resectable, 3) radiation therapy (RT), 4) chemoradiation, 5) chemotherapy, 6) regional limb therapy, 7) palliative surgery, 8) systemic therapy, 9) immunotherapy, and 10) inclusion in ongoing clinical trials. Guidelines for clinical practice are published in the National Comprehensive Cancer Network (NCCN Guidelines® & Clinical Resources, available on the World Wide Web at NCCN.org). NCCN guidelines for treatment of cancer are publically available for at least the following cancers: Acute Lymphoblastic Leukemia (both, adult and AYA), Acute Lymphoblastic Leukemia (pediatric), Acute Myeloid Leukemia, AIDS-Related Kaposi Sarcoma, Anal Carcinoma, Bladder Cancer, Bone Cancer, Breast Cancer, Central Nervous System Cancers, Cervical Cancer, Chronic Lymphocytic Leukemia/Small Lymphocytic Lymphoma, Chronic Myeloid Leukemia, Colon Cancer, Rectal Cancer, Esophageal and Esophagogastric Junction Cancers, Gastric Cancer, Gestational Trophoblastic Neoplasia, Hairy Cell Leukemia, Head and Neck Cancers, Hepatobiliary Cancers, Hodgkin Lymphoma, Kidney Cancer, Malignant Pleural Mesothelioma, Melanoma, Cutaneous Melanoma, Uveal Melanoma, Multiple Myeloma, Systemic Light Chain Amyloidosis, Waldenström's Macroglobulinemia/Lymphoplasmacytic Lymphoma, Myelodysplastic Syndromes, Myeloproliferative Neoplasms, Neuroendocrine and Adrenal Tumors, Non-Hodgkin's Lymphomas, B-Cell Lymphomas, Primary Cutaneous Lymphomas, T-Cell Lymphomas, Non-Melanoma Skin Cancers (Basal Cell Skin Cancer, Dermatofibrosarcoma Protuberans, Merkel Cell Carcinoma, Squamous Cell Skin Cancer), Non-Small Cell Lung Cancer, Occult Primary, Ovarian Cancer, Pancreatic Adenocarcinoma, Penile Cancer, Prostate Cancer, Small Cell Lung Cancer, Soft Tissue Sarcoma, Systemic Mastocytosis, Testicular Cancer, Thymomas and Thymic Carcinomas, Thyroid Carcinoma, Uterine Neoplasms, and Vulvar Cancer.

The LNM-specific classifiers disclosed herein provide a new tool for cancer treatment that provides clinically relevant information that can be used to tailor conventional therapeutic intervention (e.g., surgery, radiation, chemotherapy, immunotherapy, and/or hormone therapy, etc.) to an individual's specific needs. For example, individuals identified with Group 2 LNMs can receive an aggressive treatment regimen immediately to maximize potential treatment efficacy. Similarly, individuals with either Group 1 or Group 3 LNMs may be able to receive effective but less aggressive treatments, which can lead to a less disruptive treatment experience for such individuals, as well as their families and friends.

In certain embodiments, the disclosed methods can be used to identify and treat patients that will benefit from a more aggressive cancer treatment regimen or less aggressive cancer treatment regimen. For example, if a patient is determined to have an aggressive form (Group 2) of a head and neck squamous cell cancer, then the patient can be selected for a more aggressive cancer treatment regimen with more frequent monitoring. In contrast, if a patient is determined to have a less aggressive form (Group 1 or Group 3) of a head and neck squamous cell cancer, then the patient can be selected for a less aggressive cancer treatment regimen with less frequent monitoring. Thus, in some embodiments, the disclosed methods further include selecting and treating a patient for more or less aggressive cancer treatment regimens and/or monitoring, depending on the expression levels detected of the at least ten genes.

In certain embodiments, a less aggressive cancer treatment regimen can comprise selecting and administering a single therapy instead of a dual therapy. In certain embodiments, a less aggressive cancer treatment regimen can comprise selecting and administering a dual therapy instead of a triple therapy. In certain embodiments, a more aggressive cancer treatment regimen can comprise, for example, selecting and administering a triple therapy instead of a dual therapy. In certain embodiments, a more aggressive cancer treatment can comprise, for example, selecting and administering more potent drugs and/or treatments or drugs and/or treatments at higher doses. In certain embodiments, a less aggressive cancer treatment regimen can comprise, for example, selecting and administering a dual therapy instead of a triple therapy, and selecting and administering less potent drugs, a decreasing drug dosage, a decreasing dose schedule, and/or a shortening length of treatment.

In certain embodiments, the methods of treatment further comprise administering to the patient one or more of a chemotherapy, a radiation therapy, and/or an immunotherapy. In certain embodiments, the method further comprises a second surgery for additional removal of the primary cancer and/or a secondary tumor.

In certain embodiments, surgery can be used to treat the cancer. Surgery can include resection of all or part of cancerous tissue. Tumor resection refers to physical removal, excision, and/or destruction of at least part of a tumor. In addition to tumor resection, treatment by surgery includes laser surgery, cryosurgery, electrosurgery, and microscopically controlled surgery (e.g., Mohs' surgery). It is further contemplated that the treatment methods described herein may be used in conjunction with removal of superficial cancers, precancers, or incidental amounts of normal tissue.

Cancer treatment regimens envisioned herein include those known in the art. Therapeutic options may include, but are not limited to: 1) combination regimens such as: AD (doxorubicin, dacarbazine); AIM (doxorubicin, ifosfamide, mesna); MAID (mesna, doxorubicin, ifosfamide, dacarbazine); ifosfamide, epirubicin, mesna; gemcitabine and docetaxel; gemcitabine and vinorelbine; gemcitabine and dacarbazine; doxorubicin and olaratumab; methotrexate and vinblastine; tamoxifen and sulindac; vincristine, dactinomycin, cyclophosphamide; vincristine, doxorubicin, cyclophosphamide; vincristine, doxorubicin, cyclophosphamide with ifosfamide and etoposide; vincristine, doxorubicin, ifosfamide; cyclophosphamide topotecan; ifosfamide, doxorubicin; and/or 2) single agents, such as, cisplatin or other metallic compounds, 5-FU/capecitabine (Xeloda®), cetuximab (Erbitux®), pembrolizumab (MK-3475), panitumumab (Vectibix®), dacomitinib (PF-00299804), gefitinib (ZD1839, Iressa), doxorubicin, ifosfamide, epirubicin, gemcitabine, dacarbazine, temozolomide, vinorelbine, eribulin, trabectedin, pazopanib, imatinib, sunitinib, regorafenib, sorafenib, nilotinib, dasatinib, interferon, toremifene, methotrexate, irinotecan, topotecan, paclitaxel, nab-paclitaxel (abraxane), docetaxel, bevacizumab, temozolomide, sirolimus (Rapamune®), everolimus, temsirolimus, crizotinib, ceritinib, and palbociclib.

Additional contemplated cancer treatment regimens useful herein include those disclosed in U.S. Patent Application Publication No. US2016/0333355, which is incorporated by reference herein. For example, a treatment regimen may include administering an antineoplastic agent (e.g., chemotherapy) along with IR (or radiotherapy) to treat a resistant cancer cell. An illustrative antineoplastic agent or chemotherapeutic agent includes, for example, a standard taxane. Taxanes are produced by the plants of the genus Taxus and are classified as diterpenes and widely uses as chemotherapy agents including, for example, paclitaxel, (Taxol™, Bristol-Meyers Squibb, CAS 33069-62-4) and docetaxel (Taxotere™, Sanofi-Aventis, CAS 114977-28-5). Other chemotherapeutic agents include semi-synthetic derivatives of a natural taxoid such as cabazitaxel (Jevtana™, Sanofi-Aventis, CAS 183133-96-2). Other chemotherapeutic agents also include an androgen receptor inhibitor or mediator. Illustrative androgen receptor inhibitors include, a steroidal antiandrogen (for example, cyperterone, CAS 2098-66-0); a non-steroidal antiandrogen (for example, flutamide, Eulexin™, Schering-Plough, CAS 13311-84-7); nilutamide (Nilandron™, CAS 63612-50-0); enzalutamide (Xtandi™, Medivation™, CAS 915087-33-1); bicalutamide (Casodex, AstraZeneca, CAS 90357-06-5); a peptide antiandrogen; a small molecule antiandrogen (for example, RU58642 (Roussel-Uclaf S A, CAS 143782-63-2); LG120907 and LG105 (Ligand Pharmaceuticals); RD162 (Medivation™, CAS 915087-27-3); BMS-641988 (Bristol-Meyers Squibb, CAS 573738-99-5); and CH5137291 (Chugai Pharmaceutical Co. Ltd., CAS 104344603904)); a natural antiandrogen (for example, ataric acid (CAS 4707-47-5) and N-butylbensensulfonamide (CAS 3622-84-2); a selective androgen receptor modulator (for example, enobosarm (Ostarine™ Merck & Company, CAS 841205-47-8); BMS-564,929 (Bristol-Meyer Squibb, CAS 627530-84-1); LGD-4033 (CAS 115910-22-4); AC-262,356 (Acadia Pharmaceuticals); LGD-3303 (Ganolix Lifescience Co., Ltd., 9-chloro-2-ethyl-1-methyl-3-(2,2,2-trifluoroethyl)-3H-pyrrolo[3,2-f]quino-lin-7(6H)-one; S-40503, Kaken Pharmaceuticals, 2-[4-(dimethylamino)-6-nitro-1,2,3,4-tetrahydroquinolin-2-yl]-2-methylpro-pan-1-ol); andarine (GTx-007, S-4, GTX, Inc., CAS 401900-40-1); and S-23 (GTX, Inc., (25)-N-(4-cyano-3-trifluoromethylphenyl)-3-(3-fluoro-4-chlorophenoxy)-2-hydroxy-2-methyl-propanamide)); or those described in U.S. Patent Application No. 2009/0304663. Other neoplastic agents or chemotherapeutic agents that may be used include, for example: alkylating agents such as nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; biological response modifiers such as interferon alphenomes; other agents such as platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MTH); adrenocortical suppressant such as mitotane (o,p′-DDD) and aminoglutethimide; taxol analogues/derivatives; hormone agonists/antagonists such as flutamide and tamoxifen; and GnRH and analogues thereof. Examples of other chemotherapeutic can be found in Cancer Principles and Practice of Oncology by V. T. Devita and S. Hellman (editors), 6^{th} edition (Feb. 15, 2001), Lippincott Williams & Wilkins Publishers.

In certain embodiments, a radiation therapy can be used. Radiation therapy (also known as radiotherapy) uses high doses of radiation to kill cancer cells and shrink tumors. Dosage ranges for X-rays can range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. Different routes of administration of radiation therapy contemplated herein are familiar to the person of ordinary skill in the art.

Radiotherapy is based on ionizing radiation delivered to a target area that results in death of reproductive tumor cells. Some examples of radiotherapy include the radiation of cesium, palladium, iridium, iodine, or cobalt which is usually delivered as ionizing radiation delivered from a linear accelerator or an isotopic source such as a cobalt source. Also variations on linear accelerators are Cyberkine and Tomotherapy. Particle radiotherapy from cyclotrons such as Protons or Carbon nuclei may be employed. Also radioisotopes delivered systemically such as P32 or radium 223 may be used. The external radiotherapy may be systemic radiation in the form of stereotactic radiotherapy, total nodal radiotherapy, or whole body radiotherapy but is more likely focused to a particular site, such as the location of the tumor or the solid cancer tissues (for example, abdomen, lung, liver, lymph nodes, head, etc.). The radiation dosage regimen is generally defined in terms of Gray or Sieverts time and fractionation, and must be carefully defined by the radiation oncologist. The amount of radiation a subject receives will depend on various consideration but the two important considerations are the location of the tumor in relation to other critical structures or organs of the body, and the extent to which the tumor has spread. One illustrative course of treatment for a subject undergoing radiation therapy is a treatment schedule over a 5 to 8 week period, with a total dose of 50 to 80 Gray (Gy) administered to the subject in a single daily fraction of 1.8 to 2.0 Gy, 5 days a week. A Gy is an abbreviation for Gray and refers to 100 rad of dose.

Radiotherapy can also include implanting radioactive seeds inside or next to a site designated for radiotherapy and is termed brachytherapy (or internal radiotherapy, endocurietherapy or sealed source therapy). For prostate cancer, there are currently two types of brachytherapy: permanent and temporary. In permanent brachytherapy, radioactive (iodine-125 or palladium-103) seeds are implanted into the prostate gland using an ultrasound for guidance. Illustratively, about 40 to 100 seeds are implanted and the number and placement are generally determined by a computer-generated treatment plan known in the art specific for each subject. Temporary brachytherapy uses a hollow source placed into the prostate gland that is filled with radioactive material (iridium-192) for about 5 to about 15 minutes, for example. Following treatment, the needle and radioactive material are removed. This procedure is repeated two to three times over a course of several days.

Radiotherapy can also include radiation delivered by external beam radiation therapy (EBRT), including, for example, a linear accelerator (a type of high-powered X-ray machine that produces very powerful photons that penetrate deep into the body); proton beam therapy where photons are derived from a radioactive source such as iridium-192, caesium-137, radium-226 (no longer used clinically), or cobalt-60; Hadron therapy; multi-leaf collimator (MLC); and intensity modulated radiation therapy (IMRT). During this type of therapy, a brief exposure to the radiation is given for a duration of several minutes, and treatment is typically given once per day, 5 days per week, for about 5 to 8 weeks. No radiation remains in the subject after treatment. There are several ways to deliver EBRT, including, for example, three-dimensional conformal radiation therapy where the beam intensity of each beam is determined by the shape of the tumor. Illustrative dosages used for photon based radiation is measured in Gy, and in an otherwise healthy subject (that is, little or no other disease states present such as high blood pressure, infection, diabetes, etc.) for a solid epithelial tumor ranges from about 60 to about 80 Gy, and for a lymphoma ranges from about 20 to about 40 Gy. Illustrative preventative (adjuvant) doses are typically given at about 45 to about 60 Gy in about 1.8 to about 2 Gy fractions for breast, head, and neck cancers.

When radiation therapy is a local modality, radiation therapy as a single line of therapy is unlikely to provide a cure for those tumors that have metastasized distantly outside the zone of treatment. Thus, the use of radiation therapy with other modality regimens, including chemotherapy, has important beneficial effects for the treatment of metastasized cancers.

Radiation therapy has also been combined temporally with chemotherapy to improve the outcome of treatment. For example, radiation therapy and chemotherapy can be administered sequentially (e.g., administration of chemotherapy and radiation therapy separately in time or concomitantly (e.g., administration of chemotherapy and radiation therapy on the same day. Also, radiation therapy and chemotherapy can be administered alternately, (e.g., administration of radiation therapy on the days when chemotherapy is not administered). It should be noted that other therapeutically effective doses of radiotherapy can be determined by a radiation oncologist skilled in the art and can be based on, for example, whether the subject is receiving chemotherapy, if the radiation is given before or after surgery, the type and/or stage of cancer, the location of the tumor, and the age, weight and general health of the subject.

In certain embodiments, immunotherapies can also be used to treat the cancer. Immunotherapy typically refers to the use of immune effector cells and/or molecules to target and destroy cancer cells. An immune effector molecule may be, for example, an antibody specific for some marker on the surface of a tumor cell. The antibody alone may serve as an effector of therapy or it may recruit other cells (e.g., immune cells) to effect cancer cell killing. The antibody also may be conjugated to a drug and/or toxin (e.g., a chemotherapeutic, a radionuclide, a ricin A chain, a cholera toxin, a pertussis toxin, etc.) and serve merely as a targeting agent. Alternatively, the effector may be an immune effector cell, such as a lymphocyte carrying a surface molecule that interacts, either directly or indirectly, with a tumor cell target. Various effector cells include cytotoxic T cells and NK cells.

Examples of immunotherapies (that can be used alone or in combination with any one or more of tumor resection if a tumor is determined to be resectable, radiation therapy, chemoradiation, chemotherapy, systemic therapy, additional immunotherapeutic, or inclusion in ongoing clinical trials), can include, for example, pembrolizumab (Keytruda®) and nivolumab (Opdivo®), cemiplimab (REGN2810; a fully human monoclonal antibody to Programmed Death-1), or other PD-1 inhibitors. CTLA-4 inhibitors (for example, ipilimumab (Yervoy®)) are another class of drugs that can boost the immune response. In some instances, cytokine therapy (such as, interferon-alpha and/or interleukin-2) can be used to boost the immune system. Examples of interferon and interleukin-based treatments can include, but are not limited to, aldesleukin (Proleukin®), interferon alpha-2b (INTRON®), and pegylated interferon alpha-2b (Sylvatron®; PEG-INTRON®, PEGASYS). In another embodiment, oncolytic virus therapy can be used. Along with killing the cells directly, the oncolytic viruses can also alert the immune system to attack the cancer cells. For example, talimogene laherparepvec (Imlygic®), also known as T-VEC, is an oncolytic virus that can be used to treat melanomas. Additional immunotherapies may include CV8102.

### Examples

The Examples that follow are illustrative of specific embodiments of the invention, and various uses thereof. They are set forth for explanatory purposes only and are not taken as limiting the invention.

Purpose: In advanced stage head and neck squamous cell cancers (HNSCCs), approximately half of the patients with lymph node metastases (LNMs) are not cured. Given the heterogeneous outcomes in these patients, the expression patterns of LNMs were profiled to identify the biological factors associated with patient outcomes.

Experimental design: mRNAseq and miRNAseq was performed on 72 LNMs and 29 matched primary tumors from 34 HNSCCs patients. Unsupervised clustering identified molecular subtypes in LNMs and in matched primary tumors. Prediction Analysis of Microarrays identified a 73 gene classifier that distinguished LNM-subtypes. Gene Set Enrichment Analysis identified pathways upregulated in distinct LNMs subtypes.

Results: Integrative clustering identified 3 distinct LNMs subtypes: (i) an immune subtype (Group 1), (ii) an invasive subtype (Group 2) and (iii) a metabolic/proliferative subtype (Group 3). Group 2 subtype was associated with significantly worse locoregional control and survival. LNM-specific subtypes were not observed in matched primary tumor specimens. In HNSCCs, breast cancers and melanomas, a 73-gene classifier identified similar Group 2 LNMs subtypes that were associated with worse disease control and survival only when applied to lymph node sites, but not when applied to other primary tumors or metastatic sites. Similarly, previously proposed prognostic classifiers better distinguished patients with worse outcomes when applied to the transcriptional profiles of LNMs, but not the profiles of primary tumors.

Data Interpretation: The transcriptional profiles of LNMs better predict outcomes than transcriptional profiles of primary tumors. The LNMs display site-specific subtypes associated with worse disease control and survival across multiple cancer types

### Methods

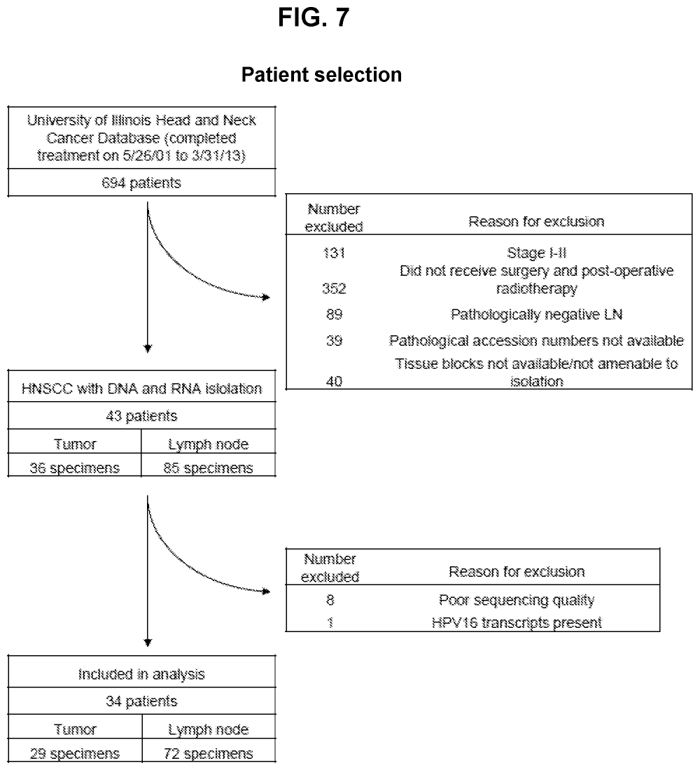

Patient population: From a database of non-metastatic HNSCC (6), 34 patients were identified with pathologic lymph node positive HNSCC treated with post-operative radiotherapy at University of Illinois between Mar. 26, 2001 and Mar. 31, 2013 (UIC HNSCC dataset; FIG. 7). The IRB waived informed consent due to the difficulty in obtaining consent of patients with archived pathological specimens. Patients were excluded due to Stage I-II disease (n=131), treatment with definitive radiation and/or did not have a therapeutic lymph node dissection prior to radiotherapy (n=352), no pathologically involved lymph nodes on dissection (n=89), no pathological accession numbers and/or tissue blocks were not available (n=79). The remaining 9 patients were excluded due to poor sequencing quality (n=8) or the presence of HPV16 transcripts (n=1). The resulting dataset is referred to as UIC HNSCC dataset. Biospecimens and de-identified linked patient data were collected in accordance with University of Illinois at Chicago Institutional Review Board guidelines (IRB 2011-1075). Data analysis was performed in accordance with University of Illinois at Chicago and University of Chicago (IRB 2015-1008) Institutional Review Board guidelines.

Clinical variables and analyses: Follow-up was calculated from the completion of radiotherapy to the date of first failure or last follow-up. Margin status, perineural invasion, lymphovascular space invasion, extracapsular extension, number of positive lymph nodes and number of lymph nodes dissected were based on the final pathology report. Treatment package time was calculated as the time from ablative surgery to the last day of radiotherapy. Events were determined from the last day of radiotherapy. Patterns of local (LF), regional (RF) or distant failure (DF) were documented as sites of first failure. Locoregional control (LRC) was calculated as the time to local or regional failure. Relapse-free survival (RFS) was calculated as the time to any failure or death from any cause. Overall survival (OS) was calculated as the time to death from any cause.

Statistical analysis was performed using JMP version 9 (SAS Institute). All tests of statistical significance were two-sided, and significance was defined as P<0.05. The chi-square test was used to compare between discrete variables and t-test between continuous variables. Differences between medians were assessed using the Wilcoxon test. Survival analysis was performed for all patients. Censoring was non-informative. All events were calculated using Kaplan Meier models with log-rank test, and the differences were compared using Cox regression models. For univariate analysis (UVA), factors known to impact oncologic outcomes were selected as well as patient and treatment characteristics. Cox proportional hazards model were used to examine the effects of these different risk factors on event outcomes for LRC. Cox multivariable analysis (MVA) was performed to adjust for explanatory confounding variables prognostic on UVA. Patient characteristics that were not recorded were not included during statistical analysis. Detailed description of patient and clinical variables are contained in Supplementary Information.

Sample isolation, Sequencing, and Analysis: Archived formalin fixed paraffin embedded (FFPE) tissue blocks of LNMs and matched primary tumors with the corresponding H&E slide were analyzed by expert head and neck pathologists (O.D. & R.J.C.). The area of greatest density of tumor cells with at least 70% cancer cells were identified on the H&E slide and mapped to the corresponding FFPE block. 2 mm punch biopsies were obtained for the indicated region. RNA was extracted from isolated specimens using the RecoverAll Total Nucleic Acid Isolation Kit (Ambion, TX) and stored at −80° C. until further analysis. mRNA and miRNA libraries were sequenced on a HiSEQ2500 machine using standard reagents and protocols provided by Illumina. Read alignment, quantification, and normalization are described in the Supplementary Information.

Detection of Differentially Expressed mRNAs or miRNAs: To identify differentially expressed mRNAs among samples grouped by iCluster method, heteroscedascity was removed from the normalized CPM data using the voomWithQualityWeights function from the limma package. A linear model for each gene was fit using the limma algorithm, adjusted for patient covariate, and ranked the genes for differential expression using the empirical Bayes method with robust option enabled. For miRNAs, the limma method was applied to identify differentially expressed miRNAs among the samples grouped by iCluster method. First the relative quality weights were estimated for each array using the arrayWeightsSimple function, and then a linear model was fit for each probeset adjusted for batch effect, followed by ranking probesets for differential expression using empirical Bayes method. The differentially expressed genes were identified with the Benjamini-Hochberg procedure for multiple testing adjustment and fold-change. The false discovery rate (FDR) controlling adjusted P-value and fold-change threshold were set at 0.05 and 1.5 for mRNAs and 0.1 and 1.5 for miRNAs, respectively.

Clustering analysis with iCluster+: An integrative clustering method named iCluster+ (19) was applied to the matched mRNA and miRNA expression data of the lymph node samples alone and primary tumor and lymph node samples combined. The algorithm was run for N=2 to 6 with the number of lambda as 144. For each N, a model that contains a deviance ratio metric was obtained. The metric was selected based on minimum Bayesian information criterion (BIC) for each lambda value, and can be interpreted as the percentage of total variation explained by the current model. The optimal number of clusters N was determined at the point when increasing N, the percent explained variation no longer significantly increases. Consensus clustering is described in the Supplementary Information.

Functional enrichment analysis: Raw gene feature counts were mapped to Entrez ID using the R/Bioconductor package org.Hs.eg.db v3.5.0. Low/non-expressed genes with less than 1 CPM across the minimum number of samples in any icluster group were excluded from subsequent analysis using edgeR. Quality weighted, TMM normalized, and log 2-transformed CPM were calculated using limma-voom (version 3.34.5). Gene set enrichment was performed with planned contrasts of one iCluster group against the remaining groups using GSEA (20) for comparison within the UIC HNSCC dataset or the R/Bioconductor package EGSEA (version 1.6.1) (21) for comparison across other publically available including HNSCC (16) (Array Express E-MEXP-44), breast cancer (22) (GSE56493) and melanoma (23) (Array Express E-GEOD-65904) databases. Independent GSEA/EGSEA analyses were performed for gene lists provided by MSigDB v5.2 containing hallmark gene set and KEGG pathway gene set. Enriched pathways were selected according to the median rank across 12 enrichment methods implemented in the package.

Classification—Data Preprocessing, Training and Testing: To build a classifier to distinguish samples between cluster 2 (C2) and cluster 1 and 3 combined (C13) identified by iCluster method, the normalized mRNA expression data of 33 patients was split into a training set, consisting of 22 cluster 2 samples and 33 cluster 1 and 3 samples, and a test set, consisting of 7 cluster 2 samples and 11 cluster 1 and 3 samples. The class ratio remained unchanged during the partition. For the training set, first genes were filtered with near zero-variance. Then highly correlated genes were identified with a pair-wise absolute correlation coefficient greater than 0.75, and those with the largest mean absolute correlation were removed. Potential linear dependencies of the data were also removed using the findLinearCombos function from the R package Caret (version 6.0). The preProcess function was applied to center and scale the training and test data by mean and standard deviation, followed by rescaling data to −1 and 1. Prediction Analysis of Microarrays (PAMR, version 1.55) was also applied—a nearest shrunken centroid classification algorithm—on the training set (24). A 10-fold cross-validation was performed to obtain the optimal threshold of 2.536 for the prediction, where the overall error rate was 0.091. The final classification model contains 73 genes and was evaluated using the held-out test data of 7 group two samples and 11 group one and three samples. Performance metrics such as accuracy, balanced accuracy, sensitivity, specificity, positive prediction value (PPV), negative prediction value (NPV), Cohen's Kappa, Matthew's correlation coefficient (MCC), and area under the curve (AUC) were calculated using the confusionMatrix function from the Caret package and an in-house script.

Independent Validation of Classifier: To evaluate the LNM classifier performance in other tumor types, lymph node metastatic tumor expression data were collected from three published studies. The studies contain microarray expression data from breast cancer (22) (GSE56493), melanoma (23) (GSE65904, 10000 most variable genes), and HNSCCs (16) (MDACC HNSCC dataset; E-TABM-114) from Gene Expression Omnibus (GEO) and ArrayExpress database. For each dataset, the normalized gene expression data was downloaded from the original studies. The probesets (Affymetrix) or probes (Illumina) were converted to Ensembl gene identifiers. The expression data of the 73 genes were scaled to −1 and 1. Values for the missing genes were set to −1. The LNM classifier was then applied to the data to predict the test samples as group two or group one and three combined.

Comparison of the LNM classifier and other published signatures: The LNM classifier's ability of distinguishing different survival group in HNSCC patients was evaluated with the comparison of four published gene signatures (25-28). For each signature, the gene signatures was collected from the original publication and converted the gene symbol or alias to Ensembl gene identifier, then computed the score based the gene expression in our data set. The following criteria was used to group the patients into two risk groups and performed the Kaplan-Meier survival estimates for the risk groups.

(1) Enokida classifier: PI>75 quartile—PI high, otherwise—PI low

(2) RSI classifier: RSI<median—Responder, otherwise—Non-responder

(3) 13 OSCC classifier: RS>median—High risk, otherwise—Low risk

(4) 172-gene classifier: RS>mean—High risk, otherwise—Low risk

### Supplementary Information

Patient and clinical variables: Patient comorbidity burden was approximated using the Charlson Comorbidity Index.¹ Performance status was assessed using the Karnofsky Performance Status (KPS).² Patients were staged according to the American Joint Committee on Cancer staging system at the time of diagnosis. Documentation of race was based on patients self-reporting on clinic or hospital intake sheets. Alcohol history was defined as ≥ or <2 alcoholic drinks per day. Smoking was defined as ≥ or <10 pack-years.

Library Construction: RNA integrity and quantity were evaluated using an Agilent 2100 Bioanalyzer (Agilent Technologies, CA). Reverse-stranded single-end 50 base-pair sequencing libraries were constructed using Illumina Total RNA Stranded Kits. Ribosomal RNAs (rRNAs) were depleted by using the Ribo-Zero rRNA Removal Kit (Illumina). Libraries were sequenced on a HiSEQ2500 machine using standard reagents and protocols provided by Illumina.

Read Alignment and Quantification: Unless otherwise specified, all data analyses were performed under the R programming and software environment for statistical computing and graphics version 3.4 (R Core Team, 2016). FastQ file for each sample was assessed for quality using the FastQC tool (version 0.11.2). For mRNAseq, raw reads were aligned to the GRCh38 primary genome assembly using Spliced Transcripts Alignment to a Reference (STAR) aligner (version 2.4.2a) 1-pass algorithm. After sorting the bam files in lexicographical order with the sambamba program3, the reads were assigned to exon features annotated in GENCODE (release 25) using the FeatureCounts tool from the subread package (version 1.4.6) and summarized the read counts by genes. The post-alignment quality control was carried out with Picard tools (version 1.117) and RSeQC package (version 2.3.1). Specifically, the QC data regarding the alignment summary, gene body coverage, read distribution, and ribosomal RNA depletion rate was examined. Alignment-free transcriptome quantification method Salmon (version 0.8.2)³ was used to estimate the transcript abundance of each sample. Then the transcript-level estimates for gene-level analysis was summarized using R/Bioconductor package tximport⁴ and GENCODE annotation (release 25). For miRNAseq, cutadapt (version 1.7) was used with the following parameter setting (-a TGGAATTCTCGG-O 3-m 17-q 20; (SEQ ID NO:88)) to remove adapter sequences and reads with low base calling quality score. Mapper module from miRDeep (version 2.0.0.8)⁵ was applied to convert trimmed reads to FASTA files and quantifier module to quantify the expression based on the predefined human miRNA precursors mature and hairpin miRNA sequences (miRBase 21).

Data Normalization: After removing the genes with zero read counts across all samples, the normalization factors were calculated to scale the raw library sizes using the calcNormFactors function in the edgeR package (v3.20.6) with trimmed mean of M-values (TMM) option enabled⁶. The normalized count per million (CPM) value was log 2-transformed for each gene. The log-CPM values were corrected for batch effect (sequencing lane effect) if needed, using removeBatchEffect function from the R/Bioconductor package limma (version 3.34.9).

Reference samples were developed to normalize samples when measuring the 73-gene signature. The list of genes to generate the reference sample is shown in Table 7. The reference oligomers (SEQ ID NOs:1-87) were synthesized at 4 nM, and resuspended at a stock concentration of 10 pmol/microliter. A batch reference sample was generated with all oligomers at a concentration of 100 fmol/microliter. The nanostring cassette detected oligomers at a concentration of 500 fmol to 8 fmol. Reference concentrations of 31.2 fmol to 125 fmol were chosen for the assay.

Consensus Clustering of Expression Data: To evaluate if a selected set of DEGs or DEmiRs between iCluster groups alone could identify patients with distinct survival outcomes, consensus clustering analysis was performed on mRNA and miRNA expression data sets using the R package ConsensusClusterPlus (version 1.38.0)⁸. The DEGs and the DEmiRs were selected across three iCluster groups, respectively. Normalized expression data from previous procedures were first standardized using the data normalization function in the R package clusterSim (version 0.45-1). To run ConsensusClusterPlus, the options were preset as a maximum evaluated cluster k=6, 80% samples per resampling, 1,000 resamplings, Euclidean distance, and k-means clustering algorithm. Complete and average linkage were chosen as the inner-linkage and final linkage, respectively. The optimal number of k clusters was inferred by inspecting the consensus cumulative distribution function (CDF) plot and the proportion of ambiguously clustered pairs (PAC)⁹ plot where the optimal k corresponds to the lowest PAC.

### Example No. 1. Integrative Analysis of LNMs Identified Three Molecular Subtypes Associated with Different Rates of HNSCC Recurrence

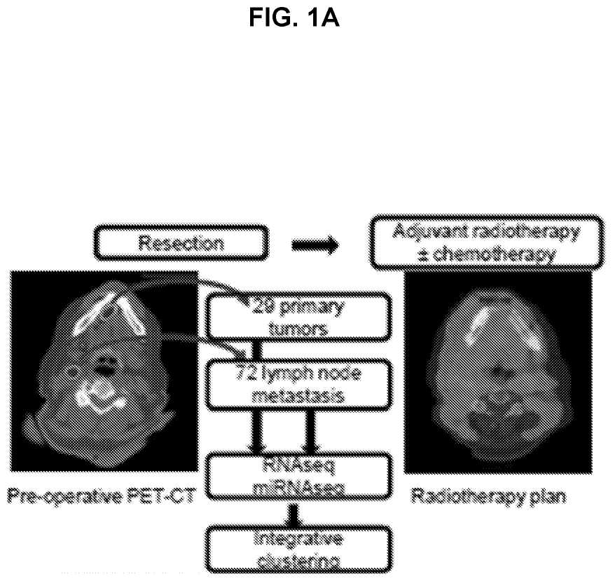

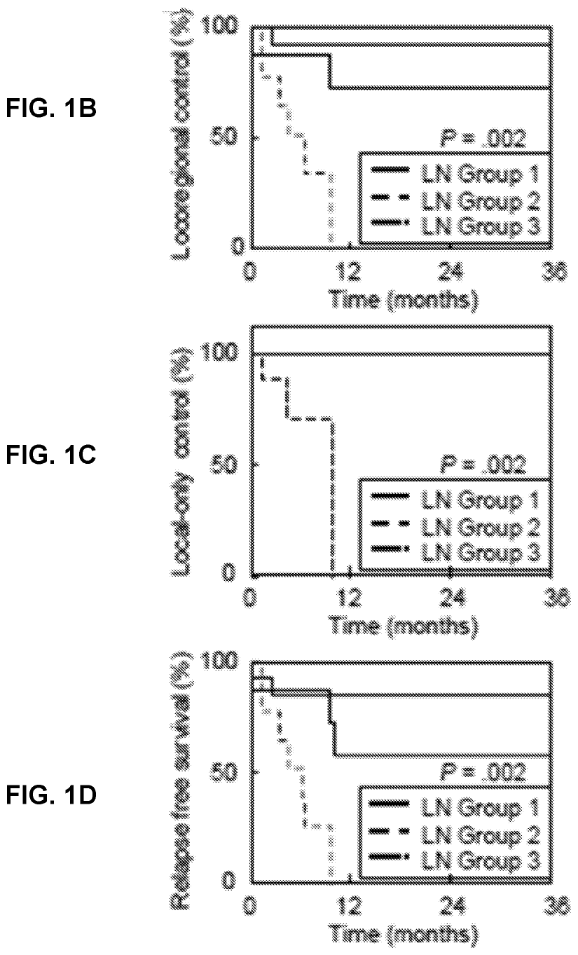

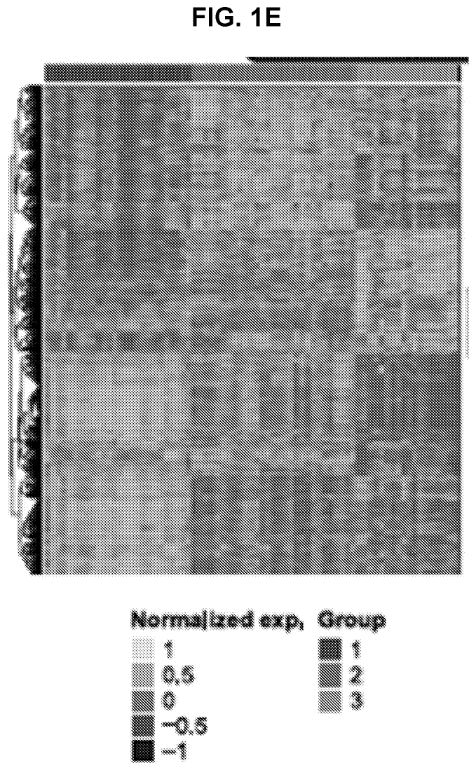

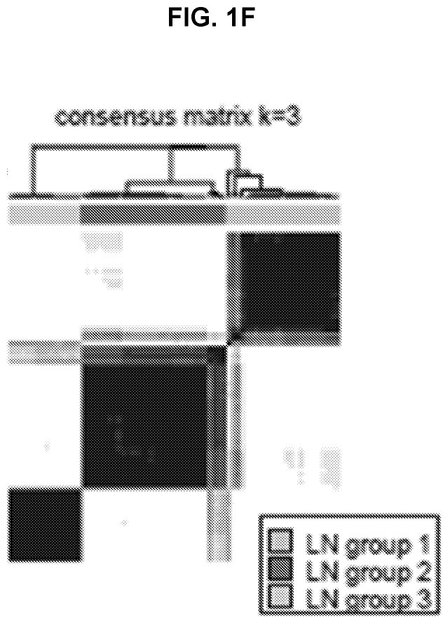

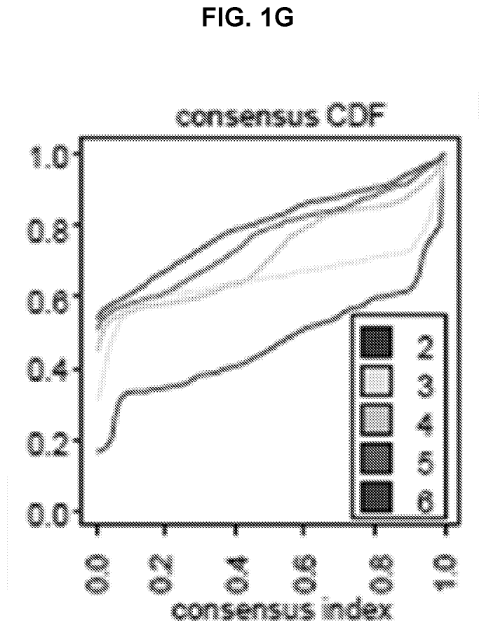

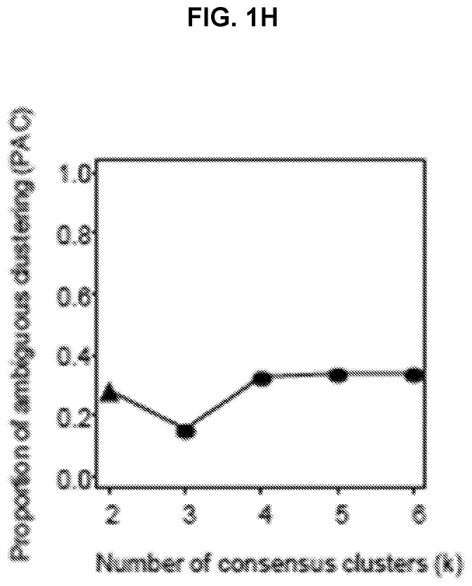

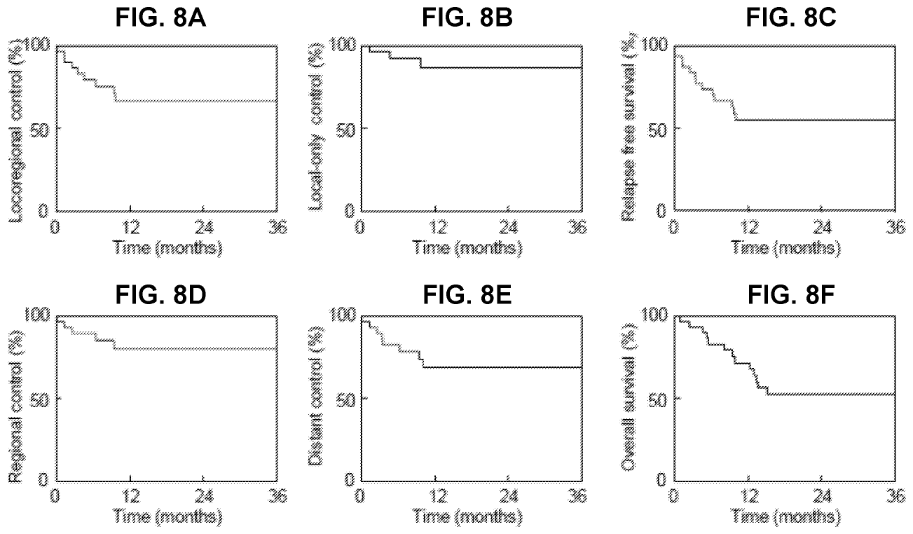

Thirty four patients were identified with human papillomavirus (HPV)-negative HNSCC who had accessible primary tumors and matched LNMs (6). These patients were treated with surgical resection of the primary tumor and lymph node dissection followed by radiotherapy with or without chemotherapy (FIG. 1A). Since differences in coding and non-coding gene expression may distinguish clinically relevant subtypes in LNMs, RNAseq and miRNAseq was performed on 29 tumors and 72 matched LNM (termed UIC HNSCC dataset). Table 1 lists the clinicopathological characteristics of this group where the majority of patients had N2-N3 disease (81.8%) and received post-operative chemoradiation (78.8%). The 2 year LRC, PFS, and OS in the entire cohort was 66.7%, 55.1%, and 52.7%, respectively, similar to previous reported outcomes in HPV-negative HNSCCs (11, 12) (FIG. 8).

To identify distinct metastatic subtypes, integrative clustering was performed on the 72 LNMs in order to identify potential lymph node-specific subtypes. Integrative clustering identified 3 distinct molecular subtypes: 27 LNMs were classified as Group 1, 29 LNMs were classified as Group 2 and 16 LNMs were classified as Group 3.

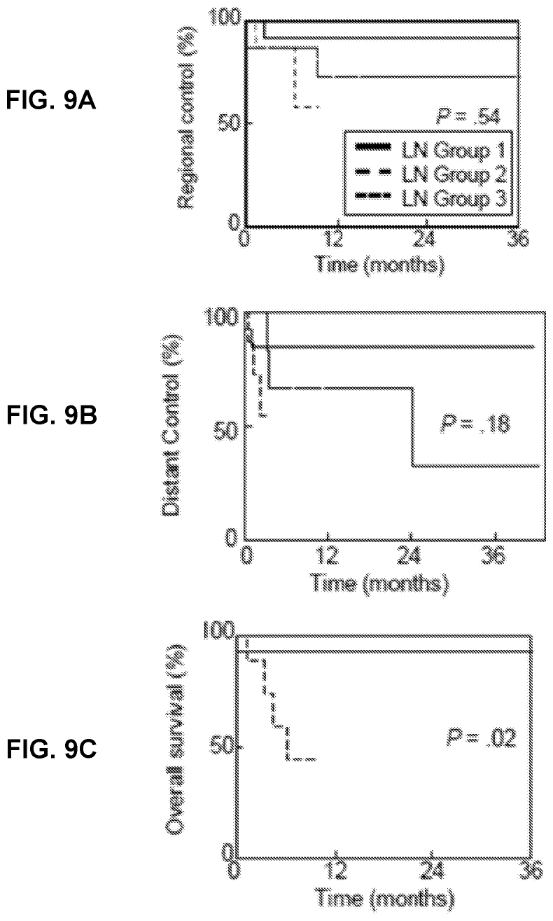

The clinical relevance of the Group 1, Group 2, and Group 3 LNM subtypes was assessed by comparing clinicopathological variables, disease recurrence, and survival. Since 11 of 17 patients with multiple LNMs displayed more than one LNM subtype, patients with multiple LNMs were classified as Group 2 if patients had ≥2 Group 2 LNMs. If patients with multiple LNMs had <2 Group 2 LNM, these patients were classified according the most prevalent LNM subtype. On univariate analysis, patients with Group 3 LNMs were associated with greater lymphovascular space invasion (P=0.02), and patients with Group 2 LNMs were associated with fewer numbers of LN removed at the time of surgery (P=0.03; Table 2). Patients with Group 2 LNMs displayed significantly worse LRC (ly LRC Group 1: 91.7% vs. Group 2: 0.0% vs. Group 3: 72.9%; P=0.002), RFS (P=0.002), and OS (ly OS Group 1: 92.9% vs. Group 2: 44.4% vs. Group 3: 100.0%; P=0.02; FIGS. 1B-1D and FIG. 9). On multivariate analyses, Group 2 subtype remained the only predictor for LRC compared to either the Group 1 subtype (HR: 0.1; 95% CI 0.01-0.71; P=0.02) or the Group 3 subtype (HR: 0.2; 95% CI 0.03-0.99; P=0.04; Table 3). There was no difference in LRC between Group 1 and Group 3 subtypes (HR: 2.04; 95% CI 0.27-17.82; P=0.48).

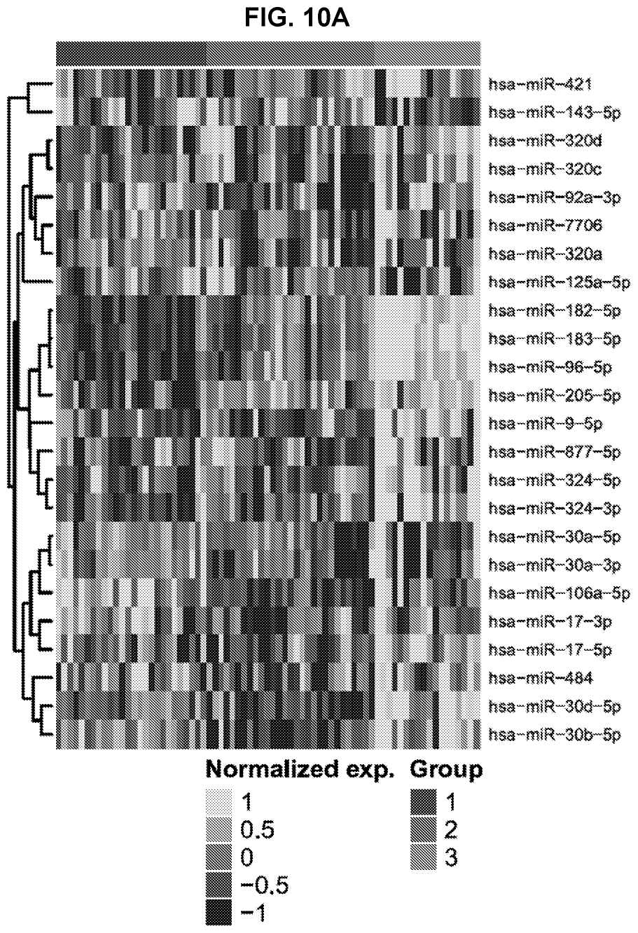

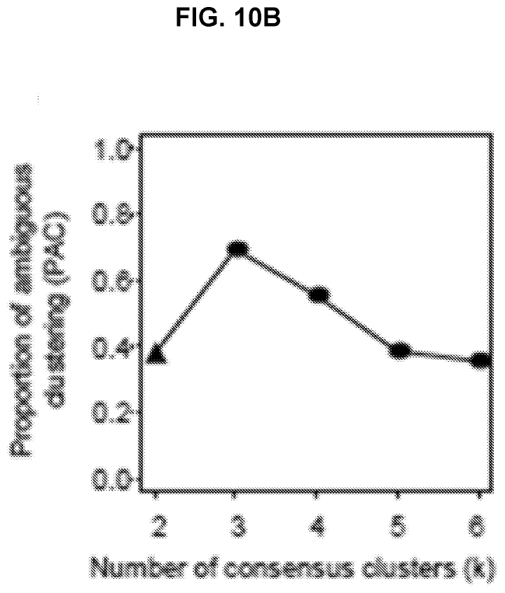

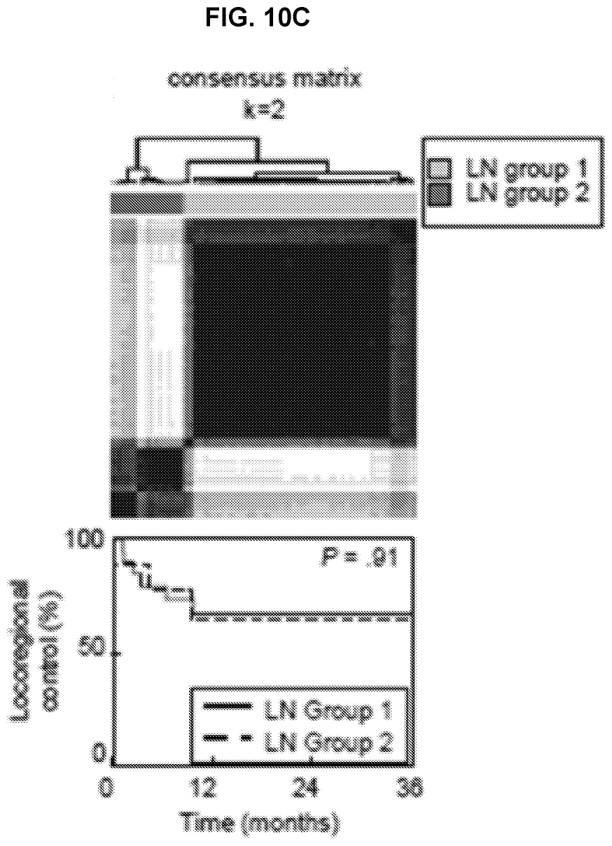

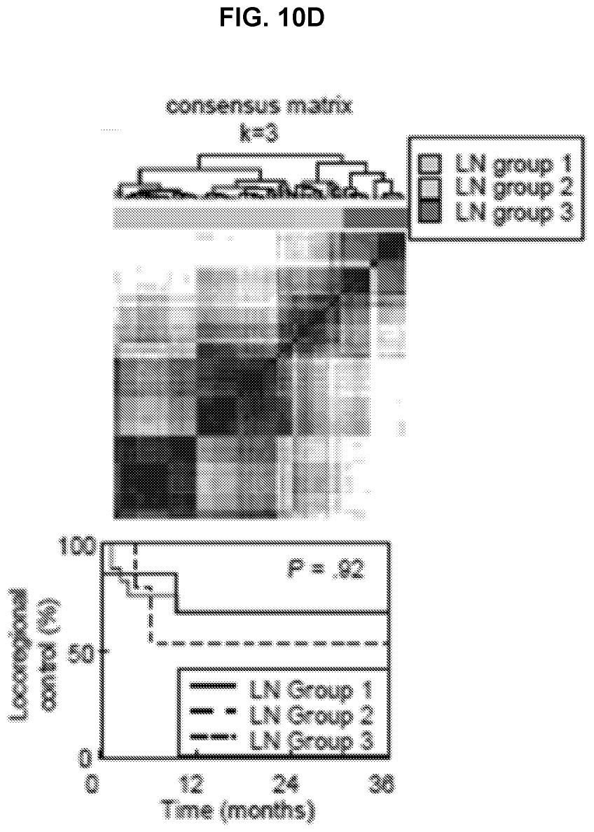

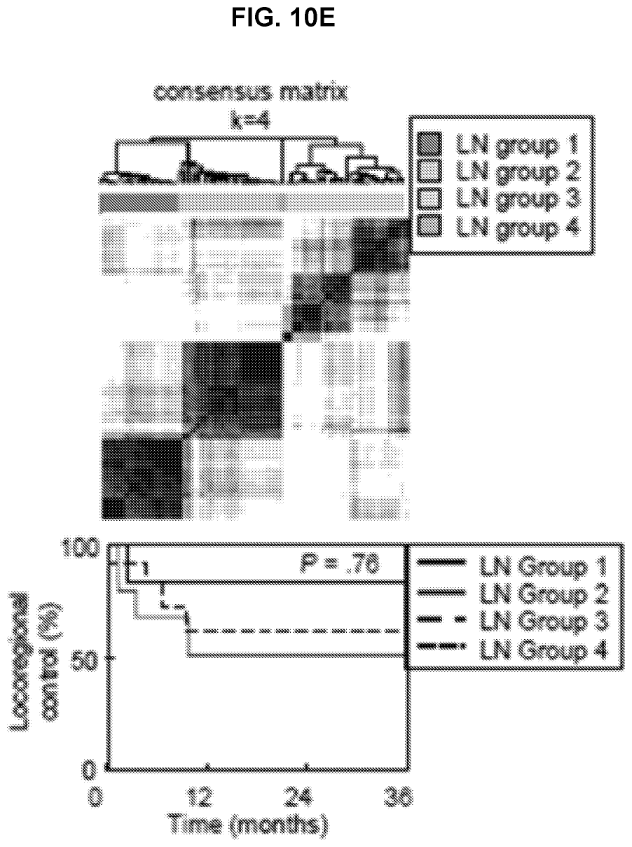

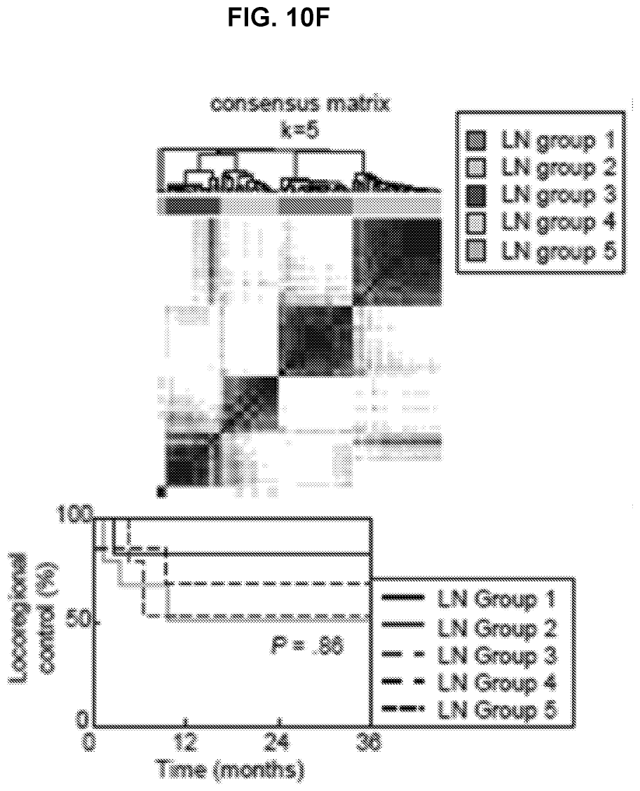

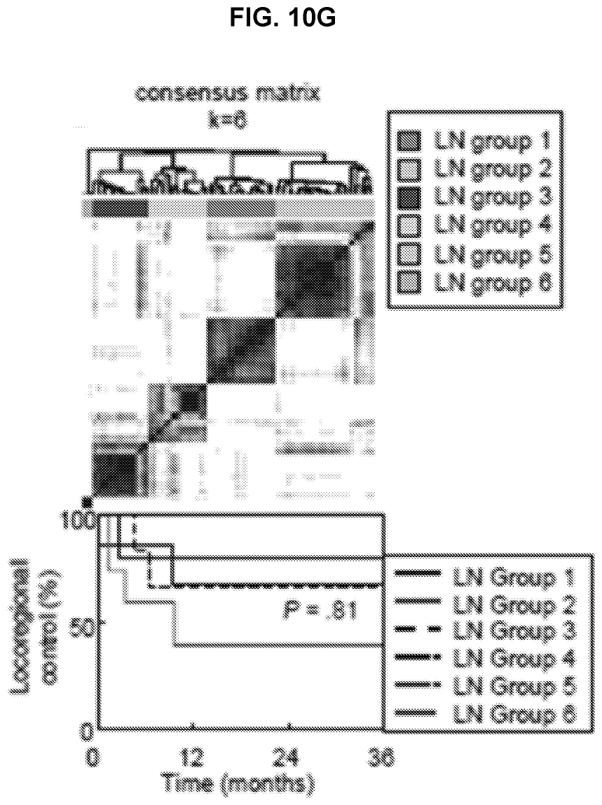

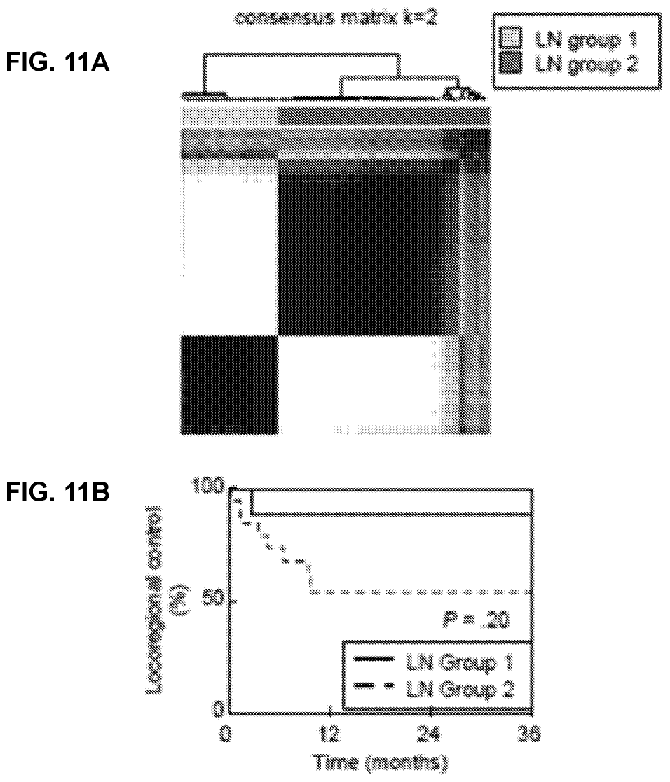

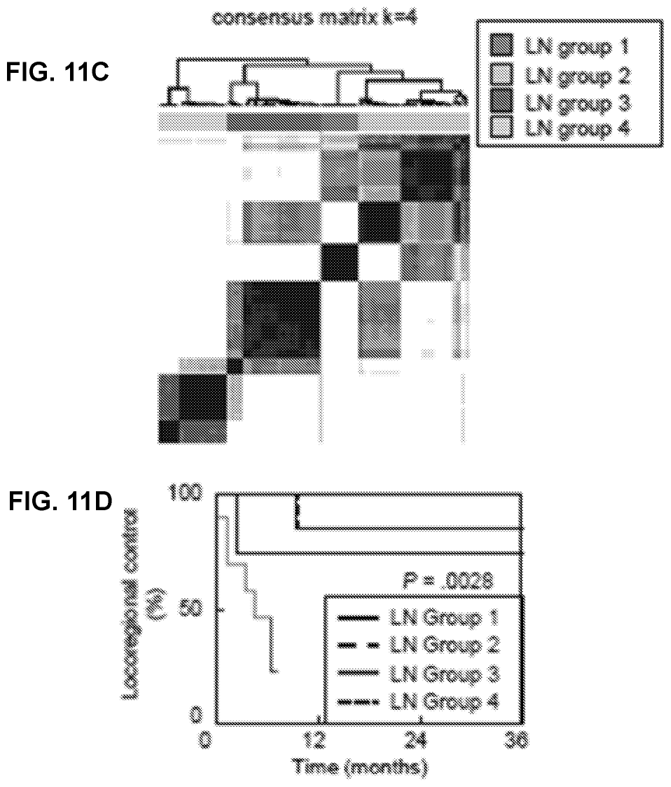

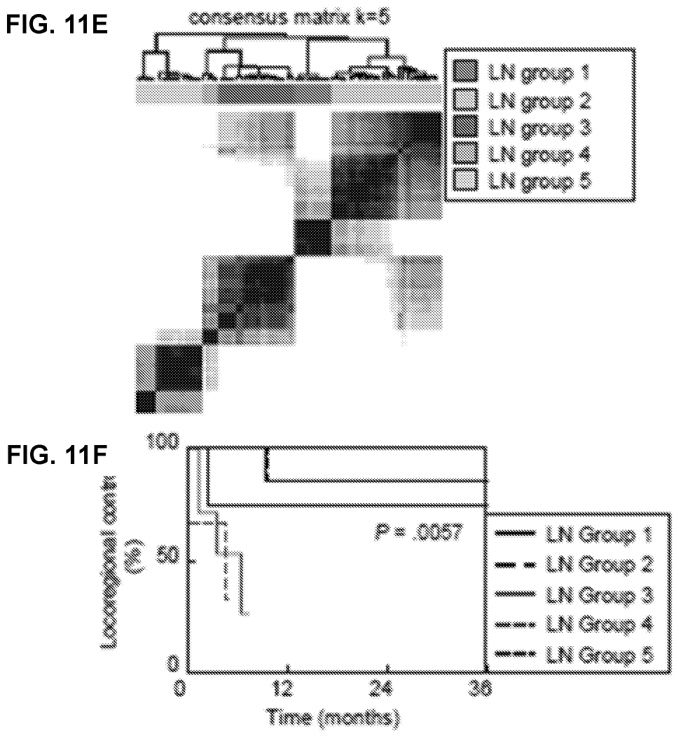

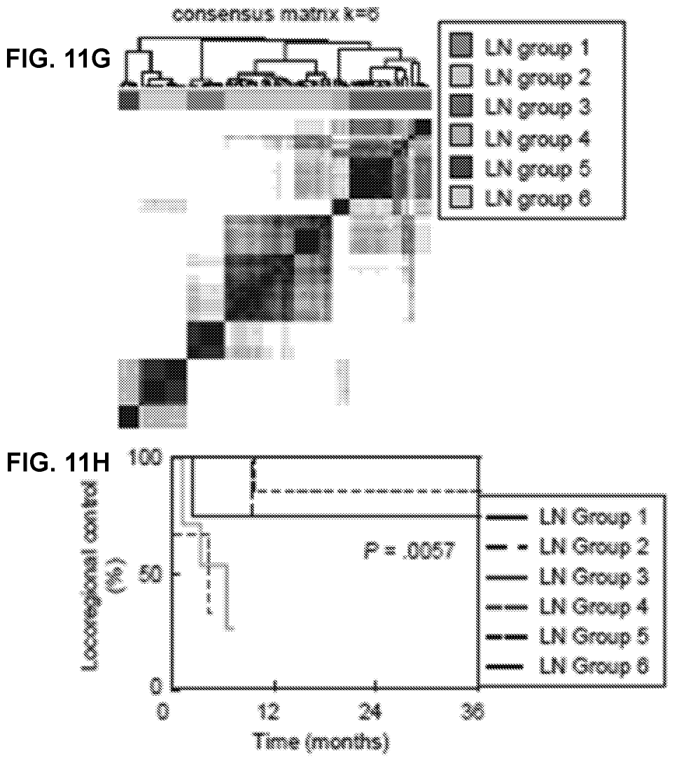

The classification of these 3 molecular subtypes was primarily due to differences in mRNA expression and not miRNA expression. Using pair-wise comparisons, 2,039 differentially expressed mRNAs (DEmRNAs) were detected and 24 differentially expressed miRNAs (DEmiRNAs; FIG. 1E; FIGS. 10A & 10B, Table 4). Consensus clustering, consensus cumulative distribution function, and proportion of ambiguous clustering of DEmRNAs confirmed that 3 molecular subtypes were the optimal classification of LMNs (FIGS. 1F-1H). By contrast, consensus clustering of DEmRNAs using other predefined cluster numbers of K=2, 4, 5 or 6 exhibited less optimal cluster groupings (FIG. 11). Furthermore, more than 3 pre-defined clusters failed to further stratify differences in LRC. Pre-defined clustering of DEmiRNAs did not identify any subtypes having differences in LRC (FIGS. 10B-10G). Therefore, molecular classification of LNMs in HPV-negative HNSCCs using DEmRNAs defined three distinct subtypes where one subtype, Group 2, had significantly worse LRC, RFS, and OS.

### Example No. 2. The Group 2 LNM Subtype was Associated with an Invasive Subtype

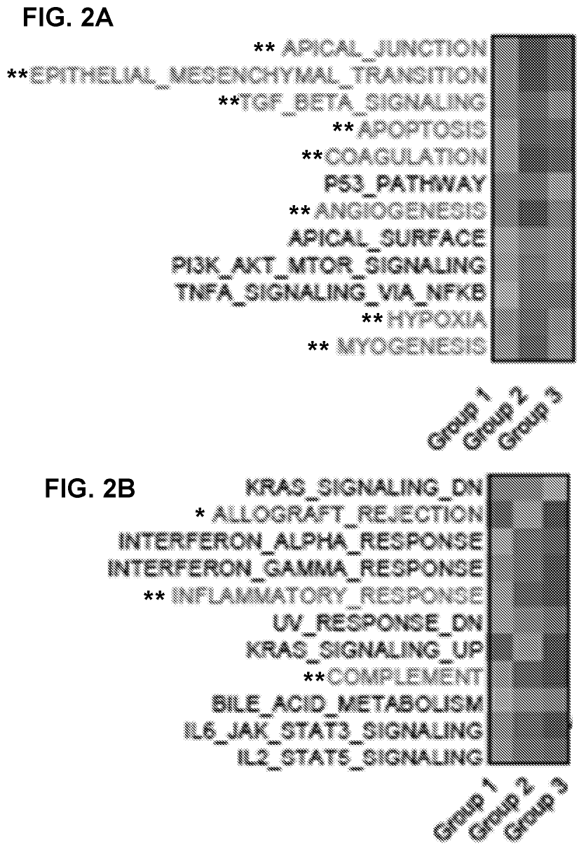

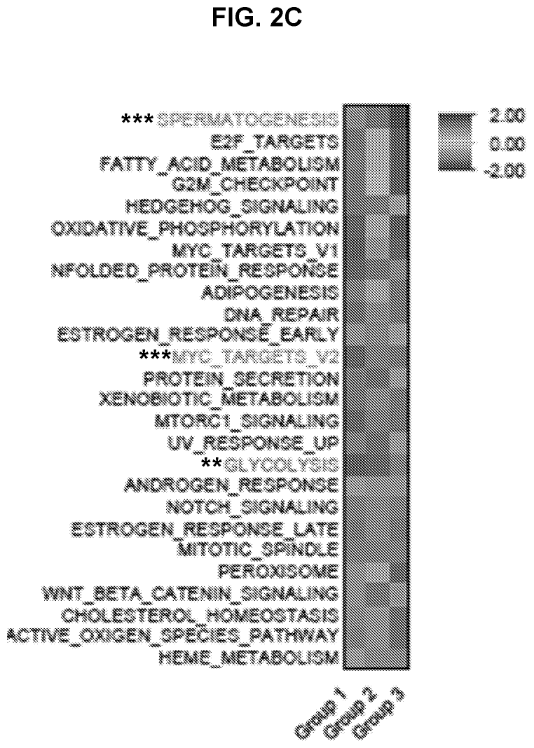

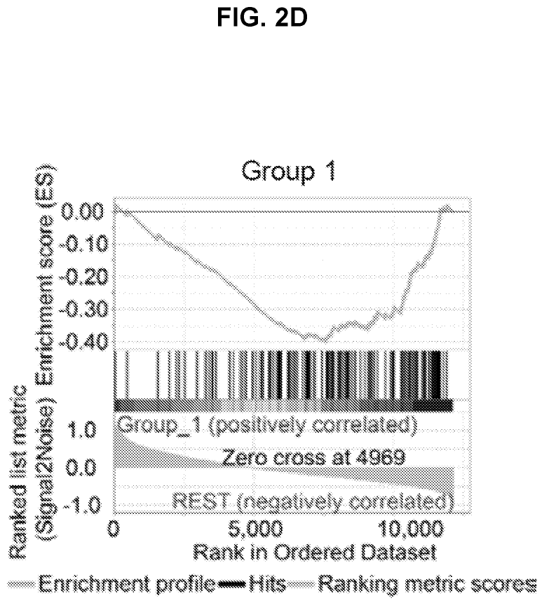

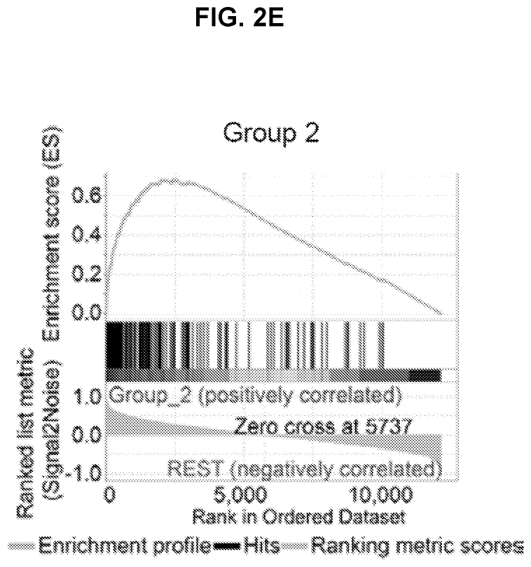

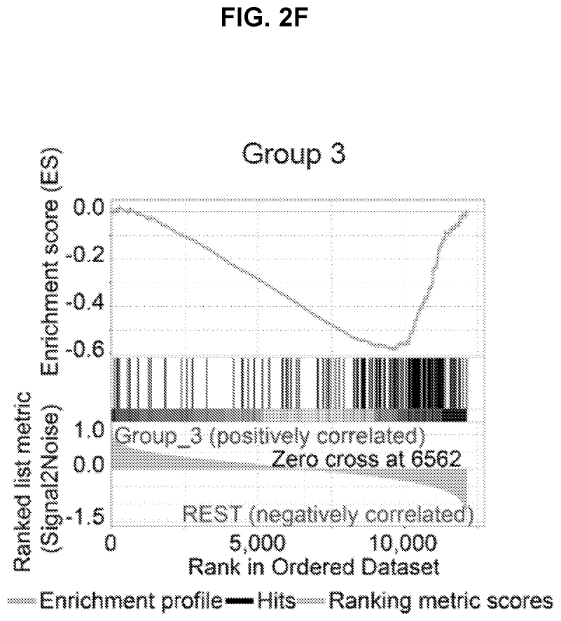

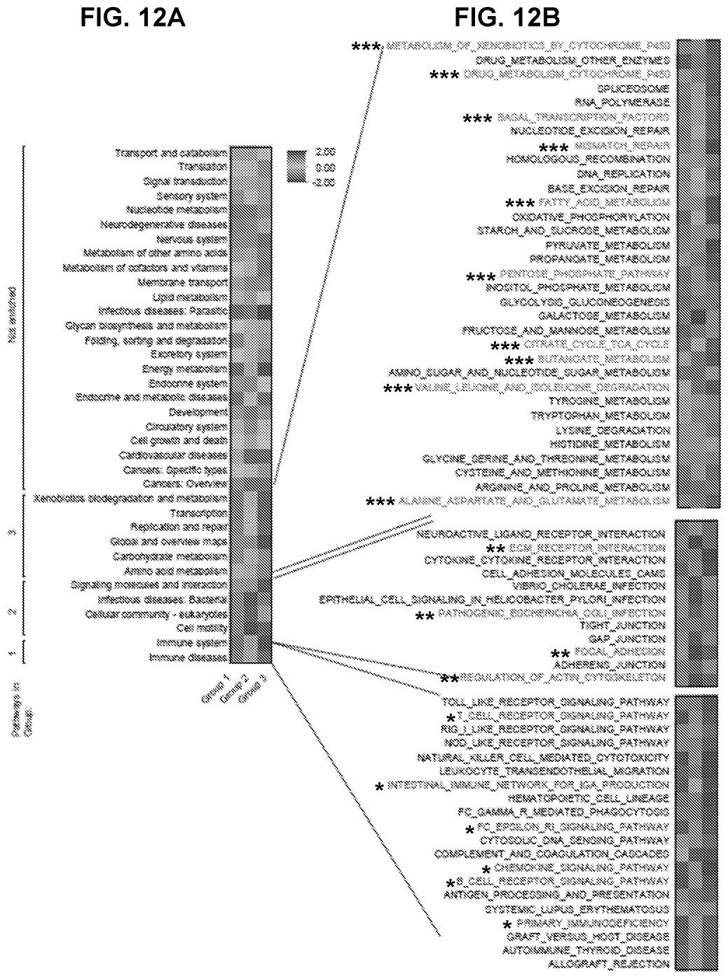

Gene Set Enrichment Analysis (GSEA) was performed to better understand the distinct biological pathways characterizing the different LNMs subtypes (FIG. 2 and FIG. 12). The Group 2 LNM subtype was associated with pathways characterizing an invasive subtype with enriched pathways including epithelial mesenchymal transition, apical junction, TGF-β signaling, angiogenesis, hypoxia, extracellular matrix receptor interactions, regulation of the actin cytoskeleton, and focal adhesion. Group 1 was associated with pathways characterizing an immune subtype with enriched pathways including allograft rejection, T cell receptor signaling pathway, and chemokine receptor signaling pathway among others. Group 3 was associated with pathways characterizing a proliferative/metabolic subtype with enriched pathways including MYC targets, basal transcription factors, and mismatch repair. Of note, Group 1 and Group 3 often displayed an inverse pattern of enriched gene sets. By contrast, Group 2 differed from Group 1 and Group 3 primarily in gene sets involved in the cell membrane and the extracellular matrix.

### Example No. 3. Lymph Node Subtypes were not Identified in the Primary Tumor

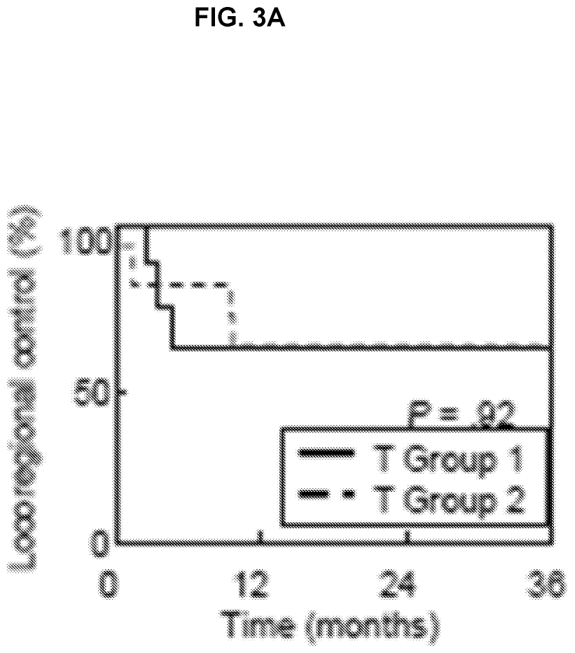

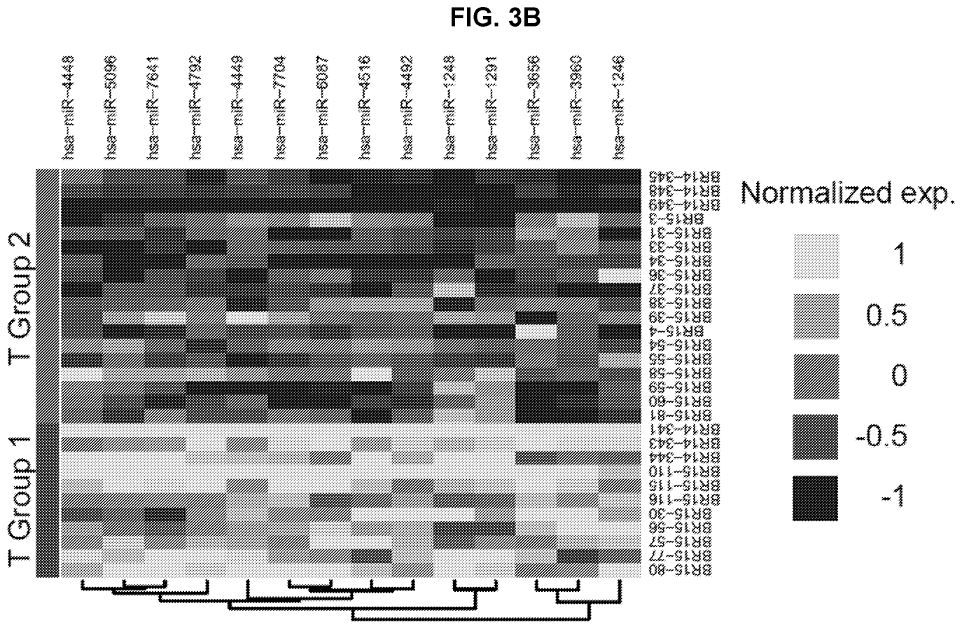

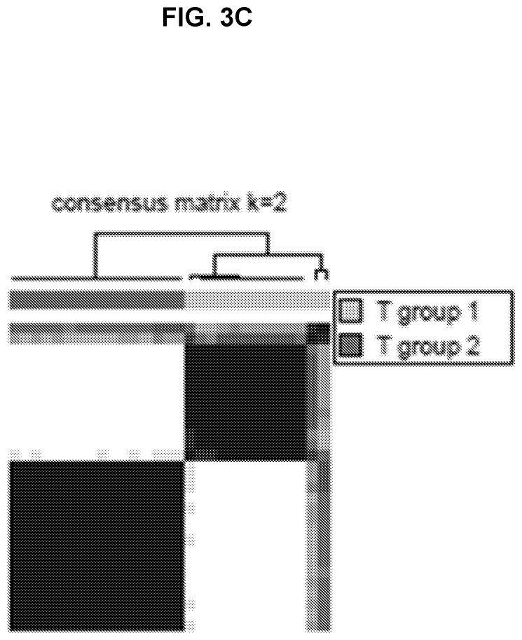

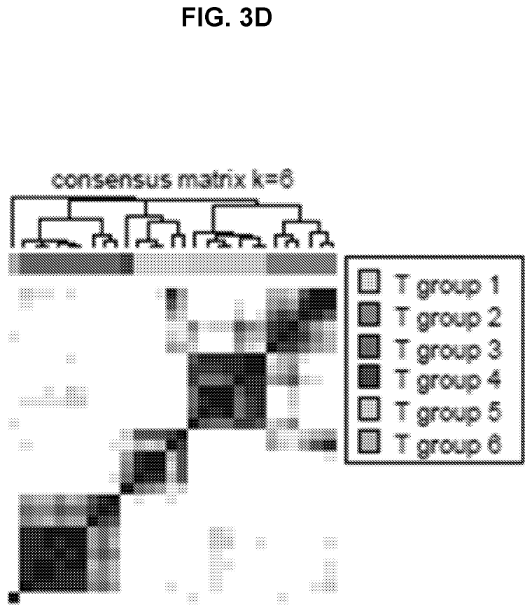

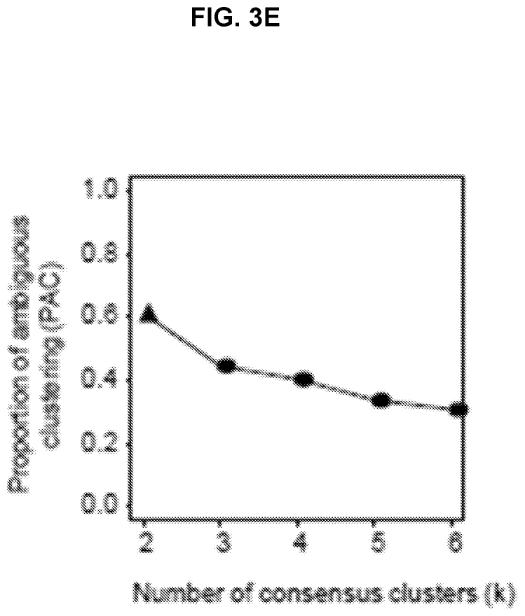

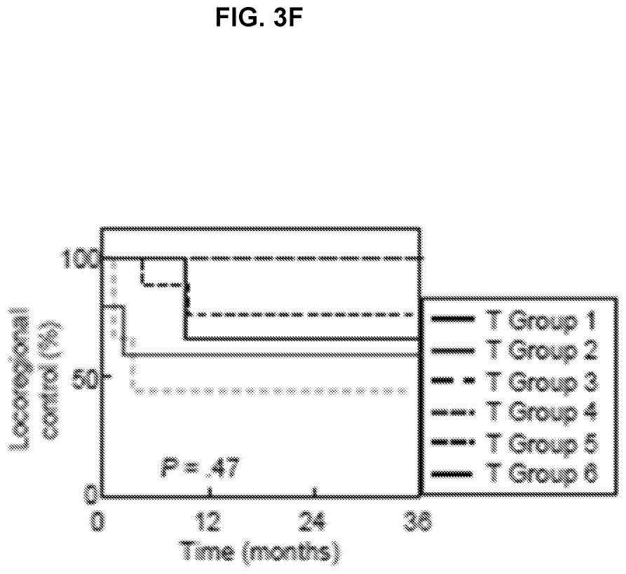

Previous reports in HNSCCs as well as in other cancers have demonstrated that the transcriptional profiles of LNMs were similar to the primary tumor (16, 17, 29, 30). Consequently, it was determined if unbiased classification methods would identify similar molecular subtypes in matched primary tumors. RNAseq and miRNAseq were performed on 29 matched primary tumors from patients with LNMs. Integrative clustering identified two molecular subtypes in primary tumors. However, these primary tumor subtypes, Tumor Group 1 and Tumor Group 2, did not differ in LRC (ly LRC: 63.5% for Tumor Group 1 vs. 64.0% for Tumor Group 2; P=0.92; FIG. 3A). Unlike LNMs, primary tumors did not have any DEmRNAs between groups, but there were 14 DEmiRNAs that confirmed the two subtypes as optimal stratification based on consensus clustering and proportion of ambiguous clusters (FIGS. 3B and 3C). Given that unbiased approaches did not identify similar molecular subtypes in primary tumors, supervised clustering of primary tumors was performed based on the DEmRNAs identified in the LNMs subtypes. Clustering based on the LNMs DEmRNAs identified 6 clusters that displayed similar rates of LRC (FIGS. 3D-3G). Consequently, the molecular subtypes identified in LNMs were not observed in the matched primary tumors.

### Example No. 4. The Gene Expression Profiles of LNMs Better Predicted for Locoregional Control and Relapse-Free Survival

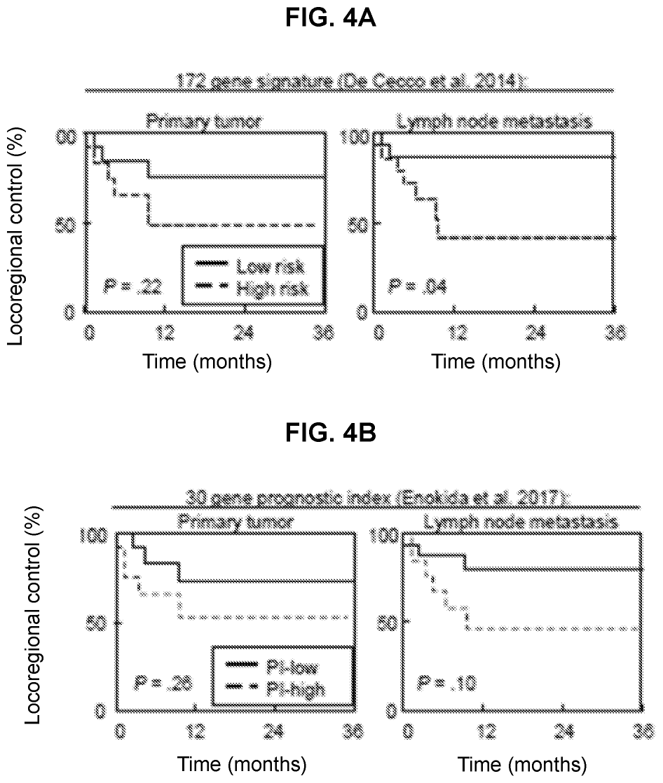

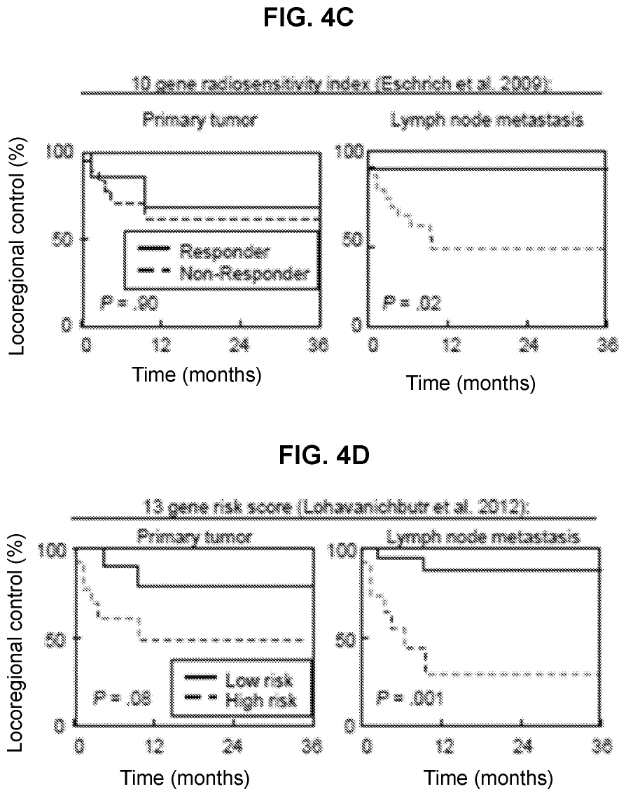

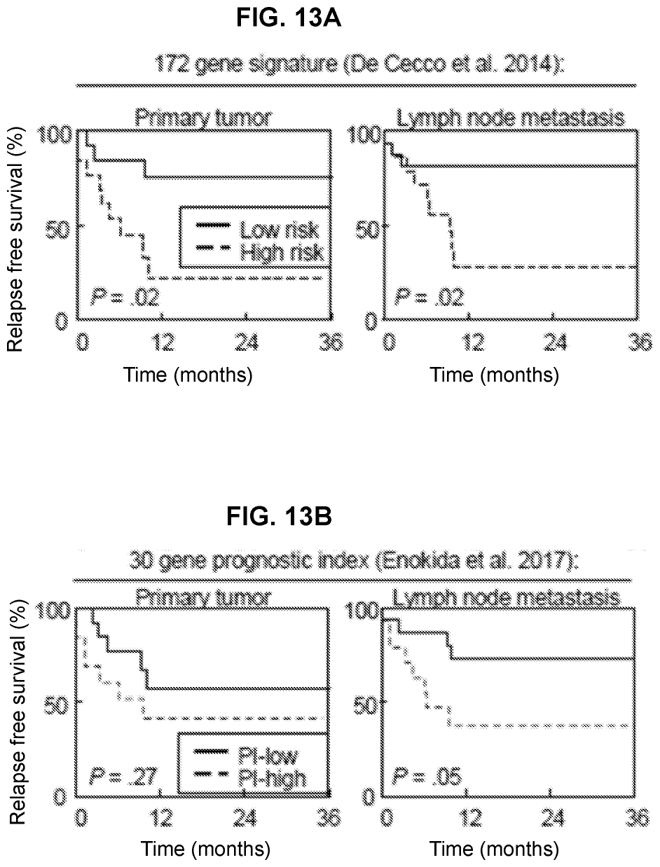

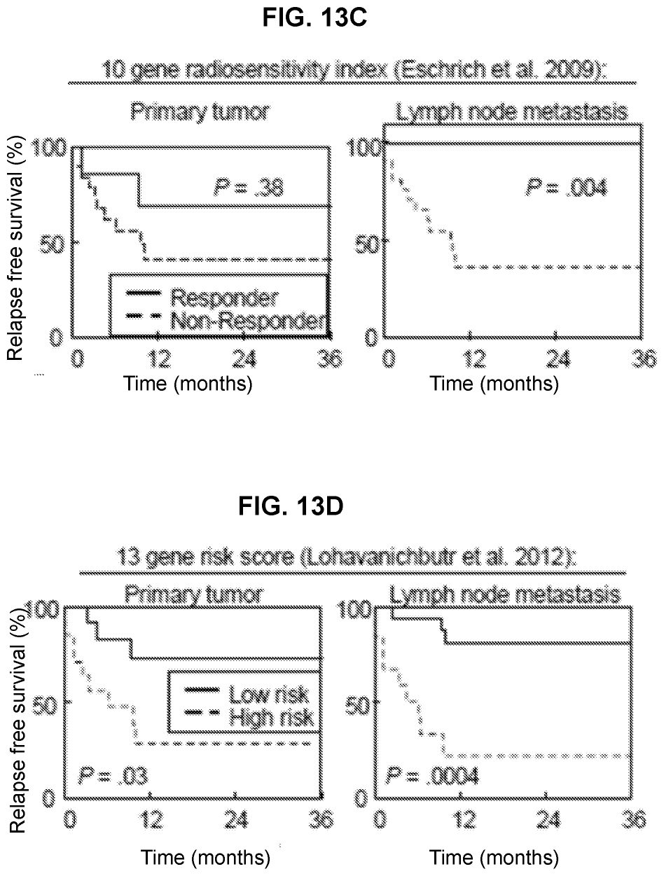

The ability to identify clinically relevant molecular subtypes in LNMs but not in primary tumors may reflect the biological selection of more aggressive subpopulations at the metastatic site. To determine if LNMs represented more aggressive variants, the ability of 4 prognostic HNSCC classifiers was assessed to predict locoregional control and relapse-free survival using the primary tumor or LNMs gene expression profiles. (1) a 172 gene signature derived from semi-supervised clustering and validated in a meta-analysis of 646 primary HNSCCs (25), (2) a 30 gene prognostic index (PI) derived from unsupervised clustering and validated in 123 primary oral cavity carcinomas (26), (3) a 10 gene radiosensitivity index (RSI) derived from a systems biology model and validated in 92 primary HNSCCs (27), and (4) 13-gene risk score derived from an L1-penalized Cox proportional hazard regression model were used and validated in 103 primary oral cavity cancers (28). When the primary tumor gene expression profiles were used in analysis, all 4 prognostic classifiers failed to stratify patients with worse LRC and 2 of 4 classifiers failed to stratify patients with worse RFS (FIG. 4 and FIG. 13).

By contrast, when the LNM gene expression profiles were used in analysis, 3 of the 4 classifiers stratified patients with worse LRC (173 gene signature P=0.04; 10 gene RSI P=0.02; 13 gene RS P=0.001) and all four classifiers stratified patients with worse RFS. Therefore, the gene expression profiles of LNMs better predicted outcomes than the gene expression profiles of primary tumors.

### Example No. 5. Identification of a Lymph Node-Specific Group 2 Classifier that Predicts Outcomes Across Multiple Cancer Types

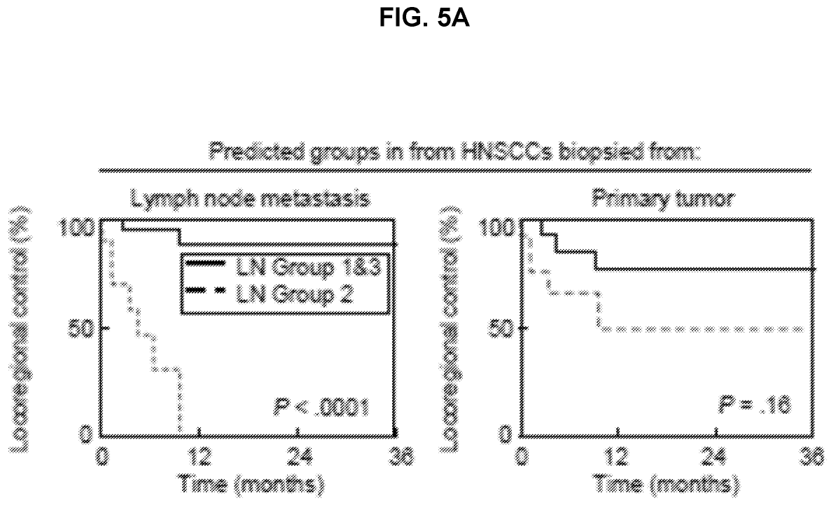

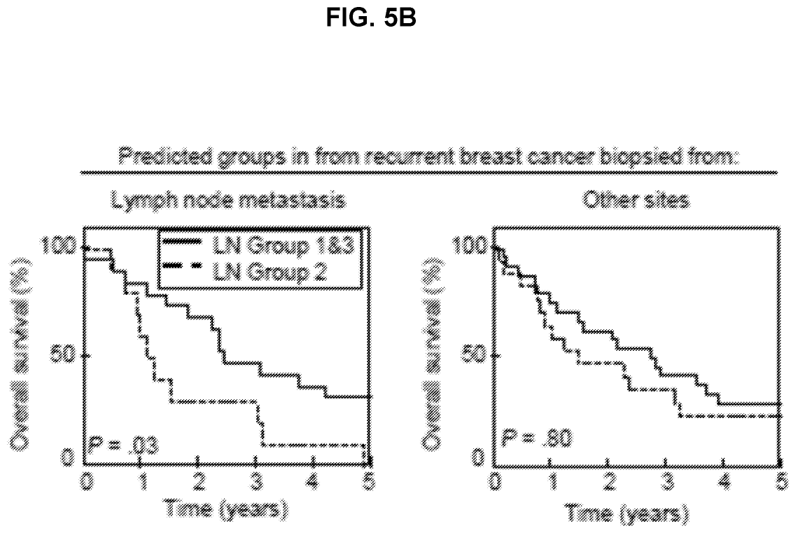

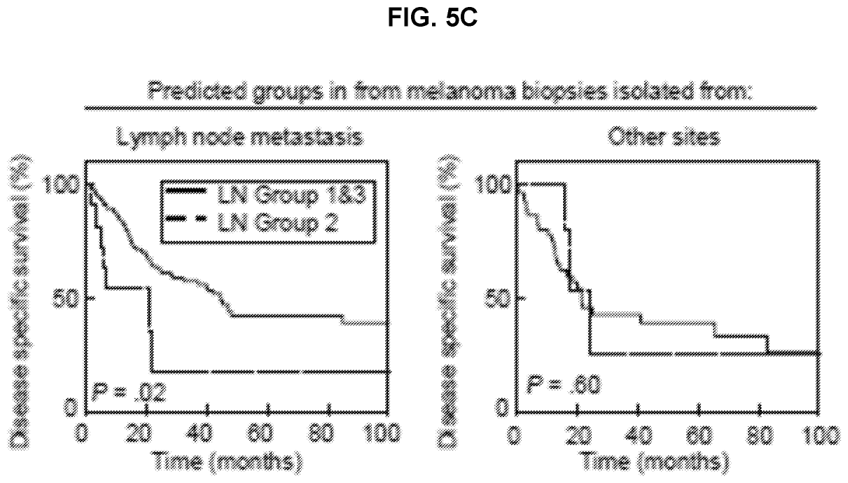

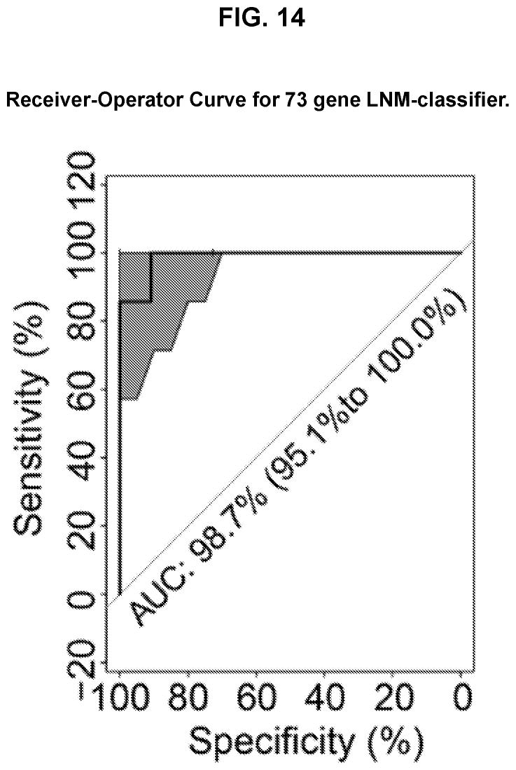

Previously published HNSCC prognostic signatures (25-28) were able to distinguish the aggressive Group 2 variant with 61.1% to 80.6% accuracy (Table 5). Consequently, classifiers developed from primary tumors to identify high risk subgroups may not adequately identify high risk subgroups in LNMs. A discovery set of 52 randomly selected LNMs were used to generate a nearest shrunken centroid-based 73 gene binary classifier that distinguished Group 2 LNMs from Group 1 and Group 3 LNMs (Group 1&3; Table 6). Using the remaining 18 LNM specimens as a testing cohort, this 73 gene classifier had an accuracy of 0.89 (95% CI: 0.65-0.99; P=0.01) with a sensitivity of 1.00, specificity of 0.82, positive predictive value of 0.78 and negative predictive value of 1.00. The ROC plot of this 73 gene classifier had an AUC of 98.7% (95% CI 95.1% to 100.0%; FIG. 14). As expected, the 73 gene LNMs classifier identified a clinically significant Group 2 subtype having worse LRC when applied to LNMs but not primary tumor specimens in the UIC HNSCC cohort (FIG. 5A). Next, the extent to which this 73 gene classifier predicted outcomes was assessed using the gene expression profiles from LNMs or non-LNMs in other cancers. A prospectively obtained breast cancer dataset (22) isolated from initial recurrences in LNMs (n=44), primary tumors (n=19), liver metastases (n=27), or other sites (n=30) was utilized. In addition, a dataset of retrospectively obtained melanoma specimens (23) isolated from the primary tumor (n=16), LNMs (n=139), distant metastases (n=23), in transit metastases (n=15) or local recurrences (n=11) was also utilized. Group 2 subtypes displayed worse overall survival and disease-specific survival when applied to the transcriptomes of breast and melanoma LNMs, respectively (FIGS. 5B and 5C). By contrast, when other non-LNM sites were analyzed, Group 2 subtypes were not associated with differences in outcomes consistent with our observations in HNSCC primary tumors.

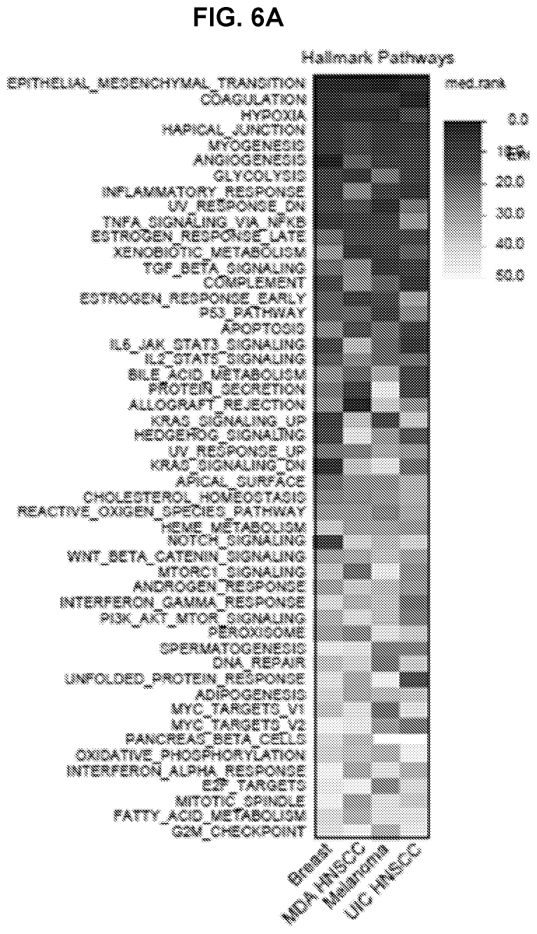

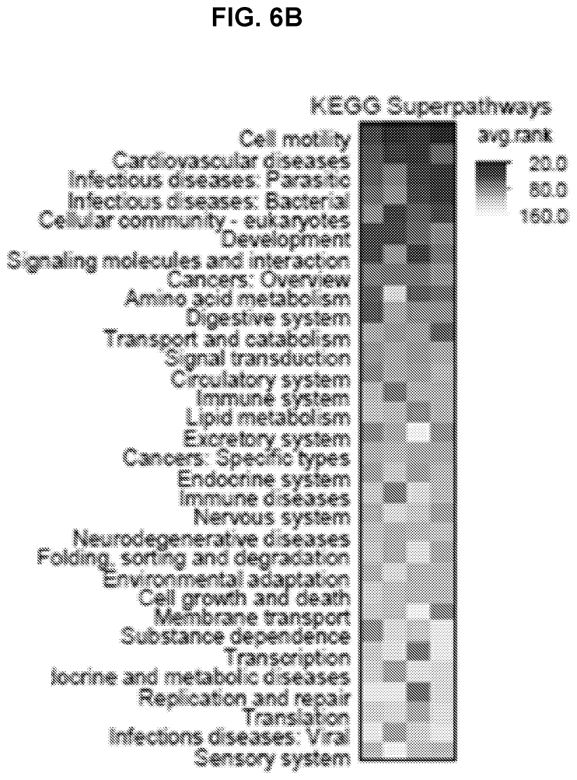

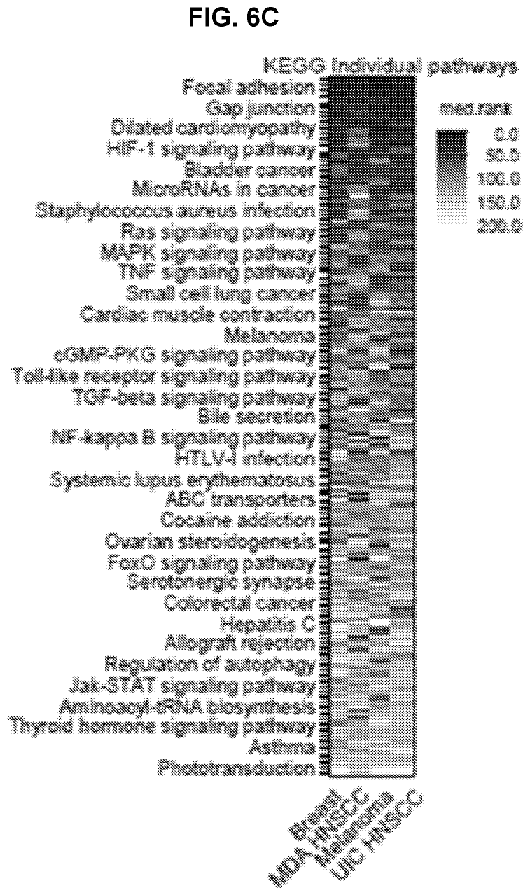

To confirm that this 73 gene classifier identified a Group 2 LNM subtype, ensemble GSEA (EGSEA) was performed to identify pathways unregulated in Group 2 subtypes in breast cancer LNM (22), melanoma LNM (23) as well as in a small retrospective series of LNMs from the MDACC HNSCC cohort (16). Group 2 subtypes identified from UIC HNSCC, MDACC, HNSCC, breast, and melanoma cohorts had a significant degree of concordance (Hallmark W=0.67; P=3.2×10⁻⁹; KEGG W=0.52; P=6.13×10⁻¹⁶). Of the 49 shared Hallmark gene sets, the top 3 median ranked pathways were epithelial mesenchymal transition (median rank: UIC HNSCC 5, Breast 3.5, Melanoma 1 and MDACC HNSCC 2), coagulation (median rank: UIC HNSCC 2, Breast 5.5, Melanoma 6 and MDACC HNSCC 7.5), and hypoxia (median rank: UIC HNSCC 14.5, Breast 5, Melanoma 4 and MDACC HNSCC 4.5; FIG. 6A). In the 187 shared gene pathways of the KEGG database, the top 3 median ranked pathways were ECM-receptor interactions (median rank: UIC HNSCC 16, Breast 5.5, Melanoma 5, and MDACC HNSCC 8), protein digestion and absorption (median rank: UIC HNSCC 16, Breast 10.5, Melanoma 3, and MDACC HNSCC 23), and focal adhesion (median rank: UIC HNSCC 15.5, Breast 10.5, Melanoma 5.5, and MDACC HNSCC 23; FIGS. 6B and 6C). Therefore, in four different datasets encompassing HNSCCs, breast cancers, and melanomas, this LNM-specific classifier identified a Group 2-like subtype displaying similar biological subtypes and clinical outcomes.

Discussion: Here, these experiments identified 3 distinct molecular subtypes of LNMs that were associated with differences in clinical outcomes after surgery and post-operative radiotherapy. These subtypes, termed Group 1, Group 2, and Group 3, displayed an immune, an invasive, and a metabolic/proliferative phenotype, respectively. In contrast to Group 1 and Group 3 LNM subtypes, the Group 2 LNM subtype was associated with significantly worse locoregional control, relapse-free survival, and overall survival. These subtypes are likely specific to LNMs for several reasons. First, LNMs subtypes were not observed in the matched primary tumors using either unsupervised or supervised approaches. Second, a similar Group 2 subtype was also identified in breast, melanoma, and MDACC HNSCC LNMs datasets and displayed similar gene expression patterns as well as increased risk for recurrence and death. Similar to the absence of these LNMs subtypes in primary HNSCCs, this Group 2 subtype was not associated with recurrence and survival differences when classifying non-LNMs sites. Even though these LNMs subtypes were identified using a single institution retrospective dataset encompassing 34 patients, similar observations were validated in existing prospective and retrospective datasets totaling 373 patients across 3 different cancer types. Thus, described herein is a novel molecular classification of LNMs metastasis, which has both biologic and prognostic significance in HNSCCs and likely extends to LNMs in other cancers.

Previous attempts to biologically classify HNSCCs have mostly relied upon supervised analysis incorporating known patient outcomes in order to identify mutations and/or differentially expressed genes that correlated with worse survival (25, 28, 31-33). By contrast, fewer studies have used unbiased strategies to classify HNSCCs into distinct biological subtypes that would predict different biological outcomes (26, 34, 35). In either case, previous reports have almost always focused on the primary tumor which likely masks small subpopulations of aggressive variants due tumor heterogeneity (25, 26, 34-39). To this end, LNMs subtypes identified here were not detected in matched primary tumors using unbiased integrative clustering, supervised clustering of DEmRNAs, or a 73-gene LNM-specific classifier. These findings are consistent with a few reports that demonstrate lymph node and/or distant metastases are molecularly distinct from the primary tumor (40). Unlike these results describing LNM-specific subtypes, most previous studies of LNMs in HNSCCs and other cancers demonstrated mutational and transcriptional profiles that were most similar to the matched primary tumors (16, 17, 29, 41-43). However, these studies were not designed to identify LNM-specific molecular subtypes because analyses combined both the primary tumors and LNMs from multiple patients. By comparing LNMs to primary tumors, the variance between patients likely confounded the identification of differences between primary tumors and LNMs and, therefore, was not optimized to identify LNM-specific differences. Furthermore, compared to previous studies, the experiments described here used 5 to 7-fold more LNMs samples that provided greater power to identify distinct LNM subtypes. Therefore, classifying the molecular differences present in LNMs provides novel biological and prognostic information to lead to more effective and/or targeted treatments of cancer.

The present LNM-specific molecular subtypes represent an invasive subtype (Group 2), an inflammatory subtype (Group 1), and a metabolic/proliferative subtype (Group 3). Others have described 3 to 6 subtypes in primary HNSCCs (35, 38, 44, 45). Pathways enriched in the Group 2 LNM subtype were similar to pathways enriched in a basal hypoxia or angiogenesis subtype in primary HNSCCs (34, 35, 38, 44, 45). Similarly, pathways enriched in the Group 2 or Group 3 LNMs subtype were similar to pathways enriched in the inflamed/mesenchymal subtype (35, 38, 44, 45) or classical-atypical subtype (34, 35, 44, 45) in primary HNSCCs, respectively. However, in contrast to the observations presented herein, differences in molecular subtypes of primary HNSCCs were not often associated with differences in clinical outcomes. Although Chung et al. did find that an intrinsic classification system in addition to other clinicopathological features were associated with worse recurrence free survival (34, 37), the LNM-subtype was observed to be the sole factor associated with cancer recurrence. Keck et al. (35) observed survival differences between HNSCC subtypes, but these differences were mainly due to differences in survival between HPV-positive and HPV-negative HNSCC subtypes. By contrast, this study identified subtypes of HPV-negative HNSCCs that were associated with significant survival differences. One explanation for these differences between the present findings and others is that LNMs represent the biological selection of aggressive variants compared to a more genetically heterogeneous primary tumor.

LNMs likely reflect biological selection of clinically relevant subpopulations as well as display LN-specific gene expression patterns. The biological selection of LNMs was supported by the observation that existing prognostic classifiers in HNSCCs (25-28) identified patients at increased risk of locoregional failure and relapse-free survival primarily when applied to the transcriptional profiles of LNMs but not to the transcriptional profiles of primary tumors.

However, these existing classifiers only predicted the Group 2 LNM subtype with 60-80% accuracy. By contrast, a novel 73 gene classifier identified Group 2-like subtypes in multiple cancer types only when applied to the gene expression patterns of LNMs but not when applied to non-lymph node sites indicating that LNMs also reflect LN-specific changes in the gene expression patterns of metastases. Similarly, in liver metastases, a selective gene signature was associated with adverse outcomes in patients with a luminal A breast cancer subtype (46). Thus, reported herein is a novel LNM-specific classifier that predicts outcomes using both retrospective and prospective data sets encompassing multiple cancer types.

TABLE 1
Overall patient characteristics.

| | All patients, No. of | |
|---|---|---|
| | patients (%) (n = 34) | |
| Follow up, median months | 25.8 | |
| (interquartile ratio) | (7.3-59.8) | |
| Median age | 59.9 | |
| (interquartile ratio) | (47.7-64.9) | |
| Gender | | |
| Male | 23 | (69.7%) |
| Female | 10 | (30.3%) |
| KPS | | |
| ≥70 | 30 | (93.8%) |
| <70 | 2 | (6.3%) |
| Cormorbidity group | | |
| Medium | 25 | (75.8%) |
| High-very high | 8 | (24.2%) |
| Ethnicity | | |
| African American | 11 | (42.3%) |
| Caucasian | 15 | (57.7%) |
| Clinical T-classification | | |
| T1-2 | 10 | (30.0%) |
| T3-4 | 23 | (69.7%) |
| Clinical N-classification | | |
| N0-1 | 17 | (51.5%) |
| N2-3 | 16 | (48.5%) |
| Primary | | |
| Oral cavity | 25 | (75.8%) |
| Oropharynx | 1 | (3.0%) |
| Larynx | 4 | (12.1%) |
| Hypopharynx | 1 | (3.0%) |
| Other | 2 | (6.1%) |
| Alcohol use | | |
| Yes | 13 | (65.0%) |
| No | 7 | (35.0%) |
| Smoking use | | |
| Yes | 21 | (63.6%) |
| No | 7 | (36.4%) |
| Pathologic T-classification | | |
| pT1-2 | 11 | (65.6%) |
| pT3-4 | 21 | (34.4%) |
| Pathologic N-classification | | |
| pN1 | 6 | (18.2%) |
| pN2-3 | 27 | (81.8%) |
| Margin status | | |
| Positive | 12 | (38.7%) |
| ≤5 mm | 11 | (34.5%) |
| Negative | 8 | (25.8%) |
| Perineural invasion | | |
| Yes | 12 | (36.4%) |
| No | 21 | (63.6%) |
| Lymphovascular space invasion | | |
| Yes | 11 | (33.3%) |
| No | 22 | (66.7%) |
| Extracapsular extension | | |
| Yes | 17 | (51.5%) |
| No | 16 | (48.5%) |
| Median number of positive LN | 3 | |
| (interquartile ratio) | (2-5) | |
| Median number of LN dissected | 28 | |
| (interquartile ratio) | (22-38) | |
| Median radiation dose to | 6600 | |
| post-op bed | | |
| (interquartile ratio) | (6300-6600) | |
| Treatment package time, days | 96 | |
| (interquartile ratio) | (86-106) | |
| Post operative chemoradiation | | |
| Yes | 26 | (78.8%) |
| No | 7 | (21.2%) |

TABLE 2
Patient characteristics by lymph node molecular classification.

| | All patients, No. of patients (%) | | | |
|---|---|---|---|---|
| | LN Group 1 | LN Group 2 | LN Group 3 | P |
| | (n = 15) | (n = 10) | (n = 8) | value |
| Median age | 52.3 | 61.7 | 61.7 | .51 |
| (interquartile ratio) | (46.9-67.6) | (51.3-65.3) | (52.6-64.8) | |
| Gender | | | | .87 |
| Male | 11 (73.3%) | 7 (70.0%) | 5 (62.5%) | |
| Female | 4 (26.7%) | 3 (30.0%) | 3 (37.5%) | |
| KPS | | | | .57 |
| ≥70 | 14 (93.3%) | 9 (90.0%) | 7 (100.0%) | |
| <70 | 1 (6.7%) | 1 (10.0%) | 0 (0.0%) | |
| Cormorbidity | | | | .85 |
| group | | | | |
| Medium | 12 (80.0%) | 7 (70.0%) | 6 (75.0%) | |
| High-very high | 3 (20.0%) | 3 (30.0%) | 2 (25.0%) | |
| Ethnicity | | | | .82 |
| African American | 5 (58.3%) | 2 (33.3%) | 4 (50.0%) | |
| Caucasian | 7 (41.7%) | 4 (66.7%) | 4 (50.0%) | |
| Clinical T- | | | | .67 |
| classification | | | | |
| T1-2 | 5 (33.3%) | 2 (20.0%) | 3 (37.5%) | |
| T3-4 | 10 (66.7%) | 8 (80.0%) | 5 (62.5%) | |
| Clinical N- | | | | .62 |
| classification | | | | |
| N0-1 | 6 (53.3%) | 6 (60.0%) | 3 (37.5%) | |
| N2-3 | 7 (46.7%) | 4 (40.0%) | 5 (62.5%) | |
| Primary | | | | .08 |
| Oral cavity | 12 (80.0%) | 10 (100.0%) | 3 (37.5%) | |
| Oropharynx | 1 (6.7%) | 0 (0.0%) | 0 (0.0%) | |
| Larynx | 1 (6.7%) | 0 (0.0%) | 3 (37.5%) | |
| Hypopharynx | 0 (0.0%) | 0 (0.0%) | 1 (6.7%) | |
| Other | 1 (6.7%) | 0 (0.0%) | 1 (6.7%) | |
| Alcohol use | | | | .98 |
| Yes | 5 (62.5%) | 4 (66.7%) | 4 (66.7%) | |
| No | 3 (37.5%) | 2 (33.3%) | 2 (33.3%) | |
| Smoking use | | | | .10 |
| Yes | 10 (66.7%) | 4 (40.0%) | 7 (87.5%) | |
| No | 5 (33.3%) | 6 (60.0%) | 1 (12.5%) | |
| Pathologic T- | | | | .11 |
| classification | | | | |
| pT1-2 | 6 (42.9%) | 1 (10.0%) | 4 (50.0%) | |
| pT3-4 | 8 (57.1%) | 9 (90.0%) | 4 (50.0%) | |
| Pathologic | | | | .53 |
| N-classification | | | | |
| pNx-1 | 2 (13.3%) | 3 (30.0%) | 1 (12.5%) | |
| pN2-3 | 13 (86.7%) | 7 (70.0%) | 7 (87.5%) | |
| Margin status | | | | .10 |
| Positive | 2 (14.3%) | 6 (60.0%) | 4 (57.1%) | |
| ≤5 mm | 6 (42.9%) | 3 (30.0%) | 2 (28.6%) | |
| Negative | 6 (42.9%) | 1 (10.0%) | 1 (14.3%) | |
| Perineural invasion | | | | .18 |
| Yes | 4 (26.7%) | 6 (60.0%) | 2 (25.0%) | |
| No | 11 (73.3%) | 4 (40.0%) | 6 (75.0%) | |
| Lymphovascular | | | | .02 |
| space invasion | | | | |
| Yes | 3 (20.0%) | 2 (20.0%) | 6 (75.0%) | |
| No | 12 (80.0%) | 8 (80.0%) | 2 (25.0%) | |
| Extracapsular | | | | .80 |
| extension | | | | |
| Yes | 7 (46.7%) | 6 (60.0%) | 4 (50.0%) | |
| No | 8 (53.3%) | 4 (40.0%) | 4 (50.0%) | |
| Median number | 3 | 4.5 | 2.5 | .56 |
| of positive LN | | | | |
| (interquartile ratio) | (2-3.5) | (1-9.5) | (1.25-4.75) | |
| Median number | 28.5 | 23.5 | 42 | .03 |
| of LN dissected | | | | |
| (interquartile ratio) | (24-35.3) | (17-32) | (29.5-62) | |
| Median radiation | 6600 | 6600 | 6200 | .25 |
| dose to post-op bed | (6300-6600) | (6300-6825) | (5985-6600) | |
| (interquartile ratio) | | | | |
| Treatment package | 91 | 97 | 96 | .64 |
| time, days | | | | |
| (interquartile ratio) | (84.5-101.5) | (82.5-110) | (87.5-125) | |
| Post operative | | | | .57 |
| chemoradiation | | | | |
| Yes | 13 (86.7%) | 7 (70.0%) | 6 (75.0%) | |
| No | 2 (13.3%) | 3 (30.0%) | 2 (25.0%) | |

TABLE 3
Univariate and multivariate analysis for locoregional control.

| | Univariate | | | Multivariate | |
|---|---|---|---|---|---|
| | analysis for survival | | | analysis for survival | |
| | | | P | | P |
| | Hazard ratio | | value | Hazard ratio | value |
| LN grouping | | | | | |
| LN Group 1 | Reference | | | Reference | |
| LN Group 2 | 12.91 | (2.52-103.23) | .002 | 10.00 (1.40-108.40) | .02 |
| LN Group 3 | 2.11 | (0.35-16.25) | .40 | 2.04 (0.27-17.82) | .48 |
| LN grouping | | | | | |
| LN Group 2 | Reference | | | Reference | |
| LN Group 1 | 0.08 | (0.009-0.40) | .002 | 0.10 (0.01-0.71) | .02 |
| LN Group 3 | 0.16 | (0.0-0.79) | .02 | 0.20 (0.03-0.99) | .04 |
| Primary site | | | | N.D. | N.D. |
| Oral cavity | Reference | | | | |
| Oropharynx | 0.86 | (0.04-6.00) | .90 | | |
| Larynx | 1.82 | (0.27-7.58) | .49 | | |
| Hypopharynx | 1.75 × 10⁻⁹ (0.0-9.82) | | .50 | | |
| Other | 1.76 × 10⁻¹⁰ (0.0-2.58) | | .21 | | |
| Smoking use | | | | | |
| No | Reference | | | N.D. | N.D. |
| Yes | 0.97 | (0.26-4.61) | .97 | | |
| Pathologic T- | | | | N.D. | N.D. |
| classification | | | | | |
| pT1-2 | Reference | | | | |
| pT3-4 | 1.70 | (0.45-8.03 | .44 | | |
| Lympho- | | | | N.D. | N.D. |
| vascular | | | | | |
| space | | | | | |
| invasion | | | | | |
| No | Reference | | | | |
| Yes | 0.50 | (0.13-2.05) | .42 | | |
| Margin status | | | | | |
| Negative | Reference | | | Reference | |
| ≤5 mm | 2.19 | (0.27-44.92) | .48 | 2.71 (0.32-57.10) | .37 |
| Positive | 5.58 | (0.95-105.81) | .06 | 2.71 (0.41-54.34) | .33 |
| Extent of LN | | | | N.D. | N.D. |
| dissection | | | | | |
| ≤28 LN | Reference | | | | |
| >28 LN | 0.49 | (0.10-2.01) | .31 | | |

Abbreviations:
S + RT, surgery and post-operative radiotherapy;
S + CRT, surgery and post operative chemoradiotherapy;
pSG, pathological Stage Grouping;
pT, pathological T classification;
pN, pathological N classification;
≥2 LN, greater than or equal the 2 metastatic lymph nodes;
LVSI, lymphovascular space invasion;
LND, lymph node dissection.

TABLE 4
DEmRNAs and DEmiRNAs in LNMs.

| | | | | | | P. | adj. | |
|---|---|---|---|---|---|---|---|---|
| Comparison | gene_id | gene_symbol | logFC | AveExpr | t | Value | P. Val | B |
| LNM Group 2 vs. Group 1 | ENSG00000167077 | MEI1 | −0.610 | 3.535 | −2.666 | 0.011 | 0.050 | −2.888 |
| LNM Group 2 vs. Group 1 | ENSG00000106278 | PTPRZ1 | 0.646 | 6.146 | 2.668 | 0.011 | 0.050 | −3.055 |
| LNM Group 2 vs. Group 1 | ENSG00000131196 | NFATC1 | −0.781 | 5.700 | −2.669 | 0.011 | 0.050 | −3.065 |
| LNM Group 2 vs. Group 1 | ENSG00000079337 | RAPGEF3 | 0.661 | 5.155 | 2.671 | 0.011 | 0.050 | −2.901 |
| LNM Group 2 vs. Group 1 | ENSG00000148357 | HMCN2 | −1.069 | 3.520 | −2.672 | 0.011 | 0.050 | −2.880 |
| LNM Group 2 vs. Group 1 | ENSG00000162105 | SHANK2 | 0.780 | 4.827 | 2.673 | 0.011 | 0.050 | −2.954 |
| LNM Group 2 vs. Group 1 | ENSG00000197863 | ZNF790 | −0.661 | 3.500 | −2.677 | 0.011 | 0.049 | −2.897 |
| LNM Group 2 vs. Group 1 | ENSG00000130592 | LSPI | −0.831 | 6.328 | −2.679 | 0.011 | 0.049 | −3.058 |
| LNM Group 2 vs. Group 1 | ENSG00000167208 | SNX20 | −0.840 | 4.543 | −2.692 | 0.010 | 0.048 | −2.898 |
| LNM Group 2 vs. Group 1 | ENSG00000092068 | SLC7A8 | 0.788 | 6.411 | 2.693 | 0.010 | 0.048 | −2.950 |
| LNM Group 2 vs. Group 1 | ENSG00000149218 | ENDOD1 | 0.615 | 5.826 | 2.694 | 0.010 | 0.048 | −2.969 |
| LNM Group 2 vs. Group 1 | ENSG00000121316 | PLBD1 | 0.586 | 4.153 | 2.707 | 0.010 | 0.046 | −2.849 |
| LNM Group 2 vs. Group 1 | ENSG00000143153 | ATPI B1 | 0.671 | 6.955 | 2.714 | 0.010 | 0.046 | −2.940 |
| LNM Group 2 vs. Group 1 | ENSG00000136960 | ENPP2 | −0.691 | 4.829 | −2.714 | 0.010 | 0.046 | −2.836 |
| LNM Group 2 vs. Group 1 | ENSG00000186684 | CYP27C1 | 0.729 | 3.973 | 2.721 | 0.009 | 0.045 | −2.766 |
| LNM Group 2 vs. Group 1 | ENSG00000167723 | TRPV3 | 0.814 | 4.786 | 2.722 | 0.009 | 0.045 | −2.839 |
| LNM Group 2 vs. Group 1 | ENSG00000090776 | EFNB1 | 0.886 | 6.034 | 2.724 | 0.009 | 0.045 | −2.895 |
| LNM Group 2 vs. Group 1 | ENSG00000088367 | EPB41L1 | 0.762 | 5.184 | 2.726 | 0.009 | 0.045 | −2.863 |
| LNM Group 2 vs. Group 1 | ENSG00000214595 | EML6 | −0.641 | 4.640 | −2.730 | 0.009 | 0.045 | −2.804 |
| LNM Group 2 vs. Group 1 | ENSG00000253159 | PCDHGA12 | 0.617 | 2.731 | 2.731 | 0.009 | 0.045 | −2.709 |
| LNM Group 2 vs. Group 1 | ENSG00000066294 | CD84 | −0.659 | 6.667 | −2.735 | 0.009 | 0.044 | −2.869 |
| LNM Group 2 vs. Group 1 | ENSG00000254995 | STX16- | −0.685 | 2.961 | −2.744 | 0.009 | 0.044 | −2.704 |
| | | NPEPL1 | | | | | | |
| LNM Group 2 vs. Group 1 | ENSG00000065320 | NTN1 | 0.589 | 4.083 | 2.747 | 0.009 | 0.044 | −2.862 |
| LNM Group 2 vs. Group 1 | ENSG00000103942 | HOMER2 | 0.755 | 5.826 | 2.748 | 0.009 | 0.043 | −2.767 |
| LNM Group 2 vs. Group 1 | ENSG00000166575 | TMEM135 | −0.610 | 4.536 | −2.750 | 0.009 | 0.043 | −2.751 |
| LNM Group 2 vs. Group 1 | ENSG00000073910 | FRY | −0.669 | 5.194 | −2.754 | 0.009 | 0.043 | −2.815 |
| LNM Group 2 vs. Group 1 | ENSG00000170456 | DENND5B | −0.756 | 5.167 | −2.755 | 0.009 | 0.043 | −2.801 |
| LNM Group 2 vs. Group 1 | ENSG00000051128 | HOMER3 | 0.869 | 4.957 | 2.759 | 0.009 | 0.043 | −2.730 |
| LNM Group 2 vs. Group 1 | ENSG00000203326 | ZNF525 | −0.964 | 4.471 | −2.762 | 0.009 | 0.042 | −2.763 |
| LNM Group 2 vs. Group 1 | ENSG00000167984 | NLRC3 | −1.125 | 5.656 | −2.762 | 0.009 | 0.042 | −2.765 |
| LNM Group 2 vs. Group 1 | ENSG00000154099 | DNAAFI | −1.023 | 3.752 | −2.762 | 0.009 | 0.042 | −2.712 |
| LNM Group 2 vs. Group 1 | ENSG00000163359 | COL6A3 | 0.740 | 10.354 | 2.763 | 0.009 | 0.042 | −2.836 |
| LNM Group 2 vs. Group 1 | ENSG00000124225 | PMEPA1 | 0.670 | 6.794 | 2.765 | 0.008 | 0.042 | −2.843 |
| LNM Group 2 vs. Group 1 | ENSG00000147394 | ZNF185 | 0.905 | 5.831 | 2.766 | 0.008 | 0.042 | −2.734 |
| LNM Group 2 vs. Group 1 | ENSG00000137841 | PLCB2 | −0.765 | 5.407 | −2.767 | 0.008 | 0.042 | −2.727 |
| LNM Group 2 vs. Group 1 | ENSG00000141013 | GAS8 | 0.653 | 4.114 | 2.769 | 0.008 | 0.042 | −2.688 |
| LNM Group 2 vs. Group 1 | ENSG00000164050 | PLXNB1 | 0.611 | 7.069 | 2.770 | 0.008 | 0.042 | −2.851 |
| LNM Group 2 vs. Group 1 | ENSG00000198853 | RUSC2 | 0.722 | 4.928 | 2.773 | 0.008 | 0.042 | −2.705 |
| LNM Group 2 vs. Group 1 | ENSG00000189195 | BTBD8 | −0.593 | 2.669 | −2.778 | 0.008 | 0.041 | −2.623 |
| LNM Group 2 vs. Group 1 | ENSG00000101901 | ALG13 | −0.602 | 6.365 | −2.784 | 0.008 | 0.041 | −2.746 |
| LNM Group 2 vs. Group 1 | ENSG00000134369 | NAV1 | 0.673 | 7.976 | 2.788 | 0.008 | 0.041 | −2.815 |
| LNM Group 2 vs. Group 1 | ENSG00000134333 | LDHA | 0.643 | 9.041 | 2.791 | 0.008 | 0.040 | −2.804 |
| LNM Group 2 vs. Group 1 | ENSG00000184058 | TBX1 | 0.646 | 3.675 | 2.793 | 0.008 | 0.040 | −2.622 |
| LNM Group 2 vs. Group 1 | ENSG00000138311 | ZNF365 | 0.863 | 2.997 | 2.793 | 0.008 | 0.040 | −2.584 |
| LNM Group 2 vs. Group 1 | ENSG00000126709 | IFI6 | 1.063 | 6.181 | 2.800 | 0.008 | 0.040 | −2.746 |
| LNM Group 2 vs. Group 1 | ENSG00000196923 | PDLIM7 | 0.685 | 6.118 | 2.801 | 0.008 | 0.040 | −2.708 |
| LNM Group 2 vs. Group 1 | ENSG00000143369 | ECM1 | 1.075 | 4.616 | 2.801 | 0.008 | 0.040 | −2.601 |
| LNM Group 2 vs. Group 1 | ENSG00000093072 | CECR1 | −0.626 | 6.003 | −2.802 | 0.008 | 0.040 | −2.728 |
| LNM Group 2 vs. Group 1 | ENSG00000137070 | IL11RA | −0.611 | 4.816 | −2.802 | 0.008 | 0.040 | −2.641 |
| LNM Group 2 vs. Group 1 | ENSG00000070404 | FSTL3 | 0.839 | 5.126 | 2.802 | 0.008 | 0.040 | −2.630 |
| LNM Group 2 vs. Group 1 | ENSG00000073792 | IGF2BP2 | 1.148 | 6.234 | 2.803 | 0.008 | 0.040 | −2.761 |
| LNM Group 2 vs. Group 1 | ENSG00000030419 | IKZF2 | −0.618 | 6.151 | −2.809 | 0.008 | 0.039 | −2.749 |
| LNM Group 2 vs. Group 1 | ENSG00000071073 | MGAT4A | −0.592 | 6.301 | −2.815 | 0.007 | 0.039 | −2.690 |
| LNM Group 2 vs. Group 1 | ENSG00000101384 | JAG1 | 0.760 | 8.293 | 2.821 | 0.007 | 0.038 | −2.728 |
| LNM Group 2 vs. Group 1 | ENSG00000169908 | TM4SF1 | 0.759 | 6.782 | 2.823 | 0.007 | 0.038 | −2.713 |
| LNM Group 2 vs. Group 1 | ENSG00000160703 | NLRX1 | 0.690 | 4.334 | 2.828 | 0.007 | 0.038 | −2.559 |
| LNM Group 2 vs. Group 1 | ENSG00000101213 | PTK6 | 0.611 | 4.586 | 2.831 | 0.007 | 0.038 | −2.549 |
| LNM Group 2 vs. Group 1 | ENSG00000164687 | FABP5 | 1.328 | 7.402 | 2.833 | 0.007 | 0.038 | −2.581 |
| LNM Group 2 vs. Group 1 | ENSG00000143297 | FCRL5 | −0.995 | 6.225 | −2.839 | 0.007 | 0.037 | −2.662 |
| LNM Group 2 vs. Group 1 | ENSG00000116147 | TNR | −0.956 | 2.582 | −2.839 | 0.007 | 0.037 | −2.479 |
| LNM Group 2 vs. Group 1 | ENSG00000090104 | RGS1 | −0.636 | 7.501 | −2.840 | 0.007 | 0.037 | −2.682 |
| LNM Group 2 vs. Group 1 | ENSG00000204262 | COL5A2 | 1.054 | 8.145 | 2.841 | 0.007 | 0.037 | −2.686 |
| LNM Group 2 vs. Group 1 | ENSG00000179583 | CIITA | −0.709 | 7.843 | −2.845 | 0.007 | 0.037 | −2.678 |
| LNM Group 2 vs. Group 1 | ENSG00000159399 | HK2 | 0.944 | 6.709 | 2.845 | 0.007 | 0.037 | −2.633 |
| LNM Group 2 vs. Group 1 | ENSG00000167123 | CERCAM | 0.777 | 5.523 | 2.846 | 0.007 | 0.037 | −2.601 |
| LNM Group 2 vs. Group 1 | ENSG00000117399 | CDC20 | 0.680 | 4.779 | 2.850 | 0.007 | 0.037 | −2.546 |
| LNM Group 2 vs. Group 1 | ENSG00000127152 | BCL11B | −0.619 | 6.280 | −2.853 | 0.007 | 0.036 | −2.619 |
| LNM Group 2 vs. Group 1 | ENSG00000182752 | PAPPA | 1.027 | 5.882 | 2.856 | 0.007 | 0.036 | −2.525 |
| LNM Group 2 vs. Group 1 | ENSG00000137877 | SPTBN5 | 0.621 | 4.638 | 2.858 | 0.007 | 0.036 | −2.554 |
| LNM Group 2 vs. Group 1 | ENSG00000145632 | PLK2 | 0.701 | 6.110 | 2.859 | 0.007 | 0.036 | −2.565 |
| LNM Group 2 vs. Group 1 | ENSG00000165995 | CACNB2 | −0.691 | 3.134 | −2.859 | 0.007 | 0.036 | −2.442 |
| LNM Group 2 vs. Group 1 | ENSG00000134057 | CCNB1 | 0.655 | 4.868 | 2.860 | 0.007 | 0.036 | −2.529 |
| LNM Group 2 vs. Group 1 | ENSG00000133816 | MICAL2 | 0.663 | 7.377 | 2.860 | 0.007 | 0.036 | −2.633 |
| LNM Group 2 vs. Group 1 | ENSG00000142945 | KIF2C | 0.587 | 4.573 | 2.863 | 0.007 | 0.036 | −2.534 |
| LNM Group 2 vs. Group 1 | ENSG00000070371 | CLTCL1 | 0.752 | 4.324 | 2.864 | 0.007 | 0.036 | −2.467 |
| LNM Group 2 vs. Group 1 | ENSG00000143367 | TUFT1 | 0.772 | 5.377 | 2.864 | 0.007 | 0.036 | −2.539 |
| LNM Group 2 vs. Group 1 | ENSG00000127084 | FGD3 | −0.950 | 5.444 | −2.867 | 0.006 | 0.036 | −2.538 |
| LNM Group 2 vs. Group 1 | ENSG00000163347 | CLDN1 | 1.052 | 6.832 | 2.869 | 0.006 | 0.036 | −2.610 |
| LNM Group 2 vs. Group 1 | ENSG00000066697 | MSANTD3 | 0.629 | 4.659 | 2.871 | 0.006 | 0.035 | −2.496 |
| LNM Group 2 vs. Group 1 | ENSG00000137269 | LRRC1 | 0.695 | 5.133 | 2.872 | 0.006 | 0.035 | −2.535 |
| LNM Group 2 vs. Group 1 | ENSG00000270149 | RP11- | 0.758 | 4.617 | 2.873 | 0.006 | 0.035 | −2.505 |
| | | 544M22.13 | | | | | | |
| LNM Group 2 vs. Group 1 | ENSG00000148498 | PARD3 | 0.687 | 5.941 | 2.873 | 0.006 | 0.035 | −2.438 |
| LNM Group 2 vs. Group 1 | ENSG00000010610 | CD4 | −0.611 | 5.993 | −2.875 | 0.006 | 0.035 | −2.552 |
| LNM Group 2 vs. Group 1 | ENSG00000185567 | AHNAK2 | 0.808 | 8.866 | 2.876 | 0.006 | 0.035 | −2.603 |
| LNM Group 2 vs. Group 1 | ENSG00000132855 | ANGPTL3 | 0.642 | 2.275 | 2.879 | 0.006 | 0.035 | −2.402 |
| LNM Group 2 vs. Group 1 | ENSG00000145703 | IQGAP2 | −0.630 | 5.472 | −2.882 | 0.006 | 0.035 | −2.474 |
| LNM Group 2 vs. Group 1 | ENSG00000184916 | JAG2 | 0.779 | 5.913 | 2.884 | 0.006 | 0.035 | −2.507 |
| LNM Group 2 vs. Group 1 | ENSG00000101670 | LIPG | 0.665 | 5.391 | 2.896 | 0.006 | 0.034 | −2.483 |
| LNM Group 2 vs. Group 1 | ENSG00000100311 | PDGFB | 0.666 | 4.381 | 2.901 | 0.006 | 0.034 | −2.426 |
| LNM Group 2 vs. Group 1 | ENSG00000185222 | TCEAL9 | 0.741 | 3.915 | 2.901 | 0.006 | 0.034 | −2.383 |
| LNM Group 2 vs. Group 1 | ENSG00000151006 | PRSS53 | 0.633 | 3.673 | 2.901 | 0.006 | 0.034 | −2.375 |
| LNM Group 2 vs. Group 1 | ENSG00000182957 | SPATA13 | −0.629 | 5.991 | −2.906 | 0.006 | 0.033 | −2.510 |
| LNM Group 2 vs. Group 1 | ENSG00000119862 | LGALSL | 1.069 | 5.054 | 2.906 | 0.006 | 0.033 | −2.451 |
| LNM Group 2 vs. Group 1 | ENSG00000108821 | COL1A1 | 1.297 | 11.587 | 2.908 | 0.006 | 0.033 | −2.478 |
| LNM Group 2 vs. Group 1 | ENSG00000154258 | ABCA9 | −0.691 | 5.028 | −2.910 | 0.006 | 0.033 | −2.413 |
| LNM Group 2 vs. Group 1 | ENSG00000109861 | CTSC | 0.652 | 8.068 | 2.910 | 0.006 | 0.033 | −2.520 |
| LNM Group 2 vs. Group 1 | ENSG00000161958 | FGF11 | 0.691 | 4.460 | 2.915 | 0.006 | 0.033 | −2.353 |
| LNM Group 2 vs. Group 1 | ENSG00000198142 | SOWAHC | 0.665 | 6.555 | 2.916 | 0.006 | 0.033 | −2.466 |
| LNM Group 2 vs. Group 1 | ENSG00000137642 | SORL1 | −0.644 | 7.898 | −2.917 | 0.006 | 0.033 | −2.512 |
| LNM Group 2 vs. Group 1 | ENSG00000235162 | C12orf75 | 0.634 | 4.765 | 2.917 | 0.006 | 0.033 | −2.390 |
| LNM Group 2 vs. Group 1 | ENSG00000138434 | SSFA2 | 0.608 | 8.007 | 2.918 | 0.006 | 0.033 | −2.510 |
| LNM Group 2 vs. Group 1 | ENSG00000072110 | ACTN1 | 0.600 | 8.384 | 2.921 | 0.006 | 0.033 | −2.504 |
| LNM Group 2 vs. Group 1 | ENSG00000266074 | BAHCC1 | 0.722 | 5.591 | 2.921 | 0.006 | 0.033 | −2.448 |
| LNM Group 2 vs. Group 1 | ENSG00000157514 | TSC22D3 | −0.679 | 7.754 | −2.933 | 0.005 | 0.032 | −2.444 |
| LNM Group 2 vs. Group 1 | ENSG00000138134 | STAMBPL1 | −0.641 | 4.427 | −2.935 | 0.005 | 0.032 | −2.328 |
| LNM Group 2 vs. Group 1 | ENSG00000164692 | COL1A2 | 1.284 | 10.783 | 2.936 | 0.005 | 0.032 | −2.433 |
| LNM Group 2 vs. Group 1 | ENSG00000181092 | ADIPOQ | −1.773 | 3.075 | −2.936 | 0.005 | 0.032 | −2.263 |
| LNM Group 2 vs. Group 1 | ENSG00000091409 | ITGA6 | 0.606 | 8.993 | 2.937 | 0.005 | 0.032 | −2.447 |
| LNM Group 2 vs. Group 1 | ENSG00000009950 | MLXIPL | −0.739 | 2.854 | −2.937 | 0.005 | 0.032 | −2.264 |
| LNM Group 2 vs. Group 1 | ENSG00000139351 | SYCP3 | −0.830 | 3.252 | −2.940 | 0.005 | 0.032 | −2.280 |
| LNM Group 2 vs. Group 1 | ENSG00000120327 | PCDHB14 | 0.654 | 3.064 | 2.941 | 0.005 | 0.031 | −2.279 |
| LNM Group 2 vs. Group 1 | ENSG00000102471 | NDFIP2 | 0.611 | 5.652 | 2.945 | 0.005 | 0.031 | −2.383 |
| LNM Group 2 vs. Group 1 | ENSG00000091136 | LAMB1 | 0.613 | 8.028 | 2.946 | 0.005 | 0.031 | −2.445 |
| LNM Group 2 vs. Group 1 | ENSG00000196482 | ESRRG | −0.737 | 2.440 | −2.947 | 0.005 | 0.031 | −2.245 |
| LNM Group 2 vs. Group 1 | ENSG00000151208 | DLG5 | 0.833 | 7.342 | 2.950 | 0.005 | 0.031 | −2.429 |
| LNM Group 2 vs. Group 1 | ENSG00000128641 | MYO1B | 0.755 | 7.660 | 2.952 | 0.005 | 0.031 | −2.412 |
| LNM Group 2 vs. Group 1 | ENSG00000110031 | LPXN | −0.604 | 4.750 | −2.951 | 0.005 | 0.031 | −2.275 |
| LNM Group 2 vs. Group 1 | ENSG00000138964 | PARVG | −0.820 | 5.810 | −2.958 | 0.005 | 0.031 | −2.338 |
| LNM Group 2 vs. Group 1 | ENSG00000114270 | COL7A1 | 0.809 | 9.645 | 2.959 | 0.005 | 0.031 | −2.387 |
| LNM Group 2 vs. Group 1 | ENSG00000119630 | PGF | 0.979 | 4.384 | 2.960 | 0.005 | 0.031 | −2.278 |
| LNM Group 2 vs. Group 1 | ENSG00000142694 | EVA1B | 0.652 | 3.218 | 2.959 | 0.005 | 0.031 | −2.225 |
| LNM Group 2 vs. Group 1 | ENSG00000233436 | BTBD18 | −0.781 | 3.385 | −2.961 | 0.005 | 0.031 | −2.244 |
| LNM Group 2 vs. Group 1 | ENSG00000103522 | IL21R | −0.975 | 5.182 | −2.967 | 0.005 | 0.030 | −2.292 |
| LNM Group 2 vs. Group 1 | ENSG00000090097 | PCBP4 | 0.641 | 3.865 | 2.966 | 0.005 | 0.030 | −2.233 |
| LNM Group 2 vs. Group 1 | ENSG00000198064 | RP11- | −0.688 | 4.845 | −2.968 | 0.005 | 0.030 | −2.272 |
| | | 347C12.1 | | | | | | |
| LNM Group 2 vs. Group 1 | ENSG00000188171 | ZNF626 | −0.744 | 3.786 | −2.970 | 0.005 | 0.030 | −2.206 |
| LNM Group 2 vs. Group 1 | ENSG00000170485 | NPAS2 | 0.723 | 5.305 | 2.972 | 0.005 | 0.030 | −2.338 |
| LNM Group 2 vs. Group 1 | ENSG00000112378 | PERP | 0.925 | 8.921 | 2.973 | 0.005 | 0.030 | −2.371 |
| LNM Group 2 vs. Group 1 | ENSG00000198736 | MSRB1 | 0.652 | 3.809 | 2.974 | 0.005 | 0.030 | −2.176 |
| LNM Group 2 vs. Group 1 | ENSG00000111732 | AICDA | −1.086 | 2.627 | −2.976 | 0.005 | 0.030 | −2.168 |
| LNM Group 2 vs. Group 1 | ENSG00000158315 | RHBDL2 | 0.680 | 3.228 | 2.977 | 0.005 | 0.030 | −2.172 |
| LNM Group 2 vs. Group 1 | ENSG00000127554 | GFER | 0.805 | 3.664 | 2.978 | 0.005 | 0.030 | −2.193 |
| LNM Group 2 vs. Group 1 | ENSG00000175866 | BAIAP2 | 0.640 | 6.342 | 2.980 | 0.005 | 0.030 | −2.346 |
| LNM Group 2 vs. Group 1 | ENSG00000137648 | TMPRSS4 | 1.489 | 5.956 | 2.982 | 0.005 | 0.029 | −2.341 |
| LNM Group 2 vs. Group 1 | ENSG00000169902 | TPST1 | 0.598 | 4.604 | 2.983 | 0.005 | 0.029 | −2.233 |
| LNM Group 2 vs. Group 1 | ENSG00000171208 | NETO2 | 0.667 | 5.173 | 2.986 | 0.005 | 0.029 | −2.198 |
| LNM Group 2 vs. Group 1 | ENSG00000213658 | LAT | −0.667 | 4.624 | −2.988 | 0.005 | 0.029 | −2.235 |
| LNM Group 2 vs. Group 1 | ENSG00000127824 | TUBA4A | 0.648 | 6.168 | 2.990 | 0.005 | 0.029 | −2.221 |
| LNM Group 2 vs. Group 1 | ENSG00000204147 | ASAH2B | 0.751 | 3.583 | 2.992 | 0.005 | 0.029 | −2.144 |
| LNM Group 2 vs. Group 1 | ENSG00000131620 | ANO1 | 0.852 | 6.926 | 2.997 | 0.005 | 0.029 | −2.268 |
| LNM Group 2 vs. Group 1 | ENSG00000008853 | RHOBTB2 | −0.585 | 5.166 | −2.998 | 0.005 | 0.029 | −2.231 |
| LNM Group 2 vs. Group 1 | ENSG00000136167 | LCP1 | −0.710 | 7.731 | −3.006 | 0.004 | 0.028 | −2.260 |
| LNM Group 2 vs. Group 1 | ENSG00000268861 | CTD- | −0.595 | 3.268 | −3.005 | 0.004 | 0.028 | −2.108 |
| | | 2207O23.3 | | | | | | |
| LNM Group 2 vs. Group 1 | ENSG00000137563 | GGH | 0.656 | 3.949 | 3.006 | 0.004 | 0.028 | −2.134 |
| LNM Group 2 vs. Group 1 | ENSG00000164142 | FAM160A1 | 0.928 | 4.893 | 3.007 | 0.004 | 0.028 | −2.197 |
| LNM Group 2 vs. Group 1 | ENSG00000114529 | C3orf52 | 0.806 | 5.283 | 3.010 | 0.004 | 0.028 | −2.206 |
| LNM Group 2 vs. Group 1 | ENSG00000039068 | CDH1 | 0.835 | 7.525 | 3.012 | 0.004 | 0.028 | −2.283 |
| LNM Group 2 vs. Group 1 | ENSG00000196329 | GIMAP5 | −0.849 | 5.025 | −3.013 | 0.004 | 0.028 | −2.170 |
| LNM Group 2 vs. Group 1 | ENSG00000221926 | TRIM16 | 0.671 | 5.957 | 3.016 | 0.004 | 0.028 | −2.198 |
| LNM Group 2 vs. Group 1 | ENSG00000175984 | DENND2C | 0.784 | 5.625 | 3.019 | 0.004 | 0.028 | −2.182 |
| LNM Group 2 vs. Group 1 | ENSG00000121903 | ZSCAN20 | 0.720 | 3.255 | 3.020 | 0.004 | 0.028 | −2.083 |
| LNM Group 2 vs. Group 1 | ENSG00000123384 | LRP1 | 0.709 | 9.251 | 3.026 | 0.004 | 0.027 | −2.240 |
| LNM Group 2 vs. Group 1 | ENSG00000179361 | ARID3B | −0.648 | 4.856 | −3.026 | 0.004 | 0.027 | −2.132 |
| LNM Group 2 vs. Group 1 | ENSG00000070729 | CNGB1 | 1.487 | 3.364 | 3.029 | 0.004 | 0.027 | −2.039 |
| LNM Group 2 vs. Group 1 | ENSG00000110436 | SLC1A2 | −1.239 | 3.190 | −3.030 | 0.004 | 0.027 | −2.047 |
| LNM Group 2 vs. Group 1 | ENSG00000196616 | ADH1B | −1.888 | 3.867 | −3.031 | 0.004 | 0.027 | −2.049 |
| LNM Group 2 vs. Group 1 | ENSG00000110324 | IL10RA | −0.755 | 6.457 | −3.032 | 0.004 | 0.027 | −2.181 |
| LNM Group 2 vs. Group 1 | ENSG00000168477 | TNXB | −0.972 | 5.254 | −3.033 | 0.004 | 0.027 | −2.155 |
| LNM Group 2 vs. Group 1 | ENSG00000188906 | LRRK2 | −0.758 | 5.285 | −3.033 | 0.004 | 0.027 | −2.122 |
| LNM Group 2 vs. Group 1 | ENSG00000162433 | AK4 | 0.848 | 6.002 | 3.037 | 0.004 | 0.027 | −2.169 |
| LNM Group 2 vs. Group 1 | ENSG00000159228 | CBR1 | 0.770 | 4.942 | 3.037 | 0.004 | 0.027 | −2.111 |
| LNM Group 2 vs. Group 1 | ENSG00000149212 | SESN3 | −0.702 | 7.940 | −3.040 | 0.004 | 0.027 | −2.215 |
| LNM Group 2 vs. Group 1 | ENSG00000147144 | CCDC120 | 0.595 | 4.898 | 3.041 | 0.004 | 0.026 | −2.093 |
| LNM Group 2 vs. Group 1 | ENSG00000189159 | HN1 | 0.669 | 5.885 | 3.045 | 0.004 | 0.026 | −2.126 |
| LNM Group 2 vs. Group 1 | ENSG00000164181 | ELOVL7 | 0.753 | 4.131 | 3.049 | 0.004 | 0.026 | −2.040 |
| LNM Group 2 vs. Group 1 | ENSG00000064687 | ABCA7 | −0.603 | 5.763 | −3.057 | 0.004 | 0.026 | −2.138 |
| LNM Group 2 vs. Group 1 | ENSG00000152952 | PLOD2 | 0.741 | 6.608 | 3.058 | 0.004 | 0.026 | −2.162 |
| LNM Group 2 vs. Group 1 | ENSG00000105639 | JAK3 | −0.670 | 6.685 | −3.063 | 0.004 | 0.025 | −2.148 |
| LNM Group 2 vs. Group 1 | ENSG00000162458 | FBLIM1 | 0.773 | 6.554 | 3.066 | 0.004 | 0.025 | −2.128 |
| LNM Group 2 vs. Group 1 | ENSG00000183066 | WBP2NL | −0.590 | 2.797 | −3.066 | 0.004 | 0.025 | −1.957 |
| LNM Group 2 vs. Group 1 | ENSG00000009790 | TRAF3IP3 | −0.864 | 5.648 | −3.069 | 0.004 | 0.025 | −2.064 |
| LNM Group 2 vs. Group 1 | ENSG00000176597 | B3GNT5 | 0.682 | 6.986 | 3.070 | 0.004 | 0.025 | −2.117 |
| LNM Group 2 vs. Group 1 | ENSG00000176619 | LMNB2 | 0.633 | 6.892 | 3.073 | 0.004 | 0.025 | −2.136 |
| LNM Group 2 vs. Group 1 | ENSG00000269190 | FBXO17 | 0.620 | 3.654 | 3.072 | 0.004 | 0.025 | −1.996 |
| LNM Group 2 vs. Group 1 | ENSG00000143126 | CELSR2 | 0.847 | 7.387 | 3.078 | 0.004 | 0.025 | −2.117 |
| LNM Group 2 vs. Group 1 | ENSG00000124243 | BCAS4 | −0.681 | 5.702 | −3.082 | 0.004 | 0.025 | −2.065 |
| LNM Group 2 vs. Group 1 | ENSG00000166145 | SPINT1 | 0.949 | 6.164 | 3.082 | 0.004 | 0.025 | −2.081 |
| LNM Group 2 vs. Group 1 | ENSG00000090020 | SLC9A1 | 0.609 | 6.178 | 3.085 | 0.004 | 0.025 | −2.066 |
| LNM Group 2 vs. Group 1 | ENSG00000088280 | ASAP3 | 0.719 | 4.772 | 3.086 | 0.004 | 0.025 | −2.006 |
| LNM Group 2 vs. Group 1 | ENSG00000176014 | TUBB6 | 0.620 | 6.420 | 3.086 | 0.004 | 0.025 | −2.060 |
| LNM Group 2 vs. Group 1 | ENSG00000139880 | CDH24 | 0.658 | 3.819 | 3.089 | 0.004 | 0.024 | −1.959 |
| LNM Group 2 vs. Group 1 | ENSG00000185010 | F8 | −0.715 | 3.242 | −3.094 | 0.004 | 0.024 | −1.896 |
| LNM Group 2 vs. Group 1 | ENSG00000128335 | APOL2 | 0.695 | 6.084 | 3.096 | 0.004 | 0.024 | −2.032 |
| LNM Group 2 vs. Group 1 | ENSG00000147804 | SLC39A4 | 0.624 | 3.759 | 3.095 | 0.004 | 0.024 | −1.944 |
| LNM Group 2 vs. Group 1 | ENSG00000166123 | GPT2 | 0.588 | 4.709 | 3.097 | 0.003 | 0.024 | −1.945 |
| LNM Group 2 vs. Group 1 | ENSG00000141338 | ABCA8 | −0.947 | 3.217 | −3.100 | 0.003 | 0.024 | −1.882 |
| LNM Group 2 vs. Group 1 | ENSG00000113758 | DBN1 | 0.686 | 5.446 | 3.100 | 0.003 | 0.024 | −2.001 |
| LNM Group 2 vs. Group 1 | ENSG00000142156 | COL6A1 | 1.008 | 8.728 | 3.106 | 0.003 | 0.024 | −2.042 |
| LNM Group 2 vs. Group 1 | ENSG00000139629 | GALNT6 | 0.893 | 5.858 | 3.109 | 0.003 | 0.024 | −1.985 |
| LNM Group 2 vs. Group 1 | ENSG00000060656 | PTPRU | 0.874 | 4.935 | 3.116 | 0.003 | 0.023 | −1.949 |
| LNM Group 2 vs. Group 1 | ENSG00000100994 | PYGB | 0.650 | 7.445 | 3.121 | 0.003 | 0.023 | −2.010 |
| LNM Group 2 vs. Group 1 | ENSG00000136002 | ARHGEF4 | 0.979 | 6.602 | 3.125 | 0.003 | 0.023 | −1.998 |
| LNM Group 2 vs. Group 1 | ENSG00000099377 | HSD3B7 | 0.624 | 4.049 | 3.126 | 0.003 | 0.023 | −1.830 |
| LNM Group 2 vs. Group 1 | ENSG00000111679 | PTPN6 | −0.626 | 5.799 | −3.128 | 0.003 | 0.023 | −1.929 |
| LNM Group 2 vs. Group 1 | ENSG00000240184 | PCDHGC3 | 0.785 | 5.779 | 3.128 | 0.003 | 0.023 | −1.922 |
| LNM Group 2 vs. Group 1 | ENSG00000065361 | ERBB3 | 1.017 | 5.836 | 3.129 | 0.003 | 0.023 | −1.915 |
| LNM Group 2 vs. Group 1 | ENSG00000184060 | ADAP2 | 0.661 | 4.480 | 3.133 | 0.003 | 0.023 | −1.829 |
| LNM Group 2 vs. Group 1 | ENSG00000066468 | FGFR2 | 0.680 | 5.366 | 3.140 | 0.003 | 0.022 | −1.916 |
| LNM Group 2 vs. Group 1 | ENSG00000181690 | PLAG1 | −0.721 | 4.011 | −3.141 | 0.003 | 0.022 | −1.872 |
| LNM Group 2 vs. Group 1 | ENSG00000161647 | MPP3 | 0.722 | 3.961 | 3.141 | 0.003 | 0.022 | −1.833 |
| LNM Group 2 vs. Group 1 | ENSG00000151014 | NOCT | 0.614 | 4.710 | 3.142 | 0.003 | 0.022 | −1.857 |
| LNM Group 2 vs. Group 1 | ENSG00000135119 | RNFT2 | −0.644 | 4.283 | −3.146 | 0.003 | 0.022 | −1.829 |
| LNM Group 2 vs. Group 1 | ENSG00000087077 | TRIP6 | 0.591 | 5.740 | 3.148 | 0.003 | 0.022 | −1.901 |
| LNM Group 2 vs. Group 1 | ENSG00000136286 | MYO1G | −0.604 | 5.435 | −3.149 | 0.003 | 0.022 | −1.853 |
| LNM Group 2 vs. Group 1 | ENSG00000113532 | ST8SIA4 | −0.644 | 5.509 | −3.156 | 0.003 | 0.022 | −1.832 |
| LNM Group 2 vs. Group 1 | ENSG00000107611 | CUBN | −0.899 | 3.953 | −3.t57 | 0.003 | 0.022 | −1.788 |
| LNM Group 2 vs. Group 1 | ENSG00000107742 | SPOCK2 | −0.967 | 6.174 | −3.157 | 0.003 | 0.022 | −1.876 |
| LNM Group 2 vs. Group 1 | ENSG00000074370 | ATP2A3 | −1.120 | 6.273 | −3.158 | 0.003 | 0.022 | −1.887 |
| LNM Group 2 vs. Group 1 | ENSG00000163701 | IL17RE | 0.856 | 3.776 | 3.161 | 0.003 | 0.022 | −1.739 |
| LNM Group 2 vs. Group 1 | ENSG00000110328 | GALNT18 | 0.684 | 4.421 | 3.161 | 0.003 | 0.022 | −1.802 |
| LNM Group 2 vs. Group 1 | ENSG00000105514 | RAB3D | 0.738 | 5.993 | 3.163 | 0.003 | 0.021 | −1.851 |
| LNM Group 2 vs. Group 1 | ENSG00000086570 | FAT2 | 0.992 | 8.556 | 3.169 | 0.003 | 0.021 | −1.892 |
| LNM Group 2 vs. Group 1 | ENSG00000158747 | NBL1 | 0.660 | 6.766 | 3.169 | 0.003 | 0.021 | −1.855 |
| LNM Group 2 vs. Group 1 | ENSG00000172379 | ARNT2 | −1.060 | 3.551 | −3.174 | 0.003 | 0.021 | −1.773 |
| LNM Group 2 vs. Group 1 | ENSG00000205269 | TMEM170B | −0.657 | 3.849 | −3.176 | 0.003 | 0.021 | −1.762 |
| LNM Group 2 vs. Group 1 | ENSG00000154102 | C16orf74 | 0.719 | 3.996 | 3.176 | 0.003 | 0.021 | −1.712 |
| LNM Group 2 vs. Group 1 | ENSG00000115137 | DNAJC27 | −0.631 | 3.926 | −3.178 | 0.003 | 0.021 | −1.713 |
| LNM Group 2 vs. Group 1 | ENSG00000166444 | ST5 | 0.680 | 6.753 | 3.185 | 0.003 | 0.021 | −1.833 |
| LNM Group 2 vs. Group 1 | ENSG00000148200 | NR6A1 | 0.825 | 3.929 | 3.187 | 0.003 | 0.020 | −1.711 |
| LNM Group 2 vs. Group 1 | ENSG00000181896 | ZNF101 | −0.829 | 6.042 | −3.188 | 0.003 | 0.020 | −1.801 |
| LNM Group 2 vs. Group 1 | ENSG00000165359 | INTS6L | −0.859 | 5.518 | −3.191 | 0.003 | 0.020 | −1.750 |
| LNM Group 2 vs. Group 1 | ENSG00000056972 | TRAF3IP2 | 0.619 | 5.913 | 3.191 | 0.003 | 0.020 | −1.785 |
| LNM Group 2 vs. Group 1 | ENSG00000072682 | P4HA2 | 0.910 | 5.644 | 3.194 | 0.003 | 0.020 | −1.700 |
| LNM Group 2 vs. Group 1 | ENSG00000198053 | SIRPA | 0.594 | 6.472 | 3.196 | 0.003 | 0.020 | −1.754 |
| LNM Group 2 vs. Group 1 | ENSG00000168542 | COL3A1 | 1.223 | 11.045 | 3.200 | 0.003 | 0.020 | −1.786 |
| LNM Group 2 vs. Group 1 | ENSG00000158517 | NCF1 | −0.854 | 4.067 | −3.201 | 0.003 | 0.020 | −1.669 |
| LNM Group 2 vs. Group 1 | ENSG00000172403 | SYNPO2 | −1.116 | 5.885 | −3.201 | 0.003 | 0.020 | −1.768 |
| LNM Group 2 vs. Group 1 | ENSG00000100342 | APOL1 | 1.056 | 6.563 | 3.202 | 0.003 | 0.020 | −1.780 |
| LNM Group 2 vs. Group 1 | ENSG00000131981 | LGALS3 | 0.662 | 6.015 | 3.203 | 0.003 | 0.020 | −1.748 |
| LNM Group 2 vs. Group 1 | ENSG00000005379 | TSPOAP1 | −0.706 | 4.770 | −3.203 | 0.003 | 0.020 | −1.728 |
| LNM Group 2 vs. Group 1 | ENSG00000088305 | DNMT3B | 0.721 | 3.470 | 3.202 | 0.003 | 0.020 | −1.642 |
| LNM Group 2 vs. Group 1 | ENSG00000163702 | ID7RC | 0.722 | 4.316 | 3.205 | 0.003 | 0.020 | −1.645 |
| LNM Group 2 vs. Group 1 | ENSG00000148926 | ADM | 0.932 | 5.508 | 3.207 | 0.003 | 0.020 | −1.746 |
| LNM Group 2 vs. Group 1 | ENSG00000158163 | DZIP1L | 0.842 | 4.371 | 3.210 | 0.003 | 0.020 | −1.643 |
| LNM Group 2 vs. Group 1 | ENSG00000187961 | KLHL17 | 0.611 | 3.662 | 3.214 | 0.003 | 0.020 | −1.614 |
| LNM Group 2 vs. Group 1 | ENSG00000075618 | FSCN1 | 0.832 | 7.964 | 3.216 | 0.003 | 0.020 | −1.769 |
| LNM Group 2 vs. Group 1 | ENSG00000185432 | METTL7A | −0.754 | 5.754 | −3.220 | 0.003 | 0.019 | −1.725 |
| LNM Group 2 vs. Group 1 | ENSG00000168675 | LDLRAD4 | −0.632 | 6.335 | −3.222 | 0.002 | 0.019 | −1.741 |
| LNM Group 2 vs. Group 1 | ENSG00000141526 | SLC16A3 | 0.633 | 6.890 | 3.223 | 0.002 | 0.019 | −1.751 |
| LNM Group 2 vs. Group 1 | ENSG00000130844 | ZNF331 | −0.948 | 5.844 | −3.224 | 0.002 | 0.019 | −1.690 |
| LNM Group 2 vs. Group 1 | ENSG00000005844 | ITGAL | −0.959 | 5.812 | −3.235 | 0.002 | 0.019 | −1.641 |
| LNM Group 2 vs. Group 1 | ENSG00000142173 | COL6A2 | 1.014 | 8.760 | 3.238 | 0.002 | 0.019 | −1.717 |
| LNM Group 2 vs. Group 1 | ENSG00000130518 | KIAA1683 | −0.693 | 4.289 | −3.239 | 0.002 | 0.019 | −1.609 |
| LNM Group 2 vs. Group 1 | ENSG00000130755 | GMFG | −0.901 | 4.241 | −3.240 | 0.002 | 0.019 | −1.569 |
| LNM Group 2 vs. Group 1 | ENSG00000067836 | ROGDI | 0.613 | 3.801 | 3.238 | 0.002 | 0.019 | −1.549 |
| LNM Group 2 vs. Group 1 | ENSG00000162777 | DENND2D | −0.640 | 5.905 | −3.242 | 0.002 | 0.019 | −1.658 |
| LNM Group 2 vs. Group 1 | ENSG00000162511 | LAPTM5 | −0.652 | 7.971 | −3.243 | 0.002 | 0.019 | −1.697 |
| LNM Group 2 vs. Group 1 | ENSG00000089820 | ARHGAP4 | −0.764 | 6.017 | −3.244 | 0.002 | 0.019 | −1.646 |
| LNM Group 2 vs. Group 1 | ENSG00000214193 | SH3D21 | 0.946 | 5.055 | 3.244 | 0.002 | 0.019 | −1.603 |
| LNM Group 2 vs. Group 1 | ENSG00000115163 | CENPA | 0.667 | 3.216 | 3.251 | 0.002 | 0.018 | −1.520 |
| LNM Group 2 vs. Group 1 | ENSG00000133574 | GIMAP4 | −0.862 | 4.506 | −3.254 | 0.002 | 0.018 | −1.535 |
| LNM Group 2 vs. Group 1 | ENSG00000146242 | TPBG | 0.760 | 5.573 | 3.256 | 0.002 | 0.018 | −1.606 |
| LNM Group 2 vs. Group 1 | ENSG00000121858 | TNFSF10 | 0.781 | 6.802 | 3.258 | 0.002 | 0.018 | −1.658 |
| LNM Group 2 vs. Group 1 | ENSG00000186081 | KRT5 | 1.037 | 10.894 | 3.260 | 0.002 | 0.018 | −1.604 |
| LNM Group 2 vs. Group 1 | ENSG00000143119 | CD53 | −0.822 | 6.129 | −3.261 | 0.002 | 0.018 | −1.582 |
| LNM Group 2 vs. Group 1 | ENSG00000140564 | FURIN | 0.609 | 7.092 | 3.261 | 0.002 | 0.018 | −1.662 |
| LNM Group 2 vs. Group 1 | ENSG00000130940 | CASZ1 | 0.815 | 6.027 | 3.261 | 0.002 | 0.018 | −1.646 |
| LNM Group 2 vs. Group 1 | ENSG00000115687 | PASK | −0.610 | 6.105 | −3.264 | 0.002 | 0.018 | −1.641 |
| LNM Group 2 vs. Group 1 | ENSG00000168389 | MFSD2A | 1.066 | 4.277 | 3.266 | 0.002 | 0.018 | −1.498 |
| LNM Group 2 vs. Group 1 | ENSG00000133477 | FAM83F | 0.950 | 6.681 | 3.274 | 0.002 | 0.018 | −1.622 |
| LNM Group 2 vs. Group 1 | ENSG00000167642 | SPINT2 | 0.665 | 6.935 | 3.275 | 0.002 | 0.018 | −1.610 |
| LNM Group 2 vs. Group 1 | ENSG00000180884 | ZNF792 | −0.593 | 3.505 | −3.285 | 0.002 | 0.017 | −1.433 |
| LNM Group 2 vs. Group 1 | ENSG00000122122 | SASH3 | −0.917 | 4.957 | −3.288 | 0.002 | 0.017 | −1.477 |
| LNM Group 2 vs. Group 1 | ENSG00000121104 | FAM117A | −0.596 | 4.152 | −3.287 | 0.002 | 0.017 | −1.470 |
| LNM Group 2 vs. Group 1 | ENSG00000141441 | GAREM1 | 0.662 | 4.653 | 3.294 | 0.002 | 0.017 | −1.487 |
| LNM Group 2 vs. Group 1 | ENSG00000196739 | COL27A1 | 0.799 | 7.510 | 3.295 | 0.002 | 0.017 | −1.574 |
| LNM Group 2 vs. Group 1 | ENSG00000174564 | IL20RB | 1.068 | 5.031 | 3.297 | 0.002 | 0.017 | −1.471 |
| LNM Group 2 vs. Group 1 | ENSG00000005884 | ITGA3 | 0.768 | 8.088 | 3.298 | 0.002 | 0.017 | −1.555 |
| LNM Group 2 vs. Group 1 | ENSG00000183963 | SMTN | 0.841 | 6.410 | 3.309 | 0.002 | 0.016 | −1.495 |
| LNM Group 2 vs. Group 1 | ENSG00000163362 | C1orf106 | 0.687 | 6.096 | 3.311 | 0.002 | 0.016 | −1.502 |
| LNM Group 2 vs. Group 1 | ENSG00000121579 | NAA50 | 0.596 | 7.985 | 3.316 | 0.002 | 0.016 | −1.522 |
| LNM Group 2 vs. Group 1 | ENSG00000104341 | LAPTM4B | 0.631 | 6.305 | 3.316 | 0.002 | 0.016 | −1.462 |
| LNM Group 2 vs. Group 1 | ENSG00000122862 | SRGN | −0.607 | 6.383 | −3.319 | 0.002 | 0.016 | −1.437 |
| LNM Group 2 vs. Group 1 | ENSG00000163472 | TMEM79 | 1.075 | 4.833 | 3.320 | 0.002 | 0.016 | −1.370 |
| LNM Group 2 vs. Group 1 | ENSG00000069020 | MAST4 | 0.704 | 7.282 | 3.323 | 0.002 | 0.016 | −1.474 |
| LNM Group 2 vs. Group 1 | ENSG00000074211 | PPP2R2C | 1.124 | 5.856 | 3.326 | 0.002 | 0.016 | −1.366 |
| LNM Group 2 vs. Group 1 | ENSG00000112576 | CCND3 | −0.761 | 6.494 | −3.326 | 0.002 | 0.016 | −1.464 |
| LNM Group 2 vs. Group 1 | ENSG00000076555 | ACACB | −0.827 | 5.486 | −3.327 | 0.002 | 0.016 | −1.414 |
| LNM Group 2 vs. Group 1 | ENSG00000109079 | TNFAIP1 | 0.688 | 6.362 | 3.327 | 0.002 | 0.016 | −1 459 |
| LNM Group 2 vs. Group 1 | ENSG00000178878 | APOLD1 | −0.736 | 5.610 | −3.330 | 0.002 | 0.016 | −1.424 |
| LNM Group 2 vs. Group 1 | ENSG00000196639 | HRH1 | 0.963 | 3.668 | 3.331 | 0.002 | 0.016 | −1.329 |
| LNM Group 2 vs. Group f | ENSG00000117215 | PLA2G2D | −1.423 | 3.360 | −3.336 | 0.002 | 0.016 | −1.331 |
| LNM Group 2 vs. Group 1 | ENSG00000153898 | MCOLN2 | −0.825 | 4.281 | −3.337 | 0.002 | 0.016 | −1.330 |
| LNM Group 2 vs. Group 1 | ENSG00000069702 | TGFBR3 | −1.169 | 5.192 | −3.339 | 0.002 | 0.016 | −1.423 |
| LNM Group 2 vs. Group 1 | ENSG00000134769 | DTNA | −0.841 | 3.506 | −3.340 | 0.002 | 0.016 | −1.315 |
| LNM Group 2 vs. Group 1 | ENSG00000141576 | RNF157 | −1.072 | 3.811 | −3.342 | 0.002 | 0.016 | −1.309 |
| LNM Group 2 vs. Group 1 | ENSG00000176358 | TAC4 | −0.736 | 2.555 | −3.342 | 0.002 | 0.016 | −1.289 |
| LNM Group 2 vs. Group 1 | ENSG00000075275 | CELSR1 | 0.627 | 7.814 | 3.349 | 0.002 | 0.015 | −1.437 |
| LNM Group 2 vs. Group 1 | ENSG00000114166 | KAT2B | −0.616 | 5.467 | −3.352 | 0.002 | 0.015 | −1.327 |
| LNM Group 2 vs. Group 1 | ENSG00000115657 | ABCB6 | 0.602 | 4.620 | 3.354 | 0.002 | 0.015 | −1.311 |
| LNM Group 2 vs. Group 1 | ENSG00000176170 | SPHK1 | 0.970 | 5.431 | 3.358 | 0.002 | 0.015 | −1.325 |
| LNM Group 2 vs. Group 1 | ENSG00000041353 | RAB27B | 1.111 | 4.986 | 3.359 | 0.002 | 0.015 | −1.299 |
| LNM Group 2 vs. Group 1 | ENSG00000074964 | ARHGEF10L | 0.696 | 5.531 | 3.359 | 0.002 | 0.015 | −1.315 |
| LNM Group 2 vs. Group 1 | ENSG00000116299 | KIAA1324 | −1.125 | 4.048 | −3.360 | 0.002 | 0.015 | −1.262 |
| LNM Group 2 vs. Group 1 | ENSG00000213859 | KCTD11 | 0.799 | 5.282 | 3.361 | 0.002 | 0.015 | −1.299 |
| LNM Group 2 vs. Group 1 | ENSG00000167306 | MYO5B | 1.523 | 5.841 | 3.362 | 0.002 | 0.015 | −1.371 |
| LNM Group 2 vs. Group 1 | ENSG00000258555 | SPECC1L- | −0.595 | 2.914 | −3.363 | 0.002 | 0.015 | −1.234 |
| | | ADORA2A | | | | | | |
| LNM Group 2 vs. Group 1 | ENSG00000115884 | SDC1 | 0.956 | 8.680 | 3.368 | 0.002 | 0.015 | −1.382 |
| LNM Group 2 vs. Group 1 | ENSG00000171444 | MCC | 0.714 | 7.023 | 3.370 | 0.002 | 0.015 | −1.367 |
| LNM Group 2 vs. Group 1 | ENSG00000261739 | GOLGA8S | −0.590 | 2.168 | −3.370 | 0.002 | 0.015 | −1.200 |
| LNM Group 2 vs. Group 1 | ENSG00000164070 | HSPA4L | 0.706 | 5.893 | 3.374 | 0.002 | 0.015 | −1.347 |
| LNM Group 2 vs. Group 1 | ENSG00000198440 | ZNF583 | −0.745 | 3.358 | −3.376 | 0.002 | 0.015 | −1.257 |
| LNM Group 2 vs. Group 1 | ENSG00000145555 | MYO10 | 0.886 | 7.441 | 3.379 | 0.002 | 0.015 | −1.354 |
| LNM Group 2 vs. Group 1 | ENSG00000140030 | GPR65 | −0.658 | 3.563 | −3.380 | 0.002 | 0.014 | −1.192 |
| LNM Group 2 vs. Group 1 | ENSG00000187764 | SEMA4D | −0.788 | 7.085 | −3.387 | 0.002 | 0.014 | −1.334 |
| LNM Group 2 vs. Group 1 | ENSG00000139116 | KIF21A | 0.755 | 5.631 | 3.387 | 0.002 | 0.014 | −1.301 |
| LNM Group 2 vs. Group 1 | ENSG00000198168 | SVIP | −0.765 | 4.362 | −3.390 | 0.002 | 0.014 | −1.242 |
| LNM Group 2 vs. Group 1 | ENSG00000221955 | SLC12A8 | 0.770 | 4.876 | 3.390 | 0.002 | 0.014 | −1.242 |
| LNM Group 2 vs. Group 1 | ENSG00000112655 | PTK7 | 0.665 | 7.125 | 3.403 | 0.001 | 0.014 | −1.282 |
| LNM Group 2 vs. Group 1 | ENSG00000241399 | CD302 | −0.618 | 4.228 | −3.402 | 0.001 | 0.014 | −1.180 |
| LNM Group 2 vs. Group 1 | ENSG00000236334 | PPIAL4G | −0.588 | 3.651 | −3.406 | 0.001 | 0.014 | −1.142 |
| LNM Group 2 vs. Group 1 | ENSG00000136541 | ERMN | −0.768 | 2.728 | −3.408 | 0.001 | 0.014 | −1.120 |
| LNM Group 2 vs. Group 1 | ENSG00000106211 | HSPB1 | 0.825 | 7.662 | 3.410 | 0.001 | 0.014 | −1.271 |
| LNM Group 2 vs. Group 1 | ENSG00000141524 | TMC6 | −0.585 | 7.071 | −3.410 | 0.001 | 0.014 | −1.275 |
| LNM Group 2 vs. Group 1 | ENSG00000162493 | PDPN | 1.082 | 5.911 | 3.413 | 0.001 | 0.014 | −1.222 |
| LNM Group 2 vs. Group 1 | ENSG00000240972 | MIF | 0.608 | 5.654 | 3.411 | 0.001 | 0.014 | −1.212 |
| LNM Group 2 vs. Group 1 | ENSG00000179163 | FUCA1 | −0.924 | 4.552 | −3.414 | 0.001 | 0.014 | −1.151 |
| LNM Group 2 vs. Group 1 | ENSG00000176845 | METRNL | 0.693 | 5.430 | 3.414 | 0.001 | 0.014 | −1.195 |
| LNM Group 2 vs. Group 1 | ENSG00000105643 | ARRDC2 | −0.641 | 5.605 | −3.415 | 0.001 | 0.014 | −1.207 |
| LNM Group 2 vs. Group 1 | ENSG00000180353 | HCLS1 | −0.729 | 6.474 | −3.418 | 0.001 | 0.014 | −1.217 |
| LNM Group 2 vs. Group 1 | ENSG00000150637 | CD226 | −0.821 | 5.227 | −3.422 | 0.001 | 0.013 | −1.153 |
| LNM Group 2 vs. Group 1 | ENSG00000172508 | CARNS1 | −0.880 | 3.664 | −3.423 | 0.001 | 0.013 | −1.122 |
| LNM Group 2 vs. Group 1 | ENSG00000103042 | SLC38A7 | 0.600 | 5.188 | 3.423 | 0.001 | 0.013 | −1.144 |
| LNM Group 2 vs. Group 1 | ENSG00000136104 | RNASEH2B | −1.091 | 6.086 | −3.426 | 0.001 | 0.013 | −1.164 |
| LNM Group 2 vs. Group 1 | ENSG00000184271 | POU6F1 | −0.932 | 4.172 | −3.427 | 0.001 | 0.013 | −1.135 |
| LNM Group 2 vs. Group 1 | ENSG00000152642 | GPD1L | −0.841 | 4.336 | −3.430 | 0.001 | 0.013 | −1.081 |
| LNM Group 2 vs. Group 1 | ENSG00000124126 | PREX1 | −0.629 | 6.531 | −3.437 | 0.001 | 0.013 | −1.164 |
| LNM Group 2 vs. Group 1 | ENSG00000172830 | SSH3 | 0.605 | 5.337 | 3.438 | 0.001 | 0.013 | −1.102 |
| LNM Group 2 vs. Group 1 | ENSG00000164674 | SYTL3 | −0.956 | 4.341 | −3.441 | 0.001 | 0.013 | −1.069 |
| LNM Group 2 vs. Group 1 | ENSG00000147813 | NAPRT | 0.636 | 5.317 | 3.441 | 0.001 | 0.013 | −1.122 |
| LNM Group 2 vs. Group 1 | ENSG00000155307 | SAMSN1 | −0.779 | 4.594 | −3.443 | 0.001 | 0.013 | −1.078 |
| LNM Group 2 vs. Group 1 | ENSG00000075826 | SEC31B | −0.635 | 5.082 | −3.442 | 0.001 | 0.013 | −1.102 |
| LNM Group 2 vs. Group 1 | ENSG00000103257 | SLC7A5 | 0.744 | 8.317 | 3.448 | 0.001 | 0.013 | −1.175 |
| LNM Group 2 vs. Group 1 | ENSG00000102879 | CORO1A | −1.061 | 6.289 | −3.449 | 0.001 | 0.013 | −1.130 |
| LNM Group 2 vs. Group 1 | ENSG00000198324 | FAM109A | 0.863 | 3.686 | 3.451 | 0.001 | 0.013 | −1.019 |
| LNM Group 2 vs. Group 1 | ENSG00000160360 | GPSM1 | 0.640 | 4.598 | 3.453 | 0.001 | 0.013 | −1.083 |
| LNM Group 2 vs. Group 1 | ENSG00000152127 | MGAT5 | −0.593 | 7.209 | −3.456 | 0.001 | 0.013 | −1.152 |
| LNM Group 2 vs. Group 1 | ENSG00000240038 | AMY2B | −0.832 | 4.752 | −3.458 | 0.001 | 0.013 | −1.040 |
| LNM Group 2 vs. Group 1 | ENSG00000135069 | PSAT1 | 0.665 | 4.241 | 3.457 | 0.001 | 0.013 | −1.072 |
| LNM Group 2 vs. Group 1 | ENSG00000145246 | ATP10D | 0.604 | 6.334 | 3.459 | 0.001 | 0.013 | −1.078 |
| LNM Group 2 vs. Group 1 | ENSG00000243364 | EFNA4 | 0.678 | 2.767 | 3.458 | 0.001 | 0.013 | −0.986 |
| LNM Group 2 vs. Group 1 | ENSG00000143545 | RAB13 | 0.587 | 5.188 | 3.461 | 0.001 | 0.013 | −1.057 |
| LNM Group 2 vs. Group 1 | ENSG00000101447 | FAM83D | 0.678 | 4.293 | 3.468 | 0.001 | 0.012 | −1.041 |
| LNM Group 2 vs. Group 1 | ENSG00000076770 | MBNL3 | −0.601 | 6.216 | −3.469 | 0.001 | 0.012 | −1.076 |
| LNM Group 2 vs. Group 1 | ENSG00000074800 | ENO1 | 0.611 | 9.193 | 3.470 | 0.001 | 0.012 | −1.106 |
| LNM Group 2 vs. Group 1 | ENSG00000127955 | GNAI1 | 0.868 | 4.967 | 3.477 | 0.001 | 0.012 | −1.000 |
| LNM Group 2 vs. Group 1 | ENSG00000138380 | CARF | −0.626 | 4.647 | −3.476 | 0.001 | 0.012 | −0.983 |
| LNM Group 2 vs. Group 1 | ENSG00000169991 | IFFO2 | 1.009 | 6.823 | 3.479 | 0.001 | 0.012 | −1.086 |
| LNM Group 2 vs. Group 1 | ENSG00000134668 | SPOCD1 | 1.268 | 3.729 | 3.482 | 0.001 | 0.012 | −0.941 |
| LNM Group 2 vs. Group 1 | ENSG00000132481 | TRIM47 | 0.615 | 4.978 | 3.490 | 0.001 | 0.012 | −0.962 |
| LNM Group 2 vs. Group 1 | ENSG00000147650 | LRP12 | 0.636 | 5.335 | 3.491 | 0.001 | 0.012 | −1.009 |
| LNM Group 2 vs. Group 1 | ENSG00000188483 | IER5L | 0.704 | 3.806 | 3.493 | 0.001 | 0.012 | −0.938 |
| LNM Group 2 vs. Group 1 | ENSG00000185905 | C16orf54 | −0.719 | 4.104 | −3.500 | 0.001 | 0.012 | −0.910 |
| LNM Group 2 vs. Group 1 | ENSG00000197077 | KIAA1671 | 0.614 | 7.340 | 3.502 | 0.001 | 0.012 | −1.028 |
| LNM Group 2 vs. Group 1 | ENSG00000111728 | ST8SIA1 | −0.780 | 4.947 | −3.504 | 0.001 | 0.012 | −0.962 |
| LNM Group 2 vs. Group 1 | ENSG00000171729 | TMEM51 | 0.710 | 3.740 | 3.510 | 0.001 | 0.012 | −0.881 |
| LNM Group 2 vs. Group 1 | ENSG00000162692 | VCAM1 | −0.978 | 4.776 | −3.512 | 0.001 | 0.012 | −0.926 |
| LNM Group 2 vs. Group 1 | ENSG00000179431 | FJX1 | 0.997 | 4.001 | 3.515 | 0.001 | 0.011 | −0.871 |
| LNM Group 2 vs. Group 1 | ENSG00000162390 | ACOT11 | 0.627 | 6.456 | 3.515 | 0.001 | 0.011 | −0.981 |
| LNM Group 2 vs. Group 1 | ENSG00000205336 | ADGRG1 | 0.803 | 6.857 | 3.516 | 0.001 | 0.011 | −0.970 |
| LNM Group 2 vs. Group 1 | ENSG00000171522 | PTGER4 | −0.881 | 4.885 | −3.518 | 0.001 | 0.011 | −0.911 |
| LNM Group 2 vs. Group 1 | ENSG00000104892 | KLC3 | 0.765 | 4.090 | 3.520 | 0.001 | 0.011 | −0.866 |
| LNM Group 2 vs. Group 1 | ENSG00000134294 | SLC38A2 | 0.604 | 9.246 | 3.520 | 0.001 | 0.011 | −0.974 |
| LNM Group 2 vs. Group 1 | ENSG00000196172 | ZNF681 | −0.868 | 3.490 | −3.523 | 0.001 | 0.011 | −0.870 |
| LNM Group 2 vs. Group 1 | ENSG00000177045 | SIX5 | 0.636 | 3.844 | 3.525 | 0.001 | 0.011 | −0.834 |
| LNM Group 2 vs. Group 1 | ENSG00000145287 | PLAC8 | −1.389 | 4.704 | −3.531 | 0.001 | 0.011 | −0.881 |
| LNM Group 2 vs. Group 1 | ENSG00000062038 | CDH3 | 1.020 | 7.105 | 3.532 | 0.001 | 0.011 | −0.933 |
| LNM Group 2 vs. Group 1 | ENSG00000187840 | EIF4EBP1 | 0.721 | 4.405 | 3.532 | 0.001 | 0.011 | −0.861 |
| LNM Group 2 vs. Group 1 | ENSG00000110934 | BIN2 | −0.919 | 4.508 | −3.533 | 0.001 | 0.011 | −0.823 |
| LNM Group 2 vs. Group 1 | ENSG00000070614 | NDST1 | 0.597 | 7.473 | 3.535 | 0.001 | 0.011 | −0.936 |
| LNM Group 2 vs. Group 1 | ENSG00000105373 | GLTSCR2 | −0.628 | 6.838 | −3.536 | 0.001 | 0.011 | −0.922 |
| LNM Group 2 vs. Group 1 | ENSG00000143851 | PTPN7 | −0.729 | 5.008 | −3.537 | 0.001 | 0.011 | −0.849 |
| LNM Group 2 vs. Group 1 | ENSG00000163032 | VSNL1 | 0.919 | 4.237 | 3.540 | 0.001 | 0.011 | −0.823 |
| LNM Group 2 vs. Group 1 | ENSG00000085063 | CD59 | 0.642 | 7.901 | 3.542 | 0.001 | 0.011 | −0.926 |
| LNM Group 2 vs. Group 1 | ENSG00000141401 | IMPA2 | 0.838 | 5.438 | 3.543 | 0.001 | 0.011 | −0.831 |
| LNM Group 2 vs. Group 1 | ENSG00000136848 | DAB2IP | 0.593 | 6.769 | 3.541 | 0.001 | 0.011 | −0.914 |
| LNM Group 2 vs. Group 1 | ENSG00000159496 | RGL4 | −0.615 | 3.332 | −3.541 | 0.001 | 0.011 | −0.791 |
| LNM Group 2 vs. Group 1 | ENSG00000164938 | TP53INP1 | −0.804 | 7.254 | −3.547 | 0.001 | 0.011 | −0.906 |
| LNM Group 2 vs. Group 1 | ENSG00000196411 | EPHB4 | 0.633 | 6.613 | 3.551 | 0.001 | 0.011 | −0.893 |
| LNM Group 2 vs. Group 1 | ENSG00000154310 | TNIK | −0.678 | 4.871 | −3.552 | 0.001 | 0.011 | −0.830 |
| LNM Group 2 vs. Group 1 | ENSG00000198720 | ANKRD13B | 0.766 | 3.620 | 3.553 | 0.001 | 0.011 | −0.795 |
| LNM Group 2 vs. Group 1 | ENSG00000178567 | EPM2AIP1 | −0.603 | 6.191 | −3.553 | 0.001 | 0.011 | −0.851 |
| LNM Group 2 vs. Group 1 | ENSG00000096696 | DSP | 1.192 | 10.538 | 3.556 | 0.001 | 0.011 | −0.848 |
| LNM Group 2 vs. Group 1 | ENSG00000120915 | EPHX2 | −0.711 | 3.732 | −3.555 | 0.001 | 0.011 | −0.762 |
| LNM Group 2 vs. Group 1 | ENSG00000072840 | EVC | 0.717 | 6.006 | 3.560 | 0.001 | 0.011 | −0.825 |
| LNM Group 2 vs. Group 1 | ENSG00000176928 | GCNT4 | −0.964 | 3.522 | −3.560 | 0.001 | 0.011 | −0.748 |
| LNM Group 2 vs. Group 1 | ENSG00000111424 | VDR | 0.587 | 5.716 | 3.558 | 0.001 | 0.011 | −0.796 |
| LNM Group 2 vs. Group 1 | ENSG00000140398 | NEIL1 | −0.752 | 4.745 | −3.562 | 0.001 | 0.011 | −0.770 |
| LNM Group 2 vs. Group 1 | ENSG00000132681 | ATP1A4 | −0.760 | 2.588 | −3.560 | 0.001 | 0.011 | −0.726 |
| LNM Group 2 vs. Group 1 | ENSG00000099849 | RASSF7 | 0.673 | 3.949 | 3.560 | 0.001 | 0.011 | −0.759 |
| LNM Group 2 vs. Group 1 | ENSG00000154027 | AK5 | −0.929 | 2.111 | −3.563 | 0.001 | 0.011 | −0.719 |
| LNM Group 2 vs. Group 1 | ENSG00000103855 | CD276 | 0.772 | 6.318 | 3.570 | 0.001 | 0.011 | −0.828 |
| LNM Group 2 vs. Group 1 | ENSG00000106479 | ZNF862 | −0.714 | 4.693 | −3.570 | 0.001 | 0.011 | −0.745 |
| LNM Group 2 vs. Group 1 | ENSG00000172216 | CEBPB | 0.677 | 6.083 | 3.577 | 0.001 | 0.010 | −0.786 |
| LNM Group 2 vs. Group 1 | ENSG00000134909 | ARHGAP32 | 0.769 | 6.750 | 3.582 | 0.001 | 0.010 | −0.784 |
| LNM Group 2 vs. Group 1 | ENSG00000157551 | KCNJ15 | 1.065 | 6.175 | 3.587 | 0.001 | 0.010 | −0.752 |
| LNM Group 2 vs. Group 1 | ENSG00000182108 | DEXI | 0.954 | 3.554 | 3.588 | 0.001 | 0.010 | −0.671 |
| LNM Group 2 vs. Group 1 | ENSG00000132793 | LPIN3 | 0.774 | 5.520 | 3.588 | 0.001 | 0.010 | −0.734 |
| LNM Group 2 vs. Group 1 | ENSG00000107099 | DOCK8 | −0.765 | 7.251 | −3.593 | 0.001 | 0.010 | −0.770 |
| LNM Group 2 vs. Group 1 | ENSG00000149573 | MPZL2 | 1.101 | 5.757 | 3.595 | 0.001 | 0.010 | −0.685 |
| LNM Group 2 vs. Group 1 | ENSG00000149257 | SERPINH1 | 0.898 | 7.086 | 3.595 | 0.001 | 0.010 | −0.771 |
| LNM Group 2 vs. Group 1 | ENSG00000127528 | KLF2 | −1.188 | 5.222 | −3.597 | 0.001 | 0.010 | −0.695 |
| LNM Group 2 vs. Group 1 | ENSG00000158292 | GPR153 | 0.923 | 5.020 | 3.598 | 0.001 | 0.010 | −0.680 |
| LNM Group 2 vs. Group 1 | ENSG00000117984 | CTSD | 0.646 | 8.610 | 3.600 | 0.001 | 0.010 | −0.768 |
| LNM Group 2 vs. Group 1 | ENSG00000117394 | SLC2A1 | 1.092 | 8.211 | 3.602 | 0.001 | 0.010 | −0.760 |
| LNM Group 2 vs. Group 1 | ENSG00000171867 | PRNP | 0.719 | 7.884 | 3.605 | 0.001 | 0.010 | −0.752 |
| LNM Group 2 vs. Group 1 | ENSG00000007384 | RHBDF1 | 0.698 | 4.916 | 3.603 | 0.001 | 0.010 | −0.652 |
| LNM Group 2 vs. Group 1 | ENSG00000103044 | HAS3 | 1.449 | 5.418 | 3.607 | 0.001 | 0.010 | −0.695 |
| LNM Group 2 vs. Group 1 | ENSG00000197747 | S100A10 | 0.731 | 7.218 | 3.608 | 0.001 | 0.010 | −0.733 |
| LNM Group 2 vs. Group 1 | ENSG00000042980 | ADAM28 | −1.122 | 6.025 | −3.609 | 0.001 | 0.010 | −0.728 |
| LNM Group 2 vs. Group 1 | ENSG00000197471 | SPN | −0.618 | 5.624 | −3.612 | 0.001 | 0.010 | −0.689 |
| LNM Group 2 vs. Group 1 | ENSG00000109684 | CLNK | −0.628 | 3.073 | −3.614 | 0.001 | 0.010 | −0.594 |
| LNM Group 2 vs. Group 1 | ENSG00000151702 | FLI1 | −0.680 | 5.747 | −3.619 | 0.001 | 0.010 | −0.654 |
| LNM Group 2 vs. Group 1 | ENSG00000157379 | DHRS1 | 0.608 | 5.200 | 3.618 | 0.001 | 0.010 | −0.600 |
| LNM Group 2 vs. Group 1 | ENSG00000106948 | AKNA | −0.857 | 7.642 | −3.624 | 0.001 | 0.010 | −0.706 |
| LNM Group 2 vs. Group 1 | ENSG00000100027 | YPEL1 | −0.668 | 3.522 | −3.622 | 0.001 | 0.010 | −0.590 |
| LNM Group 2 vs. Group 1 | ENSG00000147852 | VLDLR | 1.420 | 4.913 | 3.627 | 0.001 | 0.010 | −0.601 |
| LNM Group 2 vs. Group 1 | ENSG00000104903 | LYL1 | −0.794 | 3.187 | −3.627 | 0.001 | 0.009 | −0.566 |
| LNM Group 2 vs. Group 1 | ENSG00000197872 | FAM49A | −0.822 | 4.903 | −3.629 | 0.001 | 0.009 | −0.586 |
| LNM Group 2 vs. Group 1 | ENSG00000095015 | MAP3K1 | −0.602 | 6.758 | −3.630 | 0.001 | 0.009 | −0.655 |
| LNM Group 2 vs. Group 1 | ENSG00000143537 | ADAM15 | 0.619 | 6.774 | 3.635 | 0.001 | 0.009 | −0.647 |
| LNM Group 2 vs. Group 1 | ENSG00000134278 | SPIRE1 | 0.742 | 5.444 | 3.638 | 0.001 | 0.009 | −0.620 |
| LNM Group 2 vs. Group 1 | ENSG00000100307 | CBX7 | −0.768 | 5.837 | −3.644 | 0.001 | 0.009 | −0.623 |
| LNM Group 2 vs. Group 1 | ENSG00000184205 | TSPYL2 | −0.845 | 4.746 | −3.647 | 0.001 | 0.009 | −0.540 |
| LNM Group 2 vs. Group 1 | ENSG00000143858 | SYT2 | −0.662 | 2.571 | −3.648 | 0.001 | 0.009 | −0.501 |
| LNM Group 2 vs. Group 1 | ENSG00000160654 | CD3G | −1.024 | 4.296 | −3.651 | 0.001 | 0.009 | −0.516 |
| LNM Group 2 vs. Group 1 | ENSG00000172819 | RARG | 0.736 | 5.790 | 3.658 | 0.001 | 0.009 | −0.568 |
| LNM Group 2 vs. Group 1 | ENSG00000J85989 | RASA3 | −0.645 | 5.993 | −3.657 | 0.00J | 0.009 | −0.567 |
| LNM Group 2 vs. Group 1 | ENSG00000271303 | SRXN1 | 0.815 | 5.501 | 3.662 | 0.001 | 0.009 | −0.529 |
| LNM Group 2 vs. Group 1 | ENSG00000134247 | PTGFRN | 0.783 | 7.605 | 3.664 | 0.001 | 0.009 | −0.593 |
| LNM Group 2 vs. Group 1 | ENSG00000146950 | SHROOM2 | 0.750 | 4.600 | 3.665 | 0.001 | 0.009 | −0.518 |
| LNM Group 2 vs. Group 1 | ENSG00000029153 | ARNTL2 | 0.887 | 6.565 | 3.669 | 0.001 | 0.009 | −0.579 |
| LNM Group 2 vs. Group 1 | ENSG00000158716 | DUSP23 | 0.663 | 3.095 | 3.667 | 0.001 | 0.009 | −0.446 |
| LNM Group 2 vs. Group 1 | ENSG00000161929 | SCIMP | −0.760 | 4.708 | −3.672 | 0.001 | 0.009 | −0.471 |
| LNM Group 2 vs. Group 1 | ENSG00000171680 | PLEKHG5 | 0.784 | 6.085 | 3.677 | 0.001 | 0.009 | −0.533 |
| LNM Group 2 vs. Group 1 | ENSG00000004777 | ARHGAP33 | −0.937 | 4.471 | −3.680 | 0.001 | 0.009 | −0.470 |
| LNM Group 2 vs. Group 1 | ENSG00000146757 | ZNF92 | −0.691 | 4.281 | −3.678 | 0.001 | 0.009 | −0.444 |
| LNM Group 2 vs. Group 1 | ENSG00000127995 | CASD1 | −0.596 | 5.388 | −3.682 | 0.001 | 0.009 | −0.479 |
| LNM Group 2 vs. Group 1 | ENSG00000083857 | FAT1 | 0.748 | 8.996 | 3.687 | 0.001 | 0.009 | −0.520 |
| LNM Group 2 vs. Group 1 | ENSG00000153814 | JAZF1 | −0.620 | 4.842 | −3.688 | 0.001 | 0.008 | −0.435 |
| LNM Group 2 vs. Group 1 | ENSG00000108679 | LGALS3BP | 0.641 | 7.639 | 3.691 | 0.001 | 0.008 | −0.522 |
| LNM Group 2 vs. Group 1 | ENSG00000173801 | JUP | 1.129 | 8.792 | 3.692 | 0.001 | 0.008 | −0.511 |
| LNM Group 2 vs. Group 1 | ENSG00000152582 | SPEF2 | −0.698 | 4.379 | −3.694 | 0.001 | 0.008 | −0.419 |
| LNM Group 2 vs. Group 1 | ENSG00000173207 | CKS1B | 0.598 | 4.649 | 3.694 | 0.001 | 0.008 | −0.417 |
| LNM Group 2 vs. Group 1 | ENSG00000146021 | KLHL3 | −0.746 | 4.481 | −3.695 | 0.001 | 0.008 | −0.391 |
| LNM Group 2 vs. Group 1 | ENSG00000140859 | KIFC3 | 0.611 | 6.109 | 3.697 | 0.001 | 0.008 | −0.464 |
| LNM Group 2 vs. Group 1 | ENSG00000147383 | NSDHL | 0.604 | 3.599 | 3.699 | 0.001 | 0.008 | −0.382 |
| LNM Group 2 vs. Group 1 | ENSG00000134317 | GRHL1 | 1.516 | 5.574 | 3.707 | 0.001 | 0.008 | −0.414 |
| LNM Group 2 vs. Group 1 | ENSG00000151883 | PARP8 | −0.586 | 6.635 | −3.709 | 0.001 | 0.008 | −0.444 |
| LNM Group 2 vs. Group 1 | ENSG00000130489 | SCO2 | 0.938 | 4.765 | 3.713 | 0.001 | 0.008 | −0.373 |
| LNM Group 2 vs. Group 1 | ENSG00000156675 | RAB11FIP1 | −0.616 | 6.671 | −3.713 | 0.001 | 0.008 | −0.454 |
| LNM Group 2 vs. Group 1 | ENSG00000213977 | TAX1BP3 | 0.656 | 6.094 | 3.723 | 0.001 | 0.008 | −0.361 |
| LNM Group 2 vs. Group 1 | ENSG00000153283 | CD96 | −0.898 | 4.602 | −3.729 | 0.001 | 0.008 | −0.316 |
| LNM Group 2 vs. Group 1 | ENSG00000145545 | SRD5A1 | 0.654 | 6.465 | 3.728 | 0.001 | 0.008 | −0.382 |
| LNM Group 2 vs. Group 1 | ENSG00000163219 | ARHGAP25 | −0.715 | 5.145 | −3.729 | 0.001 | 0.008 | −0.346 |
| LNM Group 2 vs. Group 1 | ENSG00000182580 | EPHB3 | 0.841 | 4.798 | 3.731 | 0.001 | 0.008 | −0.359 |
| LNM Group 2 vs. Group 1 | ENSG00000091622 | PITPNM3 | 0.878 | 5.210 | 3.732 | 0.001 | 0.008 | −0.359 |
| LNM Group 2 vs. Group 1 | ENSG00000100320 | RBFOX2 | 0.633 | 7.099 | 3.731 | 0.001 | 0.008 | −0.409 |
| LNM Group 2 vs. Group 1 | ENSG00000198673 | FAM19A2 | −0.662 | 2.686 | −3.731 | 0.001 | 0.008 | −0.278 |
| LNM Group 2 vs. Group 1 | ENSG00000135378 | PRRG4 | 0.716 | 6.128 | 3.733 | 0.001 | 0.008 | −0.364 |
| LNM Group 2 vs. Group 1 | ENSG00000089876 | DHX32 | 0.699 | 5.022 | 3.732 | 0.001 | 0.008 | −0.313 |
| LNM Group 2 vs. Group 1 | ENSG00000165655 | ZNF503 | 0.828 | 4.082 | 3.735 | 0.001 | 0.008 | −0.290 |
| LNM Group 2 vs. Group 1 | ENSG00000197562 | RAB40C | 0.602 | 4.749 | 3.739 | 0.001 | 0.008 | −0.297 |
| LNM Group 2 vs. Group 1 | ENSG00000106571 | GLI3 | 0.866 | 5.930 | 3.745 | 0.001 | 0.008 | −0.329 |
| LNM Group 2 vs. Group 1 | ENSG00000187994 | RINL | −1.440 | 4.181 | −3.746 | 0.001 | 0.008 | −0.285 |
| LNM Group 2 vs. Group 1 | ENSG00000126803 | HSPA2 | 1.068 | 4.861 | 3.755 | 0.001 | 0.008 | −0.286 |
| LNM Group 2 vs. Group 1 | ENSG00000118257 | NRP2 | 0.697 | 6.812 | 3.755 | 0.001 | 0.008 | −0.336 |
| LNM Group 2 vs. Group 1 | ENSG00000283652 | WDFY4 | −1.245 | 5.915 | −3.757 | 0.001 | 0.008 | −0.287 |
| LNM Group 2 vs. Group 1 | ENSG00000074706 | IPCEF1 | −1.360 | 4.468 | −3.761 | 0.001 | 0.007 | −0.232 |
| LNM Group 2 vs. Group 1 | ENSG00000127564 | PKMYT1 | 0.887 | 4.627 | 3.765 | 0.001 | 0.007 | −0.227 |
| LNM Group 2 vs. Group 1 | ENSG00000169504 | CLIC4 | 0.735 | 7.964 | 3.767 | 0.001 | 0.007 | −0.316 |
| LNM Group 2 vs. Group 1 | ENSG00000130517 | PGPEP1 | −0.648 | 5.549 | −3.770 | 0.001 | 0.007 | −0.260 |
| LNM Group 2 vs. Group 1 | ENSG00000166681 | BEX3 | 0.773 | 4.529 | 3.776 | 0.001 | 0.007 | −0.198 |
| LNM Group 2 vs. Group 1 | ENSG00000158220 | ESYT3 | 0.855 | 3.295 | 3.780 | 0.000 | 0.007 | −0.174 |
| LNM Group 2 vs. Group 1 | ENSG00000141736 | ERBB2 | 0.736 | 6.726 | 3.781 | 0.000 | 0.007 | −0.263 |
| LNM Group 2 vs. Group 1 | ENSG00000152684 | PELO | 0.597 | 4.472 | 3.780 | 0.000 | 0.007 | −0.186 |
| LNM Group 2 vs. Group 1 | ENSG00000082293 | COL19A1 | −1.331 | 3.185 | −3.786 | 0.000 | 0.007 | −0.151 |
| LNM Group 2 vs. Group 1 | ENSG00000157227 | MMP14 | 0.890 | 8.070 | 3.788 | 0.000 | 0.007 | −0.249 |
| LNM Group 2 vs. Group 1 | ENSG00000078900 | TP73 | 0.797 | 3.972 | 3.790 | 0.000 | 0.007 | −0.192 |
| LNM Group 2 vs. Group 1 | ENSG00000164398 | ACSL6 | −1.329 | 3.251 | −3.791 | 0.000 | 0.007 | −0.127 |
| LNM Group 2 vs. Group 1 | ENSG00000128309 | MPST | 0.637 | 5.345 | 3.788 | 0.000 | 0.007 | −0.194 |
| LNM Group 2 vs. Group 1 | ENSG00000096996 | IL12RB1 | −0.702 | 4.014 | −3.789 | 0.000 | 0.007 | −0.144 |
| LNM Group 2 vs. Group 1 | ENSG00000168297 | PXK | −0.719 | 5.035 | −3.791 | 0.000 | 0.007 | −0.170 |
| LNM Group 2 vs. Group 1 | ENSG00000171517 | LPAR3 | 1.403 | 4.947 | 3.797 | 0.000 | 0.007 | −0.129 |
| LNM Group 2 vs. Group 1 | ENSG00000147168 | IL2RG | −0.888 | 4.862 | −3.802 | 0.000 | 0.007 | −0.132 |
| LNM Group 2 vs. Group 1 | ENSG00000168487 | BMP1 | 0.763 | 6.199 | 3.803 | 0.000 | 0.007 | −0.186 |
| LNM Group 2 vs. Group 1 | ENSG00000266714 | MYO15B | −0.683 | 6.827 | −3.803 | 0.000 | 0.007 | −0.208 |
| LNM Group 2 vs. Group 1 | ENSG00000142910 | TINAGL1 | 0.737 | 6.304 | 3.804 | 0.000 | 0.007 | −0.181 |
| LNM Group 2 vs. Group 1 | ENSG00000213654 | GPSM3 | −0.821 | 4.580 | −3.807 | 0.000 | 0.007 | −0.104 |
| LNM Group 2 vs. Group 1 | ENSG00000130635 | COL5A1 | 1.097 | 9.463 | 3.815 | 0.000 | 0.007 | −0.176 |
| LNM Group 2 vs. Group 1 | ENSG00000155380 | SLC16A1 | 1.045 | 6.332 | 3.815 | 0.000 | 0.007 | −0.136 |
| LNM Group 2 vs. Group 1 | ENSG00000087589 | CASS4 | −0.654 | 3.162 | −3.815 | 0.000 | 0.007 | −0.056 |
| LNM Group 2 vs. Group 1 | ENSG00000179933 | C14orf119 | 0.651 | 4.817 | 3.819 | 0.000 | 0.007 | −0.062 |
| LNM Group 2 vs. Group 1 | ENSG00000203950 | FAM127B | 0.593 | 3.791 | 3.822 | 0.000 | 0.007 | −0.042 |
| LNM Group 2 vs. Group 1 | ENSG00000008283 | CYB561 | 0.641 | 5.771 | 3.828 | 0.000 | 0.007 | −0.100 |
| LNM Group 2 vs. Group 1 | ENSG00000131018 | SYNE1 | −0.629 | 7.671 | −3.830 | 0.000 | 0.007 | −0.133 |
| LNM Group 2 vs. Group 1 | ENSG00000147955 | SIGMAR1 | 0.674 | 5.254 | 3.831 | 0.000 | 0.007 | −0.030 |
| LNM Group 2 vs. Group 1 | ENSG00000066322 | ELOVL1 | 0.747 | 5.529 | 3.833 | 0.000 | 0.007 | −0.050 |
| LNM Group 2 vs. Group 1 | ENSG00000182704 | TSKU | 0.620 | 6.606 | 3.834 | 0.000 | 0.007 | −0.085 |
| LNM Group 2 vs. Group 1 | ENSG00000189233 | NUGGC | −0.902 | 3.838 | −3.841 | 0.000 | 0.007 | −0.002 |
| LNM Group 2 vs. Group 1 | ENSG00000101265 | RASSF2 | −0.943 | 6.133 | −3.844 | 0.000 | 0.006 | −0.058 |
| LNM Group 2 vs. Group 1 | ENSG00000197943 | PLCG2 | −1.080 | 5.984 | −3.844 | 0.000 | 0.006 | −0.044 |
| LNM Group 2 vs. Group 1 | ENSG00000266094 | RASSF5 | −0.805 | 6.409 | −3.846 | 0.000 | 0.006 | −0.065 |
| LNM Group 2 vs. Group 1 | ENSG00000197134 | ZNF257 | −0.609 | 2.637 | −3.853 | 0.000 | 0.006 | 0.047 |
| LNM Group 2 vs. Group 1 | ENSG00000142675 | CNKSR1 | 0.727 | 4.171 | 3.855 | 0.000 | 0.006 | 0.027 |
| LNM Group 2 vs. Group 1 | ENSG00000180448 | ARHGAP45 | −0.868 | 6.594 | −3.859 | 0.000 | 0.006 | −0.041 |
| LNM Group 2 vs. Group 1 | ENSG00000134352 | IL6ST | −0.751 | 8.576 | −3.860 | 0.000 | 0.006 | −0.059 |
| LNM Group 2 vs. Group 1 | ENSG00000105520 | PLPPR2 | 0.710 | 4.426 | 3.857 | 0.000 | 0.006 | 0.043 |
| LNM Group 2 vs. Group 1 | ENSG00000196511 | TPK1 | −0.810 | 4.042 | −3.859 | 0.000 | 0.006 | 0.049 |
| LNM Group 2 vs. Group 1 | ENSG00000125170 | DOK4 | 1.029 | 3.951 | 3.862 | 0.000 | 0.006 | 0.059 |
| LNM Group 2 vs. Group 1 | ENSG00000181513 | ACBD4 | −0.622 | 3.403 | −3.872 | 0.000 | 0.006 | 0.091 |
| LNM Group 2 vs. Group 1 | ENSG00000102796 | DHRS12 | −0.717 | 3.306 | −3.874 | 0.000 | 0.006 | 0.104 |
| LNM Group 2 vs. Group 1 | ENSG00000137460 | FHDC1 | 0.875 | 4.950 | 3.878 | 0.000 | 0.006 | 0.050 |
| LNM Group 2 vs. Group 1 | ENSG00000186469 | GNG2 | −0.738 | 5.157 | −3.883 | 0.000 | 0.006 | 0.099 |
| LNM Group 2 vs. Group 1 | ENSG00000110092 | CCND1 | 0.891 | 8.273 | 3.896 | 0.000 | 0.006 | 0.092 |
| LNM Group 2 vs. Group 1 | ENSG00000120278 | PLEKHG1 | −0.620 | 5.815 | −3.893 | 0.000 | 0.006 | 0.055 |
| LNM Group 2 vs. Group 1 | ENSG00000130653 | PNPLA7 | −0.918 | 4.295 | −3.899 | 0.000 | 0.006 | 0.148 |
| LNM Group 2 vs. Group 1 | ENSG00000134242 | PTPN22 | −0.791 | 4.137 | −3.903 | 0.000 | 0.006 | 0.172 |
| LNM Group 2 vs. Group 1 | ENSG00000257950 | P2RX5- | −0.662 | 4.207 | −3.905 | 0.000 | 0.006 | 0.167 |
| | | TAX1BP3 | | | | | | |
| LNM Group 2 vs. Group 1 | ENSG00000168918 | INPP5D | −1.100 | 6.562 | −3.914 | 0.000 | 0.006 | 0.137 |
| LNM Group 2 vs. Group 1 | ENSG00000125968 | ID1 | 1.073 | 5.394 | 3.918 | 0.000 | 0.006 | 0.183 |
| LNM Group 2 vs. Group 1 | ENSG00000128815 | WDFY4 | −1.239 | 4.967 | −3.919 | 0.000 | 0.006 | 0.189 |
| LNM Group 2 vs. Group 1 | ENSG00000181035 | SLC25A42 | −0.595 | 3.901 | −3.918 | 0.000 | 0.005 | 0.208 |
| LNM Group 2 vs. Group 1 | ENSG00000185477 | GPRIN3 | −0.754 | 6.767 | −3.924 | 0.000 | 0.005 | 0.136 |
| LNM Group 2 vs. Group 1 | ENSG00000139433 | GLTP | 0.868 | 6.583 | 3.925 | 0.000 | 0.005 | 0.135 |
| LNM Group 2 vs. Group 1 | ENSG00000140968 | IRF8 | −1.305 | 5.673 | −3.927 | 0.000 | 0.005 | 0.191 |
| LNM Group 2 vs. Group 1 | ENSG00000139926 | FRMD6 | 0.977 | 7.262 | 3.931 | 0.000 | 0.005 | 0.152 |
| LNM Group 2 vs. Group 1 | ENSG00000175463 | TBC1D10C | −1.054 | 5.212 | −3.932 | 0.000 | 0.005 | 0.197 |
| LNM Group 2 vs. Group 1 | ENSG00000174607 | UGT8 | −0.816 | 4.476 | −3.933 | 0.000 | 0.005 | 0.197 |
| LNM Group 2 vs. Group 1 | ENSG00000134516 | DOCK2 | −1.038 | 6.540 | −3.936 | 0.000 | 0.005 | 0.204 |
| LNM Group 2 vs. Group 1 | ENSG00000076641 | PAG1 | −0.735 | 6.732 | −3.936 | 0.000 | 0.005 | 0.184 |
| LNM Group 2 vs. Group 1 | ENSG00000157734 | SNX22 | −0.878 | 4.420 | −3.938 | 0.000 | 0.005 | 0.229 |
| LNM Group 2 vs. Group 1 | ENSG00000138119 | MYOF | 0.693 | 7.897 | 3.941 | 0.000 | 0.005 | 0.172 |
| LNM Group 2 vs. Group 1 | ENSG00000145882 | PCYOX1L | −0.705 | 4.087 | −3.940 | 0.000 | 0.005 | 0.277 |
| LNM Group 2 vs. Group 1 | ENSG00000132470 | ITGB4 | 0.870 | 8.923 | 3.949 | 0.000 | 0.005 | 0.202 |
| LNM Group 2 vs. Group 1 | ENSG00000162729 | IGSF8 | 0.598 | 4.653 | 3.948 | 0.000 | 0.005 | 0.271 |
| LNM Group 2 vs. Group 1 | ENSG00000154845 | PPP4R1 | 0.678 | 7.340 | 3.953 | 0.000 | 0.005 | 0.214 |
| LNM Group 2 vs. Group 1 | ENSG00000131969 | ABHD12B | 0.830 | 3.533 | 3.958 | 0.000 | 0.005 | 0.276 |
| LNM Group 2 vs. Group 1 | ENSG00000007944 | MYLIP | −0.831 | 4.819 | −3.960 | 0.000 | 0.005 | 0.287 |
| LNM Group 2 vs. Group 1 | ENSG00000110723 | EXPH5 | 1.002 | 5.394 | 3.961 | 0.000 | 0.005 | 0.291 |
| LNM Group 2 vs. Group 1 | ENSG00000142611 | PRDM16 | −0.639 | 2.935 | −3.958 | 0.000 | 0.005 | 0.329 |
| LNM Group 2 vs. Group 1 | ENSG00000188177 | ZC3H6 | −0.661 | 5.787 | −3.958 | 0.000 | 0.005 | 0.284 |
| LNM Group 2 vs. Group 1 | ENSG00000106415 | GLCCI1 | −0.919 | 5.225 | −3.963 | 0.000 | 0.005 | 0.295 |
| LNM Group 2 vs. Group 1 | ENSG00000140832 | MARVELD3 | 0.631 | 4.123 | 3.965 | 0.000 | 0.005 | 0.334 |
| LNM Group 2 vs. Group 1 | ENSG00000161642 | ZNF385A | 0.818 | 5.889 | 3.968 | 0.000 | 0.005 | 0.300 |
| LNM Group 2 vs. Group 1 | ENSG00000122642 | FKBP9 | 0.720 | 6.817 | 3.970 | 0.000 | 0.005 | 0.256 |
| LNM Group 2 vs. Group 1 | ENSG00000136490 | LIMD2 | −0.956 | 5.560 | −3.970 | 0.000 | 0.005 | 0.280 |
| LNM Group 2 vs. Group 1 | ENSG00000169242 | EFNA1 | 1.139 | 4.993 | 3.971 | 0.000 | 0.005 | 0.335 |
| LNM Group 2 vs. Group 1 | ENSG00000141424 | SLC39A6 | 0.705 | 6.122 | 3.972 | 0.000 | 0.005 | 0.300 |
| LNM Group 2 vs. Group 1 | ENSG00000142627 | EPHA2 | 0.993 | 6.140 | 3.977 | 0.000 | 0.005 | 0.278 |
| LNM Group 2 vs. Group 1 | ENSG00000143842 | SOX13 | 0.631 | 5.384 | 3.975 | 0.000 | 0.005 | 0.318 |
| LNM Group 2 vs. Group 1 | ENSG00000167693 | NXN | 0.718 | 6.142 | 3.979 | 0.000 | 0.005 | 0.291 |
| LNM Group 2 vs. Group 1 | ENSG00000114770 | ABCC5 | 0.613 | 7.369 | 3.977 | 0.000 | 0.005 | 0.280 |
| LNM Group 2 vs. Group 1 | ENSG00000158258 | CLSTN2 | −1.266 | 4.276 | −3.982 | 0.000 | 0.005 | 0.370 |
| LNM Group 2 vs. Group 1 | ENSG00000072858 | SIDT1 | −0.921 | 4.705 | −3.982 | 0.000 | 0.005 | 0.367 |
| LNM Group 2 vs. Group 1 | ENSG00000122026 | RPL21 | −0.655 | 7.932 | −3.982 | 0.000 | 0.005 | 0.291 |
| LNM Group 2 vs. Group 1 | ENSG00000100523 | DDHD1 | −0.694 | 6.954 | −3.983 | 0.000 | 0.005 | 0.303 |
| LNM Group 2 vs. Group 1 | ENSG00000048740 | CELF2 | −0.962 | 7.011 | −3.985 | 0.000 | 0.005 | 0.326 |
| LNM Group 2 vs. Group 1 | ENSG00000114841 | DNAH1 | −1.114 | 6.344 | −3.987 | 0.000 | 0.005 | 0.309 |
| LNM Group 2 vs. Group 1 | ENSG00000159166 | LAD1 | 0.971 | 6.931 | 3.988 | 0.000 | 0.005 | 0.304 |
| LNM Group 2 vs. Group 1 | ENSG00000175567 | UCP2 | −0.869 | 5.557 | −3.989 | 0.000 | 0.005 | 0.362 |
| LNM Group 2 vs. Group 1 | ENSG00000135540 | NHSL1 | 0.735 | 6.040 | 3.989 | 0.000 | 0.005 | 0.316 |
| LNM Group 2 vs. Group 1 | ENSG00000130396 | MLLT4 | 0.642 | 7.511 | 3.988 | 0.000 | 0.005 | 0.307 |
| LNM Group 2 vs. Group 1 | ENSG00000077684 | JADE1 | −0.920 | 5.467 | −3.993 | 0.000 | 0.005 | 0.367 |
| LNM Group 2 vs. Group 1 | ENSG00000167130 | DOLPP1 | 0.627 | 3.848 | 3.995 | 0.000 | 0.005 | 0.420 |
| LNM Group 2 vs. Group 1 | ENSG00000142733 | MAP3K6 | 0.638 | 5.753 | 3.996 | 0.000 | 0.005 | 0.363 |
| LNM Group 2 vs. Group 1 | ENSG00000172578 | KLHL6 | −0.823 | 6.365 | −4.003 | 0.000 | 0.005 | 0.376 |
| LNM Group 2 vs. Group 1 | ENSG00000139324 | TMTC3 | 0.698 | 6.592 | 4.003 | 0.000 | 0.005 | 0.360 |
| LNM Group 2 vs. Group 1 | ENSG00000275832 | ARHGAP23 | 0.614 | 6.815 | 4.010 | 0.000 | 0.005 | 0.375 |
| LNM Group 2 vs. Group 1 | ENSG00000116141 | MARK1 | 1.249 | 4.723 | 4.014 | 0.000 | 0.005 | 0.408 |
| LNM Group 2 vs. Group 1 | ENSG00000011105 | TSPAN9 | 0.679 | 5.692 | 4.011 | 0.000 | 0.005 | 0.417 |
| LNM Group 2 vs. Group 1 | ENSG00000137177 | KIF13A | 0.589 | 7.009 | 4.011 | 0.000 | 0.005 | 0.386 |
| LNM Group 2 vs. Group 1 | ENSG00000165300 | SLITRK5 | −0.625 | 2.816 | −4.013 | 0.000 | 0.005 | 0.474 |
| LNM Group 2 vs. Group 1 | ENSG00000088986 | DYNLL1 | 0.673 | 6.175 | 4.014 | 0.000 | 0.005 | 0.414 |
| LNM Group 2 vs. Group 1 | ENSG00000175832 | ETV4 | 0.835 | 3.719 | 4.016 | 0.000 | 0.005 | 0.460 |
| LNM Group 2 vs. Group 1 | ENSG00000156136 | DCK | −0.840 | 5.740 | −4.027 | 0.000 | 0.005 | 0.453 |
| LNM Group 2 vs. Group 1 | ENSG00000166908 | PIP4K2C | 0.708 | 5.499 | 4.027 | 0.000 | 0.005 | 0.471 |
| LNM Group 2 vs. Group 1 | ENSG00000069812 | HES2 | 1.457 | 5.193 | 4.031 | 0.000 | 0.005 | 0.503 |
| LNM Group 2 vs. Group 1 | ENSG00000145416 | MARCH1 | −0.695 | 6.187 | −4.031 | 0.000 | 0.005 | 0.456 |
| LNM Group 2 vs. Group 1 | ENSG00000056558 | TRAF1 | −0.820 | 5.965 | −4.036 | 0.000 | 0.005 | 0.463 |
| LNM Group 2 vs. Group 1 | ENSG00000110448 | CD5 | −1.202 | 4.630 | −4.036 | 0.000 | 0.005 | 0.499 |
| LNM Group 2 vs. Group 1 | ENSG00000105613 | MAST1 | −0.620 | 2.463 | −4.040 | 0.000 | 0.004 | 0.554 |
| LNM Group 2 vs. Group 1 | ENSG00000198521 | ZNF43 | −0.708 | 4.885 | −4.041 | 0.000 | 0.004 | 0.538 |
| LNM Group 2 vs. Group 1 | ENSG00000141579 | ZNF750 | 0.993 | 5.138 | 4.045 | 0.000 | 0.004 | 0.513 |
| LNM Group 2 vs. Group 1 | ENSG00000215252 | GOLGA8B | −0.706 | 7.421 | −4.045 | 0.000 | 0.004 | 0.469 |
| LNM Group 2 vs. Group 1 | ENSG00000162073 | PAQR4 | 0.671 | 4.207 | 4.045 | 0.000 | 0.004 | 0.554 |
| LNM Group 2 vs. Group 1 | ENSG00000110218 | PANX1 | 0.736 | 5.132 | 4.046 | 0.000 | 0.004 | 0.544 |
| LNM Group 2 vs. Group 1 | ENSG00000148057 | IDNK | −0.825 | 2.682 | −4.047 | 0.000 | 0.004 | 0.572 |
| LNM Group 2 vs. Group 1 | ENSG00000161835 | GRASP | −0.970 | 4.481 | −4.053 | 0.000 | 0.004 | 0.539 |
| LNM Group 2 vs. Group 1 | ENSG00000135631 | RAB11FIP5 | 0.702 | 5.639 | 4.060 | 0.000 | 0.004 | 0.554 |
| LNM Group 2 vs. Group 1 | ENSG00000133392 | MYH11 | −1.138 | 6.163 | −4.069 | 0.000 | 0.004 | 0.554 |
| LNM Group 2 vs. Group 1 | ENSG00000142632 | ARHGEF19 | 0.776 | 4.527 | 4.070 | 0.000 | 0.004 | 0.600 |
| LNM Group 2 vs. Group 1 | ENSG00000150627 | WDR17 | −0.868 | 2.701 | −4.072 | 0.000 | 0.004 | 0.641 |
| LNM Group 2 vs. Group 1 | ENSG00000213809 | KLRK1 | −0.653 | 4.097 | −4.072 | 0.000 | 0.004 | 0.633 |
| LNM Group 2 vs. Group 1 | ENSG00000106089 | STX1A | 0.730 | 3.374 | 4.078 | 0.000 | 0.004 | 0.655 |
| LNM Group 2 vs. Group 1 | ENSG00000138378 | STAT4 | −1.150 | 3.936 | −4.084 | 0.000 | 0.004 | 0.660 |
| LNM Group 2 vs. Group 1 | ENSG00000170989 | S1PR1 | −1.123 | 4.546 | −4.088 | 0.000 | 0.004 | 0.665 |
| LNM Group 2 vs. Group 1 | ENSG00000144182 | LIPT1 | −0.694 | 2.905 | −4.088 | 0.000 | 0.004 | 0.686 |
| LNM Group 2 vs. Group 1 | ENSG00000047346 | FAM214A | −0.707 | 6.618 | −4.091 | 0.000 | 0.004 | 0.613 |
| LNM Group 2 vs. Group 1 | ENSG00000198771 | RCSD1 | −0.911 | 5.765 | −4.098 | 0.000 | 0.004 | 0.650 |
| LNM Group 2 vs. Group 1 | ENSG00000188112 | C6orf132 | 0.819 | 6.357 | 4.101 | 0.000 | 0.004 | 0.641 |
| LNM Group 2 vs. Group 1 | ENSG00000107338 | SHB | 0.883 | 4.763 | 4.101 | 0.000 | 0.004 | 0.677 |
| LNM Group 2 vs. Group 1 | ENSG00000162852 | CNST | −0.624 | 5.847 | −4.099 | 0.000 | 0.004 | 0.659 |
| LNM Group 2 vs. Group 1 | ENSG00000120899 | PTK2B | −0.699 | 6.900 | −4.103 | 0.000 | 0.004 | 0.652 |
| LNM Group 2 vs. Group 1 | ENSG00000167114 | SLC27A4 | 0.721 | 5.084 | 4.104 | 0.000 | 0.004 | 0.711 |
| LNM Group 2 vs. Group 1 | ENSG00000125354 | SEP16 | −0.884 | 6.122 | −4.110 | 0.000 | 0.004 | 0.684 |
| LNM Group 2 vs. Group 1 | ENSG00000124406 | ATP8A1 | −1.428 | 5.412 | −4.114 | 0.000 | 0.004 | 0.733 |
| LNM Group 2 vs. Group 1 | ENSG00000180096 | SEP11 | −1.137 | 4.486 | −4.114 | 0.000 | 0.004 | 0.733 |
| LNM Group 2 vs. Group 1 | ENSG00000174718 | KIAA1551 | −0.733 | 7.676 | −4.118 | 0.000 | 0.004 | 0.672 |
| LNM Group 2 vs. Group 1 | ENSG00000117877 | CD3EAP | 0.929 | 4.051 | 4.119 | 0.000 | 0.004 | 0.755 |
| LNM Group 2 vs. Group 1 | ENSG00000142875 | PRKACB | −0.656 | 6.062 | −4.115 | 0.000 | 0.004 | 0.708 |
| LNM Group 2 vs. Group 1 | ENSG00000135905 | DOCK10 | −0.851 | 6.688 | −4.120 | 0.000 | 0.004 | 0.703 |
| LNM Group 2 vs. Group 1 | ENSG00000111348 | ARHGDIB | −0.836 | 6.685 | −4.121 | 0.000 | 0.004 | 0.705 |
| LNM Group 2 vs. Group 1 | ENSG00000137267 | TUBB2A | 1.214 | 4.781 | 4.124 | 0.000 | 0.004 | 0.755 |
| LNM Group 2 vs. Group 1 | ENSG00000169239 | CA5B | −0.640 | 5.402 | −4.121 | 0.000 | 0.004 | 0.744 |
| LNM Group 2 vs. Group 1 | ENSG00000106080 | FKBP14 | 0.639 | 5.463 | 4.123 | 0.000 | 0.004 | 0.764 |
| LNM Group 2 vs. Group 1 | ENSG00000135525 | MAP7 | 0.919 | 4.944 | 4.129 | 0.000 | 0.004 | 0.748 |
| LNM Group 2 vs. Group 1 | ENSG00000129932 | DOHH | 0.702 | 3.120 | 4.128 | 0.000 | 0.004 | 0.795 |
| LNM Group 2 vs. Group 1 | ENSG00000015133 | CCDC88C | −0.805 | 6.904 | −4.140 | 0.000 | 0.004 | 0.735 |
| LNM Group 2 vs. Group 1 | ENSG00000182866 | LCK | −0.875 | 4.395 | −4.139 | 0.000 | 0.004 | 0.795 |
| LNM Group 2 vs. Group 1 | ENSG00000174307 | PHLDA3 | 0.811 | 5.265 | 4.142 | 0.000 | 0.004 | 0.785 |
| LNM Group 2 vs. Group 1 | ENSG00000183762 | KREMEN1 | 0.809 | 6.523 | 4.144 | 0.000 | 0.004 | 0.747 |
| LNM Group 2 vs. Group 1 | ENSG00000183813 | CCR4 | −0.866 | 4.456 | −4.147 | 0.000 | 0.004 | 0.835 |
| LNM Group 2 vs. Group 1 | ENSG00000121895 | TMEM156 | −0.959 | 3.444 | −4.149 | 0.000 | 0.004 | 0.847 |
| LNM Group 2 vs. Group 1 | ENSG00000117020 | AKT3 | −0.766 | 6.297 | −4.150 | 0.000 | 0.004 | 0.798 |
| LNM Group 2 vs. Group 1 | ENSG00000104881 | PPP1R13L | 0.819 | 6.737 | 4.152 | 0.000 | 0.004 | 0.797 |
| LNM Group 2 vs. Group 1 | ENSG00000159348 | CYB5R1 | 1.033 | 5.471 | 4.155 | 0.000 | 0.004 | 0.841 |
| LNM Group 2 vs. Group 1 | ENSG00000160223 | ICOSLG | −0.739 | 5.371 | −4.153 | 0.000 | 0.004 | 0.809 |
| LNM Group 2 vs. Group 1 | ENSG00000100100 | PIK3IP1 | −1.021 | 6.385 | −4.160 | 0.000 | 0.004 | 0.819 |
| LNM Group 2 vs. Group 1 | ENSG00000102053 | ZC3H12B | −0.620 | 3.430 | −4.157 | 0.000 | 0.004 | 0.873 |
| LNM Group 2 vs. Group 1 | ENSG00000180921 | FAM83H | 0.830 | 7.453 | 4.162 | 0.000 | 0.004 | 0.799 |
| LNM Group 2 vs. Group 1 | ENSG00000164045 | CDC25A | 0.815 | 3.298 | 4.161 | 0.000 | 0.004 | 0.882 |
| LNM Group 2 vs. Group 1 | ENSG00000182183 | FAM159A | −1.077 | 3.510 | −4.166 | 0.000 | 0.004 | 0.887 |
| LNM Group 2 vs. Group 1 | ENSG00000162522 | KIAA1522 | 0.834 | 7.300 | 4.166 | 0.000 | 0.004 | 0.810 |
| LNM Group 2 vs. Group 1 | ENSG00000167880 | EVPL | 1.008 | 6.399 | 4.166 | 0.000 | 0.004 | 0.823 |
| LNM Group 2 vs. Group 1 | ENSG00000160213 | CSTB | 1.125 | 6.592 | 4.176 | 0.000 | 0.004 | 0.860 |
| LNM Group 2 vs. Group 1 | ENSG00000205476 | CCDC85C | 0.623 | 6.726 | 4.176 | 0.000 | 0.004 | 0.866 |
| LNM Group 2 vs. Group 1 | ENSG00000125912 | NCLN | 0.652 | 6.070 | 4.178 | 0.000 | 0.004 | 0.880 |
| LNM Group 2 vs. Group 1 | ENSG00000143643 | TTC13 | −0.622 | 4.730 | −4.178 | 0.000 | 0.004 | 0.925 |
| LNM Group 2 vs. Group 1 | ENSG00000115896 | PLCL1 | −0.866 | 3.573 | −4.179 | 0.000 | 0.004 | 0.936 |
| LNM Group 2 vs. Group 1 | ENSG00000150867 | PIP4K2A | −0.715 | 6.207 | −4.191 | 0.000 | 0.003 | 0.932 |
| LNM Group 2 vs. Group 1 | ENSG00000131067 | GGT7 | −0.778 | 3.705 | −4.195 | 0.000 | 0.003 | 0.974 |
| LNM Group 2 vs. Group 1 | ENSG00000188822 | CNR2 | −1.002 | 4.132 | −4.199 | 0.000 | 0.003 | 0.974 |
| LNM Group 2 vs. Group 1 | ENSG00000167895 | TMC8 | −1.135 | 6.024 | −4.199 | 0.000 | 0.003 | 0.934 |
| LNM Group 2 vs. Group 1 | ENSG00000108179 | PPIF | 0.915 | 6.371 | 4.201 | 0.000 | 0.003 | 0.933 |
| LNM Group 2 vs. Group 1 | ENSG00000167037 | SGSM1 | −1.024 | 2.684 | −4.203 | 0.000 | 0.003 | 0.989 |
| LNM Group 2 vs. Group 1 | ENSG00000239521 | GATS | −0.935 | 4.831 | −4.207 | 0.000 | 0.003 | 0.978 |
| LNM Group 2 vs. Group 1 | ENSG00000073711 | PPP2R3A | 0.781 | 6.041 | 4.206 | 0.000 | 0.003 | 0.954 |
| LNM Group 2 vs. Group 1 | ENSG00000132704 | FCRL2 | −1.190 | 4.061 | −4.208 | 0.000 | 0.003 | 0.999 |
| LNM Group 2 vs. Group 1 | ENSG00000097021 | ACOT7 | 0.814 | 4.464 | 4.211 | 0.000 | 0.003 | 1.006 |
| LNM Group 2 vs. Group 1 | ENSG00000178199 | ZC3H12D | −0.992 | 5.381 | −4.216 | 0.000 | 0.003 | 1.003 |
| LNM Group 2 vs. Group 1 | ENSG00000123159 | GIPC1 | 0.842 | 6.206 | 4.218 | 0.000 | 0.003 | 0.983 |
| LNM Group 2 vs. Group 1 | ENSG00000112294 | ALDH5A1 | −1.003 | 4.292 | −4.219 | 0.000 | 0.003 | 1.015 |
| LNM Group 2 vs. Group 1 | ENSG00000131788 | PIAS3 | 0.652 | 6.048 | 4.215 | 0.000 | 0.003 | 0.997 |
| LNM Group 2 vs. Group 1 | ENSG00000010278 | CD9 | 0.840 | 6.755 | 4.219 | 0.000 | 0.003 | 0.968 |
| LNM Group 2 vs. Group 1 | ENSG00000152484 | USP12 | −0.626 | 6.016 | −4.215 | 0.000 | 0.003 | 0.999 |
| LNM Group 2 vs. Group 1 | ENSG00000126860 | EVI2A | −0.747 | 4.368 | −4.227 | 0.000 | 0.003 | 1.067 |
| LNM Group 2 vs. Group 1 | ENSG00000073849 | ST6GAL1 | −1.035 | 6.799 | −4.232 | 0.000 | 0.003 | 1.005 |
| LNM Group 2 vs. Group 1 | ENSG00000129675 | ARHGEF6 | −0.843 | 5.572 | −4.232 | 0.000 | 0.003 | 1.065 |
| LNM Group 2 vs. Group 1 | ENSG00000185340 | GAS2L1 | 0.867 | 4.820 | 4.233 | 0.000 | 0.003 | 1.073 |
| LNM Group 2 vs. Group 1 | ENSG00000025708 | TYMP | 0.791 | 7.828 | 4.236 | 0.000 | 0.003 | 1.023 |
| LNM Group 2 vs. Group 1 | ENSG00000136802 | LRRC8A | 0.725 | 7.046 | 4.239 | 0.000 | 0.003 | 1.028 |
| LNM Group 2 vs. Group 1 | ENSG00000104894 | CD37 | −1.219 | 5.981 | −4.245 | 0.000 | 0.003 | 1.067 |
| LNM Group 2 vs. Group 1 | ENSG00000128283 | CDC42EP1 | 0.921 | 5.637 | 4.248 | 0.000 | 0.003 | 1.087 |
| LNM Group 2 vs. Group 1 | ENSG00000114993 | RTKN | 0.841 | 5.332 | 4.247 | 0.000 | 0.003 | 1.096 |
| LNM Group 2 vs. Group 1 | ENSG00000187838 | TMEM256- | 0.741 | 5.276 | 4.249 | 0.000 | 0.003 | 1.104 |
| | | PLSCR3 | | | | | | |
| LNM Group 2 vs. Group 1 | ENSG00000007866 | TEAD3 | 0.856 | 5.445 | 4.253 | 0.000 | 0.003 | 1.105 |
| LNM Group 2 vs. Group 1 | ENSG00000179761 | PIPOX | −0.825 | 2.630 | −4.250 | 0.000 | 0.003 | 1.134 |
| LNM Group 2 vs. Group 1 | ENSG00000213853 | EMP2 | 0.629 | 7.156 | 4.250 | 0.000 | 0.003 | 1.070 |
| LNM Group 2 vs. Group 1 | ENSG00000177954 | RPS27 | −0.769 | 7.559 | −4.254 | 0.000 | 0.003 | 1.071 |
| LNM Group 2 vs. Group 1 | ENSG00000126822 | PLEKHG3 | 1.151 | 6.938 | 4.257 | 0.000 | 0.003 | 1.079 |
| LNM Group 2 vs. Group 1 | ENSG00000140577 | CRTC3 | −0.593 | 6.519 | −4.261 | 0.000 | 0.003 | 1.104 |
| LNM Group 2 vs. Group 1 | ENSG00000053702 | NRIP2 | −0.595 | 4.002 | −4.262 | 0.000 | 0.003 | 1.160 |
| LNM Group 2 vs. Group 1 | ENSG00000171467 | ZNF318 | −0.910 | 6.558 | −4.268 | 0.000 | 0.003 | 1.110 |
| LNM Group 2 vs. Group 1 | ENSG00000173698 | ADGRG2 | −0.654 | 3.974 | −4.265 | 0.000 | 0.003 | 1.173 |
| LNM Group 2 vs. Group 1 | ENSG00000023445 | BIRC3 | −1.023 | 7.738 | −4.271 | 0.000 | 0.003 | 1.112 |
| LNM Group 2 vs. Group 1 | ENSG00000141968 | VAV1 | −0.639 | 4.844 | −4.267 | 0.000 | 0.003 | 1.160 |
| LNM Group 2 vs. Group 1 | ENSG00000116852 | KIF21B | −0.760 | 5.481 | −4.269 | 0.000 | 0.003 | 1.147 |
| LNM Group 2 vs. Group 1 | ENSG00000115935 | WIPF1 | −0.752 | 7.390 | −4.272 | 0.000 | 0.003 | 1.119 |
| LNM Group 2 vs. Group 1 | ENSG00000055163 | CYFIP2 | −1.248 | 5.674 | −4.275 | 0.000 | 0.003 | 1.167 |
| LNM Group 2 vs. Group 1 | ENSG00000155629 | PIK3AP1 | −0.864 | 5.975 | −4.276 | 0.000 | 0.003 | 1.144 |
| LNM Group 2 vs. Group 1 | ENSG00000164086 | DUSP7 | 0.926 | 6.203 | 4.277 | 0.000 | 0.003 | 1.163 |
| LNM Group 2 vs. Group 1 | ENSG00000100504 | PYGL | 0.996 | 6.335 | 4.280 | 0.000 | 0.003 | 1.178 |
| LNM Group 2 vs. Group 1 | ENSG00000074966 | TXK | −1.391 | 3.750 | −4.282 | 0.000 | 0.003 | 1.215 |
| LNM Group 2 vs. Group 1 | ENSG00000138336 | TET1 | −0.720 | 3.684 | −4.279 | 0.000 | 0.003 | 1.213 |
| LNM Group 2 vs. Group 1 | ENSG00000148700 | ADD3 | −0.686 | 7.000 | −4.280 | 0.000 | 0.003 | 1.158 |
| LNM Group 2 vs. Group 1 | ENSG00000152818 | UTRN | −0.597 | 8.580 | −4.282 | 0.000 | 0.003 | 1.158 |
| LNM Group 2 vs. Group 1 | ENSG00000115165 | CYTIP | −1.146 | 5.726 | −4.290 | 0.000 | 0.003 | 1.214 |
| LNM Group 2 vs. Group 1 | ENSG00000206579 | XKR4 | −1.580 | 3.711 | −4.292 | 0.000 | 0.003 | 1.243 |
| LNM Group 2 vs. Group 1 | ENSG00000063660 | GPC1 | 0.834 | 7.396 | 4.294 | 0.000 | 0.003 | 1.182 |
| LNM Group 2 vs. Group 1 | ENSG00000249242 | TMEM150C | −0.753 | 3.301 | −4.293 | 0.000 | 0.003 | 1.253 |
| LNM Group 2 vs. Group 1 | ENSG00000132182 | NUP210 | −0.836 | 6.513 | −4.302 | 0.000 | 0.003 | 1.208 |
| LNM Group 2 vs. Group 1 | ENSG00000198482 | ZNF808 | −0.635 | 4.847 | −4.298 | 0.000 | 0.003 | 1.238 |
| LNM Group 2 vs. Group 1 | ENSG00000237440 | ZNF737 | −0.769 | 3.684 | −4.300 | 0.000 | 0.003 | 1.275 |
| LNM Group 2 vs. Group 1 | ENSG00000099814 | CEP170B | 0.906 | 6.968 | 4.305 | 0.000 | 0.003 | 1.217 |
| LNM Group 2 vs. Group 1 | ENSG00000154380 | ENAH | 0.833 | 8.030 | 4.307 | 0.000 | 0.003 | 1.216 |
| LNM Group 2 vs. Group 1 | ENSG00000008513 | ST3GAL1 | −0.640 | 6.733 | −4.306 | 0.000 | 0.003 | 1.227 |
| LNM Group 2 vs. Group 1 | ENSG00000197629 | MPEG1 | −0.957 | 6.038 | −4.313 | 0.000 | 0.003 | 1.270 |
| LNM Group 2 vs. Group 1 | ENSG00000121957 | GPSM2 | 0.776 | 6.699 | 4.313 | 0.000 | 0.003 | 1.246 |
| LNM Group 2 vs. Group 1 | ENSG00000120885 | CLU | −1.925 | 6.021 | −4.316 | 0.000 | 0.003 | 1.264 |
| LNM Group 2 vs. Group 1 | ENSG00000198018 | ENTPD7 | 0.614 | 5.635 | 4.315 | 0.000 | 0.003 | 1.306 |
| LNM Group 2 vs. Group 1 | ENSG00000179841 | AKAP5 | −0.652 | 4.433 | −4.318 | 0.000 | 0.003 | 1.323 |
| LNM Group 2 vs. Group 1 | ENSG00000197780 | TAF13 | 0.696 | 4.485 | 4.323 | 0.000 | 0.003 | 1.336 |
| LNM Group 2 vs. Group 1 | ENSG00000185862 | EVI2B | −0.892 | 5.092 | −4.332 | 0.000 | 0.003 | 1.350 |
| LNM Group 2 vs. Group 1 | ENSG00000027075 | PRKCH | −0.662 | 6.399 | −4.333 | 0.000 | 0.003 | 1.326 |
| LNM Group 2 vs. Group 1 | ENSG00000161405 | IKZF3 | −1.204 | 7.038 | −4.339 | 0.000 | 0.003 | 1.326 |
| LNM Group 2 vs. Group 1 | ENSG00000103335 | PIEZO1 | 0.593 | 8.317 | 4.336 | 0.000 | 0.003 | 1.311 |
| LNM Group 2 vs. Group 1 | ENSG00000142065 | ZFP14 | −0.848 | 5.012 | −4.338 | 0.000 | 0.003 | 1.362 |
| LNM Group 2 vs. Group 1 | ENSG00000198945 | L3MBTL3 | −0.793 | 4.120 | −4.337 | 0.000 | 0.003 | 1.373 |
| LNM Group 2 vs. Group 1 | ENSG00000158710 | TAGLN2 | 0.642 | 8.278 | 4.342 | 0.000 | 0.003 | 1.331 |
| LNM Group 2 vs. Group 1 | ENSG00000065485 | PDIA5 | 0.857 | 4.771 | 4.347 | 0.000 | 0.003 | 1.375 |
| LNM Group 2 vs. Group 1 | ENSG00000116663 | FBXO6 | 0.786 | 3.493 | 4.351 | 0.000 | 0.003 | 1.419 |
| LNM Group 2 vs. Group 1 | ENSG00000081237 | PTPRC | −1.104 | 7.711 | −4.360 | 0.000 | 0.003 | 1.377 |
| LNM Group 2 vs. Group 1 | ENSG00000137502 | RAB30 | −0.911 | 5.630 | −4.361 | 0.000 | 0.003 | 1.412 |
| LNM Group 2 vs. Group 1 | ENSG00000101695 | RNF125 | −1.042 | 5.013 | −4.374 | 0.000 | 0.003 | 1.463 |
| LNM Group 2 vs. Group 1 | ENSG00000129250 | KIF1C | 0.652 | 7.432 | 4.375 | 0.000 | 0.003 | 1.431 |
| LNM Group 2 vs. Group 1 | ENSG00000242372 | EIF6 | 0.681 | 5.571 | 4.382 | 0.000 | 0.002 | 1.489 |
| LNM Group 2 vs. Group 1 | ENSG00000065413 | ANKRD44 | −1.215 | 6.328 | −4.387 | 0.000 | 0.002 | 1.472 |
| LNM Group 2 vs. Group 1 | ENSG00000213903 | LTB4R | 1.129 | 5.911 | 4.388 | 0.000 | 0.002 | 1.510 |
| LNM Group 2 vs. Group 1 | ENSG00000178971 | CTC1 | −0.637 | 5.953 | −4.395 | 0.000 | 0.002 | 1.509 |
| LNM Group 2 vs. Group 1 | ENSG00000163644 | PPM1K | −0.894 | 6.580 | −4.400 | 0.000 | 0.002 | 1.497 |
| LNM Group 2 vs. Group 1 | ENSG00000155849 | ELMO1 | −0.856 | 5.964 | −4.409 | 0.000 | 0.002 | 1.542 |
| LNM Group 2 vs. Group 1 | ENSG00000178852 | EFCAB13 | −0.839 | 4.397 | −4.411 | 0.000 | 0.002 | 1.588 |
| LNM Group 2 vs. Group 1 | ENSG00000116191 | RALGPS2 | −0.846 | 6.321 | −4.415 | 0.000 | 0.002 | 1 560 |
| LNM Group 2 vs. Group 1 | ENSG00000126432 | PRDX5 | 0.589 | 5.608 | 4.412 | 0.000 | 0.002 | 1.585 |
| LNM Group 2 vs. Group 1 | ENSG00000181007 | ZFP82 | −0.592 | 3.630 | −4.413 | 0.000 | 0.002 | 1.596 |
| LNM Group 2 vs. Group 1 | ENSG00000111913 | FAM65B | −1.529 | 6.221 | −4.418 | 0.000 | 0.002 | 1.569 |
| LNM Group 2 vs. Group 1 | ENSG00000020633 | RUNX3 | −0.664 | 6.066 | −4.417 | 0.000 | 0.002 | 1.554 |
| LNM Group 2 vs. Group 1 | ENSG00000216937 | CCDC7 | −0.908 | 3.695 | −4.417 | 0.000 | 0.002 | 1.606 |
| LNM Group 2 vs. Group 1 | ENSG00000204103 | MAFB | 0.823 | 6.556 | 4.420 | 0.000 | 0.002 | 1.583 |
| LNM Group 2 vs. Group 1 | ENSG00000142089 | IFITM3 | 0.720 | 7.273 | 4.420 | 0.000 | 0.002 | 1.558 |
| LNM Group 2 vs. Group 1 | ENSG00000164970 | FAM219A | 0.716 | 5.216 | 4.424 | 0.000 | 0.002 | 1.619 |
| LNM Group 2 vs. Group 1 | ENSG00000111224 | PARP11 | −0.643 | 5.165 | −4.427 | 0.000 | 0.002 | 1.624 |
| LNM Group 2 vs. Group 1 | ENSG00000166974 | MAPRE2 | −0.722 | 5.277 | −4.440 | 0.000 | 0.002 | 1.663 |
| LNM Group 2 vs. Group 1 | ENSG00000081059 | TCF7 | −0.992 | 6.367 | −4.450 | 0.000 | 0.002 | 1.642 |
| LNM Group 2 vs. Group 1 | ENSG00000166394 | CYB5R2 | 1.043 | 4.792 | 4.452 | 0.000 | 0.002 | 1.671 |
| LNM Group 2 vs. Group 1 | ENSG00000075884 | ARHGAP15 | −1.209 | 5.701 | −4.455 | 0.000 | 0.002 | 1.688 |
| LNM Group 2 vs. Group 1 | ENSG00000138639 | ARHGAP24 | −1.084 | 4.763 | −4.458 | 0.000 | 0.002 | 1.696 |
| LNM Group 2 vs. Group 1 | ENSG00000142102 | ATHL1 | −0.783 | 6.653 | −4.456 | 0.000 | 0.002 | 1.669 |
| LNM Group 2 vs. Group 1 | ENSG00000144218 | AFF3 | −1.721 | 4.918 | −4.460 | 0.000 | 0.002 | 1.702 |
| LNM Group 2 vs. Group 1 | ENSG00000174428 | GTF2IRD2B | −0.691 | 4.989 | −4.459 | 0.000 | 0.002 | 1.721 |
| LNM Group 2 vs. Group 1 | ENSG00000162241 | SLC25A45 | −0.682 | 5.265 | −4.460 | 0.000 | 0.002 | 1.713 |
| LNM Group 2 vs. Group 1 | ENSG00000130733 | YIPF2 | 0.695 | 4.801 | 4.461 | 0.000 | 0.002 | 1.727 |
| LNM Group 2 vs. Group 1 | ENSG00000111276 | CDKN1B | −0.593 | 6.005 | −4.470 | 0.000 | 0.002 | 1.732 |
| LNM Group 2 vs. Group 1 | ENSG00000276023 | DUSP14 | 1.086 | 4.567 | 4.480 | 0.000 | 0.002 | 1.775 |
| LNM Group 2 vs. Group 1 | ENSG00000091490 | SEL1L3 | −1.057 | 6.742 | −4.481 | 0.000 | 0.002 | 1.738 |
| LNM Group 2 vs. Group 1 | ENSG00000189060 | H1F0 | 1.037 | 6.906 | 4.482 | 0.000 | 0.002 | 1.755 |
| LNM Group 2 vs. Group 1 | ENSG00000073282 | TP63 | 1.040 | 8.375 | 4.489 | 0.000 | 0.002 | 1.752 |
| LNM Group 2 vs. Group 1 | ENSG00000130475 | FCHO1 | −0.856 | 3.866 | −4.486 | 0.000 | 0.002 | 1.806 |
| LNM Group 2 vs. Group 1 | ENSG00000213906 | LTB4R2 | 1.154 | 5.059 | 4.497 | 0.000 | 0.002 | 1.823 |
| LNM Group 2 vs. Group 1 | ENSG00000155893 | PXYLP1 | −0.790 | 4.930 | −4.497 | 0.000 | 0.002 | 1.816 |
| LNM Group 2 vs. Group 1 | ENSG00000111669 | TPI1 | 0.625 | 7.969 | 4.503 | 0.000 | 0.002 | 1.805 |
| LNM Group 2 vs. Group 1 | ENSG00000122986 | HVCN1 | −0.940 | 3.889 | −4.506 | 0.000 | 0.002 | 1.857 |
| LNM Group 2 vs. Group 1 | ENSG00000078687 | TNRC6C | −0.839 | 6.109 | −4.510 | 0.000 | 0.002 | 1.822 |
| LNM Group 2 vs. Group 1 | ENSG00000169499 | PLEKHA2 | −0.693 | 7.164 | −4.509 | 0.000 | 0.002 | 1.824 |
| LNM Group 2 vs. Group 1 | ENSG00000084207 | GSTP1 | 0.679 | 7.830 | 4.509 | 0.000 | 0.002 | 1.822 |
| LNM Group 2 vs. Group 1 | ENSG00000108370 | RGS9 | −0.897 | 3.862 | −4.509 | 0.000 | 0.002 | 1.871 |
| LNM Group 2 vs. Group 1 | ENSG00000152270 | PDE3B | −1.391 | 5.979 | −4.534 | 0.000 | 0.002 | 1.894 |
| LNM Group 2 vs. Group 1 | ENSG00000084070 | SMAP2 | −0.936 | 7.092 | −4.537 | 0.000 | 0.002 | 1.905 |
| LNM Group 2 vs. Group 1 | ENSG00000085733 | CTTN | 0.625 | 8.342 | 4.537 | 0.000 | 0.002 | 1.919 |
| LNM Group 2 vs. Group 1 | ENSG00000083444 | PLOD1 | 0.809 | 6.368 | 4.555 | 0.000 | 0.002 | 1.959 |
| LNM Group 2 vs. Group 1 | ENSG00000013725 | CD6 | −1.284 | 5.043 | −4.557 | 0.000 | 0.002 | 1.994 |
| LNM Group 2 vs. Group 1 | ENSG00000258539 | RP11- | −0.748 | 3.370 | −4.556 | 0.000 | 0.002 | 2.006 |
| | | 12J10.3 | | | | | | |
| LNM Group 2 vs. Group 1 | ENSG00000072818 | ACAP1 | −0.948 | 6.160 | −4.562 | 0.000 | 0.002 | 1.992 |
| LNM Group 2 vs. Group 1 | ENSG00000156381 | ANKRD9 | 0.909 | 5.074 | 4.561 | 0.000 | 0.002 | 2.015 |
| LNM Group 2 vs. Group 1 | ENSG00000165929 | TC2N | −1.059 | 6.055 | −4.573 | 0.000 | 0.002 | 2.026 |
| LNM Group 2 vs. Group 1 | ENSG00000147526 | TACC1 | −0.599 | 7.942 | −4.568 | 0.000 | 0.002 | 1.997 |
| LNM Group 2 vs. Group 1 | ENSG00000141934 | PLPP2 | 1.283 | 5.943 | 4.575 | 0.000 | 0.002 | 2.021 |
| LNM Group 2 vs. Group 1 | ENSG00000104814 | MAP4K1 | −1.165 | 4.835 | −4.579 | 0.000 | 0.002 | 2.059 |
| LNM Group 2 vs. Group 1 | ENSG00000113594 | LIFR | −1.402 | 5.977 | −4.580 | 0.000 | 0.002 | 2.051 |
| LNM Group 2 vs. Group 1 | ENSG00000178307 | TMEM11 | 0.592 | 3.740 | 4.577 | 0.000 | 0.002 | 2.068 |
| LNM Group 2 vs. Group 1 | ENSG00000149115 | TNKS1BP1 | 0.720 | 8.232 | 4.579 | 0.000 | 0.002 | 2.027 |
| LNM Group 2 vs. Group 1 | ENSG00000115232 | ITGA4 | −1.084 | 5.797 | −4.588 | 0.000 | 0.002 | 2.075 |
| LNM Group 2 vs. Group 1 | ENSG00000197044 | ZNF441 | −0.785 | 4.020 | −4.584 | 0.000 | 0.002 | 2.088 |
| LNM Group 2 vs. Group 1 | ENSG00000138162 | TACC2 | 0.848 | 6.815 | 4.600 | 0.000 | 0.002 | 2.087 |
| LNM Group 2 vs. Group 1 | ENSG00000064012 | CASP8 | −0.707 | 6.034 | −4.596 | 0.000 | 0.002 | 2.105 |
| LNM Group 2 vs. Group 1 | ENSG00000050820 | BCAR1 | 0.791 | 6.294 | 4.597 | 0.000 | 0.002 | 2.108 |
| LNM Group 2 vs. Group 1 | ENSG00000117266 | CDK18 | 0.936 | 5.022 | 4.602 | 0.000 | 0.002 | 2.104 |
| LNM Group 2 vs. Group 1 | ENSG00000116133 | DHCR24 | 1.119 | 7.294 | 4.605 | 0.000 | 0.002 | 2.097 |
| LNM Group 2 vs. Group 1 | ENSG00000141298 | SSH2 | −0.603 | 7.268 | −4.607 | 0.000 | 0.002 | 2.114 |
| LNM Group 2 vs. Group 1 | ENSG00000181588 | MEX3D | 0.905 | 4.514 | 4.611 | 0.000 | 0.002 | 2.154 |
| LNM Group 2 vs. Group 1 | ENSG00000123338 | NCKAP1L | −0.829 | 6.793 | −4.612 | 0.000 | 0.002 | 2.139 |
| LNM Group 2 vs. Group 1 | ENSG00000196405 | EVL | −0.846 | 7.403 | −4.614 | 0.000 | 0.002 | 2.126 |
| LNM Group 2 vs. Group 1 | ENSG00000155016 | CYP2U1 | −0.807 | 3.194 | −4.620 | 0.000 | 0.002 | 2.186 |
| LNM Group 2 vs. Group 1 | ENSG00000076356 | PLXNA2 | 0.895 | 6.494 | 4.639 | 0.000 | 0.002 | 2.205 |
| LNM Group 2 vs. Group 1 | ENSG00000186350 | RXRA | 0.601 | 7.402 | 4.634 | 0.000 | 0.002 | 2.200 |
| LNM Group 2 vs. Group 1 | ENSG00000006704 | GTF2IRD1 | 0.828 | 5.604 | 4.641 | 0.000 | 0.002 | 2.231 |
| LNM Group 2 vs. Group 1 | ENSG00000047634 | SCML1 | −0.779 | 4.723 | −4.642 | 0.000 | 0.002 | 2.254 |
| LNM Group 2 vs. Group 1 | ENSG00000169860 | P2RY1 | 1.058 | 4.446 | 4.651 | 0.000 | 0.002 | 2.258 |
| LNM Group 2 vs. Group 1 | ENSG00000116641 | DOCK7 | 0.733 | 7.233 | 4.648 | 0.000 | 0.002 | 2.235 |
| LNM Group 2 vs. Group 1 | ENSG00000129474 | AJUBA | 0.976 | 6.117 | 4.663 | 0.000 | 0.002 | 2.285 |
| LNM Group 2 vs. Group 1 | ENSG00000020256 | ZFP64 | 0.592 | 5.005 | 4.660 | 0.000 | 0.002 | 2.296 |
| LNM Group 2 vs. Group 1 | ENSG00000116871 | MAP7D1 | 0.758 | 6.943 | 4.664 | 0.000 | 0.002 | 2.286 |
| LNM Group 2 vs. Group 1 | ENSG00000113263 | ITK | −1.428 | 6.134 | −4.669 | 0.000 | 0.002 | 2.303 |
| LNM Group 2 vs. Group 1 | ENSG00000116221 | MRPL37 | 0.612 | 5.272 | 4.667 | 0.000 | 0.002 | 2.321 |
| LNM Group 2 vs. Group 1 | ENSG00000177542 | SLC25A22 | 0.642 | 5.057 | 4.671 | 0.000 | 0.002 | 2.326 |
| LNM Group 2 vs. Group 1 | ENSG00000149418 | ST14 | 0.978 | 6.340 | 4.681 | 0.000 | 0.002 | 2.335 |
| LNM Group 2 vs. Group 1 | ENSG00000243725 | TTC4 | 0.650 | 4 410 | 4.678 | 0.000 | 0.001 | 2.364 |
| LNM Group 2 vs. Group 1 | ENSG00000100842 | EFS | 0.996 | 4.926 | 4.684 | 0.000 | 0.001 | 2.364 |
| LNM Group 2 vs. Group 1 | ENSG00000131378 | RFTN1 | −0.861 | 6.038 | −4.694 | 0.000 | 0.001 | 2.393 |
| LNM Group 2 vs. Group 1 | ENSG00000135185 | TMEM243 | −0.783 | 4.854 | −4.704 | 0.000 | 0.001 | 2.438 |
| LNM Group 2 vs. Group 1 | ENSG00000135426 | TESPA1 | −1.681 | 4.546 | −4.710 | 0.000 | 0.001 | 2.443 |
| LNM Group 2 vs. Group 1 | ENSG00000006327 | TNFRSF12A | 1.230 | 5.627 | 4.715 | 0.000 | 0.001 | 2.455 |
| LNM Group 2 vs. Group 1 | ENSG00000111666 | CHPT1 | −0.836 | 4.953 | −4.711 | 0.000 | 0.001 | 2.458 |
| LNM Group 2 vs. Group 1 | ENSG00000100300 | TSPO | 0.649 | 5.203 | 4.712 | 0.000 | 0.001 | 2.460 |
| LNM Group 2 vs. Group 1 | ENSG00000183010 | PVCR1 | 1.013 | 4.258 | 4.720 | 0.000 | 0.001 | 2.473 |
| LNM Group 2 vs. Group 1 | ENSG00000123989 | CHPF | 0.869 | 5.915 | 4.720 | 0.000 | 0.001 | 2.451 |
| LNM Group 2 vs. Group 1 | ENSG00000172336 | POP7 | 0.692 | 3.486 | 4.719 | 0.000 | 0.001 | 2.478 |
| LNM Group 2 vs. Group 1 | ENSG00000147457 | CHMP7 | −0.799 | 6.007 | −4.726 | 0.000 | 0.001 | 2.480 |
| LNM Group 2 vs. Group 1 | ENSG00000003147 | ICA1 | −0.942 | 3.575 | −4.724 | 0.000 | 0.001 | 2.490 |
| LNM Group 2 vs. Group 1 | ENSG00000141098 | GFOD2 | 0.609 | 5.815 | 4.725 | 0.000 | 0.001 | 2.490 |
| LNM Group 2 vs. Group 1 | ENSG00000164691 | TAGAP | −1.312 | 4.936 | −4.732 | 0.000 | 0.001 | 2.507 |
| LNM Group 2 vs. Group 1 | ENSG00000080546 | SESN1 | −0.869 | 5.205 | −4.728 | 0.000 | 0.001 | 2.510 |
| LNM Group 2 vs. Group 1 | ENSG00000113712 | CSNK1A1 | 0.604 | 8.311 | 4.733 | 0.000 | 0.001 | 2.491 |
| LNM Group 2 vs. Group 1 | ENSG00000185963 | BICD2 | 0.673 | 7.499 | 4.736 | 0.000 | 0.001 | 2.502 |
| LNM Group 2 vs. Group 1 | ENSG00000137693 | YAP1 | 0.636 | 7.642 | 4.742 | 0.000 | 0.001 | 2.520 |
| LNM Group 2 vs. Group 1 | ENSG00000188229 | TUBB4B | 0.731 | 7.150 | 4.744 | 0.000 | 0.001 | 2.526 |
| LNM Group 2 vs. Group 1 | ENSG00000138767 | CNOT6L | −0.848 | 8.037 | −4.767 | 0.000 | 0.001 | 2.584 |
| LNM Group 2 vs. Group 1 | ENSG00000168421 | RHOH | −1.273 | 5.625 | −4.769 | 0.000 | 0.001 | 2.602 |
| LNM Group 2 vs. Group 1 | ENSG00000111846 | GCNT2 | −0.862 | 4.368 | −4.775 | 0.000 | 0.001 | 2.643 |
| LNM Group 2 vs. Group 1 | ENSG00000142949 | PTPRF | 0.918 | 8.842 | 4.781 | 0.000 | 0.001 | 2.625 |
| LNM Group 2 vs. Group 1 | ENSG00000084636 | COL16A1 | 1.024 | 7.656 | 4.790 | 0.000 | 0.001 | 2.650 |
| LNM Group 2 vs. Group 1 | ENSG00000089775 | ZBTB25 | −0.679 | 6.437 | −4.784 | 0.000 | 0.001 | 2.661 |
| LNM Group 2 vs. Group 1 | ENSG00000117395 | EBNA1BP2 | 0.707 | 4.976 | 4.785 | 0.000 | 0.001 | 2.677 |
| LNM Group 2 vs. Group 1 | ENSG00000105355 | PLIN3 | 0.794 | 5.600 | 4.788 | 0.000 | 0.001 | 2.684 |
| LNM Group 2 vs. Group 1 | ENSG00000160753 | RUSC1 | 0.898 | 5.344 | 4.792 | 0.000 | 0.001 | 2.693 |
| LNM Group 2 vs. Group 1 | ENSG00000172270 | BSG | 0.654 | 7.195 | 4.792 | 0.000 | 0.001 | 2.670 |
| LNM Group 2 vs. Group 1 | ENSG00000187627 | RGPD1 | −0.942 | 4.324 | −4.808 | 0.000 | 0.001 | 2.734 |
| LNM Group 2 vs. Group 1 | ENSG00000177674 | AGTRAP | 0.876 | 4.846 | 4.809 | 0.000 | 0.001 | 2.747 |
| LNM Group 2 vs. Group 1 | ENSG00000176809 | LRRC37A3 | −0.618 | 5.697 | −4.810 | 0.000 | 0.001 | 2.742 |
| LNM Group 2 vs. Group 1 | ENSG00000238227 | C9orf69 | 0.638 | 5.606 | 4.815 | 0.000 | 0.001 | 2.759 |
| LNM Group 2 vs. Group 1 | ENSG00000066923 | STAG3 | −0.956 | 6.143 | −4.823 | 0.000 | 0.001 | 2.754 |
| LNM Group 2 vs. Group 1 | ENSG00000110104 | CCDC86 | 0.938 | 4.215 | 4.821 | 0.000 | 0.001 | 2.779 |
| LNM Group 2 vs. Group 1 | ENSG00000184588 | PDE4B | −1.069 | 5.742 | −4.826 | 0.000 | 0.001 | 2.772 |
| LNM Group 2 vs. Group 1 | ENSG00000116649 | SRM | 0.609 | 5.237 | 4.821 | 0.000 | 0.001 | 2.777 |
| LNM Group 2 vs. Group 1 | ENSG00000170542 | SERPINB9 | −0.811 | 6.327 | −4.827 | 0.000 | 0.001 | 2.775 |
| LNM Group 2 vs. Group 1 | ENSG00000196912 | ANKRD36B | −0.654 | 5.995 | −4.837 | 0.000 | 0.001 | 2.822 |
| LNM Group 2 vs. Group 1 | ENSG00000108622 | ICAM2 | −0.844 | 3.535 | −4.850 | 0.000 | 0.001 | 2.858 |
| LNM Group 2 vs. Group 1 | ENSG00000120910 | PPP3CC | −0.730 | 4.548 | −4.850 | 0.000 | 0.001 | 2.868 |
| LNM Group 2 vs. Group 1 | ENSG00000082781 | ITGB5 | 1.036 | 6.486 | 4.858 | 0.000 | 0.001 | 2.860 |
| LNM Group 2 vs. Group 1 | ENSG00000145743 | FBXL17 | −0.634 | 5.245 | −4.853 | 0.000 | 0.001 | 2.879 |
| LNM Group 2 vs. Group 1 | ENSG00000173890 | GPR160 | −0.852 | 3.730 | −4.853 | 0.000 | 0.001 | 2.877 |
| LNM Group 2 vs. Group 1 | ENSG00000034053 | APBA2 | −1.297 | 4.603 | −4.863 | 0.000 | 0.001 | 2.892 |
| LNM Group 2 vs. Group 1 | ENSG00000213923 | CSNK1E | 0.648 | 7.333 | 4.864 | 0.000 | 0.001 | 2.886 |
| LNM Group 2 vs. Group 1 | ENSG00000130560 | UBAC1 | 0.632 | 5.079 | 4.873 | 0.000 | 0.001 | 2.937 |
| LNM Group 2 vs. Group 1 | ENSG00000163935 | SFMBT1 | −0.629 | 5.015 | −4.875 | 0.000 | 0.001 | 2.943 |
| LNM Group 2 vs. Group 1 | ENSG00000188157 | AGRN | 1.001 | 8.450 | 4.882 | 0.000 | 0.001 | 2.930 |
| LNM Group 2 vs. Group 1 | ENSG00000187824 | TMEM220 | −0.852 | 3.219 | −4.877 | 0.000 | 0.001 | 2.933 |
| LNM Group 2 vs. Group 1 | ENSG00000112182 | BACH2 | −1.443 | 5.553 | −4.888 | 0.000 | 0.001 | 2.955 |
| LNM Group 2 vs. Group 1 | ENSG00000054219 | LY75 | −1.062 | 5.783 | −4.913 | 0.000 | 0.001 | 3.031 |
| LNM Group 2 vs. Group 1 | ENSG00000005302 | MSL3 | −0.638 | 5.615 | −4.914 | 0.000 | 0.001 | 3.055 |
| LNM Group 2 vs. Group 1 | ENSG00000050327 | ARHGEF5 | 0.808 | 5.829 | 4.916 | 0.000 | 0.001 | 3.065 |
| LNM Group 2 vs. Group 1 | ENSG00000121210 | KIAA0922 | −0.850 | 6.079 | −4.934 | 0.000 | 0.001 | 3.106 |
| LNM Group 2 vs. Group 1 | ENSG00000119333 | WDR34 | 0.815 | 4.646 | 4.941 | 0.000 | 0.001 | 3.141 |
| LNM Group 2 vs. Group 1 | ENSG00000172349 | IL16 | −1.284 | 6.119 | −4.948 | 0.000 | 0.001 | 3.137 |
| LNM Group 2 vs. Group 1 | ENSG00000129911 | KLF16 | 0.635 | 5.389 | 4.945 | 0.000 | 0.001 | 3.153 |
| LNM Group 2 vs. Group 1 | ENSG00000102100 | SLC35A2 | 0.586 | 4.437 | 4.953 | 0.000 | 0.001 | 3.174 |
| LNM Group 2 vs. Group 1 | ENSG00000173114 | LRRN3 | −0.764 | 3.598 | −4.958 | 0.000 | 0.001 | 3.181 |
| LNM Group 2 vs. Group 1 | ENSG00000223547 | ZNF844 | −0.926 | 4.732 | −4.966 | 0.000 | 0.001 | 3.209 |
| LNM Group 2 vs. Group 1 | ENSG00000118596 | SLC16A7 | −0.956 | 5.772 | −4.979 | 0.000 | 0.001 | 3.231 |
| LNM Group 2 vs. Group 1 | ENSG00000132196 | HSD17B7 | −0.604 | 4.321 | −4.976 | 0.000 | 0.001 | 3.238 |
| LNM Group 2 vs. Group 1 | ENSG00000103365 | GGA2 | −0.704 | 7.164 | −4.977 | 0.000 | 0.001 | 3.234 |
| LNM Group 2 vs. Group 1 | ENSG00000105483 | CARD8 | −0.689 | 6.511 | −4.979 | 0.000 | 0.001 | 3.248 |
| LNM Group 2 vs. Group 1 | ENSG00000162302 | RPS6KA4 | 0.645 | 6.029 | 4.982 | 0.000 | 0.001 | 3.262 |
| LNM Group 2 vs. Group 1 | ENSG00000006118 | TMEM132A | 1.234 | 6.305 | 4.995 | 0.000 | 0.001 | 3.277 |
| LNM Group 2 vs. Group 1 | ENSG00000205730 | ITPRIPL2 | 0.677 | 7.227 | 4.992 | 0.000 | 0.001 | 3.279 |
| LNM Group 2 vs. Group 1 | ENSG00000153048 | CARHSP1 | 0.804 | 6.084 | 4.999 | 0.000 | 0.001 | 3.316 |
| LNM Group 2 vs. Group 1 | ENSG00000105866 | SP4 | −0.786 | 5.031 | −5.017 | 0.000 | 0.001 | 3.367 |
| LNM Group 2 vs. Group 1 | ENSG00000116260 | QSOX1 | 0.837 | 6.954 | 5.019 | 0.000 | 0.001 | 3.361 |
| LNM Group 2 vs. Group 1 | ENSG00000179456 | ZBTB18 | −0.858 | 4.841 | −5.020 | 0.000 | 0.001 | 3.377 |
| LNM Group 2 vs. Group 1 | ENSG00000160113 | NR2F6 | 0.946 | 4.132 | 5.022 | 0.000 | 0.001 | 3.378 |
| LNM Group 2 vs. Group 1 | ENSG00000167553 | TUBA1C | 0.856 | 7.267 | 5.039 | 0.000 | 0.001 | 3.409 |
| LNM Group 2 vs. Group 1 | ENSG00000077044 | DGKD | −0.768 | 6.414 | −5.035 | 0.000 | 0.001 | 3.413 |
| LNM Group 2 vs. Group 1 | ENSG00000142684 | ZNF593 | 0.756 | 2.636 | 5.039 | 0.000 | 0.001 | 3.398 |
| LNM Group 2 vs. Group 1 | ENSG00000127080 | IPPK | 0.857 | 5.884 | 5.043 | 0.000 | 0.001 | 3.442 |
| LNM Group 2 vs. Group 1 | ENSG00000037897 | METTL1 | 0.769 | 3.248 | 5.050 | 0.000 | 0.001 | 3.448 |
| LNM Group 2 vs. Group 1 | ENSG00000151348 | EXT2 | 0.593 | 6.172 | 5.053 | 0.000 | 0.001 | 3.474 |
| LNM Group 2 vs. Group 1 | ENSG00000179909 | ZNF154 | −0.671 | 5.071 | −5.064 | 0.000 | 0.001 | 3.508 |
| LNM Group 2 vs. Group 1 | ENSG00000188452 | CERKL | −0.768 | 3.996 | −5.068 | 0.000 | 0.001 | 3.511 |
| LNM Group 2 vs. Group 1 | ENSG00000101596 | SMCHD1 | −0.733 | 8.314 | −5.070 | 0.000 | 0.001 | 3.514 |
| LNM Group 2 vs. Group 1 | ENSG00000168944 | CEP120 | −0.719 | 5.476 | −5.071 | 0.000 | 0.001 | 3.531 |
| LNM Group 2 vs. Group 1 | ENSG00000163191 | S100A11 | 0.927 | 7.252 | 5.081 | 0.000 | 0.001 | 3.538 |
| LNM Group 2 vs. Group 1 | ENSG00000197217 | ENTPD4 | −0.587 | 6.949 | −5.079 | 0.000 | 0.001 | 3.543 |
| LNM Group 2 vs. Group 1 | ENSG00000171791 | BCL2 | −1.029 | 6.515 | −5.097 | 0.000 | 0.001 | 3.581 |
| LNM Group 2 vs. Group 1 | ENSG00000198624 | CCDC69 | −0.790 | 5.502 | −5.091 | 0.000 | 0.001 | 3.590 |
| LNM Group 2 vs. Group 1 | ENSG00000134324 | LPIN1 | −0.957 | 6.846 | −5.104 | 0.000 | 0.001 | 3.600 |
| LNM Group 2 vs. Group 1 | ENSG00000187239 | FNBP1 | −0.779 | 6.844 | −5.103 | 0.000 | 0.001 | 3.620 |
| LNM Group 2 vs. Group 1 | ENSG00000089094 | KDM2B | −1.278 | 6.492 | −5.120 | 0.000 | 0.001 | 3.655 |
| LNM Group 2 vs. Group 1 | ENSG00000174013 | FBXO45 | 0.790 | 5.520 | 5.137 | 0.000 | 0.001 | 3.730 |
| LNM Group 2 vs. Group 1 | ENSG00000165209 | STRBP | −0.874 | 5.825 | −5.141 | 0.000 | 0.001 | 3.735 |
| LNM Group 2 vs. Group 1 | ENSG00000198932 | GPRASP1 | −1.047 | 4.607 | −5.144 | 0.000 | 0.001 | 3.736 |
| LNM Group 2 vs. Group 1 | ENSG00000168685 | IL7R | −1.129 | 7.463 | −5.148 | 0.000 | 0.001 | 3.738 |
| LNM Group 2 vs. Group 1 | ENSG00000144749 | LRIG1 | −0.739 | 5.807 | −5.154 | 0.000 | 0.001 | 3.777 |
| LNM Group 2 vs. Group 1 | ENSG00000178585 | CTNNBIP1 | 1.074 | 5.442 | 5.166 | 0.000 | 0.001 | 3.801 |
| LNM Group 2 vs. Group 1 | ENSG00000196459 | TRAPPC2 | −0.599 | 5.062 | −5.175 | 0.000 | 0.001 | 3.846 |
| LNM Group 2 vs. Group 1 | ENSG00000105708 | ZNF14 | −0.795 | 3.238 | −5.176 | 0.000 | 0.001 | 3.815 |
| LNM Group 2 vs. Group 1 | ENSG00000150995 | ITPR1 | −1.128 | 6.573 | −5.199 | 0.000 | 0.001 | 3.897 |
| LNM Group 2 vs. Group 1 | ENSG00000196268 | ZNF493 | −0.637 | 5.822 | −5.192 | 0.000 | 0.001 | 3.895 |
| LNM Group 2 vs. Group 1 | ENSG00000110400 | NECTIN1 | 1.230 | 7.462 | 5.199 | 0.000 | 0.001 | 3.890 |
| LNM Group 2 vs. Group 1 | ENSG00000134954 | ETS1 | −0.815 | 8.326 | −5.193 | 0.000 | 0.001 | 3.888 |
| LNM Group 2 vs. Group 1 | ENSG00000095383 | TBC1D2 | 1.038 | 5.587 | 5.213 | 0.000 | 0.001 | 3.943 |
| LNM Group 2 vs. Group 1 | ENSG00000165804 | ZNF219 | 0.666 | 4.798 | 5.210 | 0.000 | 0.001 | 3.945 |
| LNM Group 2 vs. Group 1 | ENSG00000117643 | MANIC1 | −1.050 | 4.603 | −5.219 | 0.000 | 0.001 | 3.963 |
| LNM Group 2 vs. Group 1 | ENSG00000163975 | MELTF | 0.911 | 4.972 | 5.221 | 0.000 | 0.001 | 3.980 |
| LNM Group 2 vs. Group 1 | ENSG00000181722 | ZBTB20 | −0.799 | 7.965 | −5.219 | 0.000 | 0.001 | 3.971 |
| LNM Group 2 vs. Group 1 | ENSG00000077157 | PPP1R12B | −0.706 | 7.037 | −5.223 | 0.000 | 0.001 | 3.984 |
| LNM Group 2 vs. Group 1 | ENSG00000065809 | FAM107B | −1.126 | 6.305 | −5.238 | 0.000 | 0.001 | 4.016 |
| LNM Group 2 vs. Group 1 | ENSG00000142669 | SH3BGRL3 | 0.635 | 6.631 | 5.238 | 0.000 | 0.001 | 4.037 |
| LNM Group 2 vs. Group 1 | ENSG00000136026 | CKAP4 | 0.956 | 6.922 | 5.247 | 0.000 | 0.001 | 4.041 |
| LNM Group 2 vs. Group 1 | ENSG00000170677 | SOCS6 | 0.841 | 4.864 | 5.241 | 0.000 | 0.001 | 4.046 |
| LNM Group 2 vs. Group 1 | ENSG00000155085 | AK9 | −0.769 | 3.484 | −5.244 | 0.000 | 0.001 | 4.023 |
| LNM Group 2 vs. Group 1 | ENSG00000185803 | SLC52A2 | 0.773 | 5.077 | 5.245 | 0.000 | 0.001 | 4.056 |
| LNM Group 2 vs. Group 1 | ENSG00000188522 | FAM83G | 1.078 | 6.451 | 5.253 | 0.000 | 0.001 | 4.062 |
| LNM Group 2 vs. Group 1 | ENSG00000136830 | FAM129B | 0.914 | 7.815 | 5.256 | 0.000 | 0.001 | 4.071 |
| LNM Group 2 vs. Group 1 | ENSG00000139597 | N4BP2L1 | −0.775 | 4.663 | −5.256 | 0.000 | 0.001 | 4.081 |
| LNM Group 2 vs. Group 1 | ENSG00000214160 | ALG3 | 0.591 | 4.808 | 5.270 | 0.000 | 0.001 | 4.131 |
| LNM Group 2 vs. Group 1 | ENSG00000154814 | OXNAD1 | −0.930 | 5.352 | −5.273 | 0.000 | 0.001 | 4.139 |
| LNM Group 2 vs. Group 1 | ENSG00000198382 | UVRAG | −0.617 | 5.794 | −5.272 | 0.000 | 0.001 | 4.141 |
| LNM Group 2 vs. Group 1 | ENSG00000125245 | GPR18 | −0.797 | 3.830 | −5.278 | 0.000 | 0.001 | 4.127 |
| LNM Group 2 vs. Group 1 | ENSG00000010810 | FYN | −0.855 | 6.392 | −5.286 | 0.000 | 0.001 | 4.176 |
| LNM Group 2 vs. Group 1 | ENSG00000112149 | CD83 | −1.227 | 5.120 | −5.295 | 0.000 | 0.001 | 4.191 |
| LNM Group 2 vs. Group 1 | ENSG00000100304 | TTLL12 | 0.784 | 5.941 | 5.291 | 0.000 | 0.001 | 4.198 |
| LNM Group 2 vs. Group 1 | ENSG00000185697 | MYBL1 | −1.282 | 3.984 | −5.304 | 0.000 | 0.001 | 4.172 |
| LNM Group 2 vs. Group 1 | ENSG00000100804 | PSMB5 | 0.840 | 5.277 | 5.305 | 0.000 | 0.001 | 4.228 |
| LNM Group 2 vs. Group 1 | ENSG00000182230 | FAM153B | −1.295 | 3.641 | −5.329 | 0.000 | 0.001 | 4.264 |
| LNM Group 2 vs. Group 1 | ENSG00000111860 | CEP85L | −0.897 | 5.181 | −5.326 | 0.000 | 0.001 | 4.303 |
| LNM Group 2 vs. Group 1 | ENSG00000131653 | TRAF7 | 0.617 | 6.577 | 5.337 | 0.000 | 0.001 | 4.339 |
| LNM Group 2 vs. Group 1 | ENSG00000121966 | CXCR4 | −1.446 | 6.918 | −5.351 | 0.000 | 0.001 | 4.357 |
| LNM Group 2 vs. Group 1 | ENSG00000118482 | PHF3 | −0.611 | 7.322 | −5.378 | 0.000 | 0.001 | 4.460 |
| LNM Group 2 vs. Group 1 | ENSG00000082512 | TRAF5 | −0.904 | 6.182 | −5.394 | 0.000 | 0.001 | 4.511 |
| LNM Group 2 vs. Group 1 | ENSG00000116212 | LRRC42 | 0.909 | 4.193 | 5.393 | 0.000 | 0.001 | 4.481 |
| LNM Group 2 vs. Group 1 | ENSG00000081320 | STK17B | −0.923 | 7.868 | −5.408 | 0.000 | 0.001 | 4.541 |
| LNM Group 2 vs. Group 1 | ENSG00000269404 | SPIB | −1.375 | 4.499 | −5.412 | 0.000 | 0.001 | 4.545 |
| LNM Group 2 vs. Group 1 | ENSG00000157077 | ZFYVE9 | 0.952 | 5.526 | 5.418 | 0.000 | 0.001 | 4.581 |
| LNM Group 2 vs. Group 1 | ENSG00000132819 | RBM38 | −0.935 | 5.325 | −5.455 | 0.000 | 0.000 | 4.695 |
| LNM Group 2 vs. Group 1 | ENSG00000138795 | LEF1 | −1.398 | 5.114 | −5.463 | 0.000 | 0.000 | 4.696 |
| LNM Group 2 vs. Group 1 | ENSG00000173200 | PARP15 | −1.051 | 7.023 | −5.483 | 0.000 | 0.000 | 4.764 |
| LNM Group 2 vs. Group 1 | ENSG00000153885 | KCTD15 | 0.862 | 5.747 | 5.488 | 0.000 | 0.000 | 4.802 |
| LNM Group 2 vs. Group 1 | ENSG00000114491 | UMPS | 0.784 | 5.786 | 5.495 | 0.000 | 0.000 | 4.825 |
| LNM Group 2 vs. Group 1 | ENSG00000188643 | S100A16 | 1.114 | 6.648 | 5.510 | 0.000 | 0.000 | 4.847 |
| LNM Group 2 vs. Group 1 | ENSG00000240849 | TMEM189 | 0.730 | 6.643 | 5.505 | 0.000 | 0.000 | 4.854 |
| LNM Group 2 vs. Group 1 | ENSG00000206053 | HN1L | 0.708 | 6.640 | 5.506 | 0.000 | 0.000 | 4.860 |
| LNM Group 2 vs. Group 1 | ENSG00000224051 | CPTP | 0.677 | 4.410 | 5.539 | 0.000 | 0.000 | 4.932 |
| LNM Group 2 vs. Group 1 | ENSG00000104660 | LEPROTL1 | −0.792 | 5.703 | −5.551 | 0.000 | 0.000 | 4.991 |
| LNM Group 2 vs. Group 1 | ENSG00000186854 | TRABD2A | −1.446 | 4.775 | −5.564 | 0.000 | 0.000 | 4.988 |
| LNM Group 2 vs. Group 1 | ENSG00000118922 | KLF12 | −1.093 | 6.090 | −5.565 | 0.000 | 0.000 | 5.015 |
| LNM Group 2 vs. Group 1 | ENSG00000166025 | AMOTL1 | 1.035 | 7.486 | 5.569 | 0.000 | 0.000 | 5.029 |
| LNM Group 2 vs. Group 1 | ENSG00000157911 | PEX10 | 0.685 | 3.995 | 5.567 | 0.000 | 0.000 | 5.002 |
| LNM Group 2 vs. Group 1 | ENSG00000100139 | MICALL1 | 1.006 | 7.181 | 5.576 | 0.000 | 0.000 | 5.052 |
| LNM Group 2 vs. Group 1 | ENSG00000167695 | FAM57A | 0.869 | 4.784 | 5.600 | 0.000 | 0.000 | 5.132 |
| LNM Group 2 vs. Group 1 | ENSG00000159023 | EPB41 | −0.902 | 6.816 | −5.627 | 0.000 | 0.000 | 5.227 |
| LNM Group 2 vs. Group 1 | ENSG00000167483 | FAM129C | −1.416 | 5.554 | −5.635 | 0.000 | 0.000 | 5.226 |
| LNM Group 2 vs. Group 1 | ENSG00000182568 | SATB1 | −1.347 | 6.057 | −5.662 | 0.000 | 0.000 | 5.314 |
| LNM Group 2 vs. Group 1 | ENSG00000205268 | PDE7A | −0.736 | 7.026 | −5.654 | 0.000 | 0.000 | 5.317 |
| LNM Group 2 vs. Group 1 | ENSG00000174485 | DENND4A | −0.842 | 6.688 | −5.685 | 0.000 | 0.000 | 5.411 |
| LNM Group 2 vs. Group 1 | ENSG00000189337 | KAZN | 1.295 | 5.006 | 5.697 | 0.000 | 0.000 | 5.413 |
| LNM Group 2 vs. Group 1 | ENSG00000133740 | E2F5 | −1.597 | 4.656 | −5.702 | 0.000 | 0.000 | 5.411 |
| LNM Group 2 vs. Group 1 | ENSG00000070731 | ST6GALNAC2 | 0.996 | 5.029 | 5.695 | 0.000 | 0.000 | 5.428 |
| LNM Group 2 vs. Group 1 | ENSG00000133639 | BTG1 | −0.962 | 8.026 | −5.700 | 0.000 | 0.000 | 5.446 |
| LNM Group 2 vs. Group 1 | ENSG00000152520 | PAN3 | −0.654 | 6.651 | −5.707 | 0.000 | 0.000 | 5.480 |
| LNM Group 2 vs. Group 1 | ENSG00000198270 | TMEM116 | −0.970 | 4.975 | −5.720 | 0.000 | 0.000 | 5.497 |
| LNM Group 2 vs. Group 1 | ENSG00000160691 | SHC1 | 0.631 | 7.619 | 5.731 | 0.000 | 0.000 | 5.554 |
| LNM Group 2 vs. Group 1 | ENSG00000069493 | CLEC2D | −1.155 | 6.220 | −5.767 | 0.000 | 0.000 | 5.636 |
| LNM Group 2 vs. Group 1 | ENSG00000169508 | GPR183 | −1.164 | 5.105 | −5.789 | 0.000 | 0.000 | 5.685 |
| LNM Group 2 vs. Group 1 | ENSG00000198625 | MDM4 | −0.715 | 8.224 | −5.796 | 0.000 | 0.000 | 5.758 |
| LNM Group 2 vs. Group 1 | ENSG00000163293 | NIPAL1 | 1.391 | 4.501 | 5.831 | 0.000 | 0.000 | 5.794 |
| LNM Group 2 vs. Group 1 | ENSG00000145241 | CENPC | −0.663 | 5.966 | −5.823 | 0.000 | 0.000 | 5.837 |
| LNM Group 2 vs. Group 1 | ENSG00000177873 | ZNF619 | −0.786 | 4.047 | −5.882 | 0.000 | 0.000 | 5.937 |
| LNM Group 2 vs. Group 1 | ENSG00000143321 | HDGF | 0.797 | 7.794 | 5.907 | 0.000 | 0.000 | 6.104 |
| LNM Group 2 vs. Group 1 | ENSG00000162413 | KLHL21 | 0.828 | 6.699 | 5.949 | 0.000 | 0.000 | 6.233 |
| LNM Group 2 vs. Group 1 | ENSG00000158985 | CDC42SE2 | −0.960 | 6.699 | −5.971 | 0.000 | 0.000 | 6.296 |
| LNM Group 2 vs. Group 1 | ENSG00000169972 | PUSL1 | 0.751 | 3.307 | 6.028 | 0.000 | 0.000 | 6.366 |
| LNM Group 2 vs. Group 1 | ENSG00000143772 | ITPKB | −0.844 | 6.898 | −6.061 | 0.000 | 0.000 | 6.581 |
| LNM Group 2 vs. Group 1 | ENSG00000166432 | ZMAT1 | −1.402 | 4.478 | −6.088 | 0.000 | 0.000 | 6.561 |
| LNM Group 2 vs. Group 1 | ENSG00000154217 | PITPNC1 | −0.831 | 5.134 | −6.105 | 0.000 | 0.000 | 6.678 |
| LNM Group 2 vs. Group 1 | ENSG00000196911 | KPNA5 | −0.672 | 7.718 | −6.154 | 0.000 | 0.000 | 6.874 |
| LNM Group 2 vs. Group 1 | ENSG00000149311 | ATM | −0.980 | 8.318 | −6.207 | 0.000 | 0.000 | 7.032 |
| LNM Group 2 vs. Group 1 | ENSG00000111321 | LTBR | 0.773 | 6.858 | 6.338 | 0.000 | 0.000 | 7.445 |
| LNM Group 2 vs. Group 1 | ENSG00000113441 | LNPEP | −0.887 | 8.260 | −6.342 | 0.000 | 0.000 | 7.457 |
| LNM Group 2 vs. Group 1 | ENSG00000133872 | SARAF | −0.771 | 7.323 | −6.348 | 0.000 | 0.000 | 7.477 |
| LNM Group 2 vs. Group 1 | ENSG00000169762 | TAPT1 | −0.786 | 4.892 | −6.364 | 0.000 | 0.000 | 7.459 |
| LNM Group 2 vs. Group 1 | ENSG00000114867 | EIF4G1 | 0.607 | 9.184 | 6.399 | 0.000 | 0.000 | 7.624 |
| LNM Group 2 vs. Group 1 | ENSG00000107404 | DVL1 | 0.915 | 6.014 | 6.438 | 0.000 | 0.000 | 7.734 |
| LNM Group 2 vs. Group 1 | ENSG00000104177 | MYEF2 | −1.210 | 4.015 | −6.533 | 0.000 | 0.000 | 7.953 |
| LNM Group 2 vs. Group 1 | ENSG00000117411 | B4GALT2 | 1.040 | 5.379 | 6.698 | 0.000 | 0.000 | 8.521 |
| LNM Group 2 vs. Group 1 | ENSG00000205189 | ZBTB10 | −1.170 | 5.508 | −6.729 | 0.000 | 0.000 | 8.623 |
| LNM Group 2 vs. Group 1 | ENSG00000101109 | STK4 | −0.867 | 7.643 | −6.952 | 0.000 | 0.000 | 9.349 |
| LNM Group 2 vs. Group 1 | ENSG00000103657 | HERC1 | −0.688 | 7.923 | −7.062 | 0.000 | 0.000 | 9.692 |
| LNM Group 2 vs. Group 1 | ENSG00000128000 | ZNF780B | −0.755 | 5.830 | −7.077 | 0.000 | 0.000 | 9.698 |
| LNM Group 2 vs. Group 1 | ENSG00000167962 | ZNF598 | 0.989 | 5.911 | 7.535 | 0.000 | 0.000 | 11.077 |
| LNM Group 2 vs. Group 3 | ENSG00000140320 | BAHD1 | −0.696 | 5.343 | −3.250 | 0.002 | 0.050 | −1.577 |
| LNM Group 2 vs. Group 3 | ENSG00000137474 | MYO7A | 0.990 | 4.279 | 3.254 | 0.002 | 0.050 | −1.485 |
| LNM Group 2 vs. Group 3 | ENSG00000151292 | CSNK1G3 | −0.720 | 5.789 | −3.254 | 0.002 | 0.050 | −1.560 |
| LNM Group 2 vs. Group 3 | ENSG00000203747 | FCGR3A | 2.306 | 5.467 | 3.260 | 0.002 | 0.049 | −1.496 |
| LNM Group 2 vs. Group 3 | ENSG00000163686 | ABHD6 | 0.879 | 3.150 | 3.256 | 0.002 | 0.049 | −1.472 |
| LNM Group 2 vs. Group 3 | ENSG00000110047 | EHD1 | 0.756 | 6.373 | 3.259 | 0.002 | 0.049 | −1.572 |
| LNM Group 2 vs. Group 3 | ENSG00000108604 | SMARCD2 | −0.658 | 6.178 | −3.257 | 0.002 | 0.049 | −1.573 |
| LNM Group 2 vs. Group 3 | ENSG00000138757 | G3BP2 | −0.694 | 7.036 | −3.259 | 0.002 | 0.049 | −1.597 |
| LNM Group 2 vs. Group 3 | ENSG00000151748 | SAV1 | −0.835 | 5.495 | −3.256 | 0.002 | 0.049 | −1.538 |
| LNM Group 2 vs. Group 3 | ENSG00000163131 | CTSS | 1.239 | 6.658 | 3.264 | 0.002 | 0.049 | −1.528 |
| LNM Group 2 vs. Group 3 | ENSG00000051523 | CYBA | 0.915 | 6.100 | 3.264 | 0.002 | 0.049 | −1.525 |
| LNM Group 2 vs. Group 3 | ENSG00000116871 | MAP7D1 | 0.911 | 6.943 | 3.263 | 0.002 | 0.049 | −1.582 |
| LNM Group 2 vs. Group 3 | ENSG00000125166 | GOT2 | 0.586 | 6.083 | 3.263 | 0.002 | 0.049 | −1.553 |
| LNM Group 2 vs. Group 3 | ENSG00000197608 | ZNF841 | 0.887 | 5.508 | 3.266 | 0.002 | 0.049 | −1.531 |
| LNM Group 2 vs. Group 3 | ENSG00000117984 | CTSD | 1.009 | 8.610 | 3.272 | 0.002 | 0.049 | −1.570 |
| LNM Group 2 vs. Group 3 | ENSG00000166813 | KIF7 | −1.379 | 3.863 | −3.274 | 0.002 | 0.049 | −1.479 |
| LNM Group 2 vs. Group 3 | ENSG00000131724 | ILI3RA1 | 0.925 | 6.731 | 3.274 | 0.002 | 0.048 | −1.530 |
| LNM Group 2 vs. Group 3 | ENSG00000141644 | MBD1 | 0.766 | 6.316 | 3.275 | 0.002 | 0.048 | −1.544 |
| LNM Group 2 vs. Group 3 | ENSG00000228570 | NUTM2E | 0.903 | 2.282 | 3.276 | 0.002 | 0.048 | −1.410 |
| LNM Group 2 vs. Group 3 | ENSG00000004777 | ARHGAP33 | −1.405 | 4.471 | −3.279 | 0.002 | 0.048 | −1.477 |
| LNM Group 2 vs. Group 3 | ENSG00000111666 | CHPT1 | −0.988 | 4.953 | −3.280 | 0.002 | 0.048 | −1.465 |
| LNM Group 2 vs. Group 3 | ENSG00000232388 | LINC00493 | −0.728 | 3.589 | −3.281 | 0.002 | 0.048 | −1.413 |
| LNM Group 2 vs. Group 3 | ENSG00000179627 | ZBTB42 | −0.865 | 3.343 | −3.284 | 0.002 | 0.048 | −1.402 |
| LNM Group 2 vs. Group 3 | ENSG00000146192 | FGD2 | 1.348 | 6.172 | 3.291 | 0.002 | 0.048 | −1.456 |
| LNM Group 2 vs. Group 3 | ENSG00000110675 | ELMOD1 | 1.282 | 1.844 | 3.288 | 0.002 | 0.048 | −1.379 |
| LNM Group 2 vs. Group 3 | ENSG00000054965 | FAM168A | 0.660 | 6.732 | 3.289 | 0.002 | 0.048 | −1.501 |
| LNM Group 2 vs. Group 3 | ENSG00000105372 | RPS19 | −0.777 | 8.221 | −3.290 | 0.002 | 0.048 | −1.520 |
| LNM Group 2 vs. Group 3 | ENSG00000120549 | KIAA1217 | −0.926 | 6.908 | −3.290 | 0.002 | 0.048 | −1.517 |
| LNM Group 2 vs. Group 3 | ENSG00000203880 | PCMTD2 | −0.627 | 6.261 | −3.295 | 0.002 | 0.047 | −1.486 |
| LNM Group 2 vs. Group 3 | ENSG00000178726 | THBD | 1.537 | 6.440 | 3.300 | 0.002 | 0 047 | −1 405 |
| LNM Group 2 vs. Group 3 | ENSG00000136960 | ENPP2 | 1.482 | 4.829 | 3.304 | 0.002 | 0.047 | −1.379 |
| LNM Group 2 vs. Group 3 | ENSG00000188641 | DPYD | 1.127 | 6.028 | 3.302 | 0.002 | 0.047 | −1.400 |
| LNM Group 2 vs. Group 3 | ENSG00000110719 | TCIRG1 | 0.859 | 6.715 | 3.306 | 0.002 | 0.047 | −1.466 |
| LNM Group 2 vs. Group 3 | ENSG00000189403 | HMGB1 | −0.601 | 7.914 | −3.303 | 0.002 | 0.047 | −1.491 |
| LNM Group 2 vs. Group 3 | ENSG00000116128 | BCL9 | −1.081 | 4.976 | −3.305 | 0.002 | 0.047 | −1.418 |
| LNM Group 2 vs. Group 3 | ENSG00000070404 | FSTL3 | 1.716 | 5.126 | 3.309 | 0.002 | 0.047 | −1.368 |
| LNM Group 2 vs. Group 3 | ENSG00000184497 | TMEM255B | 1.352 | 3.912 | 3.308 | 0.002 | 0.047 | −1.353 |
| LNM Group 2 vs. Group 3 | ENSG00000067704 | IARS2 | −0.731 | 6.373 | −3.308 | 0.002 | 0.047 | −1.444 |
| LNM Group 2 vs. Group 3 | ENSG00000196329 | GIMAP5 | 1.666 | 5.025 | 3.315 | 0.002 | 0.046 | −1.363 |
| LNM Group 2 vs. Group 3 | ENSG00000221995 | TIAF1 | 0.688 | 4.552 | 3.313 | 0.002 | 0.046 | −1.353 |
| LNM Group 2 vs. Group 3 | ENSG00000182004 | SNRPE | −0.906 | 4.387 | −3.312 | 0.002 | 0.046 | −1.362 |
| LNM Group 2 vs. Group 3 | ENSG00000103066 | PLA2G15 | 0.595 | 4.434 | 3.316 | 0.002 | 0.046 | −1.345 |
| LNM Group 2 vs. Group 3 | ENSG00000243244 | STON1 | −1.178 | 5.027 | −3.319 | 0.002 | 0.046 | −1.373 |
| LNM Group 2 vs. Group 3 | ENSG00000141506 | PIK3R5 | 1.618 | 5.404 | 3.321 | 0.002 | 0.046 | −1.360 |
| LNM Group 2 vs. Group 3 | ENSG00000124688 | MAD2L1BP | −0.684 | 3.675 | −3.318 | 0.002 | 0.046 | −1.334 |
| LNM Group 2 vs. Group 3 | ENSG00000149357 | LAMTOR1 | 0.585 | 5.545 | 3.321 | 0.002 | 0.046 | −1.358 |
| LNM Group 2 vs. Group 3 | ENSG00000176463 | SLCO3A1 | −0.928 | 5.862 | −3.322 | 0.002 | 0.046 | −1.426 |
| LNM Group 2 vs. Group 3 | ENSG00000164692 | COL1A2 | 2.484 | 10.783 | 3.327 | 0.002 | 0.046 | −1.408 |
| LNM Group 2 vs. Group 3 | ENSG00000113721 | PDGFRB | 1.249 | 7.338 | 3.329 | 0.002 | 0.046 | −1.410 |
| LNM Group 2 vs. Group 3 | ENSG00000124766 | SOX4 | −1.224 | 6.606 | −3.331 | 0.002 | 0.045 | −1.411 |
| LNM Group 2 vs. Group 3 | ENSG00000181830 | SLC35C1 | 0.833 | 4.744 | 3.330 | 0.002 | 0.045 | −1.315 |
| LNM Group 2 vs. Group 3 | ENSG00000175727 | MLXIP | 0.874 | 7.832 | 3.331 | 0.002 | 0.045 | −1.418 |
| LNM Group 2 vs. Group 3 | ENSG00000198909 | MAP3K3 | 0.747 | 5.826 | 3.333 | 0.002 | 0.045 | −1.372 |
| LNM Group 2 vs. Group 3 | ENSG00000115137 | DNAJC27 | −1.123 | 3.926 | −3.335 | 0.002 | 0.045 | −1.292 |
| LNM Group 2 vs. Group 3 | ENSG00000169957 | ZNF768 | −0.719 | 4.412 | −3.338 | 0.002 | 0.045 | −1.319 |
| LNM Group 2 vs. Group 3 | ENSG00000154133 | ROBO4 | 1.300 | 4.075 | 3.344 | 0.002 | 0.045 | −1.268 |
| LNM Group 2 vs. Group 3 | ENSG00000134830 | C5AR2 | 1.287 | 4.874 | 3.344 | 0.002 | 0.045 | −1.293 |
| LNM Group 2 vs. Group 3 | ENSG00000125827 | TMX4 | −0.781 | 6.112 | −3.344 | 0.002 | 0.045 | −1.357 |
| LNM Group 2 vs. Group 3 | ENSG00000125652 | ALKBH7 | −0.901 | 3.115 | −3.341 | 0.002 | 0.045 | −1.261 |
| LNM Group 2 vs. Group 3 | ENSG00000155893 | PXYLP1 | −0.992 | 4.930 | −3.342 | 0.002 | 0.045 | −1.345 |
| LNM Group 2 vs. Group 3 | ENSG00000160691 | SHC1 | 0.632 | 7.619 | 3.347 | 0.002 | 0.044 | −1.371 |
| LNM Group 2 vs. Group 3 | ENSG00000163125 | RPRD2 | −0.596 | 6.520 | −3.347 | 0.002 | 0.044 | −1.361 |
| LNM Group 2 vs. Group 3 | ENSG00000170604 | IRF2BP1 | −0.759 | 4.024 | −3.349 | 0.002 | 0.044 | −1.275 |
| LNM Group 2 vs. Group 3 | ENSG00000136636 | KCTD3 | −0.861 | 6.019 | −3.347 | 0.002 | 0.044 | −1.348 |
| LNM Group 2 vs. Group 3 | ENSG00000124440 | HIF3A | −1.490 | 4.340 | −3.350 | 0.002 | 0.044 | −1.283 |
| LNM Group 2 vs. Group 3 | ENSG00000163513 | TGFBR2 | 1.017 | 7.259 | 3.352 | 0.002 | 0.044 | −1.334 |
| LNM Group 2 vs. Group 3 | ENSG00000162377 | COA7 | 0.843 | 5.401 | 3.353 | 0.002 | 0.044 | −1.306 |
| LNM Group 2 vs. Group 3 | ENSG00000204070 | SYS1 | 0.593 | 5.270 | 3.355 | 0.002 | 0.044 | −1.300 |
| LNM Group 2 vs. Group 3 | ENSG00000069275 | NUCKS1 | −0.885 | 8.703 | −3.357 | 0.002 | 0.044 | −1.332 |
| LNM Group 2 vs. Group 3 | ENSG00000138080 | EMILIN1 | 1.533 | 5.519 | 3.361 | 0.002 | 0.044 | −1.271 |
| LNM Group 2 vs. Group 3 | ENSG00000138641 | HERC3 | 0.886 | 6.303 | 3.360 | 0.002 | 0.044 | −1.294 |
| LNM Group 2 vs. Group 3 | ENSG00000216937 | CCDC7 | −1.182 | 3.695 | −3.362 | 0.002 | 0.044 | −1.210 |
| LNM Group 2 vs. Group 3 | ENSG00000187474 | FPR3 | 1.319 | 4.801 | 3.367 | 0.002 | 0.043 | −1.220 |
| LNM Group 2 vs. Group 3 | ENSG00000059377 | TBXAS1 | 0.916 | 4.305 | 3.366 | 0.002 | 0.043 | −1.210 |
| LNM Group 2 vs. Group 3 | ENSG00000110931 | CAMKK2 | 0.639 | 6.140 | 3.364 | 0.002 | 0.043 | −1.316 |
| LNM Group 2 vs. Group 3 | ENSG00000063601 | MTMR1 | −0.647 | 6.308 | −3.366 | 0.002 | 0.043 | −1.309 |
| LNM Group 2 vs. Group 3 | ENSG00000138119 | MYOF | 1.021 | 7.897 | 3.371 | 0.002 | 0.043 | −1.313 |
| LNM Group 2 vs. Group 3 | ENSG00000257093 | KIAA1147 | −0.784 | 6.997 | −3.372 | 0.002 | 0.043 | −1.311 |
| LNM Group 2 vs. Group 3 | ENSG00000182899 | RPL35A | −0.864 | 7.557 | −3.371 | 0.002 | 0.043 | −1.315 |
| LNM Group 2 vs. Group 3 | ENSG00000159403 | C1R | 1.047 | 7.164 | 3.375 | 0.002 | 0.043 | −1.268 |
| LNM Group 2 vs. Group 3 | ENSG00000116199 | FAM2OB | −0.812 | 6.664 | −3.374 | 0.002 | 0.043 | −1.297 |
| LNM Group 2 vs. Group 3 | ENSG00000168542 | COL3A1 | 2.204 | 11.045 | 3.379 | 0.002 | 0.043 | −1.276 |
| LNM Group 2 vs. Group 3 | ENSG00000145990 | GFOD1 | 0.986 | 6.415 | 3.377 | 0.002 | 0.043 | −1.251 |
| LNM Group 2 vs. Group 3 | ENSG00000078237 | TIGAR | 1.712 | 4.361 | 3.383 | 0.002 | 0.043 | −1.185 |
| LNM Group 2 vs. Group 3 | ENSG00000078070 | MCCC1 | −0.867 | 5.043 | −3.382 | 0.002 | 0.043 | −1.240 |
| LNM Group 2 vs. Group 3 | ENSG00000112149 | CD83 | −1.334 | 5.120 | −3.383 | 0.002 | 0.043 | −1.249 |
| LNM Group 2 vs. Group 3 | ENSG00000112541 | PDE10A | −1.442 | 3.825 | −3.385 | 0.002 | 0.043 | −1.182 |
| LNM Group 2 vs. Group 3 | ENSG00000164684 | ZNF704 | −1.652 | 5.657 | −3.386 | 0.002 | 0.043 | −1.247 |
| LNM Group 2 vs. Group 3 | ENSG00000102871 | TRADD | 0.696 | 4.713 | 3.386 | 0.002 | 0.042 | −1.171 |
| LNM Group 2 vs. Group 3 | ENSG00000113742 | CPEB4 | 1.134 | 6.574 | 3.390 | 0.002 | 0.042 | −1.210 |
| LNM Group 2 vs. Group 3 | ENSG00000159733 | ZFYVE28 | 1.193 | 3.826 | 3.390 | 0.002 | 0.042 | −1.140 |
| LNM Group 2 vs. Group 3 | ENSG00000259207 | ITGB3 | 1.985 | 3.076 | 3.397 | 0.002 | 0.042 | −1.122 |
| LNM Group 2 vs. Group 3 | ENSG00000139629 | GALNT6 | 1.699 | 5.858 | 3.400 | 0.002 | 0.042 | −1.191 |
| LNM Group 2 vs. Group 3 | ENSG00000162769 | FLVCR1 | −0.854 | 5.445 | −3.398 | 0.002 | 0.042 | −1.187 |
| LNM Group 2 vs. Group 3 | ENSG00000135338 | LCA5 | −0.959 | 2.683 | −3.397 | 0.002 | 0.042 | −1.150 |
| LNM Group 2 vs. Group 3 | ENSG00000118298 | CA14 | −1.078 | 2.553 | −3.399 | 0.001 | 0.042 | −1.112 |
| LNM Group 2 vs. Group 3 | ENSG00000007237 | GAS7 | 1.337 | 5.701 | 3.406 | 0.001 | 0.041 | −1.168 |
| LNM Group 2 vs. Group 3 | ENSG00000147905 | ZCCHC7 | −0.595 | 5.368 | −3.404 | 0.001 | 0.041 | −1.191 |
| LNM Group 2 vs. Group 3 | ENSG00000114988 | LMAN2L | −0.826 | 3.777 | −3.406 | 0.001 | 0.041 | −1.110 |
| LNM Group 2 vs. Group 3 | ENSG00000144445 | KANSL1L | −0.593 | 5.622 | −3.409 | 0.001 | 0.041 | −1.172 |
| LNM Group 2 vs. Group 3 | ENSG00000111711 | GOLT1B | 0.885 | 5.773 | 3.412 | 0.001 | 0.041 | −1.149 |
| LNM Group 2 vs. Group 3 | ENSG00000165512 | ZNF22 | −0.731 | 4.449 | −3.412 | 0.001 | 0.041 | −1.125 |
| LNM Group 2 vs. Group 3 | ENSG00000196912 | ANKRD36B | −0.788 | 5.995 | −3.413 | 0.001 | 0.041 | −1.171 |
| LNM Group 2 vs. Group 3 | ENSG00000204287 | HLA-DRA | 1.148 | 8.392 | 3.421 | 0.001 | 0.041 | −1.174 |
| LNM Group 2 vs. Group 3 | ENSG00000033327 | GAB2 | 1.234 | 5.449 | 3.424 | 0.001 | 0.041 | −1.104 |
| LNM Group 2 vs. Group 3 | ENSG00000118369 | USP35 | 1.266 | 4.301 | 3.424 | 0.001 | 0.041 | −1.069 |
| LNM Group 2 vs. Group 3 | ENSG00000088682 | COQ9 | 0.797 | 4.454 | 3.423 | 0.001 | 0.041 | −1.070 |
| LNM Group 2 vs. Group 3 | ENSG00000146197 | SCUBE3 | −0.662 | 4.473 | −3.424 | 0.001 | 0.041 | −1.145 |
| LNM Group 2 vs. Group 3 | ENSG00000056277 | ZNF280C | −0.759 | 4.238 | −3.424 | 0.001 | 0.041 | −1.072 |
| LNM Group 2 vs. Group 3 | ENSG00000129538 | RNASE1 | 2.520 | 4.986 | 3.431 | 0.001 | 0.040 | −1.046 |
| LNM Group 2 vs. Group 3 | ENSG00000205423 | CNEP1R1 | 0.637 | 4.539 | 3.428 | 0.001 | 0.040 | −1.063 |
| LNM Group 2 vs. Group 3 | ENSG00000166503 | RP11- | −0.916 | 6.556 | −3.431 | 0.001 | 0.040 | −1.149 |
| | | 382A20.3 | | | | | | |
| LNM Group 2 vs. Group 3 | ENSG00000128228 | SDF2L1 | 0.962 | 3.008 | 3.434 | 0.001 | 0.040 | −1.026 |
| LNM Group 2 vs. Group 3 | ENSG00000174669 | SLC29A2 | −1.331 | 3.120 | −3.438 | 0.001 | 0.040 | −1.042 |
| LNM Group 2 vs. Group 3 | ENSG00000112992 | NNT | −0.671 | 6.532 | −3.439 | 0.001 | 0.040 | −1.120 |
| LNM Group 2 vs. Group 3 | ENSG00000175155 | YPEL2 | 0.914 | 5.977 | 3.445 | 0.001 | 0.039 | −1.068 |
| LNM Group 2 vs. Group 3 | ENSG00000162885 | B3GALNT2 | −0.655 | 5.346 | −3.446 | 0.001 | 0.039 | −1.082 |
| LNM Group 2 vs. Group 3 | ENSG00000074696 | HACD3 | −0.713 | 6.575 | −3.445 | 0.001 | 0.039 | −1.113 |
| LNM Group 2 vs. Group 3 | ENSG00000103222 | ABCC1 | −0.671 | 7.732 | −3.448 | 0.001 | 0.039 | −1.114 |
| LNM Group 2 vs. Group 3 | ENSG00000197044 | ZNF441 | −1.006 | 4.020 | −3.451 | 0.001 | 0.039 | −1.001 |
| LNM Group 2 vs. Group 3 | ENSG00000001167 | NFYA | −0.699 | 5.384 | −3.453 | 0.001 | 0.039 | −1.061 |
| LNM Group 2 vs. Group 3 | ENSG00000121068 | TBX2 | 1.866 | 3.947 | 3.458 | 0.001 | 0.039 | −0.991 |
| LNM Group 2 vs. Group 3 | ENSG00000031081 | ARHGAP31 | 0.871 | 6.390 | 3.455 | 0.001 | 0.039 | −1.079 |
| LNM Group 2 vs. Group 3 | ENSG00000170004 | CHD3 | −0.867 | 8.232 | −3.460 | 0.001 | 0.038 | −1.073 |
| LNM Group 2 vs. Group 3 | ENSG00000179456 | ZBTB18 | −1.003 | 4.841 | −3.462 | 0.001 | 0.038 | −1.017 |
| LNM Group 2 vs. Group 3 | ENSG00000010327 | STAB1 | 1.437 | 7.455 | 3.466 | 0.001 | 0.038 | −1.055 |
| LNM Group 2 vs. Group 3 | ENSG00000004399 | PLXND1 | 0.779 | 7.289 | 3.465 | 0.001 | 0.038 | −1.065 |
| LNM Group 2 vs. Group 3 | ENSG00000138175 | ARL3 | −0.728 | 3.153 | −3.465 | 0.001 | 0.038 | −0.954 |
| LNM Group 2 vs. Group 3 | ENSG00000185989 | RASA3 | 1.081 | 5.993 | 3.469 | 0.001 | 0.038 | −1.005 |
| LNM Group 2 vs. Group 3 | ENSG00000111424 | VDR | 0.989 | 5.716 | 3.470 | 0.001 | 0.038 | −0.983 |
| LNM Group 2 vs. Group 3 | ENSG00000100284 | TOM1 | 0.792 | 5.827 | 3.468 | 0.001 | 0.038 | −1.000 |
| LNM Group 2 vs. Group 3 | ENSG00000078043 | PIAS2 | −0.725 | 6.289 | −3.468 | 0.001 | 0.038 | −1.043 |
| LNM Group 2 vs. Group 3 | ENSG00000109099 | PMP22 | 1.621 | 5.421 | 3.479 | 0.001 | 0.037 | −0.973 |
| LNM Group 2 vs. Group 3 | ENSG00000176845 | METRNL | 1.219 | 5.430 | 3.478 | 0.001 | 0.037 | −0.981 |
| LNM Group 2 vs. Group 3 | ENSG00000110442 | COMMD9 | 0.712 | 4.764 | 3.477 | 0.001 | 0.037 | −0.960 |
| LNM Group 2 vs. Group 3 | ENSG00000166946 | CCNDBP1 | 0.746 | 5.713 | 3.479 | 0.001 | 0.037 | −0.966 |
| LNM Group 2 vs. Group 3 | ENSG00000125843 | AP5S1 | −0.911 | 5.039 | −3.483 | 0.001 | 0.037 | −0.973 |
| LNM Group 2 vs. Group 3 | ENSG00000171115 | GIMAP8 | 1.210 | 4.344 | 3.486 | 0.001 | 0.037 | −0.906 |
| LNM Group 2 vs. Group 3 | ENSG00000169403 | PTAFR | 1.031 | 4.914 | 3.489 | 0.001 | 0.037 | −0.905 |
| LNM Group 2 vs. Group 3 | ENSG00000187607 | ZNF286A | −0.901 | 4.927 | −3.490 | 0.001 | 0.037 | −0.955 |
| LNM Group 2 vs. Group 3 | ENSG00000107779 | BMPR1A | −0.716 | 5.699 | −3.492 | 0.001 | 0.037 | −0.950 |
| LNM Group 2 vs. Group 3 | ENSG00000196743 | GM2A | 1.103 | 7.342 | 3.499 | 0.001 | 0.036 | −0.945 |
| LNM Group 2 vs. Group 3 | ENSG00000119711 | ALDH6A1 | −0.679 | 5.178 | −3.498 | 0.001 | 0.036 | −0.915 |
| LNM Group 2 vs. Group 3 | ENSG00000126217 | MCF2L | −1.412 | 5.564 | −3.499 | 0.001 | 0.036 | −0.949 |
| LNM Group 2 vs. Group 3 | ENSG00000168404 | MLKL | 1.035 | 5.151 | 3.503 | 0.001 | 0.036 | −0.881 |
| LNM Group 2 vs. Group 3 | ENSG00000131100 | ATP6V1E1 | 0.595 | 5.617 | 3.502 | 0.001 | 0.036 | −0.923 |
| LNM Group 2 vs. Group 3 | ENSG00000086619 | ERO1B | −0.881 | 4.742 | −3.501 | 0.001 | 0.036 | −0.902 |
| LNM Group 2 vs. Group 3 | ENSG00000168758 | SEMA4C | −0.897 | 6.018 | −3.502 | 0.001 | 0.036 | −0.952 |
| LNM Group 2 vs. Group 3 | ENSG00000205363 | C150rf59 | −2.208 | 3.251 | −3.514 | 0.001 | 0.035 | −0.842 |
| LNM Group 2 vs. Group 3 | ENSG00000160796 | NBEAL2 | 0.866 | 7.603 | 3.516 | 0.001 | 0.035 | −0.928 |
| LNM Group 2 vs. Group 3 | ENSG00000112144 | ICK | −0.824 | 5.453 | −3.517 | 0.001 | 0.035 | −0.897 |
| LNM Group 2 vs. Group 3 | ENSG00000141384 | TAF4B | −0.803 | 4.539 | −3.527 | 0.001 | 0.034 | −0.858 |
| LNM Group 2 vs. Group 3 | ENSG00000115523 | GNLY | 1.725 | 3.611 | 3.534 | 0.001 | 0.034 | −0.783 |
| LNM Group 2 vs. Group 3 | ENSG00000177954 | RPS27 | −1.094 | 7.559 | −3.534 | 0.001 | 0.034 | −0.888 |
| LNM Group 2 vs. Group 3 | ENSG00000144591 | GMPPA | 0.706 | 4.906 | 3.535 | 0.001 | 0.034 | −0.805 |
| LNM Group 2 vs. Group 3 | ENSG00000169744 | LDB2 | 1.359 | 3.759 | 3.540 | 0.001 | 0.033 | −0.764 |
| LNM Group 2 vs. Group 3 | ENSG00000174600 | CMKLR1 | 1.587 | 4.770 | 3.546 | 0.001 | 0.033 | −0.759 |
| LNM Group 2 vs. Group 3 | ENSG00000164733 | CTSB | 1.111 | 9.580 | 3.548 | 0.001 | 0.033 | −0.835 |
| LNM Group 2 vs. Group 3 | ENSG00000167635 | ZNF146 | −0.604 | 6.698 | −3.543 | 0.001 | 0.033 | −0.854 |
| LNM Group 2 vs. Group 3 | ENSG00000123607 | TTC21B | −0.664 | 5.188 | −3.545 | 0.001 | 0.033 | −0.824 |
| LNM Group 2 vs. Group 3 | ENSG00000066651 | TRMT11 | −0.722 | 3.866 | −3.543 | 0.001 | 0.033 | −0.772 |
| LNM Group 2 vs. Group 3 | ENSG00000106333 | PCOLCE | 1.902 | 5.110 | 3.558 | 0.001 | 0.033 | −0.737 |
| LNM Group 2 vs. Group 3 | ENSG00000145022 | TCTA | 0.820 | 4.121 | 3.552 | 0.001 | 0.033 | −0.742 |
| LNM Group 2 vs. Group 3 | ENSG00000103248 | MTHFSD | 0.698 | 4.540 | 3.552 | 0.001 | 0.033 | −0.751 |
| LNM Group 2 vs. Group 3 | ENSG00000050344 | NFE2L3 | −0.700 | 6.014 | −3.553 | 0.001 | 0.033 | −0.820 |
| LNM Group 2 vs. Group 3 | ENSG00000109654 | TRIM2 | −1.146 | 5.906 | −3.555 | 0.001 | 0.033 | −0.821 |
| LNM Group 2 vs. Group 3 | ENSG00000099998 | GGT5 | 1.554 | 5.447 | 3.560 | 0.001 | 0.033 | −0.740 |
| LNM Group 2 vs. Group 3 | ENSG00000160255 | ITGB2 | 1.300 | 6.600 | 3.561 | 0.001 | 0.033 | −0.780 |
| LNM Group 2 vs. Group 3 | ENSG00000107819 | SFXN3 | 1.029 | 5.841 | 3.557 | 0.001 | 0.033 | −0.753 |
| LNM Group 2 vs. Group 3 | ENSG00000204592 | HLA-E | 0.801 | 9.225 | 3.559 | 0.001 | 0.033 | −0.819 |
| LNM Group 2 vs. Group 3 | ENSG00000151689 | INPP1 | 0.993 | 3.938 | 3.561 | 0.001 | 0.033 | −0.724 |
| LNM Group 2 vs. Group 3 | ENSG00000070759 | TESK2 | −0.782 | 3.250 | −3.563 | 0.001 | 0.032 | −0.707 |
| LNM Group 2 vs. Group 3 | ENSG00000010017 | RANBP9 | −0.620 | 6.133 | −3.566 | 0.001 | 0.032 | −0.769 |
| LNM Group 2 vs. Group 3 | ENSG00000083720 | OXCT1 | −1.080 | 5.273 | −3.566 | 0.001 | 0.032 | −0.727 |
| LNM Group 2 vs. Group 3 | ENSG00000159128 | IFNGR2 | 0.808 | 5.982 | 3.570 | 0.001 | 0.032 | −0.759 |
| LNM Group 2 vs. Group 3 | ENSG00000156531 | PHF6 | −0.590 | 5.965 | −3.570 | 0.001 | 0.032 | −0.762 |
| LNM Group 2 vs. Group 3 | ENSG00000114796 | KLHL24 | −0.788 | 7.292 | −3.571 | 0.001 | 0.032 | −0.788 |
| LNM Group 2 vs. Group 3 | ENSG00000100036 | SLC35E4 | 1.050 | 2.833 | 3.573 | 0.001 | 0.032 | −0.677 |
| LNM Group 2 vs. Group 3 | ENSG00000105738 | SIPA1L3 | −0.793 | 7.401 | −3.573 | 0.001 | 0.032 | −0.778 |
| LNM Group 2 vs. Group 3 | ENSG00000103335 | PIEZO1 | 0.841 | 8.317 | 3.576 | 0.001 | 0.032 | −0.777 |
| LNM Group 2 vs. Group 3 | ENSG00000128052 | KDR | 1.410 | 4.276 | 3.584 | 0.001 | 0.032 | −0.677 |
| LNM Group 2 vs. Group 3 | ENSG00000025708 | TYMP | 1.152 | 7.828 | 3.586 | 0.001 | 0.032 | −0.740 |
| LNM Group 2 vs. Group 3 | ENSG00000153187 | HNRNPU | −0.623 | 9.521 | −3.580 | 0.001 | 0.032 | −0.746 |
| LNM Group 2 vs. Group 3 | ENSG00000101236 | RNF24 | −0.728 | 5.965 | −3.579 | 0.001 | 0.032 | −0.742 |
| LNM Group 2 vs. Group 3 | ENSG00000132196 | HSD17B7 | −0.739 | 4.321 | −3.579 | 0.001 | 0.032 | −0.679 |
| LNM Group 2 vs. Group 3 | ENSG00000266074 | BAHCC1 | −1.497 | 5.591 | −3.582 | 0.001 | 0.032 | −0.735 |
| LNM Group 2 vs. Group 3 | ENSG00000143149 | ALDH9A1 | −0.810 | 5.481 | −3.596 | 0.001 | 0.031 | −0.670 |
| LNM Group 2 vs. Group 3 | ENSG00000241839 | PLEKHO2 | 0.832 | 5.640 | 3.598 | 0.001 | 0.031 | −0.663 |
| LNM Group 2 vs. Group 3 | ENSG00000082805 | ERC1 | 0.601 | 7.227 | 3.599 | 0.001 | 0.031 | −0.715 |
| LNM Group 2 vs. Group 3 | ENSG00000079215 | SLC1A3 | 1.654 | 5.467 | 3.607 | 0.001 | 0.031 | −0.622 |
| LNM Group 2 vs. Group 3 | ENSG00000173442 | EHBP1L1 | 0.799 | 7.917 | 3.604 | 0.001 | 0.031 | −0.697 |
| LNM Group 2 vs. Group 3 | ENSG00000119669 | IRF2BPL | −0.666 | 5.943 | −3.609 | 0.001 | 0.030 | −0.649 |
| LNM Group 2 vs. Group 3 | ENSG00000178163 | ZNF518B | −0.949 | 5.931 | −3.610 | 0.001 | 0.030 | −0.665 |
| LNM Group 2 vs. Group 3 | ENSG00000130489 | SCO2 | 1.585 | 4.765 | 3.616 | 0.001 | 0.030 | −0.595 |
| LNM Group 2 vs. Group 3 | ENSG00000134470 | IL15RA | 1.086 | 3.674 | 3.612 | 0.001 | 0.030 | −0.579 |
| LNM Group 2 vs. Group 3 | ENSG00000152382 | TADA1 | −0.739 | 3.995 | −3.617 | 0.001 | 0.030 | −0.583 |
| LNM Group 2 vs. Group 3 | ENSG00000171045 | TSNARE1 | −0.800 | 4.465 | −3.621 | 0.001 | 0.030 | −0.593 |
| LNM Group 2 vs. Group 3 | ENSG00000131018 | SYNE1 | 1.035 | 7.671 | 3.626 | 0.001 | 0.030 | −0.633 |
| LNM Group 2 vs. Group 3 | ENSG00000162511 | LAPTM5 | 1.280 | 7.971 | 3.636 | 0.001 | 0.029 | −0.604 |
| LNM Group 2 vs. Group 3 | ENSG00000146070 | PLA2G7 | 1.398 | 3.797 | 3.637 | 0.001 | 0.029 | −0.518 |
| LNM Group 2 vs. Group 3 | ENSG00000196126 | HLA-DRB1 | 1.310 | 7.074 | 3.643 | 0.001 | 0.029 | −0.562 |
| LNM Group 2 vs. Group 3 | ENSG00000095139 | ARCN1 | 0.595 | 7.074 | 3.640 | 0.001 | 0.029 | −0.589 |
| LNM Group 2 vs. Group 3 | ENSG00000116957 | TBCE | −0.606 | 4.423 | −3.642 | 0.001 | 0.029 | −0.527 |
| LNM Group 2 vs. Group 3 | ENSG00000160111 | CPAMD8 | −1.192 | 4.049 | −3.648 | 0.001 | 0.028 | −0.500 |
| LNM Group 2 vs. Group 3 | ENSG00000019144 | PHLDB1 | 1.101 | 7.590 | 3.654 | 0.001 | 0.028 | −0.566 |
| LNM Group 2 vs. Group 3 | ENSG00000023572 | GLRX2 | 0.971 | 1.996 | 3.651 | 0.001 | 0.028 | −0.474 |
| LNM Group 2 vs. Group 3 | ENSG00000105866 | SP4 | −0.972 | 5.031 | −3.651 | 0.001 | 0.028 | −0.515 |
| LNM Group 2 vs. Group 3 | ENSG00000177628 | GBA | 0.798 | 4.994 | 3.656 | 0.001 | 0.028 | −0.484 |
| LNM Group 2 vs. Group 3 | ENSG00000153786 | ZDHHC7 | 0.620 | 6.114 | 3.657 | 0.001 | 0.028 | −0.510 |
| LNM Group 2 vs. Group 3 | ENSG00000140391 | TSPAN3 | −0.754 | 6.447 | −3.659 | 0.001 | 0.028 | −0.540 |
| LNM Group 2 vs. Group 3 | ENSG00000155592 | ZKSCAN2 | −0.817 | 3.578 | −3.664 | 0.001 | 0.028 | −0.447 |
| LNM Group 2 vs. Group 3 | ENSG00000177575 | CD163 | 1.968 | 6.905 | 3.670 | 0.001 | 0.027 | −0.473 |
| LNM Group 2 vs. Group 3 | ENSG00000131165 | CHMP1A | 0.693 | 6.033 | 3.674 | 0.001 | 0.027 | −0.461 |
| LNM Group 2 vs. Group 3 | ENSG00000107738 | C100rf54 | 1.217 | 6.132 | 3.678 | 0.001 | 0.027 | −0.449 |
| LNM Group 2 vs. Group 3 | ENSG00000161921 | CXCL16 | 1.121 | 5.517 | 3.677 | 0.001 | 0.027 | −0.443 |
| LNM Group 2 vs. Group 3 | ENSG00000080298 | RFX3 | −0.861 | 5.079 | −3.676 | 0.001 | 0.027 | −0.459 |
| LNM Group 2 vs. Group 3 | ENSG00000144857 | BOC | −1.922 | 4.934 | −3.683 | 0.001 | 0.027 | −0.436 |
| LNM Group 2 vs. Group 3 | ENSG00000184232 | OAF | 1.066 | 4.375 | 3.687 | 0.001 | 0.026 | −0.402 |
| LNM Group 2 vs. Group 3 | ENSG00000128000 | ZNF780B | −0.671 | 5.830 | −3.689 | 0.001 | 0.026 | −0.445 |
| LNM Group 2 vs. Group 3 | ENSG00000116962 | NID1 | −1.540 | 6.963 | −3.698 | 0.001 | 0.026 | −0.438 |
| LNM Group 2 vs. Group 3 | ENSG00000069974 | RAB27A | 0.759 | 5.335 | 3.703 | 0.001 | 0.025 | −0.373 |
| LNM Group 2 vs. Group 3 | ENSG00000184470 | TXNRD2 | 0.742 | 4.944 | 3.704 | 0.001 | 0.025 | −0.371 |
| LNM Group 2 vs. Group 3 | ENSG00000143842 | SOX13 | −0.988 | 5.384 | −3.707 | 0.001 | 0.025 | −0.388 |
| LNM Group 2 vs. Group 3 | ENSG00000182578 | CSF1R | 1.148 | 6.325 | 3.710 | 0.001 | 0.025 | −0.377 |
| LNM Group 2 vs. Group 3 | ENSG00000110619 | CARS | 0.613 | 6.508 | 3.709 | 0.001 | 0.025 | −0.414 |
| LNM Group 2 vs. Group 3 | ENSG00000143379 | SETDB1 | −0.588 | 6.028 | −3.709 | 0.001 | 0.025 | −0.390 |
| LNM Group 2 vs. Group 3 | ENSG00000101004 | NINL | −1.243 | 4.878 | −3.715 | 0.001 | 0.025 | −0.349 |
| LNM Group 2 vs. Group 3 | ENSG00000176014 | TUBB6 | 1.299 | 6.420 | 3.732 | 0.001 | 0.024 | −0.331 |
| LNM Group 2 vs. Group 3 | ENSG00000177311 | ZBTB38 | 0.768 | 7.309 | 3.726 | 0.001 | 0.024 | −0.363 |
| LNM Group 2 vs. Group 3 | ENSG00000101290 | CDS2 | −0.606 | 6.874 | −3.732 | 0.001 | 0.024 | −0.344 |
| LNM Group 2 vs. Group 3 | ENSG00000079462 | PAFAH1B3 | −1.104 | 2.958 | −3.729 | 0.001 | 0.024 | −0.275 |
| LNM Group 2 vs. Group 3 | ENSG00000105202 | FBL | −1.154 | 6.110 | −3.729 | 0.001 | 0.024 | −0.354 |
| LNM Group 2 vs. Group 3 | ENSG00000244274 | DBNDD2 | 1.059 | 4.028 | 3.753 | 0.001 | 0.023 | −0.211 |
| LNM Group 2 vs. Group 3 | ENSG00000165997 | ARL5B | −0.887 | 6.324 | −3.753 | 0.001 | 0.023 | −0.274 |
| LNM Group 2 vs. Group 3 | ENSG00000174606 | ANGEL2 | −0.605 | 6.051 | −3.758 | 0.001 | 0.023 | −0.256 |
| LNM Group 2 vs. Group 3 | ENSG00000150764 | DIXDC1 | 1.764 | 4.325 | 3.766 | 0.001 | 0.023 | −0.187 |
| LNM Group 2 vs. Group 3 | ENSG00000126107 | HECTD3 | 0.740 | 5.308 | 3.764 | 0.001 | 0.023 | −0.204 |
| LNM Group 2 vs. Group 3 | ENSG00000140948 | ZCCHC14 | 0.815 | 6.196 | 3.767 | 0.001 | 0.022 | −0.235 |
| LNM Group 2 vs. Group 3 | ENSG00000166741 | NNMT | 2.171 | 5.332 | 3.776 | 0.001 | 0.022 | −0.172 |
| LNM Group 2 vs. Group 3 | ENSG00000101439 | CST3 | 1.166 | 6.765 | 3.776 | 0.001 | 0.022 | −0.204 |
| LNM Group 2 vs. Group 3 | ENSG00000121281 | ADCY7 | 1.031 | 6.971 | 3.772 | 0.001 | 0.022 | −0.223 |
| LNM Group 2 vs. Group 3 | ENSG00000168010 | ATG16L2 | 0.906 | 6.296 | 3.771 | 0.001 | 0.022 | −0.209 |
| LNM Group 2 vs. Group 3 | ENSG00000143258 | USP21 | −0.708 | 4.629 | −3.769 | 0.001 | 0.022 | −0.190 |
| LNM Group 2 vs. Group 3 | ENSG00000172037 | LAMB2 | 1.219 | 6.653 | 3.781 | 0.000 | 0.022 | −0.195 |
| LNM Group 2 vs. Group 3 | ENSG00000167515 | TRAPPC2L | 0.859 | 3.830 | 3.780 | 0.000 | 0.022 | −0.140 |
| LNM Group 2 vs. Group 3 | ENSG00000198945 | L3MBTL3 | −1.167 | 4.120 | −3.780 | 0.000 | 0.022 | −0.159 |
| LNM Group 2 vs. Group 3 | ENSG00000163866 | SMIM12 | 0.775 | 5.540 | 3.785 | 0.000 | 0.022 | −0.163 |
| LNM Group 2 vs. Group 3 | ENSG00000158711 | ELK4 | −0.651 | 8.230 | −3.787 | 0.000 | 0.022 | −0.201 |
| LNM Group 2 vs. Group 3 | ENSG00000186174 | BCL9L | 0.590 | 7.473 | 3.804 | 0.000 | 0.021 | −0.158 |
| LNM Group 2 vs. Group 3 | ENSG00000164638 | SLC29A4 | −1.932 | 4.079 | −3.809 | 0.000 | 0.021 | −0.092 |
| LNM Group 2 vs. Group 3 | ENSG00000166454 | ATMIN | 0.861 | 6.131 | 3.813 | 0.000 | 0.021 | −0.097 |
| LNM Group 2 vs. Group 3 | ENSG00000233276 | GPX1 | 0.789 | 6.163 | 3.816 | 0.000 | 0.021 | −0.085 |
| LNM Group 2 vs. Group 3 | ENSG00000150347 | ARID5B | −0.895 | 7.587 | −3.813 | 0.000 | 0.021 | −0.132 |
| LNM Group 2 vs. Group 3 | ENSG00000143013 | LMO4 | −1.126 | 5.581 | −3.815 | 0.000 | 0.021 | −0.108 |
| LNM Group 2 vs. Group 3 | ENSG00000171843 | MLLT3 | −1.180 | 4.755 | −3.813 | 0.000 | 0.021 | −0.084 |
| LNM Group 2 vs. Group 3 | ENSG00000127329 | PTPRB | 1.263 | 5.478 | 3.823 | 0.000 | 0.021 | −0.065 |
| LNM Group 2 vs. Group 3 | ENSG00000173011 | TADA2B | 0.782 | 5.005 | 3.820 | 0.000 | 0.021 | −0.049 |
| LNM Group 2 vs. Group 3 | ENSG00000143742 | SRP9 | −0.685 | 7.103 | −3.821 | 0.000 | 0.021 | −0.108 |
| LNM Group 2 vs. Group 3 | ENSG00000141480 | ARRB2 | 0.957 | 5.844 | 3.831 | 0.000 | 0.020 | −0.041 |
| LNM Group 2 vs. Group 3 | ENSG00000128567 | PODXL | −1.378 | 6.146 | −3.837 | 0.000 | 0.020 | −0.060 |
| LNM Group 2 vs. Group 3 | ENSG00000130766 | SESN2 | 0.928 | 4.154 | 3.841 | 0.000 | 0.020 | 0.014 |
| LNM Group 2 vs. Group 3 | ENSG00000163568 | AIM2 | 1.578 | 3.740 | 3.846 | 0.000 | 0.020 | 0.030 |
| LNM Group 2 vs. Group 3 | ENSG00000146376 | ARHGAP18 | 1.051 | 5.226 | 3.846 | 0.000 | 0.020 | 0.023 |
| LNM Group 2 vs. Group 3 | ENSG00000204186 | ZDBF2 | −1.004 | 5.053 | −3.847 | 0.000 | 0.020 | −0.001 |
| LNM Group 2 vs. Group 3 | ENSG00000150687 | PRSS23 | 1.637 | 6.678 | 3.858 | 0.000 | 0.019 | 0.020 |
| LNM Group 2 vs. Group 3 | ENSG00000215251 | FASTKD5 | −0.634 | 4.543 | −3.855 | 0.000 | 0.019 | 0.038 |
| LNM Group 2 vs. Group 3 | ENSG00000198838 | RYR3 | −2.452 | 5.479 | −3.861 | 0.000 | 0.019 | 0.032 |
| LNM Group 2 vs. Group 3 | ENSG00000143815 | LBR | −0.807 | 6.569 | −3.861 | 0.000 | 0.019 | 0.004 |
| LNM Group 2 vs. Group 3 | ENSG00000159164 | SV2A | −1.221 | 2.810 | −3.861 | 0.000 | 0.019 | 0.073 |
| LNM Group 2 vs. Group 3 | ENSG00000153071 | DAB2 | 1.320 | 6.322 | 3.878 | 0.000 | 0.019 | 0.068 |
| LNM Group 2 vs. Group 3 | ENSG00000129250 | KIF1C | 0.994 | 7.432 | 3.874 | 0.000 | 0.019 | 0.040 |
| LNM Group 2 vs. Group 3 | ENSG00000196189 | SEMA4A | −1.214 | 5.880 | −3.878 | 0.000 | 0.019 | 0.057 |
| LNM Group 2 vs. Group 3 | ENSG00000100280 | AP1B1 | 0.659 | 7.158 | 3.882 | 0.000 | 0.018 | 0.061 |
| LNM Group 2 vs. Group 3 | ENSG00000182150 | ERCC6L2 | −0.627 | 5.968 | −3.882 | 0.000 | 0.018 | 0.082 |
| LNM Group 2 vs. Group 3 | ENSG00000130203 | APOE | 1.717 | 6.301 | 3.890 | 0.000 | 0.018 | 0.117 |
| LNM Group 2 vs. Group 3 | ENSG00000107551 | RASSF4 | 1.477 | 6.337 | 3.890 | 0.000 | 0.018 | 0.119 |
| LNM Group 2 vs. Group 3 | ENSG00000160703 | NLRX1 | 1.657 | 4.334 | 3.899 | 0.000 | 0.018 | 0.166 |
| LNM Group 2 vs. Group 3 | ENSG00000146094 | DOK3 | 1.584 | 4.438 | 3.897 | 0.000 | 0.018 | 0.162 |
| LNM Group 2 vs. Group 3 | ENSG00000174306 | ZHX3 | 1.103 | 5.962 | 3.893 | 0.000 | 0.018 | 0.123 |
| LNM Group 2 vs. Group 3 | ENSG00000185551 | NR2F2 | −0.800 | 5.224 | −3.893 | 0.000 | 0.018 | 0.123 |
| LNM Group 2 vs. Group 3 | ENSG00000103415 | HMOX2 | 0.781 | 5.241 | 3.903 | 0.000 | 0.018 | 0.169 |
| LNM Group 2 vs. Group 3 | ENSG00000161040 | FBXL13 | −1.424 | 2.510 | −3.902 | 0.000 | 0.018 | 0.183 |
| LNM Group 2 vs. Group 3 | ENSG00000157833 | GAREM2 | −2.325 | 3.944 | −3.911 | 0.000 | 0.018 | 0.188 |
| LNM Group 2 vs. Group 3 | ENSG00000100038 | TOP3B | 0.653 | 5.160 | 3.909 | 0.000 | 0.018 | 0.179 |
| LNM Group 2 vs. Group 3 | ENSG00000258890 | CEP95 | −0.604 | 5.941 | −3.910 | 0.000 | 0.018 | 0.153 |
| LNM Group 2 vs. Group 3 | ENSG00000144597 | EAF1 | 0.730 | 5.327 | 3.913 | 0.000 | 0.018 | 0.200 |
| LNM Group 2 vs. Group 3 | ENSG00000130830 | MPP1 | 1.303 | 4.241 | 3.919 | 0.000 | 0.018 | 0.227 |
| LNM Group 2 vs. Group 3 | ENSG00000161791 | FMNL3 | 0.847 | 7.528 | 3.920 | 0.000 | 0.018 | 0.165 |
| LNM Group 2 vs. Group 3 | ENSG00000174428 | GTF2IRD2B | −1.028 | 4.989 | −3.918 | 0.000 | 0.018 | 0.208 |
| LNM Group 2 vs. Group 3 | ENSG00000163453 | IGFBP7 | 1.129 | 7.045 | 3.928 | 0.000 | 0.017 | 0.198 |
| LNM Group 2 vs. Group 3 | ENSG00000088812 | ATRN | −0.898 | 6.782 | −3.934 | 0.000 | 0.017 | 0.210 |
| LNM Group 2 vs. Group 3 | ENSG00000204103 | MAFB | 1.265 | 6.556 | 3.938 | 0.000 | 0.017 | 0.243 |
| LNM Group 2 vs. Group 3 | ENSG00000261371 | PECAM1 | 1.131 | 6.787 | 3.937 | 0.000 | 0.017 | 0.242 |
| LNM Group 2 vs. Group 3 | ENSG00000066336 | SPI1 | 1.197 | 4.610 | 3.942 | 0.000 | 0.017 | 0.287 |
| LNM Group 2 vs. Group 3 | ENSG00000182093 | WRB | −0.688 | 3.575 | −3.961 | 0.000 | 0.016 | 0.341 |
| LNM Group 2 vs. Group 3 | ENSG00000005156 | LIG3 | −0.775 | 6.498 | −3.970 | 0.000 | 0.016 | 0.316 |
| LNM Group 2 vs. Group 3 | ENSG00000175489 | LRRC25 | 1.029 | 3.420 | 3.973 | 0.000 | 0.016 | 0.373 |
| LNM Group 2 vs. Group 3 | ENSG00000019582 | CD74 | 1.285 | 10.000 | 3.979 | 0.000 | 0.016 | 0.331 |
| LNM Group 2 vs. Group 3 | ENSG00000173546 | CSPG4 | −1.951 | 6.339 | −3.981 | 0.000 | 0.016 | 0.347 |
| LNM Group 2 vs. Group 3 | ENSG00000124702 | KLHDC3 | −0.624 | 5.308 | −3.980 | 0.000 | 0.016 | 0.367 |
| LNM Group 2 vs. Group 3 | ENSG00000077238 | IL4R | 1.477 | 7.293 | 3.987 | 0.000 | 0.016 | 0.356 |
| LNM Group 2 vs. Group 3 | ENSG00000196954 | CASP4 | 1.251 | 6.337 | 3.988 | 0.000 | 0.015 | 0.399 |
| LNM Group 2 vs. Group 3 | ENSG00000155465 | SLC7A7 | 1.180 | 4.553 | 3.986 | 0.000 | 0.015 | 0.408 |
| LNM Group 2 vs. Group 3 | ENSG00000177663 | ID7RA | 0.933 | 6.232 | 3.986 | 0.000 | 0.015 | 0.374 |
| LNM Group 2 vs. Group 3 | ENSG00000198959 | TGM2 | 1.788 | 7.010 | 3.995 | 0.000 | 0.015 | 0.388 |
| LNM Group 2 vs. Group 3 | ENSG00000068028 | RASSF1 | 0.870 | 4.918 | 3.991 | 0.000 | 0.015 | 0.419 |
| LNM Group 2 vs. Group 3 | ENSG00000155760 | FZD7 | −1.251 | 4.506 | −4.001 | 0.000 | 0.015 | 0.429 |
| LNM Group 2 vs. Group 3 | ENSG00000170248 | PDCD6IP | 0.704 | 7.192 | 4.008 | 0.000 | 0.015 | 0.420 |
| LNM Group 2 vs. Group 3 | ENSG00000095002 | MSH2 | −1.205 | 5.261 | −4.012 | 0.000 | 0.015 | 0.464 |
| LNM Group 2 vs. Group 3 | ENSG00000161638 | ITGA5 | 1.769 | 7.393 | 4.026 | 0.000 | 0.014 | 0.462 |
| LNM Group 2 vs. Group 3 | ENSG00000172572 | PDE3A | 1.744 | 4.914 | 4.031 | 0.000 | 0.014 | 0.517 |
| LNM Group 2 vs. Group 3 | ENSG00000100109 | TFIP11 | 0.721 | 5.200 | 4.027 | 0.000 | 0.014 | 0.507 |
| LNM Group 2 vs. Group 3 | ENSG00000125871 | MGME1 | −0.821 | 5.194 | −4.030 | 0.000 | 0.014 | 0.517 |
| LNM Group 2 vs. Group 3 | ENSG00000181690 | PLAG1 | −1.548 | 4.011 | −4.035 | 0.000 | 0.014 | 0.514 |
| LNM Group 2 vs. Group 3 | ENSG00000021645 | NRXN3 | −2.548 | 3.544 | −4.037 | 0.000 | 0.014 | 0.537 |
| LNM Group 2 vs. Group 3 | ENSG00000139436 | GIT2 | 0.663 | 6.684 | 4.038 | 0.000 | 0.014 | 0.507 |
| LNM Group 2 vs. Group 3 | ENSG00000123684 | LPGAT1 | −0.846 | 6.922 | −4.040 | 0.000 | 0.014 | 0.506 |
| LNM Group 2 vs. Group 3 | ENSG00000067064 | IDI1 | −1.078 | 5.279 | −4.040 | 0.000 | 0.014 | 0.537 |
| LNM Group 2 vs. Group 3 | ENSG00000204387 | C6orf48 | −0.820 | 5.573 | −4.056 | 0.000 | 0.014 | 0.577 |
| LNM Group 2 vs. Group 3 | ENSG00000204308 | RNF5 | −0.838 | 4.156 | −4.057 | 0.000 | 0.014 | 0.602 |
| LNM Group 2 vs. Group 3 | ENSG00000174010 | KLHL15 | −0.857 | 4.561 | −4.055 | 0.000 | 0.014 | 0.572 |
| LNM Group 2 vs. Group 3 | ENSG00000114126 | TFDP2 | −0.908 | 6.410 | −4.055 | 0.000 | 0.014 | 0.549 |
| LNM Group 2 vs. Group 3 | ENSG00000167037 | SGSM1 | −1.658 | 2.684 | −4.065 | 0.000 | 0.014 | 0.611 |
| LNM Group 2 vs. Group 3 | ENSG00000156990 | RPUSD3 | 0.713 | 4.639 | 4.066 | 0.000 | 0.014 | 0.622 |
| LNM Group 2 vs. Group 3 | ENSG00000141012 | GALNS | 0.694 | 6.063 | 4.064 | 0.000 | 0.014 | 0.595 |
| LNM Group 2 vs. Group 3 | ENSG00000054392 | HHAT | −1.569 | 3.797 | −4.068 | 0.000 | 0.014 | 0 625 |
| LNM Group 2 vs. Group 3 | ENSG00000099377 | HSD3B7 | 1.410 | 4.049 | 4.073 | 0.000 | 0.014 | 0.643 |
| LNM Group 2 vs. Group 3 | ENSG00000161091 | MFSD12 | 0.824 | 6.190 | 4.071 | 0.000 | 0.014 | 0.614 |
| LNM Group 2 vs. Group 3 | ENSG00000137821 | LRRC49 | −0.978 | 3.597 | −4.071 | 0.000 | 0.014 | 0.633 |
| LNM Group 2 vs. Group 3 | ENSG00000116857 | TMEM9 | −0.947 | 4.676 | −4.078 | 0.000 | 0.014 | 0.645 |
| LNM Group 2 vs. Group 3 | ENSG00000169554 | ZEB2 | 1.602 | 7.836 | 4.086 | 0.000 | 0.013 | 0.626 |
| LNM Group 2 vs. Group 3 | ENSG00000003402 | CFLAR | 0.771 | 9.324 | 4.088 | 0.000 | 0.013 | 0.640 |
| LNM Group 2 vs. Group 3 | ENSG00000043462 | LCP2 | 1.329 | 5.996 | 4.094 | 0.000 | 0.013 | 0.686 |
| LNM Group 2 vs. Group 3 | ENSG00000101294 | HM13 | 0.720 | 6.940 | 4.091 | 0.000 | 0.013 | 0.649 |
| LNM Group 2 vs. Group 3 | ENSG00000125743 | SNRPD2 | −0.775 | 5.122 | −4.090 | 0.000 | 0.013 | 0.675 |
| LNM Group 2 vs. Group 3 | ENSG00000148841 | ITPRIP | 1.311 | 6.528 | 4.104 | 0.000 | 0.013 | 0.695 |
| LNM Group 2 vs. Group 3 | ENSG00000141098 | GFOD2 | 0.911 | 5.815 | 4.103 | 0.000 | 0.013 | 0.709 |
| LNM Group 2 vs. Group 3 | ENSG00000130429 | ARPC1B | 0.759 | 7.152 | 4.099 | 0.000 | 0.013 | 0.684 |
| LNM Group 2 vs. Group 3 | ENSG00000148143 | ZNF462 | −1.348 | 6.566 | −4.103 | 0.000 | 0.013 | 0.679 |
| LNM Group 2 vs. Group 3 | ENSG00000059588 | TARBP1 | −0.958 | 6.775 | −4.110 | 0.000 | 0.013 | 0.702 |
| LNM Group 2 vs. Group 3 | ENSG00000177042 | TMEM80 | 0.871 | 4.810 | 4.117 | 0.000 | 0.013 | 0.760 |
| LNM Group 2 vs. Group 3 | ENSG00000213648 | SULT1A4 | 1.562 | 2.810 | 4.128 | 0.000 | 0.013 | 0.785 |
| LNM Group 2 vs. Group 3 | ENSG00000160613 | PCSK7 | 0.752 | 7.889 | 4.129 | 0.000 | 0.013 | 0.751 |
| LNM Group 2 vs. Group 3 | ENSG00000113504 | SLC12A7 | −1.018 | 6.370 | −4.128 | 0.000 | 0.013 | 0.754 |
| LNM Group 2 vs. Group 3 | ENSG00000197249 | SERPINA1 | 2.096 | 4.599 | 4.140 | 0.000 | 0.013 | 0.819 |
| LNM Group 2 vs. Group 3 | ENSG00000197892 | KIF13B | 1.024 | 6.143 | 4.135 | 0.000 | 0.013 | 0.796 |
| LNM Group 2 vs. Group 3 | ENSG00000138002 | IFT172 | −0.899 | 4.913 | −4.153 | 0.000 | 0.012 | 0.854 |
| LNM Group 2 vs. Group 3 | ENSG00000166250 | CLMP | 1.918 | 5.567 | 4.166 | 0.000 | 0.012 | 0.890 |
| LNM Group 2 vs. Group 3 | ENSG00000158869 | FCER1G | 1.508 | 4.294 | 4.168 | 0.000 | 0.012 | 0.902 |
| LNM Group 2 vs. Group 3 | ENSG00000112419 | PHACTR2 | 0.872 | 6.741 | 4.165 | 0.000 | 0.012 | 0.861 |
| LNM Group 2 vs. Group 3 | ENSG00000079459 | FDFT1 | −0.998 | 5.779 | −4.171 | 0.000 | 0.012 | 0.881 |
| LNM Group 2 vs. Group 3 | ENSG00000105538 | RASIP1 | 1.477 | 3.235 | 4.175 | 0.000 | 0.012 | 0.913 |
| LNM Group 2 vs. Group 3 | ENSG00000163703 | CRELD1 | 1.178 | 3.910 | 4.178 | 0.000 | 0.012 | 0.930 |
| LNM Group 2 vs. Group 3 | ENSG00000140829 | DHX38 | 0.652 | 6.096 | 4.175 | 0.000 | 0.012 | 0.906 |
| LNM Group 2 vs. Group 3 | ENSG00000035687 | ADSS | −0.668 | 6.078 | −4.184 | 0.000 | 0.012 | 0.919 |
| LNM Group 2 vs. Group 3 | ENSG00000213930 | GALT | −1.003 | 4.948 | −4.174 | 0.000 | 0.012 | 0.922 |
| LNM Group 2 vs. Group 3 | ENSG00000110436 | SLC1A2 | −2.900 | 3.190 | −4.184 | 0.000 | 0.012 | 0.933 |
| LNM Group 2 vs. Group 3 | ENSG00000010539 | ZNF200 | 0.734 | 4.670 | 4.188 | 0.000 | 0.012 | 0.960 |
| LNM Group 2 vs. Group 3 | ENSG00000138604 | GLCE | −1.033 | 4.922 | −4.192 | 0.000 | 0.012 | 0.951 |
| LNM Group 2 vs. Group 3 | ENSG00000030582 | GRN | 1.002 | 7.747 | 4.197 | 0.000 | 0.012 | 0.948 |
| LNM Group 2 vs. Group 3 | ENSG00000120860 | CCDC53 | 0.854 | 4.073 | 4.196 | 0.000 | 0.012 | 0.981 |
| LNM Group 2 vs. Group 3 | ENSG00000103496 | STX4 | 0.733 | 5.452 | 4.195 | 0.000 | 0.012 | 0.979 |
| LNM Group 2 vs. Group 3 | ENSG00000048471 | SNX29 | 0.956 | 6.604 | 4.205 | 0.000 | 0.011 | 0.990 |
| LNM Group 2 vs. Group 3 | ENSG00000271503 | CCL5 | 1.562 | 4.877 | 4.227 | 0.000 | 0.011 | 1.065 |
| LNM Group 2 vs. Group 3 | ENSG00000149577 | SIDT2 | 0.839 | 6.053 | 4.224 | 0.000 | 0.011 | 1.051 |
| LNM Group 2 vs. Group 3 | ENSG00000136205 | TNS3 | 1.116 | 7.632 | 4.230 | 0.000 | 0.011 | 1.044 |
| LNM Group 2 vs. Group 3 | ENSG00000166927 | MS4A7 | 2.144 | 5.592 | 4.247 | 0.000 | 0.011 | 1.115 |
| LNM Group 2 vs. Group 3 | ENSG00000159640 | ACE | 1.889 | 4.406 | 4.250 | 0.000 | 0.011 | 1.124 |
| LNM Group 2 vs. Group 3 | ENSG00000135709 | KIAA0513 | 1.633 | 5.999 | 4.254 | 0.000 | 0.011 | 1.138 |
| LNM Group 2 vs. Group 3 | ENSG00000175899 | A2M | 1.433 | 7.554 | 4.242 | 0.000 | 0.011 | 1.089 |
| LNM Group 2 vs. Group 3 | ENSG00000183864 | TOB2 | 0.977 | 6.160 | 4.235 | 0.000 | 0.011 | 1.070 |
| LNM Group 2 vs. Group 3 | ENSG00000128294 | TPST2 | 0.944 | 5.303 | 4.249 | 0.000 | 0.011 | 1.128 |
| LNM Group 2 vs. Group 3 | ENSG00000039523 | FAM65A | 0.932 | 6.034 | 4.235 | 0.000 | 0.011 | 1.080 |
| LNM Group 2 vs. Group 3 | ENSG00000135090 | TAOK3 | 0.698 | 6.128 | 4.233 | 0.000 | 0.011 | 1.079 |
| LNM Group 2 vs. Group 3 | ENSG00000172379 | ARNT2 | −2.357 | 3.551 | −4.247 | 0.000 | 0.011 | 1.112 |
| LNM Group 2 vs. Group 3 | ENSG00000159189 | C1QC | 1.799 | 5.953 | 4.261 | 0.000 | 0.011 | 1.154 |
| LNM Group 2 vs. Group 3 | ENSG00000090861 | AARS | 0.795 | 6.435 | 4.265 | 0.000 | 0.010 | 1.157 |
| LNM Group 2 vs. Group 3 | ENSG00000186818 | LILRB4 | 1.838 | 4.655 | 4.274 | 0.000 | 0.010 | 1.192 |
| LNM Group 2 vs. Group 3 | ENSG00000140859 | KIFC3 | 1.225 | 6.109 | 4.283 | 0.000 | 0.010 | 1.214 |
| LNM Group 2 vs. Group 3 | ENSG00000152492 | CCDC50 | −0.767 | 6.881 | −4.281 | 0.000 | 0.010 | 1.190 |
| LNM Group 2 vs. Group 3 | ENSG00000011523 | CEP68 | −1.141 | 6.164 | −4.296 | 0.000 | 0.010 | 1.242 |
| LNM Group 2 vs. Group 3 | ENSG00000105426 | PTPRS | −1.434 | 6.684 | −4.299 | 0.000 | 0.010 | 1.234 |
| LNM Group 2 vs. Group 3 | ENSG00000170458 | CD14 | 1.843 | 4.771 | 4.304 | 0.000 | 0.010 | 1.276 |
| LNM Group 2 vs. Group 3 | ENSG00000176927 | EFCAB5 | 1.110 | 2.790 | 4.310 | 0.000 | 0.010 | 1.288 |
| LNM Group 2 vs. Group 3 | ENSG00000121904 | CSMD2 | 2.667 | 3.400 | 4.317 | 0.000 | 0.010 | 1.304 |
| LNM Group 2 vs. Group 3 | ENSG00000177383 | MAGEF1 | −0.843 | 4.429 | −4.325 | 0.000 | 0.009 | 1.343 |
| LNM Group 2 vs. Group 3 | ENSG00000121989 | ACVR2A | −1.153 | 4.973 | −4.327 | 0.000 | 0.009 | 1.350 |
| LNM Group 2 vs. Group 3 | ENSG00000103005 | USB1 | 1.043 | 5.695 | 4.337 | 0.000 | 0.009 | 1.377 |
| LNM Group 2 vs. Group 3 | ENSG00000136867 | SLC31A2 | 1.456 | 5.009 | 4.361 | 0.000 | 0.009 | 1.445 |
| LNM Group 2 vs. Group 3 | ENSG00000151632 | AKR1C2 | −1.842 | 5.150 | −4.368 | 0.000 | 0.009 | 1.454 |
| LNM Group 2 vs. Group 3 | ENSG00000161955 | TNFSF13 | 1.263 | 4.314 | 4.367 | 0.000 | 0.009 | 1.465 |
| LNM Group 2 vs. Group 3 | ENSG00000135636 | DYSF | 2.136 | 5.415 | 4.377 | 0.000 | 0.009 | 1.481 |
| LNM Group 2 vs. Group 3 | ENSG00000100055 | CYTH4 | 1.355 | 5.662 | 4.379 | 0.000 | 0.009 | 1.497 |
| LNM Group 2 vs. Group 3 | ENSG00000196418 | ZNF124 | −0.856 | 5.095 | −4.379 | 0.000 | 0.009 | 1.492 |
| LNM Group 2 vs. Group 3 | ENSG00000173597 | SULT1B1 | −0.919 | 8.530 | −4.374 | 0.000 | 0.009 | 1.458 |
| LNM Group 2 vs. Group 3 | ENSG00000144749 | LRIG1 | −1.070 | 5.807 | −4.372 | 0.000 | 0.009 | 1.460 |
| LNM Group 2 vs. Group 3 | ENSG00000087266 | SH3BP2 | 1.010 | 7.638 | 4.399 | 0.000 | 0.008 | 1.531 |
| LNM Group 2 vs. Group 3 | ENSG00000160223 | ICOSLG | −1.324 | 5.371 | −4.399 | 0.000 | 0.008 | 1.540 |
| LNM Group 2 vs. Group 3 | ENSG00000216490 | IFI30 | 1.351 | 6.374 | 4.410 | 0.000 | 0.008 | 1.579 |
| LNM Group 2 vs. Group 3 | ENSG00000155393 | HEATR3 | 0.968 | 5.028 | 4.412 | 0.000 | 0.008 | 1.593 |
| LNM Group 2 vs. Group 3 | ENSG00000101160 | CTSZ | 0.881 | 6.724 | 4.413 | 0.000 | 0.008 | 1.581 |
| LNM Group 2 vs. Group 3 | ENSG00000110077 | MS4A6A | 1.827 | 5.824 | 4.430 | 0.000 | 0.008 | 1.631 |
| LNM Group 2 vs. Group 3 | ENSG00000133961 | NUMB | 0.787 | 7.036 | 4.427 | 0.000 | 0.008 | 1.622 |
| LNM Group 2 vs. Group 3 | ENSG00000142669 | SH3BGRL3 | 0.926 | 6.631 | 4.444 | 0.000 | 0.008 | 1.676 |
| LNM Group 2 vs. Group 3 | ENSG00000149131 | SERPING1 | 2.053 | 6.271 | 4.460 | 0.000 | 0.007 | 1.718 |
| LNM Group 2 vs. Group 3 | ENSG00000088930 | XRN2 | −0.812 | 6.663 | −4.459 | 0.000 | 0.007 | 1.714 |
| LNM Group 2 vs. Group 3 | ENSG00000122778 | KIAA1549 | −1.107 | 4.167 | −4.457 | 0.000 | 0.007 | 1.720 |
| LNM Group 2 vs. Group 3 | ENSG00000011600 | TYROBP | 1.536 | 3.824 | 4.466 | 0.000 | 0.007 | 1.724 |
| LNM Group 2 vs. Group 3 | ENSG00000104177 | MYEF2 | −1.397 | 4.015 | −4.469 | 0.000 | 0.007 | 1.757 |
| LNM Group 2 vs. Group 3 | ENSG00000131042 | LILRB2 | 2.149 | 3.751 | 4.509 | 0.000 | 0.007 | 1.838 |
| LNM Group 2 vs. Group 3 | ENSG00000135723 | FHOD1 | 0.797 | 5.640 | 4.513 | 0.000 | 0.007 | 1.882 |
| LNM Group 2 vs. Group 3 | ENSG00000107186 | MPDZ | −1.977 | 5.315 | −4.520 | 0.000 | 0.007 | 1.888 |
| LNM Group 2 vs. Group 3 | ENSG00000168461 | RAB31 | 1.537 | 7.199 | 4.554 | 0.000 | 0.006 | 1.982 |
| LNM Group 2 vs. Group 3 | ENSG00000173559 | NABP1 | 1.120 | 8.027 | 4.571 | 0.000 | 0.006 | 2.036 |
| LNM Group 2 vs. Group 3 | ENSG00000185630 | PBX1 | −1.172 | 6.170 | −4.569 | 0.000 | 0.006 | 2.032 |
| LNM Group 2 vs. Group 3 | ENSG00000163359 | COL6A3 | 2.093 | 10.354 | 4.583 | 0.000 | 0.006 | 2.065 |
| LNM Group 2 vs. Group 3 | ENSG00000125810 | CD93 | 1.526 | 6.515 | 4.603 | 0.000 | 0.005 | 2.132 |
| LNM Group 2 vs. Group 3 | ENSG00000141837 | CACNA1A | −1.247 | 5.706 | −4.603 | 0.000 | 0.005 | 2.141 |
| LNM Group 2 vs. Group 3 | ENSG00000173372 | C1QA | 2.246 | 5.769 | 4.633 | 0.000 | 0.005 | 2.210 |
| LNM Group 2 vs. Group 3 | ENSG00000173369 | C1QB | 2.189 | 5.963 | 4.628 | 0.000 | 0.005 | 2.201 |
| LNM Group 2 vs. Group 3 | ENSG00000158714 | SLAMF8 | 1.849 | 4.665 | 4.623 | 0.000 | 0.005 | 2.164 |
| LNM Group 2 vs. Group 3 | ENSG00000140350 | ANP32A | −0.817 | 6.484 | −4.616 | 0.000 | 0.005 | 2.172 |
| LNM Group 2 vs. Group 3 | ENSG00000154930 | ACSS1 | −1.394 | 5.683 | −4.622 | 0.000 | 0.005 | 2.188 |
| LNM Group 2 vs. Group 3 | ENSG00000187134 | AKR1C1 | −2.106 | 5.668 | −4.631 | 0.000 | 0.005 | 2.210 |
| LNM Group 2 vs. Group 3 | ENSG00000166278 | C2 | 1.936 | 5.364 | 4.654 | 0.000 | 0.005 | 2.268 |
| LNM Group 2 vs. Group 3 | ENSG00000198315 | ZKSCAN8 | −0.681 | 6.353 | −4.661 | 0.000 | 0.005 | 2.304 |
| LNM Group 2 vs. Group 3 | ENSG00000109861 | CTSC | 1.813 | 8.068 | 4.677 | 0.000 | 0.005 | 2.338 |
| LNM Group 2 vs. Group 3 | ENSG00000167613 | LAIR1 | 1.686 | 5.707 | 4.673 | 0.000 | 0.005 | 2.335 |
| LNM Group 2 vs. Group 3 | ENSG00000172215 | CXCR6 | 1.407 | 3.773 | 4.695 | 0.000 | 0.005 | 2.379 |
| LNM Group 2 vs. Group 3 | ENSG00000013364 | MVP | 1.074 | 7.190 | 4.685 | 0.000 | 0.005 | 2.379 |
| LNM Group 2 vs. Group 3 | ENSG00000124120 | TTPAL | 1.052 | 6.379 | 4.683 | 0.000 | 0.005 | 2.375 |
| LNM Group 2 vs. Group 3 | ENSG00000247315 | ZCCHC3 | −0.837 | 4.571 | −4.677 | 0.000 | 0.005 | 2.360 |
| LNM Group 2 vs. Group 3 | ENSG00000196391 | ZNF774 | −1.133 | 3.976 | −4.678 | 0.000 | 0.005 | 2.359 |
| LNM Group 2 vs. Group 3 | ENSG00000198075 | SULT1C4 | −1.220 | 2.070 | −4.689 | 0.000 | 0.005 | 2.338 |
| LNM Group 2 vs. Group 3 | ENSG00000066468 | FGFR2 | −1.727 | 5.366 | −4.710 | 0.000 | 0.005 | 2.442 |
| LNM Group 2 vs. Group 3 | ENSG00000173726 | TOMM20 | −0.947 | 7.329 | −4.711 | 0.000 | 0.005 | 2.451 |
| LNM Group 2 vs. Group 3 | ENSG00000171860 | C3AR1 | 2.128 | 3.620 | 4.728 | 0.000 | 0.005 | 2.443 |
| LNM Group 2 vs. Group 3 | ENSG00000198805 | PNP | 1.459 | 5.417 | 4.731 | 0.000 | 0.005 | 2.493 |
| LNM Group 2 vs. Group 3 | ENSG00000072786 | STK10 | 1.046 | 6.579 | 4.749 | 0.000 | 0.005 | 2.568 |
| LNM Group 2 vs. Group 3 | ENSG00000129566 | TEP1 | 0.987 | 7.063 | 4.724 | 0.000 | 0.005 | 2.495 |
| LNM Group 2 vs. Group 3 | ENSG00000196535 | MYO18A | 0.926 | 7.449 | 4.722 | 0.000 | 0.005 | 2.487 |
| LNM Group 2 vs. Group 3 | ENSG00000138131 | LOXL4 | −1.749 | 3.008 | −4.751 | 0.000 | 0.005 | 2.559 |
| LNM Group 2 vs. Group 3 | ENSG00000185920 | PTCH1 | −2.062 | 5.125 | −4.742 | 0.000 | 0.005 | 2.536 |
| LNM Group 2 vs. Group 3 | ENSG00000102575 | ACP5 | 1.843 | 5.001 | 4.768 | 0.000 | 0.005 | 2.594 |
| LNM Group 2 vs. Group 3 | ENSG00000145685 | LHFPL2 | 1.293 | 6.653 | 4.775 | 0.000 | 0.005 | 2.647 |
| LNM Group 2 vs. Group 3 | ENSG00000073921 | PICALM | 0.673 | 7.903 | 4.762 | 0.000 | 0.005 | 2.601 |
| LNM Group 2 vs. Group 3 | ENSG00000147862 | NFIB | −1.897 | 7.469 | −4.777 | 0.000 | 0.005 | 2.633 |
| LNM Group 2 vs. Group 3 | ENSG00000114738 | MAPKAPK3 | 1.008 | 4.995 | 4.792 | 0.000 | 0.005 | 2.682 |
| LNM Group 2 vs. Group 3 | ENSG00000181744 | C3orf58 | −1.245 | 5.562 | −4.790 | 0.000 | 0.005 | 2.688 |
| LNM Group 2 vs. Group 3 | ENSG00000129226 | CD68 | 1.802 | 6.067 | 4.815 | 0.000 | 0.004 | 2.742 |
| LNM Group 2 vs. Group 3 | ENSG00000171262 | FAM98B | −1.022 | 5.614 | −4.833 | 0.000 | 0.004 | 2.820 |
| LNM Group 2 vs. Group 3 | ENSG00000172243 | CLEC7A | 1.499 | 5.358 | 4.848 | 0.000 | 0.004 | 2.849 |
| LNM Group 2 vs. Group 3 | ENSG00000106991 | ENG | 1.096 | 6.195 | 4.878 | 0.000 | 0.004 | 2.953 |
| LNM Group 2 vs. Group 3 | ENSG00000205189 | ZBTB10 | −1.445 | 5.508 | −4.886 | 0.000 | 0.004 | 2.970 |
| LNM Group 2 vs. Group 3 | ENSG00000125779 | PANK2 | −0.777 | 6.065 | −4.904 | 0.000 | 0.004 | 3.030 |
| LNM Group 2 vs. Group 3 | ENSG00000008441 | NFIX | −1.695 | 6.265 | −4.930 | 0.000 | 0.003 | 3.092 |
| LNM Group 2 vs. Group 3 | ENSG00000100600 | LGMN | 1.297 | 6.142 | 4.938 | 0.000 | 0.003 | 3.126 |
| LNM Group 2 vs. Group 3 | ENSG00000175220 | ARHGAP1 | 0.876 | 7.060 | 4.952 | 0.000 | 0.003 | 3.171 |
| LNM Group 2 vs. Group 3 | ENSG00000117625 | RCOR3 | −0.839 | 6.259 | −4.939 | 0.000 | 0.003 | 3.134 |
| LNM Group 2 vs. Group 3 | ENSG00000168795 | ZBTB5 | −0.897 | 5.293 | −4.954 | 0.000 | 0.003 | 3.178 |
| LNM Group 2 vs. Group 3 | ENSG00000117906 | RCN2 | −1.183 | 6.177 | −4.956 | 0.000 | 0.003 | 3.187 |
| LNM Group 2 vs. Group 3 | ENSG00000160439 | RDH13 | 1.329 | 5.578 | 4.979 | 0.000 | 0.003 | 3.252 |
| LNM Group 2 vs. Group 3 | ENSG00000081913 | PHLPP1 | −1.185 | 5.292 | −4.981 | 0.000 | 0.003 | 3.260 |
| LNM Group 2 vs. Group 3 | ENSG00000171310 | CHST11 | 1.208 | 6.371 | 5.001 | 0.000 | 0.003 | 3.319 |
| LNM Group 2 vs. Group 3 | ENSG00000120451 | SNX19 | 0.737 | 6.775 | 5.013 | 0.000 | 0.003 | 3.356 |
| LNM Group 2 vs. Group 3 | ENSG00000164758 | MED30 | −1.180 | 2.905 | −5.003 | 0.000 | 0.003 | 3.257 |
| LNM Group 2 vs. Group 3 | ENSG00000160679 | CHTOP | −0.706 | 6.573 | −5.027 | 0.000 | 0.003 | 3.399 |
| LNM Group 2 vs. Group 3 | ENSG00000118007 | STAG1 | −0.769 | 6.758 | −5.061 | 0.000 | 0.003 | 3.500 |
| LNM Group 2 vs. Group 3 | ENSG00000092421 | SEMA6A | −1.880 | 5.149 | −5.068 | 0.000 | 0.003 | 3.504 |
| LNM Group 2 vs. Group 3 | ENSG00000136158 | SPRY2 | −1.931 | 3.968 | −5.093 | 0.000 | 0.003 | 3.562 |
| LNM Group 2 vs. Group 3 | ENSG00000143226 | FCGR2A | 2.541 | 5.987 | 5.117 | 0.000 | 0.003 | 3.615 |
| LNM Group 2 vs. Group 3 | ENSG00000154127 | UBASH3B | 1.717 | 5.263 | 5.137 | 0.000 | 0.003 | 3.691 |
| LNM Group 2 vs. Group 3 | ENSG00000145246 | ATP10D | 1.572 | 6.334 | 5.155 | 0.000 | 0.003 | 3.769 |
| LNM Group 2 vs. Group 3 | ENSG00000163041 | H3F3A | −0.838 | 7.837 | −5.152 | 0.000 | 0.003 | 3.775 |
| LNM Group 2 vs. Group 3 | ENSG00000118707 | TGIF2 | −1.402 | 5.349 | −5.167 | 0.000 | 0.003 | 3.819 |
| LNM Group 2 vs. Group 3 | ENSG00000103043 | VAC14 | 0.827 | 6.196 | 5.194 | 0.000 | 0.002 | 3.896 |
| LNM Group 2 vs. Group 3 | ENSG00000112379 | ARFGEF3 | −1.509 | 3.894 | −5.199 | 0.000 | 0.002 | 3.907 |
| LNM Group 2 vs. Group 3 | ENSG00000198668 | CALM1 | 1.110 | 8.268 | 5.241 | 0.000 | 0.002 | 4.046 |
| LNM Group 2 vs. Group 3 | ENSG00000159720 | ATP6V0D1 | 0.942 | 6.186 | 5.232 | 0.000 | 0.002 | 4.010 |
| LNM Group 2 vs. Group 3 | ENSG00000137070 | IL11RA | −1.935 | 4.816 | −5.274 | 0.000 | 0.002 | 4.090 |
| LNM Group 2 vs. Group 3 | ENSG00000205250 | E2F4 | 0.870 | 6.046 | 5.340 | 0.000 | 0.002 | 4.336 |
| LNM Group 2 vs. Group 3 | ENSG00000137103 | TMEM8B | −1.908 | 3.219 | −5.355 | 0.000 | 0.002 | 4.324 |
| LNM Group 2 vs. Group 3 | ENSG00000137491 | SLCO2B1 | 1.939 | 7.133 | 5.387 | 0.000 | 0.002 | 4.448 |
| LNM Group 2 vs. Group 3 | ENSG00000071575 | TRIB2 | −1.148 | 6.350 | −5.379 | 0.000 | 0.002 | 4.464 |
| LNM Group 2 vs. Group 3 | ENSG00000136869 | TLR4 | 2.678 | 5.010 | 5.616 | 0.000 | 0.002 | 5.045 |
| LNM Group 2 vs. Group 3 | ENSG00000184060 | ADAP2 | 2.061 | 4.480 | 5.590 | 0.000 | 0.002 | 4.972 |
| LNM Group 2 vs. Group 3 | ENSG00000135047 | CTSL | 1.842 | 5.471 | 5.640 | 0.000 | 0.002 | 5.196 |
| LNM Group 2 vs. Group 3 | ENSG00000139567 | ACVRL1 | 1.840 | 4.570 | 5.527 | 0.000 | 0.002 | 4.801 |
| LNM Group 2 vs. Group 3 | ENSG00000135077 | HAVCR2 | 1.579 | 5.005 | 5.467 | 0.000 | 0.002 | 4.668 |
| LNM Group 2 vs. Group 3 | ENSG00000064601 | CTSA | 1.146 | 6.657 | 5.457 | 0.000 | 0.002 | 4.698 |
| LNM Group 2 vs. Group 3 | ENSG00000188997 | KCTD21 | 1.071 | 4.129 | 5.493 | 0.000 | 0.002 | 4.721 |
| LNM Group 2 vs. Group 3 | ENSG00000099949 | LZTR1 | 0.951 | 6.255 | 5.516 | 0.000 | 0.002 | 4.874 |
| LNM Group 2 vs. Group 3 | ENSG00000154144 | TBRG1 | 0.827 | 5.684 | 5.643 | 0.000 | 0.002 | 5.231 |
| LNM Group 2 vs. Group 3 | ENSG00000169105 | CHST14 | −1.246 | 3.970 | −5.505 | 0.000 | 0.002 | 4.783 |
| LNM Group 2 vs. Group 3 | ENSG00000165434 | PGM2L1 | −1.426 | 5.364 | −5.448 | 0.000 | 0.002 | 4.639 |
| LNM Group 2 vs. Group 3 | ENSG00000125247 | TMTC4 | −1.500 | 4.180 | −5.560 | 0.000 | 0.002 | 4.912 |
| LNM Group 2 vs. Group 3 | ENSG00000138336 | TET1 | −1.591 | 3.684 | −5.639 | 0.000 | 0.002 | 5.173 |
| LNM Group 2 vs. Group 3 | ENSG00000260314 | MRC1 | 3.604 | 5.521 | 5.928 | 0.000 | 0.001 | 5.965 |
| LNM Group 2 vs. Group 3 | ENSG00000021574 | SPAST | −1.080 | 5.644 | −6.288 | 0.000 | 0.000 | 7.239 |
| LNM Group 2 vs. Group 3 | ENSG00000154359 | LONRF1 | −1.921 | 5.042 | −6.382 | 0.000 | 0.000 | 7.487 |
| LNM Group 2 vs. Group 3 | ENSG00000064651 | SLC12A2 | −1.988 | 6.081 | −6.238 | 0.000 | 0.000 | 7.058 |
| LNM Group 2 vs. Group 3 | ENSG00000180730 | SHISA2 | −2.880 | 2.447 | −6.519 | 0.000 | 0.000 | 7.848 |
| LNM Group 2 vs. Group 3 | ENSG00000116016 | EPAS1 | 2.009 | 7.912 | 9.032 | 0.000 | 0.000 | 15.323 |
| LNM Group 3 vs. Group 1 | ENSG00000198838 | RYR3 | 2.928 | 5.479 | 4.916 | 0.000 | 0.002 | 3.048 |
| LNM Group 3 vs. Group 1 | ENSG00000180730 | SHISA2 | 2.838 | 2.447 | 7.158 | 0.000 | 0.000 | 9.888 |
| LNM Group 3 vs. Group 1 | ENSG00000066468 | FGFR2 | 2.408 | 5.366 | 7.084 | 0.000 | 0.000 | 9.679 |
| LNM Group 3 vs. Group 1 | ENSG00000173546 | CSPG4 | 2.388 | 6.339 | 5.167 | 0.000 | 0.001 | 3.800 |
| LNM Group 3 vs. Group 1 | ENSG00000157064 | NMNAT2 | 2.331 | 3.243 | 3.699 | 0.001 | 0.013 | −0.394 |
| LNM Group 3 vs. Group 1 | ENSG00000177839 | PCDHB9 | 2.312 | 2.610 | 3.696 | 0.001 | 0.013 | −0.384 |
| LNM Group 3 vs. Group 1 | ENSG00000205363 | C15orf59 | 2.309 | 3.251 | 3.942 | 0.000 | 0.009 | 0.270 |
| LNM Group 3 vs. Group 1 | ENSG00000266074 | BAHCC1 | 2.219 | 5.591 | 5.687 | 0.000 | 0.001 | 5.389 |
| LNM Group 3 vs. Group 1 | ENSG00000008441 | NFIX | 2.075 | 6.265 | 6.411 | 0.000 | 0.000 | 7.622 |
| LNM Group 3 vs. Group 1 | ENSG00000144857 | BOC | 2.071 | 4.934 | 4.239 | 0.000 | 0.006 | 1.071 |
| LNM Group 3 vs. Group 1 | ENSG00000164638 | SLC29A4 | 2.065 | 4.079 | 4.425 | 0.000 | 0.004 | 1.608 |
| LNM Group 3 vs. Group 1 | ENSG00000168389 | MFSD2A | 2.036 | 4.277 | 3.931 | 0.000 | 0.009 | 0.238 |
| LNM Group 3 vs. Group 1 | ENSG00000147862 | NFIB | 1.945 | 7.469 | 5.187 | 0.000 | 0.001 | 3.858 |
| LNM Group 3 vs. Group 1 | ENSG00000088836 | SLC4A11 | 1.932 | 3.943 | 4.146 | 0.000 | 0.007 | 0.803 |
| LNM Group 3 vs. Group 1 | ENSG00000116962 | NID1 | 1.878 | 6.963 | 4.782 | 0.000 | 0.002 | 2.634 |
| LNM Group 3 vs. Group 1 | ENSG00000064651 | SLC12A2 | 1.876 | 6.081 | 6.260 | 0.000 | 0.000 | 7.163 |
| LNM Group 3 vs. Group 1 | ENSG00000021645 | NRXN3 | 1.873 | 3.544 | 3.154 | 0.003 | 0.035 | −1.763 |
| LNM Group 3 vs. Group 1 | ENSG00000138696 | BMPR1B | 1.854 | 3.687 | 3.332 | 0.002 | 0.026 | −1.374 |
| LNM Group 3 vs. Group 1 | ENSG00000155380 | SLC16A1 | 1.845 | 6.332 | 4.245 | 0.000 | 0.006 | 1.069 |
| LNM Group 3 vs. Group 1 | ENSG00000114115 | RBP1 | 1.832 | 4.600 | 4.439 | 0.000 | 0.004 | 1.635 |
| LNM Group 3 vs. Group 1 | ENSG00000151967 | SCHIP1 | 1.797 | 4.715 | 3.778 | 0.001 | 0.012 | −0.225 |
| LNM Group 3 vs. Group 1 | ENSG00000161040 | FBXL13 | 1.791 | 2.510 | 5.247 | 0.000 | 0.001 | 4.014 |
| LNM Group 3 vs. Group 1 | ENSG00000187134 | AKR1C1 | 1.756 | 5.668 | 4.154 | 0.000 | 0.006 | 0.821 |
| LNM Group 3 vs. Group 1 | ENSG00000148143 | ZNF462 | 1.750 | 6.566 | 5.657 | 0.000 | 0.001 | 5.299 |
| LNM Group 3 vs. Group 1 | ENSG00000107186 | MPDZ | 1.726 | 5.315 | 4.225 | 0.000 | 0.006 | 1.015 |
| LNM Group 3 vs. Group 1 | ENSG00000157833 | GAREM2 | 1.684 | 3.944 | 3.080 | 0.004 | 0.040 | −2.003 |
| LNM Group 3 vs. Group 1 | ENSG00000185565 | LSAMP | 1.682 | 5.469 | 3.140 | 0.003 | 0.036 | −1.946 |
| LNM Group 3 vs. Group 1 | ENSG00000107338 | SHB | 1.652 | 4.763 | 4.921 | 0.000 | 0.002 | 3.062 |
| LNM Group 3 vs. Group 1 | ENSG00000136158 | SPRY2 | 1.648 | 3.968 | 4.714 | 0.000 | 0.003 | 2.452 |
| LNM Group 3 vs. Group 1 | ENSG00000130821 | SLC6A8 | 1.642 | 6.543 | 2.941 | 0.005 | 0.049 | −2.437 |
| LNM Group 3 vs. Group 1 | ENSG00000157240 | FZD1 | 1.638 | 5.242 | 4.276 | 0.000 | 0.006 | 1.167 |
| LNM Group 3 vs. Group 1 | ENSG00000117394 | SLC2A1 | 1.636 | 8.211 | 3.296 | 0.002 | 0.027 | −1.554 |
| LNM Group 3 vs. Group 1 | ENSG00000172986 | GXYLT2 | 1.632 | 4.575 | 3.212 | 0.003 | 0.032 | −1.691 |
| LNM Group 3 vs. Group 1 | ENSG00000100994 | PYGB | 1.629 | 7.445 | 4.802 | 0.000 | 0.002 | 2.689 |
| LNM Group 3 vs. Group 1 | ENSG00000164684 | ZNF704 | 1.624 | 5.657 | 3.558 | 0.001 | 0.017 | −0.849 |
| LNM Group 3 vs. Group 1 | ENSG00000092068 | SLC7A8 | 1.619 | 6.411 | 3.478 | 0.001 | 0.020 | −1.037 |
| LNM Group 3 vs. Group 1 | ENSG00000143842 | SOX13 | 1.618 | 5.384 | 6.491 | 0.000 | 0.000 | 7.893 |
| LNM Group 3 vs. Group 1 | ENSG00000119326 | CTNNAL1 | 1.617 | 4.654 | 3.576 | 0.001 | 0.016 | −0.769 |
| LNM Group 3 vs. Group 1 | ENSG00000141934 | PLPP2 | 1.609 | 5.943 | 3.596 | 0.001 | 0.016 | −0.754 |
| LNM Group 3 vs. Group 1 | ENSG00000187244 | BCAM | 1.600 | 6.078 | 3.962 | 0.000 | 0.009 | 0.243 |
| LNM Group 3 vs. Group 1 | ENSG00000128567 | PODXL | 1.598 | 6.146 | 4.739 | 0.000 | 0.003 | 2.505 |
| LNM Group 3 vs. Group 1 | ENSG00000155760 | FZD7 | 1.579 | 4.506 | 5.447 | 0.000 | 0.001 | 4.668 |
| LNM Group 3 vs. Group 1 | ENSG00000166813 | KIF7 | 1.572 | 3.863 | 4.053 | 0.000 | 0.008 | 0.549 |
| LNM Group 3 vs. Group 1 | ENSG00000175832 | ETV4 | 1.560 | 3.719 | 4.927 | 0.000 | 0.002 | 3.089 |
| LNM Group 3 vs. Group 1 | ENSG00000109654 | TRIM2 | 1.554 | 5.906 | 5.129 | 0.000 | 0.001 | 3.683 |
| LNM Group 3 vs. Group 1 | ENSG00000101004 | NINL | 1.551 | 4.878 | 4.996 | 0.000 | 0.002 | 3.295 |
| LNM Group 3 vs. Group 1 | ENSG00000143153 | ATP1B1 | 1.549 | 6.955 | 3.923 | 0.000 | 0.009 | 0.147 |
| LNM Group 3 vs. Group 1 | ENSG00000116128 | BCL9 | 1.534 | 4.976 | 5.022 | 0.000 | 0.002 | 3.376 |
| LNM Group 3 vs. Group 1 | ENSG00000205213 | LGR4 | 1.512 | 5.154 | 5.076 | 0.000 | 0.001 | 3.535 |
| LNM Group 3 vs. Group 1 | ENSG00000106484 | MEST | 1.499 | 4.857 | 3.461 | 0.001 | 0.021 | −1.069 |
| LNM Group 3 vs. Group 1 | ENSG00000053108 | FSTL4 | 1.495 | 3.588 | 4.274 | 0.000 | 0.006 | 1.186 |
| LNM Group 3 vs. Group 1 | ENSG00000141542 | RAB40B | 1.495 | 3.896 | 4.064 | 0.000 | 0.007 | 0.584 |
| LNM Group 3 vs. Group 1 | ENSG00000151208 | DLG5 | 1.481 | 7.342 | 3.222 | 0.002 | 0.031 | −1.746 |
| LNM Group 3 vs. Group 1 | ENSG00000179431 | FJX1 | 1.479 | 4.001 | 3.318 | 0.002 | 0.026 | −1.385 |
| LNM Group 3 vs. Group 1 | ENSG00000125841 | NRSN2 | 1.478 | 4.000 | 5.332 | 0.000 | 0.001 | 4.315 |
| LNM Group 3 vs. Group 1 | ENSG00000142632 | ARHGEF19 | 1.475 | 4.527 | 5.011 | 0.000 | 0.002 | 3.344 |
| LNM Group 3 vs. Group 1 | ENSG00000188153 | COL4A5 | 1.469 | 6.757 | 3.853 | 0.000 | 0.010 | −0.063 |
| LNM Group 3 vs. Group 1 | ENSG00000162337 | LRP5 | 1.466 | 5.883 | 4.304 | 0.000 | 0.005 | 1.227 |
| LNM Group 3 vs. Group 1 | ENSG00000162738 | VANGL2 | 1.466 | 5.578 | 3.797 | 0.000 | 0.011 | −0.202 |
| LNM Group 3 vs. Group 1 | ENSG00000169908 | TM4SF1 | 1.456 | 6.782 | 3.345 | 0.002 | 0.025 | −1.420 |
| LNM Group 3 vs. Group 1 | ENSG00000006468 | ETV1 | 1.451 | 3.276 | 3.442 | 0.001 | 0.021 | −1.106 |
| LNM Group 3 vs. Group 1 | ENSG00000185920 | PTCH1 | 1.437 | 5.125 | 3.580 | 0.001 | 0.016 | −0.782 |
| LNM Group 3 vs. Group 1 | ENSG00000164171 | ITGA2 | 1.422 | 7.280 | 4.134 | 0.000 | 0.007 | 0.730 |
| LNM Group 3 vs. Group 1 | ENSG00000092421 | SEMA6A | 1.420 | 5.149 | 4.125 | 0.000 | 0.007 | 0.713 |
| LNM Group 3 vs. Group 1 | ENSG00000137103 | TMEM8B | 1.419 | 3.219 | 4.325 | 0.000 | 0.005 | 1.334 |
| LNM Group 3 vs. Group 1 | ENSG00000124766 | SOX4 | 1.417 | 6.606 | 4.098 | 0.000 | 0.007 | 0.625 |
| LNM Group 3 vs. Group 1 | ENSG00000185842 | DNAH14 | 1.415 | 4.374 | 3.887 | 0.000 | 0.010 | 0.045 |
| LNM Group 3 vs. Group 1 | ENSG00000151917 | BEND6 | 1.406 | 3.101 | 4.030 | 0.000 | 0.008 | 0.503 |
| LNM Group 3 vs. Group 1 | ENSG00000154359 | LONRF1 | 1.406 | 5.042 | 5.052 | 0.000 | 0.002 | 3.467 |
| LNM Group 3 vs. Group 1 | ENSG00000072682 | P4HA2 | 1.404 | 5.644 | 3.065 | 0.004 | 0.041 | −2.012 |
| LNM Group 3 vs. Group 1 | ENSG00000204580 | DDR1 | 1.399 | 8.303 | 3.558 | 0.001 | 0.017 | −0.863 |
| LNM Group 3 vs. Group 1 | ENSG00000112715 | VEGFA | 1.396 | 8.135 | 3.382 | 0.002 | 0.024 | −1.324 |
| LNM Group 3 vs. Group 1 | ENSG00000178896 | EXOSC4 | 1.389 | 3.234 | 3.413 | 0.001 | 0.022 | −1.149 |
| LNM Group 3 vs. Group 1 | ENSG00000181588 | MEX3D | 1.389 | 4.514 | 4.559 | 0.000 | 0.003 | 1.995 |
| LNM Group 3 vs. Group 1 | ENSG00000079462 | PAFAH1B3 | 1.375 | 2.958 | 4.948 | 0.000 | 0.002 | 3.136 |
| LNM Group 3 vs. Group 1 | ENSG00000114270 | COL7A1 | 1.374 | 9.645 | 3.056 | 0.004 | 0.041 | −2.135 |
| LNM Group 3 vs. Group 1 | ENSG00000070087 | PFN2 | 1.371 | 5.885 | 3.542 | 0.001 | 0.018 | −0.905 |
| LNM Group 3 vs. Group 1 | ENSG00000105426 | PTPRS | 1.371 | 6.684 | 4.375 | 0.000 | 0.005 | 1.424 |
| LNM Group 3 vs. Group 1 | ENSG00000117266 | CDK18 | 1.366 | 5.022 | 4.282 | 0.000 | 0.005 | 1.160 |
| LNM Group 3 vs. Group 1 | ENSG00000114315 | HES1 | 1.361 | 4.740 | 3.561 | 0.001 | 0.017 | −0.790 |
| LNM Group 3 vs. Group 1 | ENSG00000006327 | TNFRSF12A | 1.358 | 5.627 | 3.281 | 0.002 | 0.028 | −1.527 |
| LNM Group 3 vs. Group 1 | ENSG00000186314 | PRELID2 | 1.356 | 3.508 | 3.760 | 0.001 | 0.012 | −0.222 |
| LNM Group 3 vs. Group 1 | ENSG00000074590 | NUAK1 | 1.340 | 5.420 | 3.182 | 0.003 | 0.033 | −1.807 |
| LNM Group 3 vs. Group 1 | ENSG00000145632 | PLK2 | 1.331 | 6.110 | 3.396 | 0.002 | 0.023 | −1.241 |
| LNM Group 3 vs. Group 1 | ENSG00000006210 | CX3CL1 | 1.324 | 4.591 | 2.990 | 0.005 | 0.046 | −2.285 |
| LNM Group 3 vs. Group 1 | ENSG00000197763 | TXNRD3 | 1.324 | 3.503 | 3.627 | 0.001 | 0.015 | −0.585 |
| LNM Group 3 vs. Group 1 | ENSG00000137070 | IL11RA | 1.324 | 4.816 | 3.832 | 0.000 | 0.011 | −0.048 |
| LNM Group 3 vs. Group 1 | ENSG00000173320 | STOX2 | 1.321 | 4.633 | 3.652 | 0.001 | 0.014 | −0.582 |
| LNM Group 3 vs. Group 1 | ENSG00000115457 | IGFBP2 | 1.318 | 5.247 | 3.083 | 0.004 | 0.040 | −2.085 |
| LNM Group 3 vs. Group 1 | ENSG00000143367 | TUFT1 | 1.316 | 5.377 | 3.094 | 0.004 | 0.039 | −2.001 |
| LNM Group 3 vs. Group 1 | ENSG00000143147 | GPR161 | 1.310 | 4.831 | 4.285 | 0.000 | 0.005 | 1.198 |
| LNM Group 3 vs. Group 1 | ENSG00000244405 | ETV5 | 1.309 | 5.173 | 3.316 | 0.002 | 0.026 | −1.497 |
| LNM Group 3 vs. Group 1 | ENSG00000006118 | TMEM132A | 1.303 | 6.305 | 3.315 | 0.002 | 0.026 | −1.493 |
| LNM Group 3 vs. Group 1 | ENSG00000138131 | LOXL4 | 1.302 | 3.008 | 3.872 | 0.000 | 0.010 | 0.043 |
| LNM Group 3 vs. Group 1 | ENSG00000164050 | PLXNB1 | 1.299 | 7.069 | 3.615 | 0.001 | 0.015 | −0.718 |
| LNM Group 3 vs. Group 1 | ENSG00000158769 | F11R | 1.298 | 7.648 | 3.831 | 0.000 | 0.011 | −0.128 |
| LNM Group 3 vs. Group 1 | ENSG00000151632 | AKR1C2 | 1.297 | 5.150 | 3.334 | 0.002 | 0.026 | −1.410 |
| LNM Group 3 vs. Group 1 | ENSG00000120549 | KIAA1217 | 1.292 | 6.908 | 4.868 | 0.000 | 0.002 | 2.901 |
| LNM Group 3 vs. Group 1 | ENSG00000172893 | DHCR7 | 1.291 | 4.568 | 3.843 | 0.000 | 0.010 | −0.040 |
| LNM Group 3 vs. Group 1 | ENSG00000152104 | PTPN14 | 1.274 | 7.852 | 4.868 | 0.000 | 0.002 | 2.904 |
| LNM Group 3 vs. Group 1 | ENSG00000114023 | FAM162A | 1.260 | 4.891 | 3.335 | 0.002 | 0.026 | −1.383 |
| LNM Group 3 vs. Group 1 | ENSG00000131788 | PIAS3 | 1.259 | 6.048 | 5.116 | 0.000 | 0.001 | 3.666 |
| LNM Group 3 vs. Group 1 | ENSG00000111057 | KRT18 | 1.257 | 5.463 | 3.330 | 0.002 | 0.026 | −1.452 |
| LNM Group 3 vs. Group 1 | ENSG00000140443 | IGF1R | 1.251 | 7.946 | 4.422 | 0.000 | 0.004 | 1.574 |
| LNM Group 3 vs. Group 1 | ENSG00000110723 | EXPH5 | 1.248 | 5.394 | 3.122 | 0.003 | 0.037 | −1.923 |
| LNM Group 3 vs. Group 1 | ENSG00000101773 | RBBP8 | 1.248 | 5.858 | 4.367 | 0.000 | 0.005 | 1.412 |
| LNM Group 3 vs. Group 1 | ENSG00000243244 | STON1 | 1.243 | 5.027 | 3.720 | 0.001 | 0.013 | −0.364 |
| LNM Group 3 vs. Group 1 | ENSG00000154358 | OBSCN | 1.234 | 7.572 | 3.228 | 0.002 | 0.031 | −1.728 |
| LNM Group 3 vs. Group 1 | ENSG00000112655 | PTK7 | 1.232 | 7.125 | 3.888 | 0.000 | 0.010 | 0.036 |
| LNM Group 3 vs. Group 1 | ENSG00000116857 | TMEM9 | 1.225 | 4.676 | 5.681 | 0.000 | 0.001 | 5.386 |
| LNM Group 3 vs. Group 1 | ENSG00000171867 | PRNP | 1.224 | 7.884 | 3.783 | 0.000 | 0.012 | −0.261 |
| LNM Group 3 vs. Group 1 | ENSG00000271303 | SRXN1 | 1.214 | 5.501 | 3.449 | 0.001 | 0.021 | −1.100 |
| LNM Group 3 vs. Group 1 | ENSG00000165264 | NDUFB6 | 1.212 | 3.568 | 3.658 | 0.001 | 0.014 | −0.466 |
| LNM Group 3 vs. Group 1 | ENSG00000138604 | GLCE | 1.209 | 4.922 | 5.306 | 0.000 | 0.001 | 4.242 |
| LNM Group 3 vs. Group 1 | ENSG00000099203 | TMED1 | 1.205 | 3.096 | 3.117 | 0.003 | 0.037 | −1.839 |
| LNM Group 3 vs. Group 1 | ENSG00000067141 | NEO1 | 1.202 | 5.800 | 3.941 | 0.000 | 0.009 | 1.186 |
| LNM Group 3 vs. Group 1 | ENSG00000187775 | DNAH17 | 1.201 | 5.901 | 3.112 | 0.003 | 0.038 | −1.975 |
| LNM Group 3 vs. Group 1 | ENSG00000155254 | MARVELD1 | 1.200 | 5.663 | 3.964 | 0.000 | 0.009 | 0.319 |
| LNM Group 3 vs. Group 1 | ENSG00000164070 | HSPA4L | 1.200 | 5.893 | 3.580 | 0.001 | 0.016 | −0.805 |
| LNM Group 3 vs. Group 1 | ENSG00000243364 | EFNA4 | 1.199 | 2.767 | 3.836 | 0.000 | 0.010 | −0.001 |
| LNM Group 3 vs. Group 1 | ENSG00000169957 | ZNF768 | 1.193 | 4 412 | 5.953 | 0.000 | 0.000 | 6.207 |
| LNM Group 3 vs. Group 1 | ENSG00000091622 | PITPNM3 | 1.192 | 5.210 | 3.199 | 0.003 | 0.033 | −1.761 |
| LNM Group 3 vs. Group 1 | ENSG00000102230 | PCYT1B | 1.192 | 2.302 | 3.191 | 0.003 | 0.033 | −1.693 |
| LNM Group 3 vs. Group 1 | ENSG00000168502 | MTCL1 | 1.189 | 5.459 | 3.992 | 0.000 | 0.008 | 0.339 |
| LNM Group 3 vs. Group 1 | ENSG00000117139 | KDM5B | 1.188 | 7.261 | 5.069 | 0.000 | 0.001 | 3.516 |
| LNM Group 3 vs. Group 1 | ENSG00000166503 | RP11- | 1.184 | 6.556 | 4.709 | 0.000 | 0.003 | 2.426 |
| | | 382A20.3 | | | | | | |
| LNM Group 3 vs. Group 1 | ENSG00000007866 | TEAD3 | 1.179 | 5.445 | 3.697 | 0.001 | 0.013 | −0.450 |
| LNM Group 3 vs. Group 1 | ENSG00000063660 | GPC1 | 1.166 | 7.396 | 3.659 | 0.001 | 0.014 | −0.596 |
| LNM Group 3 vs. Group 1 | ENSG00000135525 | MAP7 | 1.163 | 4.944 | 3.317 | 0.002 | 0.026 | −1.461 |
| LNM Group 3 vs. Group 1 | ENSG00000186638 | KIF24 | 1.162 | 3.928 | 4.356 | 0.000 | 0.005 | 1.431 |
| LNM Group 3 vs. Group 1 | ENSG00000106780 | MEGF9 | 1.151 | 5.922 | 3.849 | 0.000 | 0.010 | −0.031 |
| LNM Group 3 vs. Group 1 | ENSG00000170412 | GPRC5C | 1.149 | 3.744 | 3.056 | 0.004 | 0.041 | −2.061 |
| LNM Group 3 vs. Group 1 | ENSG00000118200 | CAMSAP2 | 1.148 | 6.796 | 4.168 | 0.000 | 0.006 | 0.834 |
| LNM Group 3 vs. Group 1 | ENSG00000081913 | PHLPP1 | 1.142 | 5.292 | 5.159 | 0.000 | 0.001 | 3.795 |
| LNM Group 3 vs. Group 1 | ENSG00000177732 | SOX12 | 1.141 | 4.882 | 4.635 | 0.000 | 0.003 | 2.234 |
| LNM Group 3 vs. Group 1 | ENSG00000100842 | EFS | 1.141 | 4.926 | 3.430 | 0.001 | 0.022 | −1.157 |
| LNM Group 3 vs. Group 1 | ENSG00000125843 | AP5S1 | 1.139 | 5.039 | 4.672 | 0.000 | 0.003 | 2.335 |
| LNM Group 3 vs. Group 1 | ENSG00000088812 | ATRN | 1.139 | 6.782 | 5.295 | 0.000 | 0.001 | 4.209 |
| LNM Group 3 vs. Group 1 | ENSG00000154380 | ENAH | 1.130 | 8.030 | 3.578 | 0.001 | 0.016 | −0.820 |
| LNM Group 3 vs. Group 1 | ENSG00000115657 | ABCB6 | 1.128 | 4.620 | 3.974 | 0.000 | 0.009 | 0.341 |
| LNM Group 3 vs. Group 1 | ENSG00000073282 | TP63 | 1.127 | 8.375 | 2.969 | 0.005 | 0.047 | −2.369 |
| LNM Group 3 vs. Group 1 | ENSG00000166681 | BEX3 | 1.124 | 4.529 | 3.494 | 0.001 | 0.019 | −0.956 |
| LNM Group 3 vs. Group 1 | ENSG00000197586 | ENTPD6 | 1.121 | 6.144 | 4.774 | 0.000 | 0.002 | 2.625 |
| LNM Group 3 vs. Group 1 | ENSG00000106789 | CORO2A | 1.120 | 5.676 | 4.566 | 0.000 | 0.003 | 2.010 |
| LNM Group 3 vs. Group 1 | ENSG00000136026 | CKAP4 | 1.118 | 6.922 | 3.777 | 0.001 | 0.012 | −0.278 |
| LNM Group 3 vs. Group 1 | ENSG00000125247 | TMTC4 | 1.118 | 4.180 | 4.403 | 0.000 | 0.004 | 1.567 |
| LNM Group 3 vs. Group 1 | ENSG00000132640 | BTBD3 | 1.115 | 5.484 | 3.895 | 0.000 | 0.010 | 0.060 |
| LNM Group 3 vs. Group 1 | ENSG00000143013 | LMO4 | 1.113 | 5.581 | 4.052 | 0.000 | 0.007 | 0.510 |
| LNM Group 3 vs. Group 1 | ENSG00000160111 | CPAMD8 | 1.110 | 4.049 | 3.636 | 0.001 | 0.015 | −0.562 |
| LNM Group 3 vs. Group 1 | ENSG00000083857 | FAT1 | 1.110 | 8.996 | 3.325 | 0.002 | 0.026 | −1.465 |
| LNM Group 3 vs. Group 1 | ENSG00000127564 | PKMYT1 | 1.109 | 4.627 | 2.987 | 0.005 | 0.046 | −2.219 |
| LNM Group 3 vs. Group 1 | ENSG00000090447 | TFAP4 | 1.108 | 5.040 | 3.960 | 0.000 | 0.009 | 0.279 |
| LNM Group 3 vs. Group 1 | ENSG00000154447 | SH3RF1 | 1.106 | 5.305 | 3.844 | 0.000 | 0.010 | −0.034 |
| LNM Group 3 vs. Group 1 | ENSG00000165655 | ZNF503 | 1.091 | 4.082 | 3.106 | 0.003 | 0.038 | −1.905 |
| LNM Group 3 vs. Group 1 | ENSG00000107731 | UNC5B | 1.091 | 6.298 | 2.942 | 0.005 | 0.049 | −2.395 |
| LNM Group 3 vs. Group 1 | ENSG00000169105 | CHST14 | 1.090 | 3.970 | 5.183 | 0.000 | 0.001 | 3.860 |
| LNM Group 3 vs. Group 1 | ENSG00000151929 | BAG3 | 1.088 | 5.800 | 3.655 | 0.001 | 0.014 | −0.550 |
| LNM Group 3 vs. Group 1 | ENSG00000104341 | LAPTM4B | 1.088 | 6.305 | 3.581 | 0.001 | 0.016 | −0.771 |
| LNM Group 3 vs. Group 1 | ENSG00000082458 | DLG3 | 1.085 | 4.809 | 3.939 | 0.000 | 0.009 | 0.236 |
| LNM Group 3 vs. Group 1 | ENSG00000136854 | STXBP1 | 1.081 | 4.877 | 3.659 | 0.001 | 0.014 | −0.528 |
| LNM Group 3 vs. Group 1 | ENSG00000069849 | ATP1B3 | 1.080 | 7.022 | 3.378 | 0.002 | 0.024 | −1.345 |
| LNM Group 3 vs. Group 1 | ENSG00000169255 | B3GALNT1 | 1.079 | 3.611 | 3.900 | 0.000 | 0.009 | 0.147 |
| LNM Group 3 vs. Group 1 | ENSG00000171603 | CLSTN1 | 1.077 | 8.118 | 5.354 | 0.000 | 0.001 | 4.390 |
| LNM Group 3 vs. Group 1 | ENSG00000101871 | MID1 | 1.077 | 5.466 | 4.095 | 0.000 | 0.007 | 0.646 |
| LNM Group 3 vs. Group 1 | ENSG00000142949 | PTPRF | 1.066 | 8.842 | 3.394 | 0.002 | 0.023 | −1.297 |
| LNM Group 3 vs. Group 1 | ENSG00000196411 | EPHB4 | 1.065 | 6.613 | 3.689 | 0.001 | 0.013 | −0.515 |
| LNM Group 3 vs. Group 1 | ENSG00000112379 | ARFGEF3 | 1.063 | 3.894 | 4.036 | 0.000 | 0.008 | 0.479 |
| LNM Group 3 vs. Group 1 | ENSG00000137135 | ARHGEF39 | 1.062 | 3.994 | 3.396 | 0.002 | 0.023 | −1.167 |
| LNM Group 3 vs. Group 1 | ENSG00000138463 | DIRC2 | 1.061 | 4.939 | 4.666 | 0.000 | 0.003 | 2.326 |
| LNM Group 3 vs. Group 1 | ENSG00000054392 | HHAT | 1.058 | 3.797 | 2.943 | 0.005 | 0.049 | −2.297 |
| LNM Group 3 vs. Group 1 | ENSG00000125871 | MGME1 | 1.054 | 5.194 | 5.491 | 0.000 | 0.001 | 4.802 |
| LNM Group 3 vs. Group 1 | ENSG00000101236 | RNF24 | 1.053 | 5.965 | 5.518 | 0.000 | 0.001 | 4.895 |
| LNM Group 3 vs. Group 1 | ENSG00000132664 | POLR3F | 1.053 | 3.867 | 4.589 | 0.000 | 0.003 | 2.101 |
| LNM Group 3 vs. Group 1 | ENSG00000118707 | TGIF2 | 1.050 | 5.349 | 4.169 | 0.000 | 0.006 | 0.846 |
| LNM Group 3 vs. Group 1 | ENSG00000117906 | RCN2 | 1.048 | 6.177 | 4.680 | 0.000 | 0.003 | 2.344 |
| LNM Group 3 vs. Group 1 | ENSG00000103855 | CD276 | 1.047 | 6.318 | 3.006 | 0.004 | 0.045 | −2.268 |
| LNM Group 3 vs. Group 1 | ENSG00000188070 | C11orf95 | 1.047 | 5.081 | 3.160 | 0.003 | 0.034 | −1.861 |
| LNM Group 3 vs. Group 1 | ENSG00000148120 | C9orf3 | 1.047 | 6.791 | 4.222 | 0.000 | 0.006 | 0.998 |
| LNM Group 3 vs. Group 1 | ENSG00000136379 | ABHD17C | 1.046 | 4.956 | 3.430 | 0.001 | 0.022 | −1.175 |
| LNM Group 3 vs. Group 1 | ENSG00000101255 | TRIB3 | 1.042 | 4.037 | 3.178 | 0.003 | 0.033 | −1.816 |
| LNM Group 3 vs. Group 1 | ENSG00000125835 | SNRPB | 1.041 | 6.144 | 4.371 | 0.000 | 0.005 | 1.432 |
| LNM Group 3 vs. Group 1 | ENSG00000183779 | ZNF703 | 1.039 | 5.083 | 3.989 | 0.000 | 0.008 | 0.335 |
| LNM Group 3 vs. Group 1 | ENSG00000165304 | MELK | 1.037 | 4.516 | 4.038 | 0.000 | 0.008 | 0.498 |
| LNM Group 3 vs. Group 1 | ENSG00000117632 | STMN1 | 1.036 | 6.544 | 3.691 | 0.001 | 0.013 | −0.513 |
| LNM Group 3 vs. Group 1 | ENSG00000010278 | CD9 | 1.034 | 6.755 | 3.187 | 0.003 | 0.033 | −1.832 |
| LNM Group 3 vs. Group 1 | ENSG00000084636 | COL16A1 | 1.029 | 7.656 | 2.976 | 0.005 | 0.047 | −2.360 |
| LNM Group 3 vs. Group 1 | ENSG00000131652 | THOC6 | 1.027 | 3.900 | 4.178 | 0.000 | 0.006 | 0.930 |
| LNM Group 3 vs. Group 1 | ENSG00000120327 | PCDHB14 | 1.026 | 3.064 | 2.940 | 0.005 | 0.049 | −2.294 |
| LNM Group 3 vs. Group 1 | ENSG00000101220 | C200rf27 | 1.025 | 4.497 | 3.913 | 0.000 | 0.009 | 0.173 |
| LNM Group 3 vs. Group 1 | ENSG00000141837 | CACNA1A | 1.021 | 5.706 | 4.020 | 0.000 | 0.008 | 0.441 |
| LNM Group 3 vs. Group 1 | ENSG00000182004 | SNRPE | 1.020 | 4.387 | 3.972 | 0.000 | 0.009 | 0.350 |
| LNM Group 3 vs. Group 1 | ENSG00000083307 | GRHL2 | 1.020 | 5.924 | 3.819 | 0.000 | 0.011 | −0.137 |
| LNM Group 3 vs. Group 1 | ENSG00000198720 | ANKRD13B | 1.019 | 3.620 | 3.059 | 0.004 | 0 041 | −2.064 |
| LNM Group 3 vs. Group 1 | ENSG00000104723 | TUSC3 | 1.018 | 4.719 | 3.399 | 0.002 | 0.023 | −1.255 |
| LNM Group 3 vs. Group 1 | ENSG00000095002 | MSH2 | 1.016 | 5.261 | 3.607 | 0.001 | 0.015 | −0.673 |
| LNM Group 3 vs. Group 1 | ENSG00000143641 | GALNT2 | 1.012 | 7.024 | 5.240 | 0.000 | 0.001 | 4.039 |
| LNM Group 3 vs. Group 1 | ENSG00000167695 | FAM57A | 1.006 | 4.784 | 4.116 | 0.000 | 0.007 | 0.727 |
| LNM Group 3 vs. Group 1 | ENSG00000177045 | SIX5 | 1.005 | 3.844 | 3.516 | 0.001 | 0.018 | −0.862 |
| LNM Group 3 vs. Group 1 | ENSG00000012211 | PRICKLE3 | 1.003 | 4.021 | 5.221 | 0.000 | 0.001 | 3.960 |
| LNM Group 3 vs. Group 1 | ENSG00000178078 | STAP2 | 1.003 | 4.509 | 2.971 | 0.005 | 0.047 | −2.263 |
| LNM Group 3 vs. Group 1 | ENSG00000109084 | TMEM97 | 1.001 | 3.933 | 3.023 | 0.004 | 0.043 | −2.134 |
| LNM Group 3 vs. Group 1 | ENSG00000188157 | AGRN | 1.000 | 8.450 | 2.975 | 0.005 | 0.047 | −2.348 |
| LNM Group 3 vs. Group 1 | ENSG00000000460 | C1orf112 | 1.000 | 4.410 | 3.589 | 0.001 | 0.016 | −0.692 |
| LNM Group 3 vs. Group 1 | ENSG00000173726 | TOMM20 | 0.999 | 7.329 | 5.259 | 0.000 | 0.001 | 4.095 |
| LNM Group 3 vs. Group 1 | ENSG00000070731 | ST6GALNAC2 | 0.999 | 5.029 | 3.613 | 0.001 | 0.015 | −0.671 |
| LNM Group 3 vs. Group 1 | ENSG00000158169 | FANCC | 0.998 | 5.521 | 4.647 | 0.000 | 0.003 | 2.254 |
| LNM Group 3 vs. Group 1 | ENSG00000172296 | SPTLC3 | 0.995 | 5.568 | 3.273 | 0.002 | 0.029 | −1.544 |
| LNM Group 3 vs. Group 1 | ENSG00000198075 | SULT1C4 | 0.991 | 2.070 | 4.056 | 0.000 | 0.007 | 0.597 |
| LNM Group 3 vs. Group 1 | ENSG00000117650 | NEK2 | 0.990 | 3.955 | 3.041 | 0.004 | 0.042 | −2.099 |
| LNM Group 3 vs. Group 1 | ENSG00000165434 | PGM2L1 | 0.990 | 5.364 | 4.018 | 0.000 | 0.008 | 0.461 |
| LNM Group 3 vs. Group 1 | ENSG00000168795 | ZBTB5 | 0.988 | 5.293 | 5.873 | 0.000 | 0.000 | 5.993 |
| LNM Group 3 vs. Group 1 | ENSG00000125817 | CENPB | 0.984 | 6.263 | 4.742 | 0.000 | 0.002 | 2.530 |
| LNM Group 3 vs. Group 1 | ENSG00000132646 | PCNA | 0.981 | 5.329 | 3.639 | 0.001 | 0.015 | −0.607 |
| LNM Group 3 vs. Group 1 | ENSG00000069275 | NUCKS1 | 0.979 | 8.703 | 3.911 | 0.000 | 0.009 | 0.116 |
| LNM Group 3 vs. Group 1 | ENSG00000139880 | CDH24 | 0.977 | 3.819 | 2.949 | 0.005 | 0.048 | −2.315 |
| LNM Group 3 vs. Group 1 | ENSG00000078900 | TP73 | 0.973 | 3.972 | 3.013 | 0.004 | 0.044 | −2.225 |
| LNM Group 3 vs. Group 1 | ENSG00000122778 | KIAA1549 | 0.965 | 4.167 | 4.237 | 0.000 | 0.006 | 1.068 |
| LNM Group 3 vs. Group 1 | ENSG00000136636 | KCTD3 | 0.963 | 6.019 | 3.990 | 0.000 | 0.008 | 0.342 |
| LNM Group 3 vs. Group 1 | ENSG00000099364 | FBXL19 | 0.962 | 5.432 | 3.340 | 0.002 | 0.025 | −1.379 |
| LNM Group 3 vs. Group 1 | ENSG00000134504 | KCTD1 | 0.961 | 4.839 | 4.329 | 0.000 | 0.005 | 1.334 |
| LNM Group 3 vs. Group 1 | ENSG00000179627 | ZBTB42 | 0.961 | 3.343 | 3.872 | 0.000 | 0.010 | 0.095 |
| LNM Group 3 vs. Group 1 | ENSG00000168758 | SEMA4C | 0.958 | 6.018 | 3.993 | 0.000 | 0.008 | 0.344 |
| LNM Group 3 vs. Group 1 | ENSG00000165283 | STOML2 | 0.952 | 5.125 | 3.798 | 0.000 | 0.011 | −0.125 |
| LNM Group 3 vs. Group 1 | ENSG00000181744 | C3orf58 | 0.949 | 5.562 | 3.941 | 0.000 | 0.009 | 0.188 |
| LNM Group 3 vs. Group 1 | ENSG00000078246 | TULP3 | 0.944 | 4.577 | 3.084 | 0.004 | 0.039 | −1.978 |
| LNM Group 3 vs. Group 1 | ENSG00000165282 | PIGO | 0.941 | 5.498 | 4.044 | 0.000 | 0.008 | 0.539 |
| LNM Group 3 vs. Group 1 | ENSG00000187535 | IFT140 | 0.940 | 4.575 | 3.936 | 0.000 | 0.009 | 0.252 |
| LNM Group 3 vs. Group 1 | ENSG00000180611 | MB21D2 | 0.938 | 3.437 | 2.987 | 0.005 | 0.046 | −2.175 |
| LNM Group 3 vs. Group 1 | ENSG00000141198 | TOM1L1 | 0.937 | 4.264 | 3.564 | 0.001 | 0.017 | −0.786 |
| LNM Group 3 vs. Group 1 | ENSG00000006704 | GTF2IRD1 | 0.935 | 5.604 | 3.298 | 0.002 | 0.027 | −1.525 |
| LNM Group 3 vs. Group 1 | ENSG00000125901 | MRPS26 | 0.935 | 4.118 | 4.429 | 0.000 | 0.004 | 1.635 |
| LNM Group 3 vs. Group 1 | ENSG00000137821 | LRRC49 | 0.935 | 3.597 | 4.231 | 0.000 | 0.006 | 1.059 |
| LNM Group 3 vs. Group 1 | ENSG00000158470 | B4GALT5 | 0.935 | 6.323 | 3.589 | 0.001 | 0.016 | −0.775 |
| LNM Group 3 vs. Group 1 | ENSG00000071575 | TRIB2 | 0.933 | 6.350 | 4.662 | 0.000 | 0.003 | 2.288 |
| LNM Group 3 vs. Group 1 | ENSG00000149639 | SOGA1 | 0.933 | 7.716 | 4.408 | 0.000 | 0.004 | 1.532 |
| LNM Group 3 vs. Group 1 | ENSG00000133026 | MYH10 | 0.932 | 6.900 | 3.429 | 0.001 | 0.022 | −1.209 |
| LNM Group 3 vs. Group 1 | ENSG00000083444 | PLOD1 | 0.931 | 6.368 | 3.248 | 0.002 | 0.030 | −1.675 |
| LNM Group 3 vs. Group 1 | ENSG00000021574 | SPAST | 0.929 | 5.644 | 5.797 | 0.000 | 0.000 | 5.757 |
| LNM Group 3 vs. Group 1 | ENSG00000113161 | HMGCR | 0.929 | 5.892 | 3.031 | 0.004 | 0.043 | −2.189 |
| LNM Group 3 vs. Group 1 | ENSG00000143258 | USP21 | 0.929 | 4.629 | 5.281 | 0.000 | 0.001 | 4.160 |
| LNM Group 3 vs. Group 1 | ENSG00000118257 | NRP2 | 0.925 | 6.812 | 3.093 | 0.004 | 0.039 | −2.066 |
| LNM Group 3 vs. Group 1 | ENSG00000114126 | TFDP2 | 0.925 | 6.410 | 4.395 | 0.000 | 0.004 | 1.494 |
| LNM Group 3 vs. Group 1 | ENSG00000116199 | FAM20B | 0.921 | 6.664 | 4.066 | 0.000 | 0.007 | 0.541 |
| LNM Group 3 vs. Group 1 | ENSG00000221829 | FANCG | 0.921 | 4.386 | 4.128 | 0.000 | 0.007 | 0.791 |
| LNM Group 3 vs. Group 1 | ENSG00000086061 | DNAJA1 | 0.918 | 6.454 | 2.944 | 0.005 | 0.049 | −2.410 |
| LNM Group 3 vs. Group 1 | ENSG00000213930 | GALT | 0.916 | 4.948 | 4.020 | 0.000 | 0.008 | 0.484 |
| LNM Group 3 vs. Group 1 | ENSG00000019485 | PRDM11 | 0.915 | 5.015 | 3.591 | 0.001 | 0.016 | −0.754 |
| LNM Group 3 vs. Group 1 | ENSG00000151914 | DST | 0.915 | 10.012 | 3.604 | 0.001 | 0.016 | −0.713 |
| LNM Group 3 vs. Group 1 | ENSG00000114770 | ABCC5 | 0.915 | 7.369 | 3.642 | 0.001 | 0.015 | −0.641 |
| LNM Group 3 vs. Group 1 | ENSG00000167815 | PRDX2 | 0.914 | 5.114 | 3.244 | 0.002 | 0.030 | −1.650 |
| LNM Group 3 vs. Group 1 | ENSG00000179869 | ABCA13 | 0.913 | 4.705 | 2.962 | 0.005 | 0.047 | −2.364 |
| LNM Group 3 vs. Group 1 | ENSG00000196189 | SEMA4A | 0.912 | 5.880 | 3.112 | 0.003 | 0.038 | −2.012 |
| LNM Group 3 vs. Group 1 | ENSG00000164970 | FAM219A | 0.912 | 5.216 | 3.544 | 0.001 | 0.018 | −0.831 |
| LNM Group 3 vs. Group 1 | ENSG00000169925 | BRD3 | 0.911 | 6.092 | 4.527 | 0.000 | 0.004 | 1.889 |
| LNM Group 3 vs. Group 1 | ENSG00000104835 | SARS2 | 0.909 | 3.949 | 3.373 | 0.002 | 0.024 | −1.256 |
| LNM Group 3 vs. Group 1 | ENSG00000142675 | CNKSR1 | 0.903 | 4.171 | 3.043 | 0.004 | 0.042 | −2.078 |
| LNM Group 3 vs. Group 1 | ENSG00000132623 | ANKEF1 | 0.902 | 4.365 | 3.736 | 0.001 | 0.012 | −0.318 |
| LNM Group 3 vs. Group 1 | ENSG00000143149 | ALDH9A1 | 0.900 | 5.481 | 4.241 | 0.000 | 0.006 | 1.079 |
| LNM Group 3 vs. Group 1 | ENSG00000105202 | FBL | 0.898 | 6.110 | 3.104 | 0.003 | 0.038 | −2.034 |
| LNM Group 3 vs. Group 1 | ENSG00000133619 | KRBA1 | 0.897 | 4.606 | 2.967 | 0.005 | 0.047 | −2.291 |
| LNM Group 3 vs. Group 1 | ENSG00000155755 | TMEM237 | 0.896 | 4.856 | 3.660 | 0.001 | 0.014 | −0.529 |
| LNM Group 3 vs. Group 1 | ENSG00000186522 | SEPT10 | 0.890 | 6.367 | 3.058 | 0.004 | 0.041 | −2.115 |
| LNM Group 3 vs. Group 1 | ENSG00000187164 | SHTN1 | 0.889 | 5.622 | 3.231 | 0.002 | 0.031 | −1.643 |
| LNM Group 3 vs. Group 1 | ENSG00000101856 | PGRMC1 | 0.889 | 5.770 | 4.379 | 0.000 | 0.005 | 1.467 |
| LNM Group 3 vs. Group 1 | ENSG00000158467 | AHCYL2 | 0.888 | 5.108 | 3.019 | 0.004 | 0.044 | −2.142 |
| LNM Group 3 vs. Group 1 | ENSG00000204536 | CCHCR1 | 0.886 | 4.520 | 3.427 | 0.001 | 0.022 | −1.122 |
| LNM Group 3 vs. Group 1 | ENSG00000205476 | CCDC85C | 0.885 | 6.726 | 3.693 | 0.00) | 0.013 | −0.467 |
| LNM Group 3 vs. Group 1 | ENSG00000143321 | HDGF | 0.882 | 7.794 | 4.009 | 0.000 | 0.008 | 0.375 |
| LNM Group 3 vs. Group 1 | ENSG00000122378 | FAM213A | 0.881 | 6.102 | 2.934 | 0.005 | 0.049 | −2.417 |
| LNM Group 3 vs. Group 1 | ENSG00000162714 | ZNF496 | 0.881 | 6.216 | 3.728 | 0.001 | 0.013 | −0.405 |
| LNM Group 3 vs. Group 1 | ENSG00000106367 | AP1S1 | 0.880 | 4.473 | 3.720 | 0.001 | 0.013 | −0.332 |
| LNM Group 3 vs. Group 1 | ENSG00000196391 | ZNF774 | 0.877 | 3.976 | 3.910 | 0.000 | 0.009 | 0.161 |
| LNM Group 3 vs. Group 1 | ENSG00000197077 | KIAA1671 | 0.876 | 7.340 | 3.047 | 0.004 | 0.042 | −2.177 |
| LNM Group 3 vs. Group 1 | ENSG00000168275 | COA6 | 0.876 | 3.290 | 3.176 | 0.003 | 0.033 | −1.694 |
| LNM Group 3 vs. Group 1 | ENSG00000151748 | SAV1 | 0.875 | 5.495 | 3.616 | 0.001 | 0.015 | −0.647 |
| LNM Group 3 vs. Group 1 | ENSG00000070018 | LRP6 | 0.875 | 6.056 | 3.829 | 0.000 | 0.011 | −0.127 |
| LNM Group 3 vs. Group 1 | ENSG00000147874 | HAUS6 | 0.875 | 5.794 | 3.480 | 0.001 | 0.020 | −1.021 |
| LNM Group 3 vs. Group 1 | ENSG00000138336 | TET1 | 0.870 | 3.684 | 3.326 | 0.002 | 0.026 | −1.371 |
| LNM Group 3 vs. Group 1 | ENSG00000183814 | LIN9 | 0.870 | 3.205 | 3.288 | 0.002 | 0.028 | −1.444 |
| LNM Group 3 vs. Group 1 | ENSG00000125484 | GTF3C4 | 0.867 | 6.463 | 5.010 | 0.000 | 0.002 | 3.335 |
| LNM Group 3 vs. Group 1 | ENSG00000175768 | TOMM5 | 0.866 | 5.007 | 3.954 | 0.000 | 0.009 | 0.273 |
| LNM Group 3 vs. Group 1 | ENSG00000074800 | ENO1 | 0.866 | 9.193 | 3.009 | 0.004 | 0.045 | −2.268 |
| LNM Group 3 vs. Group 1 | ENSG00000176463 | SLCO3A1 | 0.863 | 5.862 | 3.295 | 0.002 | 0.027 | −1.551 |
| LNM Group 3 vs. Group 1 | ENSG00000147650 | LRP12 | 0.861 | 5.335 | 2.999 | 0.005 | 0.045 | −2.267 |
| LNM Group 3 vs. Group 1 | ENSG00000196230 | TUBB | 0.861 | 9.131 | 3.965 | 0.000 | 0.009 | 0.266 |
| LNM Group 3 vs. Group 1 | ENSG00000125868 | DSTN | 0.860 | 7.368 | 3.188 | 0.003 | 0.033 | −1.818 |
| LNM Group 3 vs. Group 1 | ENSG00000173207 | CKS1B | 0.860 | 4.649 | 3.367 | 0.002 | 0.024 | −1.284 |
| LNM Group 3 vs. Group 1 | ENSG00000108679 | LGALS3BP | 0.856 | 7.639 | 3.032 | 0.004 | 0.043 | −2.225 |
| LNM Group 3 vs. Group 1 | ENSG00000144320 | KIAA1715 | 0.856 | 6.146 | 3.296 | 0.002 | 0.027 | −1.513 |
| LNM Group 3 vs. Group 1 | ENSG00000000003 | TSPAN6 | 0.856 | 4.498 | 3.269 | 0.002 | 0.029 | −1.539 |
| LNM Group 3 vs. Group 1 | ENSG00000143499 | SMYD2 | 0.855 | 5.652 | 3.583 | 0.001 | 0.016 | −0.755 |
| LNM Group 3 vs. Group 1 | ENSG00000088930 | XRN2 | 0.855 | 6.663 | 4.975 | 0.000 | 0.002 | 3.234 |
| LNM Group 3 vs. Group 1 | ENSG00000135205 | CCDC146 | 0.854 | 3.642 | 2.995 | 0.005 | 0.045 | −2.170 |
| LNM Group 3 vs. Group 1 | ENSG00000136944 | LMX1B | 0.854 | 3.197 | 3.197 | 0.003 | 0.033 | −1.695 |
| LNM Group 3 vs. Group 1 | ENSG00000162851 | TFB2M | 0.853 | 3.247 | 3.857 | 0.000 | 0.010 | 0.047 |
| LNM Group 3 vs. Group 1 | ENSG00000204308 | RNF5 | 0.849 | 4.156 | 4.359 | 0.000 | 0.005 | 1.442 |
| LNM Group 3 vs. Group 1 | ENSG00000124098 | FAM210B | 0.848 | 5.772 | 3.381 | 0.002 | 0.024 | −1.327 |
| LNM Group 3 vs. Group 1 | ENSG00000050327 | ARHGEF5 | 0.842 | 5.829 | 3.202 | 0.003 | 0.032 | −1.741 |
| LNM Group 3 vs. Group 1 | ENSG00000101003 | GINS1 | 0.842 | 4.851 | 3.000 | 0.005 | 0.045 | −2.222 |
| LNM Group 3 vs. Group 1 | ENSG00000108468 | CBX1 | 0.841 | 5.661 | 4.507 | 0.000 | 0.004 | 1.837 |
| LNM Group 3 vs. Group 1 | ENSG00000165997 | ARL5B | 0.840 | 6.324 | 3.774 | 0.001 | 0.012 | −0.258 |
| LNM Group 3 vs. Group 1 | ENSG00000100300 | TSPO | 0.838 | 5.203 | 3.840 | 0.000 | 0.010 | −0.044 |
| LNM Group 3 vs. Group 1 | ENSG00000119669 | IRF2BPL | 0.836 | 5.943 | 4.804 | 0.000 | 0.002 | 2.726 |
| LNM Group 3 vs. Group 1 | ENSG00000197562 | RAB40C | 0.832 | 4.749 | 3.263 | 0.002 | 0.029 | −1.548 |
| LNM Group 3 vs. Group 1 | ENSG00000143256 | PFDN2 | 0.831 | 3.485 | 3.085 | 0.004 | 0.039 | −1.943 |
| LNM Group 3 vs. Group 1 | ENSG00000182704 | TSKU | 0.830 | 6.606 | 3.201 | 0.003 | 0.032 | −1.765 |
| LNM Group 3 vs. Group 1 | ENSG00000171262 | FAM98B | 0.827 | 5.614 | 4.175 | 0.000 | 0.006 | 0.873 |
| LNM Group 3 vs. Group 1 | ENSG00000157216 | SSBP3 | 0.827 | 5.661 | 4.079 | 0.000 | 0.007 | 0.593 |
| LNM Group 3 vs. Group 1 | ENSG00000187607 | ZNF286A | 0.827 | 4.927 | 3.443 | 0.001 | 0.021 | −1.130 |
| LNM Group 3 vs. Group 1 | ENSG00000089050 | RBBP9 | 0.825 | 5.026 | 3.070 | 0.004 | 0.040 | −2.045 |
| LNM Group 3 vs. Group 1 | ENSG00000149925 | ALDOA | 0.818 | 8.684 | 2.962 | 0.005 | 0.047 | −2.393 |
| LNM Group 3 vs. Group 1 | ENSG00000164877 | MICALL2 | 0.818 | 6.519 | 3.036 | 0.004 | 0.043 | −2.194 |
| LNM Group 3 vs. Group 1 | ENSG00000005156 | LIG3 | 0.817 | 6.498 | 4.450 | 0.000 | 0.004 | 1.662 |
| LNM Group 3 vs. Group 1 | ENSG00000114054 | PCCB | 0.813 | 4.891 | 3.729 | 0.001 | 0.013 | −0.356 |
| LNM Group 3 vs. Group 1 | ENSG00000105135 | ILVBL | 0.813 | 5.015 | 3.852 | 0.000 | 0.010 | −0.008 |
| LNM Group 3 vs. Group 1 | ENSG00000017260 | ATP2C1 | 0.810 | 7.598 | 3.933 | 0.000 | 0.009 | 0.161 |
| LNM Group 3 vs. Group 1 | ENSG00000168264 | IRF2BP2 | 0.805 | 7.766 | 4.182 | 0.000 | 0.006 | 0.872 |
| LNM Group 3 vs. Group 1 | ENSG00000074696 | HACD3 | 0.805 | 6.575 | 4.137 | 0.000 | 0.007 | 0.744 |
| LNM Group 3 vs. Group 1 | ENSG00000100246 | DNAL4 | 0.804 | 3.194 | 3.868 | 0.000 | 0.010 | 0.070 |
| LNM Group 3 vs. Group 1 | ENSG00000127586 | CHTF18 | 0.804 | 5.099 | 2.972 | 0.005 | 0.047 | −2.310 |
| LNM Group 3 vs. Group 1 | ENSG00000116212 | LRRC42 | 0.802 | 4.193 | 2.992 | 0.005 | 0.045 | −2.185 |
| LNM Group 3 vs. Group 1 | ENSG00000122705 | CLTA | 0.802 | 6.008 | 4.513 | 0.000 | 0.004 | 1.854 |
| LNM Group 3 vs. Group 1 | ENSG00000125534 | PPDPF | 0.801 | 6.403 | 4.586 | 0.000 | 0.003 | 2.062 |
| LNM Group 3 vs. Group 1 | ENSG00000177868 | SVBP | 0.800 | 2.388 | 3.312 | 0.002 | 0.026 | −1.360 |
| LNM Group 3 vs. Group 1 | ENSG00000132661 | NXT1 | 0.799 | 3.265 | 3.251 | 0.002 | 0.029 | −1.520 |
| LNM Group 3 vs. Group 1 | ENSG00000147400 | CETN2 | 0.799 | 3.789 | 3.802 | 0.000 | 0.011 | −0.107 |
| LNM Group 3 vs. Group 1 | ENSG00000152990 | ADGRA3 | 0.797 | 5.417 | 3.258 | 0.002 | 0.029 | −1.600 |
| LNM Group 3 vs. Group 1 | ENSG00000184897 | H1FX | 0.796 | 6.281 | 4.122 | 0.000 | 0.007 | 0.706 |
| LNM Group 3 vs. Group 1 | ENSG00000183828 | NUDT14 | 0.792 | 3.562 | 2.973 | 0.005 | 0.047 | −2.195 |
| LNM Group 3 vs. Group 1 | ENSG00000160767 | FAM189B | 0.791 | 4.980 | 3.360 | 0.002 | 0.024 | −1.321 |
| LNM Group 3 vs. Group 1 | ENSG00000162585 | FAAP20 | 0.788 | 5.537 | 3.569 | 0.001 | 0.017 | −0.814 |
| LNM Group 3 vs. Group 1 | ENSG00000162885 | B3GALNT2 | 0.788 | 5.346 | 4.444 | 0.000 | 0.004 | 1.654 |
| LNM Group 3 vs. Group 1 | ENSG00000160271 | RALGDS | 0.787 | 7.165 | 3.133 | 0.003 | 0.036 | −1.969 |
| LNM Group 3 vs. Group 1 | ENSG00000164758 | MED30 | 0.787 | 2.905 | 3.551 | 0.001 | 0.017 | −0.757 |
| LNM Group 3 vs. Group 1 | ENSG00000179958 | DCTPP1 | 0.786 | 3.897 | 3.209 | 0.003 | 0.032 | −1.659 |
| LNM Group 3 vs. Group 1 | ENSG00000180992 | MRPL14 | 0.784 | 3.808 | 3.266 | 0.002 | 0.029 | −1.504 |
| LNM Group 3 vs. Group 1 | ENSG00000240849 | TMEM189 | 0.784 | 6.643 | 3.660 | 0.001 | 0.014 | −0.588 |
| LNM Group 3 vs. Group 1 | ENSG00000114125 | RNF7 | 0.783 | 5.600 | 4.150 | 0.000 | 0.006 | 0.794 |
| LNM Group 3 vs. Group 1 | ENSG00000131116 | ZNF428 | 0.782 | 3.732 | 3.096 | 0.004 | 0.039 | −1.934 |
| LNM Group 3 vs. Group 1 | ENSG00000139350 | NEDD1 | 0.781 | 5.453 | 2.948 | 0.005 | 0.048 | −2.353 |
| LNM Group 3 vs. Group 1 | ENSG00000109113 | RAB34 | 0.781 | 4.980 | 3.186 | 0.003 | 0.033 | −1.779 |
| LNM Group 3 vs. Group 1 | ENSG00000198876 | DCAF12 | 0.780 | 6.113 | 4.194 | 0.000 | 0.006 | 0.923 |
| LNM Group 3 vs. Group 1 | ENSG00000163041 | H3F3A | 0.775 | 7.837 | 5.038 | 0.000 | 0.002 | 3.420 |
| LNM Group 3 vs. Group 1 | ENSG00000088854 | C20orf194 | 0.773 | 5.956 | 3.040 | 0.004 | 0.042 | −2.131 |
| LNM Group 3 vs. Group 1 | ENSG00000185630 | PBX1 | 0.772 | 6.170 | 3.221 | 0.002 | 0.031 | −1.751 |
| LNM Group 3 vs. Group 1 | ENSG00000020256 | ZFP64 | 0.772 | 5.005 | 3.875 | 0.000 | 0.010 | 0.025 |
| LNM Group 3 vs. Group 1 | ENSG00000178163 | ZNF518B | 0.770 | 5.931 | 3.123 | 0.003 | 0.037 | −1.977 |
| LNM Group 3 vs. Group 1 | ENSG00000177383 | MAGEF1 | 0.768 | 4.429 | 4.251 | 0.000 | 0.006 | 1.113 |
| LNM Group 3 vs. Group 1 | ENSG00000137124 | ALDH1B1 | 0.767 | 4.475 | 3.017 | 0.004 | 0.044 | −2.172 |
| LNM Group 3 vs. Group 1 | ENSG00000114988 | LMAN2L | 0.766 | 3.777 | 3.357 | 0.002 | 0.025 | −1.266 |
| LNM Group 3 vs. Group 1 | ENSG00000188229 | TUBB4B | 0.763 | 7.150 | 3.047 | 0.004 | 0.042 | −2.184 |
| LNM Group 3 vs. Group 1 | ENSG00000137074 | APTX | 0.763 | 4.734 | 3.090 | 0.004 | 0.039 | −1.961 |
| LNM Group 3 vs. Group 1 | ENSG00000125652 | ALKBH7 | 0.762 | 3.115 | 3.000 | 0.005 | 0.045 | −2.117 |
| LNM Group 3 vs. Group 1 | ENSG00000163382 | NAXE | 0.760 | 4.573 | 4.537 | 0.000 | 0.004 | 1.950 |
| LNM Group 3 vs. Group 1 | ENSG00000081692 | JMJD4 | 0.758 | 4.762 | 3.772 | 0.001 | 0.012 | −0.234 |
| LNM Group 3 vs. Group 1 | ENSG00000143553 | SNAPIN | 0.756 | 3.833 | 3.863 | 0.000 | 0.010 | 0.062 |
| LNM Group 3 vs. Group 1 | ENSG00000143493 | INTS7 | 0.754 | 5.317 | 3.255 | 0.002 | 0.029 | −1.602 |
| LNM Group 3 vs. Group 1 | ENSG00000175130 | MARCKSL1 | 0.754 | 6.052 | 3.103 | 0.003 | 0.038 | −2.036 |
| LNM Group 3 vs. Group 1 | ENSG00000143368 | SF3B4 | 0.753 | 5.240 | 3.612 | 0.001 | 0.015 | −0.650 |
| LNM Group 3 vs. Group 1 | ENSG00000018408 | WWTR1 | 0.752 | 6.583 | 3.011 | 0.004 | 0.044 | −2.266 |
| LNM Group 3 vs. Group 1 | ENSG00000136522 | MRPL47 | 0.752 | 4.389 | 3.483 | 0.001 | 0.020 | −0.990 |
| LNM Group 3 vs. Group 1 | ENSG00000165804 | ZNF219 | 0.751 | 4.798 | 3.698 | 0.001 | 0.013 | −0.414 |
| LNM Group 3 vs. Group 1 | ENSG00000168778 | TCTN2 | 0.750 | 4.447 | 3.180 | 0.003 | 0.033 | −1.759 |
| LNM Group 3 vs. Group 1 | ENSG00000059573 | ALDH18A1 | 0.746 | 5.726 | 3.884 | 0.000 | 0.010 | 0.068 |
| LNM Group 3 vs. Group 1 | ENSG00000107779 | BMPR1A | 0.746 | 5.699 | 3.864 | 0.000 | 0.010 | 0.012 |
| LNM Group 3 vs. Group 1 | ENSG00000103404 | USP31 | 0.744 | 6.813 | 3.130 | 0.003 | 0.037 | −1.968 |
| LNM Group 3 vs. Group 1 | ENSG00000196418 | ZNF124 | 0.742 | 5.095 | 4.080 | 0.000 | 0.007 | 0.609 |
| LNM Group 3 vs. Group 1 | ENSG00000007047 | MARK4 | 0.742 | 5.703 | 3.981 | 0.000 | 0.008 | 0.314 |
| LNM Group 3 vs. Group 1 | ENSG00000154640 | BTG3 | 0.742 | 4.568 | 3.037 | 0.004 | 0.043 | −2.154 |
| LNM Group 3 vs. Group 1 | ENSG00000143578 | CREB3L4 | 0.741 | 2.904 | 2.944 | 0.005 | 0.049 | −2.254 |
| LNM Group 3 vs. Group 1 | ENSG00000139197 | PEX5 | 0.740 | 5.241 | 4.082 | 0.000 | 0.007 | 0.608 |
| LNM Group 3 vs. Group 1 | ENSG00000121989 | ACVR2A | 0.738 | 4.973 | 2.952 | 0.005 | 0.048 | −2.321 |
| LNM Group 3 vs. Group 1 | ENSG00000126432 | PRDX5 | 0.736 | 5.608 | 3.431 | 0.001 | 0.022 | −1.120 |
| LNM Group 3 vs. Group 1 | ENSG00000157985 | AGAP1 | 0.735 | 5.617 | 3.288 | 0.002 | 0.028 | −1.562 |
| LNM Group 3 vs. Group 1 | ENSG00000143742 | SRP9 | 0.733 | 7.103 | 4.334 | 0.000 | 0.005 | 1.312 |
| LNM Group 3 vs. Group 1 | ENSG00000112144 | ICK | 0.731 | 5.453 | 3.330 | 0.002 | 0.026 | −1.433 |
| LNM Group 3 vs. Group 1 | ENSG00000111845 | PAK1IP1 | 0.730 | 3.610 | 3.454 | 0.001 | 0.021 | −1.037 |
| LNM Group 3 vs. Group 1 | ENSG00000089091 | DZANK1 | 0.730 | 3.535 | 2.949 | 0.005 | 0.048 | −2.266 |
| LNM Group 3 vs. Group 1 | ENSG00000163964 | PIGX | 0.729 | 4.982 | 3.492 | 0.001 | 0.019 | −1.009 |
| LNM Group 3 vs. Group 1 | ENSG00000164663 | USP49 | 0.726 | 5.208 | 3.067 | 0.004 | 0.041 | −2.072 |
| LNM Group 3 vs. Group 1 | ENSG00000113300 | CNOT6 | 0.725 | 6.466 | 3.954 | 0.000 | 0.009 | 0.252 |
| LNM Group 3 vs. Group 1 | ENSG00000178409 | BEND3 | 0.725 | 3.802 | 3.324 | 0.002 | 0.026 | −1.373 |
| LNM Group 3 vs. Group 1 | ENSG00000158623 | COPG2 | 0.720 | 4.713 | 4.136 | 0.000 | 0.007 | 0.796 |
| LNM Group 3 vs. Group 1 | ENSG00000130382 | MLLT1 | 0.716 | 6.107 | 3.300 | 0.002 | 0.027 | −1.539 |
| LNM Group 3 vs. Group 1 | ENSG00000137166 | FOXP4 | 0.716 | 5.508 | 3.343 | 0.002 | 0.025 | −1.418 |
| LNM Group 3 vs. Group 1 | ENSG00000067704 | IARS2 | 0.715 | 6.373 | 3.429 | 0.001 | 0.022 | −1.181 |
| LNM Group 3 vs. Group 1 | ENSG00000256349 | CTD- | 0.710 | 2.593 | 3.065 | 0.004 | 0.041 | −1.970 |
| | | 307407.11 | | | | | | |
| LNM Group 3 vs. Group 1 | ENSG00000135912 | TILL4 | 0.709 | 5.416 | 3.462 | 0.001 | 0.020 | −1.087 |
| LNM Group 3 vs. Group 1 | ENSG00000163918 | RFC4 | 0.708 | 4.149 | 3.376 | 0.002 | 0.024 | −1.264 |
| LNM Group 3 vs. Group 1 | ENSG00000161996 | WDR90 | 0.705 | 6.119 | 4.221 | 0.000 | 0.006 | 0.996 |
| LNM Group 3 vs. Group 1 | ENSG00000099800 | TIMM13 | 0.702 | 4.664 | 3.349 | 0.002 | 0.025 | −1.339 |
| LNM Group 3 vs. Group 1 | ENSG00000116586 | LAMTOR2 | 0.701 | 3.476 | 3.198 | 0.003 | 0.033 | −1.643 |
| LNM Group 3 vs. Group 1 | ENSG00000137804 | NUSAP1 | 0.700 | 5.405 | 2.942 | 0.005 | 0.049 | −2.398 |
| LNM Group 3 vs. Group 1 | ENSG00000101266 | CSNK2A1 | 0.699 | 7.014 | 4.716 | 0.000 | 0.003 | 2.445 |
| LNM Group 3 vs. Group 1 | ENSG00000120008 | WDR11 | 0.696 | 6.829 | 3.826 | 0.000 | 0.011 | −0.124 |
| LNM Group 3 vs. Group 1 | ENSG00000247315 | ZCCHC3 | 0.696 | 4.571 | 4.172 | 0.000 | 0.006 | 0.891 |
| LNM Group 3 vs. Group 1 | ENSG00000163785 | RYK | 0.694 | 6.706 | 4.063 | 0.000 | 0.007 | 0.530 |
| LNM Group 3 vs. Group 1 | ENSG00000143621 | ILF2 | 0.694 | 6.366 | 4.682 | 0.000 | 0.003 | 2.350 |
| LNM Group 3 vs. Group 1 | ENSG00000132694 | ARHGEF11 | 0.692 | 6.513 | 3.174 | 0.003 | 0.033 | −1.851 |
| LNM Group 3 vs. Group 1 | ENSG00000168118 | RAB4A | 0.692 | 5.048 | 3.103 | 0.003 | 0.038 | −1.976 |
| LNM Group 3 vs. Group 1 | ENSG00000135778 | NTPCR | 0.690 | 5.737 | 3.895 | 0.000 | 0.009 | 0.106 |
| LNM Group 3 vs. Group 1 | ENSG00000228300 | C19orf24 | 0.688 | 3.645 | 3.400 | 0.001 | 0.023 | −1.181 |
| LNM Group 3 vs. Group 1 | ENSG00000176396 | EID2 | 0.687 | 2.944 | 2.950 | 0.005 | 0.048 | −2.222 |
| LNM Group 3 vs. Group 1 | ENSG00000124688 | MAD2L1BP | 0.687 | 3.675 | 3.545 | 0.001 | 0.017 | −0.785 |
| LNM Group 3 vs. Group 1 | ENSG00000138175 | ARL3 | 0.687 | 3.153 | 3.474 | 0.001 | 0.020 | −0.957 |
| LNM Group 3 vs. Group 1 | ENSG00000089123 | TASP1 | 0.687 | 4.290 | 3.305 | 0.002 | 0.027 | −1.432 |
| LNM Group 3 vs. Group 1 | ENSG00000141994 | DUS3L | 0.686 | 4.327 | 3.114 | 0.003 | 0.038 | −1.927 |
| LNM Group 3 vs. Group 1 | ENSG00000138002 | IFT172 | 0.685 | 4.913 | 3.392 | 0.002 | 0.023 | −1.246 |
| LNM Group 3 vs. Group 1 | ENSG00000185236 | RAB11B | 0.685 | 5.473 | 3.382 | 0.002 | 0.023 | −1.285 |
| LNM Group 3 vs. Group 1 | ENSG00000125779 | PANK2 | 0.685 | 6.065 | 4.595 | 0.000 | 0.003 | 2.096 |
| LNM Group 3 vs. Group 1 | ENSG00000099810 | MTAP | 0.683 | 6.736 | 3.022 | 0.004 | 0.043 | −2.205 |
| LNM Group 3 vs. Group 1 | ENSG00000114491 | UMPS | 0.679 | 5.786 | 2.989 | 0.005 | 0.046 | −2.304 |
| LNM Group 3 vs. Group 1 | ENSG00000118007 | STAG1 | 0.679 | 6.758 | 4.741 | 0.000 | 0.002 | 2.520 |
| LNM Group 3 vs. Group 1 | ENSG00000164889 | SLC4A2 | 0.678 | 6.030 | 3.400 | 0.001 | 0.023 | −1.264 |
| LNM Group 3 vs. Group 1 | ENSG00000180891 | CUEDC1 | 0.678 | 4.969 | 3.209 | 0.003 | 0.032 | −1.742 |
| LNM Group 3 vs. Group 1 | ENSG00000163655 | GMPS | 0.675 | 6.928 | 4.226 | 0.000 | 0.006 | 0.999 |
| LNM Group 3 vs. Group 1 | ENSG00000125834 | STK35 | 0.674 | 6.495 | 3.780 | 0.000 | 0.012 | −0.264 |
| LNM Group 3 vs. Group 1 | ENSG00000137054 | POLR1E | 0.673 | 4.192 | 3.264 | 0.002 | 0.029 | −1.547 |
| LNM Group 3 vs. Group 1 | ENSG00000114107 | CEP70 | 0.673 | 4.401 | 3.024 | 0.004 | 0.043 | −2.195 |
| LNM Group 3 vs. Group 1 | ENSG00000035687 | ADSS | 0.672 | 6.078 | 4.481 | 0.000 | 0.004 | 1.749 |
| LNM Group 3 vs. Group 1 | ENSG00000197345 | MRPL21 | 0.672 | 4.513 | 3.690 | 0.001 | 0.013 | −0.472 |
| LNM Group 3 vs. Group 1 | ENSG00000143436 | MRPL9 | 0.671 | 4.477 | 3.463 | 0.001 | 0.020 | −1.021 |
| LNM Group 3 vs. Group 1 | ENSG00000132475 | H3F3B | 0.671 | 8.660 | 3.743 | 0.001 | 0.012 | −0.360 |
| LNM Group 3 vs. Group 1 | ENSG00000073803 | MAP3K13 | 0.669 | 5.921 | 3.324 | 0.002 | 0.026 | −1.467 |
| LNM Group 3 vs. Group 1 | ENSG00000265241 | RBM8A | 0.668 | 6.502 | 3.609 | 0.001 | 0.015 | −0.720 |
| LNM Group 3 vs. Group 1 | ENSG00000124702 | KLHDC3 | 0.666 | 5.308 | 4.516 | 0.000 | 0.004 | 1.877 |
| LNM Group 3 vs. Group 1 | ENSG00000170604 | IRF2BP1 | 0.665 | 4.024 | 3.159 | 0.003 | 0.034 | −1.798 |
| LNM Group 3 vs. Group 1 | ENSG00000116957 | TBCE | 0.664 | 4.423 | 4.275 | 0.000 | 0.006 | 1.194 |
| LNM Group 3 vs. Group 1 | ENSG00000146858 | ZC3HAV1L | 0.663 | 3.965 | 3.392 | 0.002 | 0.023 | −1.208 |
| LNM Group 3 vs. Group 1 | ENSG00000127616 | SMARCA4 | 0.663 | 7.453 | 3.619 | 0.001 | 0.015 | −0.708 |
| LNM Group 3 vs. Group 1 | ENSG00000133422 | MORC2 | 0.663 | 6.613 | 3.277 | 0.002 | 0.028 | −1.606 |
| LNM Group 3 vs. Group 1 | ENSG00000125898 | FAM110A | 0.662 | 4.268 | 3.031 | 0.004 | 0.043 | −2.090 |
| LNM Group 3 vs. Group 1 | ENSG00000140391 | TSPAN3 | 0.661 | 6.447 | 3.407 | 0.001 | 0.022 | −1.257 |
| LNM Group 3 vs. Group 1 | ENSG00000125743 | SNRPD2 | 0.661 | 5.122 | 3.727 | 0.001 | 0.013 | −0.359 |
| LNM Group 3 vs. Group 1 | ENSG00000145284 | SCD5 | 0.658 | 5.856 | 3.792 | 0.000 | 0.011 | −0.212 |
| LNM Group 3 vs. Group 1 | ENSG00000101343 | CRNKL1 | 0.656 | 5.166 | 3.195 | 0.003 | 0.033 | −1.735 |
| LNM Group 3 vs. Group 1 | ENSG00000159217 | IGF2BP1 | 0.656 | 4.207 | 3.287 | 0.002 | 0.028 | −1.508 |
| LNM Group 3 vs. Group 1 | ENSG00000152904 | GGPS1 | 0.655 | 4.946 | 4.303 | 0.000 | 0.005 | 1.261 |
| LNM Group 3 vs. Group 1 | ENSG00000163913 | IFT122 | 0.654 | 5.199 | 3.414 | 0.001 | 0.022 | −1.214 |
| LNM Group 3 vs. Group 1 | ENSG00000161202 | DVL3 | 0.654 | 7.184 | 3.194 | 0.003 | 0.033 | −1.818 |
| LNM Group 3 vs. Group 1 | ENSG00000096384 | HSP90AB1 | 0.653 | 8.939 | 2.983 | 0.005 | 0.046 | −2.326 |
| LNM Group 3 vs. Group 1 | ENSG00000101365 | IDH3B | 0.652 | 5.333 | 3.337 | 0.002 | 0.026 | −1.383 |
| LNM Group 3 vs. Group 1 | ENSG00000197965 | MPZL1 | 0.652 | 7.263 | 3.587 | 0.001 | 0.016 | −0.776 |
| LNM Group 3 vs. Group 1 | ENSG00000107341 | UBE2R2 | 0.652 | 6.362 | 3.104 | 0.003 | 0.038 | −1.997 |
| LNM Group 3 vs. Group 1 | ENSG00000107175 | CREB3 | 0.651 | 4.283 | 4.051 | 0.000 | 0.007 | 0.570 |
| LNM Group 3 vs. Group 1 | ENSG00000167635 | ZNF146 | 0.651 | 6.698 | 4.055 | 0.000 | 0.007 | 0.510 |
| LNM Group 3 vs. Group 1 | ENSG00000186350 | RXRA | 0.649 | 7.402 | 3.093 | 0.004 | 0.039 | −2.067 |
| LNM Group 3 vs. Group 1 | ENSG00000188060 | RAB42 | 0.649 | 2.828 | 3.043 | 0.004 | 0.042 | −2.014 |
| LNM Group 3 vs. Group 1 | ENSG00000167645 | YIF1B | 0.647 | 4.481 | 3.338 | 0.002 | 0.026 | −1.332 |
| LNM Group 3 vs. Group 1 | ENSG00000182768 | NGRN | 0.647 | 6.656 | 4.267 | 0.000 | 0.006 | 1.121 |
| LNM Group 3 vs. Group 1 | ENSG00000198700 | IPO9 | 0.646 | 7.147 | 4.008 | 0.000 | 0.008 | 0.372 |
| LNM Group 3 vs. Group 1 | ENSG00000125870 | SNRPB2 | 0.645 | 4.436 | 3.522 | 0.001 | 0.018 | −0.848 |
| LNM Group 3 vs. Group 1 | ENSG00000112658 | SRF | 0.645 | 5.868 | 3.106 | 0.003 | 0.038 | −2.015 |
| LNM Group 3 vs. Group 1 | ENSG00000103495 | MAZ | 0.644 | 6.950 | 3.334 | 0.002 | 0.026 | −1.455 |
| LNM Group 3 vs. Group 1 | ENSG00000137404 | NRM | 0.641 | 3.816 | 2.939 | 0.005 | 0.049 | −2.283 |
| LNM Group 3 vs. Group 1 | ENSG00000160783 | PMF1 | 0.641 | 4.635 | 3.295 | 0.002 | 0.027 | −1.475 |
| LNM Group 3 vs. Group 1 | ENSG00000124535 | WRNIP1 | 0.639 | 5.399 | 3.693 | 0.001 | 0.013 | −0.471 |
| LNM Group 3 vs. Group 1 | ENSG00000257315 | ZBED6 | 0.637 | 6.880 | 4.910 | 0.000 | 0.002 | 3.033 |
| LNM Group 3 vs. Group 1 | ENSG00000143363 | PRUNE1 | 0.636 | 4.453 | 3.179 | 0.003 | 0.033 | −1.741 |
| LNM Group 3 vs. Group 1 | ENSG00000106400 | ZNHIT1 | 0.635 | 5.825 | 3.526 | 0.001 | 0.018 | −0.894 |
| LNM Group 3 vs. Group 1 | ENSG00000001167 | NFYA | 0.633 | 5.384 | 3.341 | 0.002 | 0.025 | −1.404 |
| LNM Group 3 vs. Group 1 | ENSG00000165688 | PMPCA | 0.632 | 5.693 | 4.136 | 0.000 | 0.007 | 0.777 |
| LNM Group 3 vs. Group 1 | ENSG00000165006 | UBAP1 | 0.628 | 5.933 | 3.020 | 0.004 | 0.044 | −2.201 |
| LNM Group 3 vs. Group 1 | ENSG00000117625 | RCOR3 | 0.626 | 6.259 | 3.912 | 0.000 | 0.009 | 0.121 |
| LNM Group 3 vs. Group 1 | ENSG00000103222 | ABCC1 | 0.622 | 7.732 | 3.386 | 0.002 | 0.023 | −1.327 |
| LNM Group 3 vs. Group 1 | ENSG00000125877 | ITPA | 0.621 | 4.880 | 3.087 | 0.004 | 0.039 | −1.994 |
| LNM Group 3 vs. Group 1 | ENSG00000173950 | XXYLT1 | 0.620 | 4.459 | 3.300 | 0.002 | 0.027 | −1.499 |
| LNM Group 3 vs. Group 1 | ENSG00000160679 | CHTOP | 0.620 | 6.573 | 4.688 | 0.000 | 0.003 | 2.366 |
| LNM Group 3 vs. Group 1 | ENSG00000182095 | TNRC18 | 0.619 | 8.200 | 3.013 | 0.004 | 0.044 | −2.260 |
| LNM Group 3 vs. Group 1 | ENSG00000160752 | FDPS | 0.619 | 5.958 | 3.711 | 0.001 | 0.013 | −0.449 |
| LNM Group 3 vs. Group 1 | ENSG00000089048 | ESF1 | 0.618 | 4.713 | 3.372 | 0.002 | 0.024 | −1.297 |
| LNM Group 3 vs. Group 1 | ENSG00000108953 | YWHAE | 0.618 | 7.797 | 3.124 | 0.003 | 0.037 | −1.997 |
| LNM Group 3 vs. Group 1 | ENSG00000139146 | FAM60A | 0.618 | 6.427 | 3.262 | 0.002 | 0.029 | −1.647 |
| LNM Group 3 vs. Group 1 | ENSG00000158850 | B4GALT3 | 0.617 | 5.147 | 3.479 | 0.001 | 0.020 | −0.996 |
| LNM Group 3 vs. Group 1 | ENSG00000071564 | TCF3 | 0.616 | 6.617 | 3.050 | 0.004 | 0.042 | −2.179 |
| LNM Group 3 vs. Group 1 | ENSG00000185019 | UBOX5 | 0.616 | 4.656 | 2.965 | 0.005 | 0.047 | −2.290 |
| LNM Group 3 vs. Group 1 | ENSG00000257365 | FNTB | 0.615 | 4.412 | 3.024 | 0.004 | 0.043 | −2.100 |
| LNM Group 3 vs. Group 1 | ENSG00000224531 | SMIM13 | 0.615 | 4.666 | 3.116 | 0.003 | 0.037 | −1.951 |
| LNM Group 3 vs. Group 1 | ENSG00000166886 | NAB2 | 0.612 | 5.117 | 2.998 | 0.005 | 0.045 | −2.242 |
| LNM Group 3 vs. Group 1 | ENSG00000206053 | HN1L | 0.612 | 6.640 | 2.958 | 0.005 | 0.048 | −2.380 |
| LNM Group 3 vs. Group 1 | ENSG00000123684 | LPGAT1 | 0.607 | 6.922 | 3.076 | 0.004 | 0.040 | −2.106 |
| LNM Group 3 vs. Group 1 | ENSG00000143420 | ENSA | 0.606 | 7.312 | 3.972 | 0.000 | 0.009 | 0.271 |
| LNM Group 3 vs. Group 1 | ENSG00000143514 | TP53BP2 | 0.606 | 6.103 | 3.143 | 0.003 | 0.036 | −1.926 |
| LNM Group 3 vs. Group 1 | ENSG00000171453 | POLR1C | 0.604 | 4.271 | 3.180 | 0.003 | 0.033 | −1.756 |
| LNM Group 3 vs. Group 1 | ENSG00000188739 | RBM34 | 0.602 | 5.226 | 3.984 | 0.000 | 0.008 | 0.336 |
| LNM Group 3 vs. Group 1 | ENSG00000215251 | FASTKD5 | 0.602 | 4.543 | 3.925 | 0.000 | 0.009 | 0.200 |
| LNM Group 3 vs. Group 1 | ENSG00000160803 | UBQLN4 | 0.598 | 5.632 | 3.127 | 0.003 | 0.037 | −1.951 |
| LNM Group 3 vs. Group 1 | ENSG00000101247 | NDUFAF5 | 0.598 | 4.775 | 3.040 | 0.004 | 0.042 | −2.108 |
| LNM Group 3 vs. Group 1 | ENSG0OO00163479 | SSR2 | 0.597 | 6.584 | 3.092 | 0.004 | 0.039 | −2.050 |
| LNM Group 3 vs. Group 1 | ENSG00000197557 | TTC30A | 0.595 | 3.055 | 3.101 | 0.003 | 0.038 | −1.917 |
| LNM Group 3 vs. Group 1 | ENSG00000107874 | CUEDC2 | 0.593 | 3.648 | 3.120 | 0.003 | 0.037 | −1.843 |
| LNM Group 3 vs. Group 1 | ENSG00000198171 | DDRGK1 | 0.593 | 4.677 | 3.082 | 0.004 | 0.040 | −2.028 |
| LNM Group 3 vs. Group 1 | ENSG00000173597 | SULT1B1 | 0.591 | 8.530 | 2.975 | 0.005 | 0.047 | −2.361 |
| LNM Group 3 vs. Group 1 | ENSG00000101474 | APMAP | 0.590 | 5.991 | 3.675 | 0.001 | 0.014 | −0.520 |
| LNM Group 3 vs. Group 1 | ENSG00000163781 | TOPBP1 | 0.590 | 6.540 | 3.919 | 0.000 | 0.009 | 0.124 |
| LNM Group 3 vs. Group 1 | ENSG00000166508 | MCM7 | 0.590 | 6.696 | 3.039 | 0.004 | 0.043 | −2.205 |
| LNM Group 3 vs. Group 1 | ENSG00000130402 | ACTN4 | 0.590 | 8.608 | 2.968 | 0.005 | 0.047 | −2.371 |
| LNM Group 3 vs. Group 1 | ENSG00000166415 | WDR72 | 0.589 | 5.022 | 3.400 | 0.001 | 0.023 | −1.245 |
| LNM Group 3 vs. Group 1 | ENSG00000121057 | AKAP1 | 0.589 | 5.606 | 3.212 | 0.003 | 0.032 | −1.747 |
| LNM Group 3 vs. Group 1 | ENSG00000065518 | NDUFB4 | 0.589 | 5.097 | 2.974 | 0.005 | 0.047 | −2.276 |
| LNM Group 3 vs. Group 1 | ENSG00000004975 | DVL2 | 0.588 | 5.471 | 3.443 | 0.001 | 0.021 | −1.134 |
| LNM Group 3 vs. Group 1 | ENSG00000006756 | ARSD | 0.588 | 5.304 | 3.865 | 0.000 | 0.010 | 0.020 |
| LNM Group 3 vs. Group 1 | ENSG00000163608 | NEPRO | 0.586 | 6.122 | 3.991 | 0.000 | 0.008 | 0.348 |
| LNM Group 3 vs. Group 1 | ENSG00000161091 | MFSD12 | −0.586 | 6.190 | −3.053 | 0.004 | 0.041 | −2.121 |
| LNM Group 3 vs. Group 1 | ENSG00000166479 | TMX3 | −0.588 | 6.159 | −3.148 | 0.003 | 0.035 | −1.917 |
| LNM Group 3 vs. Group 1 | ENSG00000144468 | RHBDD1 | −0.588 | 5.581 | −3.980 | 0.000 | 0.008 | 0.337 |
| LNM Group 3 vs. Group 1 | ENSG00000144746 | ARL6IP5 | −0.590 | 5.907 | −2.959 | 0.005 | 0.047 | −2.294 |
| LNM Group 3 vs. Group 1 | ENSG00000144559 | TAMM41 | −0.595 | 4.598 | −3.154 | 0.003 | 0.035 | −1.789 |
| LNM Group 3 vs. Group 1 | ENSG00000102699 | PARP4 | −0.596 | 6.816 | −2.998 | 0.005 | 0.045 | −2.274 |
| LNM Group 3 vs. Group 1 | ENSG00000109771 | LRP2BP | −0.596 | 4.248 | −3.091 | 0.004 | 0.039 | −1.937 |
| LNM Group 3 vs. Group 1 | ENSG00000215883 | CYB5RL | −0.597 | 5.013 | −2.983 | 0.005 | 0.046 | −2.273 |
| LNM Group 3 vs. Group 1 | ENSG00000101473 | ACOT8 | −0.598 | 4.405 | −3.112 | 0.003 | 0.038 | −1.888 |
| LNM Group 3 vs. Group 1 | ENSG00000159720 | ATP6VOD1 | −0.598 | 6.186 | −3.481 | 0.001 | 0.020 | −1.016 |
| LNM Group 3 vs. Group 1 | ENSG00000132600 | PRMT7 | −0.599 | 5.270 | −3.068 | 0.004 | 0.041 | −2.041 |
| LNM Group 3 vs. Group 1 | ENSG00000127311 | HELB | −0.599 | 6.692 | −3.179 | 0.003 | 0.033 | −1.845 |
| LNM Group 3 vs. Group 1 | ENSG00000103043 | VAC14 | −0.602 | 6.196 | −3.979 | 0.000 | 0.008 | 0.329 |
| LNM Group 3 vs. Group 1 | ENSG00000004534 | RBM6 | −0.605 | 7.094 | −3.988 | 0.000 | 0.008 | 0.316 |
| LNM Group 3 vs. Group 1 | ENSG00000110344 | UBE4A | −0.606 | 6.667 | −4.211 | 0.000 | 0.006 | 0.977 |
| LNM Group 3 vs. Group 1 | ENSG00000118058 | KMT2A | −0.607 | 8.255 | −4.667 | 0.000 | 0.003 | 2.296 |
| LNM Group 3 vs. Group 1 | ENSG00000198252 | STYX | −0.608 | 6.039 | −3.181 | 0.003 | 0.033 | −1.799 |
| LNM Group 3 vs. Group 1 | ENSG00000160799 | CCDC12 | −0.610 | 4.311 | −3.619 | 0.001 | 0.015 | −0.600 |
| LNM Group 3 vs. Group 1 | ENSG00000184470 | TXNRD2 | −0.612 | 4.944 | −3.218 | 0.002 | 0.031 | −1.664 |
| LNM Group 3 vs. Group 1 | ENSG00000099949 | LZTR1 | −0.614 | 6.255 | −3.744 | 0.001 | 0.012 | −0.326 |
| LNM Group 3 vs. Group 1 | ENSG00000204923 | FBXO48 | −0.615 | 3.727 | −3.812 | 0.000 | 0.011 | −0.075 |
| LNM Group 3 vs. Group 1 | ENSG00000156990 | RPUSD3 | −0.615 | 4.639 | −3.700 | 0.001 | 0.013 | −0.398 |
| LNM Group 3 vs. Group 1 | ENSG00000078814 | MYH7B | −0.615 | 4.119 | −2.951 | 0.005 | 0.048 | −2.305 |
| LNM Group 3 vs. Group 1 | ENSG00000165526 | RPUSD4 | −0.616 | 4.739 | −3.153 | 0.003 | 0.035 | −1.817 |
| LNM Group 3 vs. Group 1 | ENSG00000072135 | PTPN18 | −0.617 | 6.126 | −3.455 | 0.001 | 0.021 | −1.107 |
| LNM Group 3 vs. Group 1 | ENSG00000100150 | DEPDC5 | −0.619 | 5.375 | −3.145 | 0.003 | 0.035 | −1.865 |
| LNM Group 3 vs. Group 1 | ENSG00000142188 | TMEM50B | −0.620 | 5.099 | −3.422 | 0.001 | 0.022 | −1.137 |
| LNM Group 3 vs. Group 1 | ENSG00000144791 | LIMD1 | −0.622 | 6.887 | −3.009 | 0.004 | 0.044 | −2.271 |
| LNM Group 3 vs. Group 1 | ENSG00000140829 | DHX38 | −0.625 | 6.096 | −4.218 | 0.000 | 0.006 | 1.004 |
| LNM Group 3 vs. Group 1 | ENSG00000163867 | ZMYM6 | −0.626 | 5.872 | −3.910 | 0.000 | 0.009 | 0.133 |
| LNM Group 3 vs. Group 1 | ENSG00000141337 | ARSG | −0.626 | 4.784 | −3.314 | 0.002 | 0.026 | −1.426 |
| LNM Group 3 vs. Group 1 | ENSG00000104133 | SPG11 | −0.627 | 7.432 | −4.181 | 0.000 | 0.006 | 0.869 |
| LNM Group 3 vs. Group 1 | ENSG00000004399 | PLXND1 | −0.632 | 7.289 | −2.974 | 0.005 | 0.047 | −2.360 |
| LNM Group 3 vs. Group 1 | ENSG00000114353 | GNAI2 | −0.633 | 7.463 | −4.184 | 0.000 | 0.006 | 0.878 |
| LNM Group 3 vs. Group 1 | ENSG00000170776 | AKAP13 | −0.633 | 8.969 | −3.688 | 0.001 | 0.013 | −0.516 |
| LNM Group 3 vs. Group 1 | ENSG00000159256 | MORC3 | −0.639 | 6.338 | −3.719 | 0.001 | 0.013 | −0.413 |
| LNM Group 3 vs. Group 1 | ENSG00000081014 | AP4E1 | −0.641 | 5.496 | −3.190 | 0.003 | 0.033 | −1.748 |
| LNM Group 3 vs. Group 1 | ENSG00000114857 | NKTR | −0.642 | 8.398 | −3.627 | 0.001 | 0.015 | −0.688 |
| LNM Group 3 vs. Group 1 | ENSG00000013523 | ANGEL1 | −0.643 | 5.372 | −2.940 | 0.005 | 0.049 | −2.335 |
| LNM Group 3 vs. Group 1 | ENSG00000238083 | LRRC37A2 | −0.648 | 5.684 | −3.199 | 0.003 | 0.032 | −1.758 |
| LNM Group 3 vs. Group 1 | ENSG00000177873 | ZNF619 | −0.648 | 4.047 | −2.969 | 0.005 | 0.047 | −2.213 |
| LNM Group 3 vs. Group 1 | ENSG00000088298 | EDEM2 | −0.649 | 4.295 | −3.622 | 0.001 | 0.015 | −0.573 |
| LNM Group 3 vs. Group 1 | ENSG00000184857 | TMEM186 | −0.649 | 2.934 | −3.122 | 0.003 | 0.037 | −1.823 |
| LNM Group 3 vs. Group 1 | ENSG00000186812 | ZNF397 | −0.652 | 6.069 | −3.224 | 0.002 | 0.031 | −1.733 |
| LNM Group 3 vs. Group 1 | ENSG00000169372 | CRADD | −0.654 | 2.880 | −3.044 | 0.004 | 0.042 | −2.015 |
| LNM Group 3 vs. Group 1 | ENSG00000173442 | EHBP1L1 | −0.655 | 7.917 | −3.124 | 0.003 | 0.037 | −1.995 |
| LNM Group 3 vs. Group 1 | ENSG00000124570 | SERPINB6 | −0.656 | 5.285 | −3.051 | 0.004 | 0.042 | −2.056 |
| LNM Group 3 vs. Group 1 | ENSG00000134899 | ERCC5 | −0.656 | 5.387 | −3.657 | 0.001 | 0.014 | −0.515 |
| LNM Group 3 vs. Group 1 | ENSG00000149196 | HIKESHI | −0.657 | 4.329 | −3.073 | 0.004 | 0.040 | −1.954 |
| LNM Group 3 vs. Group 1 | ENSG00000103064 | SLC7A6 | −0.658 | 6.999 | −3.263 | 0.002 | 0.029 | −1.611 |
| LNM Group 3 vs. Group 1 | ENSG00000156171 | DRAM2 | −0.658 | 4.993 | −3.063 | 0.004 | 0.041 | −2.007 |
| LNM Group 3 vs. Group 1 | ENSG00000197976 | AKAP17A | −0.662 | 4.832 | −3.764 | 0.001 | 0.012 | −0.241 |
| LNM Group 3 vs. Group 1 | ENSG00000131381 | RBSN | −0.665 | 5.775 | −3.970 | 0.000 | 0.009 | 0.308 |
| LNM Group 3 vs. Group 1 | ENSG00000154240 | CEP112 | −0.667 | 4.096 | −3.152 | 0.003 | 0.035 | −1.797 |
| LNM Group 3 vs. Group 1 | ENSG00000066855 | MTFR1 | −0.667 | 5.546 | −3.716 | 0.001 | 0.013 | −0.360 |
| LNM Group 3 vs. Group 1 | ENSG00000133943 | C14orf159 | −0.668 | 5.982 | −3.370 | 0.002 | 0.024 | −1.316 |
| LNM Group 3 vs. Group 1 | ENSG00000198382 | UVRAG | −0.668 | 5.794 | −3.486 | 0.001 | 0.020 | −1.005 |
| LNM Group 3 vs. Group 1 | ENSG00000177042 | TMEM80 | −0.668 | 4.810 | −3.332 | 0.002 | 0.026 | −1.367 |
| LNM Group 3 vs. Group 1 | ENSG00000138756 | BMP2K | −0.670 | 7.175 | −3.003 | 0.005 | 0.045 | −2.277 |
| LNM Group 3 vs. Group 1 | ENSG00000122435 | TRMT13 | −0.671 | 4.624 | −3.041 | 0.004 | 0.042 | −2.077 |
| LNM Group 3 vs. Group 1 | ENSG00000100280 | AP1B1 | −0.674 | 7.158 | −4.203 | 0.000 | 0.006 | 0.934 |
| LNM Group 3 vs. Group 1 | ENSG00000221883 | ARIH2OS | −0.675 | 1.845 | −3.269 | 0.002 | 0.029 | −1.450 |
| LNM Group 3 vs. Group 1 | ENSG00000170802 | FOXN2 | −0.676 | 6.422 | −2.973 | 0.005 | 0.047 | −2.310 |
| LNM Group 3 vs. Group 1 | ENSG00000003756 | RBM5 | −0.679 | 7.663 | −4.710 | 0.000 | 0.003 | 2.426 |
| LNM Group 3 vs. Group 1 | ENSG00000133872 | SARAF | −0.680 | 7.323 | −3.436 | 0.001 | 0.021 | −1.193 |
| LNM Group 3 vs. Group 1 | ENSG00000104205 | SGK3 | −0.681 | 5.347 | −3.583 | 0.001 | 0.016 | −0.720 |
| LNM Group 3 vs. Group 1 | ENSG00000162512 | SDC3 | −0.681 | 6.528 | −3.044 | 0.004 | 0.042 | −2.185 |
| LNM Group 3 vs. Group 1 | ENSG00000236383 | LINC00854 | −0.684 | 4.371 | −3.533 | 0.001 | 0.018 | −0.845 |
| LNM Group 3 vs. Group 1 | ENSG00000198373 | WWP2 | −0.684 | 6.105 | −4.593 | 0.000 | 0.003 | 2.106 |
| LNM Group 3 vs. Group 1 | ENSG00000204611 | ZNF616 | −0.686 | 4.148 | −3.445 | 0.001 | 0.021 | −1.059 |
| LNM Group 3 vs. Group 1 | ENSG00000159314 | ARHGAP27 | −0.689 | 6.435 | −3.004 | 0.004 | 0.045 | −2.277 |
| LNM Group 3 vs. Group 1 | ENSG00000172725 | CORO1B | −0.690 | 6.324 | −3.426 | 0.001 | 0.022 | −1.190 |
| LNM Group 3 vs. Group 1 | ENSG00000100109 | TFIP11 | −0.690 | 5.200 | −4.060 | 0.000 | 0.007 | 0.578 |
| LNM Group 3 vs. Group 1 | ENSG00000153179 | RASSF3 | −0.690 | 5.707 | −3.669 | 0.001 | 0.014 | −0.527 |
| LNM Group 3 vs. Group 1 | ENSG00000173540 | GMPPB | −0.691 | 5.258 | −2.959 | 0.005 | 0.047 | −2.297 |
| LNM Group 3 vs. Group 1 | ENSG00000168228 | ZCCHC4 | −0.691 | 4.081 | −3.114 | 0.003 | 0.038 | −1.869 |
| LNM Group 3 vs. Group 1 | ENSG00000104731 | KLHDC4 | −0.693 | 6.011 | −3.337 | 0.002 | 0.026 | −1.393 |
| LNM Group 3 vs. Group 1 | ENSG00000128891 | C15orf57 | −0.694 | 5.503 | −3.527 | 0.001 | 0.018 | −0.879 |
| LNM Group 3 vs. Group 1 | ENSG00000188167 | TMPPE | −0.694 | 4.178 | −3.532 | 0.001 | 0.018 | −0.811 |
| LNM Group 3 vs. Group 1 | ENSG00000205423 | CNEP1R1 | −0.694 | 4.539 | −3.925 | 0.000 | 0.009 | 0.229 |
| LNM Group 3 vs. Group 1 | ENSG00000163412 | EIF4E3 | −0.695 | 5.497 | −3.511 | 0.001 | 0.019 | −0.930 |
| LNM Group 3 vs. Group 1 | ENSG00000197928 | ZNF677 | −0.698 | 4.092 | −3.029 | 0.004 | 0.043 | −2.074 |
| LNM Group 3 vs. Group 1 | ENSG00000128699 | ORMDL1 | −0.699 | 5.708 | −4.181 | 0.000 | 0.006 | 0.910 |
| LNM Group 3 vs. Group 1 | ENSG00000181523 | SGSH | −0.700 | 5.696 | −3.494 | 0.001 | 0.019 | −0.953 |
| LNM Group 3 vs. Group 1 | ENSG00000172375 | C2CD2L | −0.703 | 4.651 | −3.293 | 0.002 | 0.027 | −1.434 |
| LNM Group 3 vs. Group 1 | ENSG00000137496 | ID8BP | −0.704 | 4.841 | −3.163 | 0.003 | 0.034 | −1.778 |
| LNM Group 3 vs. Group 1 | ENSG00000073969 | NSF | −0.704 | 5.682 | −3.013 | 0.004 | 0.044 | −2.183 |
| LNM Group 3 vs. Group 1 | ENSG00000185088 | RPS27L | −0.705 | 5.689 | −3.188 | 0.003 | 0.033 | −1.732 |
| LNM Group 3 vs. Group 1 | ENSG00000064601 | CTSA | −0.707 | 6.657 | −3.547 | 0.001 | 0.017 | −0.874 |
| LNM Group 3 vs. Group 1 | ENSG00000079950 | STX7 | −0.712 | 7.347 | −2.960 | 0.005 | 0.047 | −2.387 |
| LNM Group 3 vs. Group 1 | ENSG00000204084 | INPP5B | −0.713 | 5.880 | −4.273 | 0.000 | 0.006 | 1.163 |
| LNM Group 3 vs. Group 1 | ENSG00000180089 | TMEM86B | −0.714 | 2.819 | −3.058 | 0.004 | 0.041 | −1.989 |
| LNM Group 3 vs. Group 1 | ENSG00000180376 | CCDC66 | −0.715 | 5.256 | −3.965 | 0.000 | 0.009 | 0.307 |
| LNM Group 3 vs. Group 1 | ENSG00000279244 | AL645728.1 | −0.716 | 2.017 | −2.961 | 0.005 | 0.047 | −2.188 |
| LNM Group 3 vs. Group 1 | ENSG00000170248 | PDCD6IP | −0.716 | 7.192 | −4.298 | 0.000 | 0.005 | 1.220 |
| LNM Group 3 vs. Group 1 | ENSG00000147526 | TACC1 | −0.717 | 7.942 | −3.374 | 0.002 | 0.024 | −1.361 |
| LNM Group 3 vs. Group 1 | ENSG00000166848 | TERF2IP | −0.718 | 5.440 | −3.654 | 0.001 | 0.014 | −0.539 |
| LNM Group 3 vs. Group 1 | ENSG00000135655 | USP15 | −0.719 | 7.237 | −4.217 | 0.000 | 0.006 | 0.978 |
| LNM Group 3 vs. Group 1 | ENSG00000178971 | CTC1 | −0.719 | 5.953 | −3.032 | 0.004 | 0.043 | −2.171 |
| LNM Group 3 vs. Group 1 | ENSG00000167202 | TBC1D2B | −0.719 | 6.979 | −3.961 | 0.000 | 0.009 | 0.261 |
| LNM Group 3 vs. Group 1 | ENSG00000087266 | SH3BP2 | −0.719 | 7.638 | −3.313 | 0.002 | 0.026 | −1.517 |
| LNM Group 3 vs. Group 1 | ENSG00000114026 | OGG1 | −0.719 | 5.147 | −3.520 | 0.001 | 0.018 | −0.892 |
| LNM Group 3 vs. Group 1 | ENSG00000205707 | LYRM5 | −0.721 | 3.253 | −3.541 | 0.001 | 0.018 | −0.782 |
| LNM Group 3 vs. Group 1 | ENSG00000159082 | SYNJ1 | −0.722 | 4.955 | −2.994 | 0.005 | 0.045 | −2.183 |
| LNM Group 3 vs. Group 1 | ENSG00000163320 | CGGBP1 | −0.722 | 6.698 | −3.752 | 0.001 | 0.012 | −0.315 |
| LNM Group 3 vs. Group 1 | ENSG00000213533 | TMEM110 | −0.723 | 5.112 | −3.784 | 0.000 | 0.011 | −0.168 |
| LNM Group 3 vs. Group 1 | ENSG00000137509 | PRCP | −0.723 | 5.835 | −3.485 | 0.001 | 0.020 | −0.998 |
| LNM Group 3 vs. Group 1 | ENSG00000172530 | BANP | −0.724 | 5.002 | −4.058 | 0.000 | 0.007 | 0.570 |
| LNM Group 3 vs. Group 1 | ENSG00000197860 | SGTB | −0.725 | 5.122 | −3.444 | 0.001 | 0.021 | −1.090 |
| LNM Group 3 vs. Group 1 | ENSG00000040933 | INPP4A | −0.725 | 6.871 | −3.282 | 0.002 | 0.028 | −1.583 |
| LNM Group 3 vs. Group 1 | ENSG00000140577 | CRTC3 | −0.725 | 6.519 | −3.202 | 0.003 | 0.032 | −1.783 |
| LNM Group 3 vs. Group 1 | ENSG00000168876 | ANKRD49 | −0.727 | 4.186 | −3.358 | 0.002 | 0.025 | −1.265 |
| LNM Group 3 vs. Group 1 | ENSG00000055147 | FAM114A2 | −0.730 | 4.511 | −3.368 | 0.002 | 0.024 | −1.236 |
| LNM Group 3 vs. Group 1 | ENSG00000112419 | PHACTR2 | −0.731 | 6.741 | −3.694 | 0.001 | 0.013 | −0.497 |
| LNM Group 3 vs. Group 1 | ENSG00000114395 | CYB561D2 | −0.732 | 2.971 | −2.945 | 0.005 | 0.048 | −2.230 |
| LNM Group 3 vs. Group 1 | ENSG00000135686 | KLHL36 | −0.733 | 6.418 | −3.495 | 0.001 | 0.019 | −0.991 |
| LNM Group 3 vs. Group 1 | ENSG00000103248 | MTHFSD | −0.734 | 4.540 | −3.934 | 0.000 | 0.009 | 0.249 |
| LNM Group 3 vs. Group 1 | ENSG00000175309 | PHYKPL | −0.735 | 6.768 | −4.150 | 0.000 | 0.006 | 0.793 |
| LNM Group 3 vs. Group 1 | ENSG00000184178 | SCFD2 | −0.736 | 4.365 | −3.334 | 0.002 | 0.026 | −1.350 |
| LNM Group 3 vs. Group 1 | ENSG00000102871 | TRADD | −0.736 | 4.713 | −3.751 | 0.001 | 0.012 | −0.243 |
| LNM Group 3 vs. Group 1 | ENSG00000114316 | USP4 | −0.736 | 6.195 | −4.435 | 0.000 | 0.004 | 1.637 |
| LNM Group 3 vs. Group 1 | ENSG00000155393 | HEATR3 | −0.740 | 5.028 | −3.534 | 0.001 | 0.018 | −0.838 |
| LNM Group 3 vs. Group 1 | ENSG00000173757 | STAT5B | −0.741 | 6.586 | −3.412 | 0.001 | 0.022 | −1.243 |
| LNM Group 3 vs. Group 1 | ENSG00000090857 | PDPR | −0.745 | 6.858 | −3.572 | 0.001 | 0.016 | −0.814 |
| LNM Group 3 vs. Group 1 | ENSG00000104660 | LEPROTL1 | −0.745 | 5.703 | −3.177 | 0.003 | 0.033 | −1.790 |
| LNM Group 3 vs. Group 1 | ENSG00000147010 | SH3KBP1 | −0.747 | 5.990 | −3.037 | 0.004 | 0.043 | −2.154 |
| LNM Group 3 vs. Group 1 | ENSG00000185404 | SP140L | −0.747 | 5.838 | −3.231 | 0.002 | 0.031 | −1.681 |
| LNM Group 3 vs. Group 1 | ENSG00000163932 | PRKCD | −0.750 | 5.025 | −3.028 | 0.004 | 0.043 | −2.114 |
| LNM Group 3 vs. Group 1 | ENSG00000078304 | PPP2R5C | −0.750 | 7.554 | −3.731 | 0.001 | 0.013 | −0.397 |
| LNM Group 3 vs. Group 1 | ENSG00000110719 | TCIRG1 | −0.753 | 6.715 | −3.062 | 0.004 | 0.041 | −2.129 |
| LNM Group 3 vs. Group 1 | ENSG00000184939 | ZFP90 | −0.754 | 5.500 | −3.473 | 0.001 | 0.020 | −1.029 |
| LNM Group 3 vs. Group 1 | ENSG00000165494 | PCF11 | −0.754 | 7.134 | −3.747 | 0.001 | 0.012 | −0.335 |
| LNM Group 3 vs. Group 1 | ENSG00000217555 | CKLF | −0.756 | 3.246 | −3.152 | 0.003 | 0.035 | −1.740 |
| LNM Group 3 vs. Group 1 | ENSG00000198001 | IRAK4 | −0.756 | 5.564 | −3.170 | 0.003 | 0.034 | −1.807 |
| LNM Group 3 vs. Group 1 | ENSG00000164081 | TEX264 | −0.758 | 4.464 | −3.092 | 0.004 | 0.039 | −1.920 |
| LNM Group 3 vs. Group 1 | ENSG00000204070 | SYS1 | −0.758 | 5.270 | −4.531 | 0.000 | 0.004 | 1.921 |
| LNM Group 3 vs. Group 1 | ENSG00000140280 | LYSMD2 | −0.758 | 2.595 | −2.955 | 0.005 | 0.048 | −2.212 |
| LNM Group 3 vs. Group 1 | ENSG00000127419 | TMEM175 | −0.759 | 4.538 | −2.959 | 0.005 | 0.047 | −2.231 |
| LNM Group 3 vs. Group 1 | ENSG00000204592 | HLA-E | −0.759 | 9.225 | −3.591 | 0.001 | 0.016 | −0.783 |
| LNM Group 3 vs. Group 1 | ENSG00000130429 | ARPC1B | −0.763 | 7.152 | −4.335 | 0.000 | 0.005 | 1.338 |
| LNM Group 3 vs. Group 1 | ENSG00000135272 | MDFIC | −0.766 | 6.273 | −3.000 | 0.005 | 0.045 | −2.261 |
| LNM Group 3 vs. Group 1 | ENSG00000054611 | TBC1D22A | −0.768 | 6.114 | −3.499 | 0.001 | 0.019 | −1.000 |
| LNM Group 3 vs. Group 1 | ENSG00000076513 | ANKRD13A | −0.770 | 6.726 | −3.782 | 0.000 | 0.012 | −0.227 |
| LNM Group 3 vs. Group 1 | ENSG00000163635 | ATXN7 | −0.771 | 6.929 | −3.959 | 0.000 | 0.009 | 0.253 |
| LNM Group 3 vs. Group 1 | ENSG00000139597 | N4BP2L1 | −0.775 | 4.663 | −3.201 | 0.003 | 0.032 | −1.681 |
| LNM Group 3 vs. Group 1 | ENSG00000110047 | EHD1 | −0.777 | 6.373 | −3.533 | 0.001 | 0.018 | −0.906 |
| LNM Group 3 vs. Group 1 | ENSG00000177311 | ZBTB38 | −0.777 | 7.309 | −3.978 | 0.000 | 0.008 | 0.295 |
| LNM Group 3 vs. Group 1 | ENSG00000138744 | NAAA | −0.777 | 4.258 | −3.386 | 0.002 | 0.023 | −1.217 |
| LNM Group 3 vs. Group 1 | ENSG00000136770 | DNAJC1 | −0.778 | 4.180 | −3.257 | 0.002 | 0.029 | −1.510 |
| LNM Group 3 vs. Group 1 | ENSG00000187239 | FNBP1 | −0.780 | 6.844 | −3.131 | 0.003 | 0.037 | −1.962 |
| LNM Group 3 vs. Group 1 | ENSG00000149177 | PTPRJ | −0.780 | 6.279 | −3.466 | 0.001 | 0.020 | −1.086 |
| LNM Group 3 vs. Group 1 | ENSG00000150433 | TMEM218 | −0.780 | 4.085 | −3.224 | 0.002 | 0.031 | −1.589 |
| LNM Group 3 vs. Group 1 | ENSG00000140853 | NLRC5 | −0.781 | 7.910 | −3.033 | 0.004 | 0.043 | −2.211 |
| LNM Group 3 vs. Group 1 | ENSG00000188033 | ZNF490 | −0.784 | 4.213 | −3.999 | 0.000 | 0.008 | 0.426 |
| LNM Group 3 vs. Group 1 | ENSG00000205045 | SLFN12L | −0.785 | 5.245 | −3.351 | 0.002 | 0.025 | −1.348 |
| LNM Group 3 vs. Group 1 | ENSG00000102893 | PHKB | −0.785 | 6.733 | −4.752 | 0.000 | 0.002 | 2.567 |
| LNM Group 3 vs. Group 1 | ENSG00000144843 | ADPRH | −0.785 | 3.779 | −3.171 | 0.003 | 0.034 | −1.714 |
| LNM Group 3 vs. Group 1 | ENSG00000188997 | KCTD21 | −0.786 | 4.129 | −4.249 | 0.000 | 0.006 | 1.131 |
| LNM Group 3 vs. Group 1 | ENSG00000123104 | ITPR2 | −0.792 | 7.340 | −3.848 | 0.000 | 0.010 | −0.074 |
| LNM Group 3 vs. Group 1 | ENSG00000006638 | TBXA2R | −0.792 | 2.448 | −3.131 | 0.003 | 0.037 | −1.789 |
| LNM Group 3 vs. Group 1 | ENSG00000069943 | PIGB | −0.793 | 4.501 | −4.623 | 0.000 | 0.003 | 2.201 |
| LNM Group 3 vs. Group 1 | ENSG00000145022 | TCTA | −0.796 | 4.121 | −3.626 | 0.001 | 0.015 | −0.569 |
| LNM Group 3 vs. Group 1 | ENSG00000135048 | TMEM2 | −0.800 | 7.455 | −3.469 | 0.001 | 0.020 | −1.110 |
| LNM Group 3 vs. Group 1 | ENSG00000167377 | ZNF23 | −0.801 | 4.091 | −3.001 | 0.005 | 0.045 | −2.134 |
| LNM Group 3 vs. Group 1 | ENSG00000054967 | RELT | −0.802 | 5.891 | −2.957 | 0.005 | 0.048 | −2.361 |
| LNM Group 3 vs. Group 1 | ENSG00000196199 | MPHOSPH8 | −0.807 | 5.963 | −4.237 | 0.000 | 0.006 | 1.064 |
| LNM Group 3 vs. Group 1 | ENSG00000130307 | USHBP1 | −0.807 | 3.379 | −3.187 | 0.003 | 0.033 | −1.679 |
| LNM Group 3 vs. Group 1 | ENSG00000198223 | CSF2RA | −0.809 | 3.989 | −3.119 | 0.003 | 0.037 | −1.857 |
| LNM Group 3 vs. Group 1 | ENSG00000141298 | SSH2 | −0.809 | 7.268 | −3.798 | 0.000 | 0.011 | −0.216 |
| LNM Group 3 vs. Group 1 | ENSG00000178917 | ZNF852 | −0.810 | 3.369 | −3.333 | 0.002 | 0.026 | −1.313 |
| LNM Group 3 vs. Group 1 | ENSG00000160439 | RDH13 | −0.810 | 5.578 | −3.204 | 0.003 | 0.032 | −1.732 |
| LNM Group 3 vs. Group 1 | ENSG00000164414 | SLC35A1 | −0.813 | 3.860 | −3.724 | 0.001 | 0.013 | −0.306 |
| LNM Group 3 vs. Group 1 | ENSG00000181513 | ACBD4 | −0.815 | 3.403 | −3.105 | 0.003 | 0.038 | −1.871 |
| LNM Group 3 vs. Group 1 | ENSG00000262246 | CORO7 | −0.816 | 5.814 | −3.243 | 0.002 | 0.030 | −1.619 |
| LNM Group 3 vs. Group 1 | ENSG00000103061 | SLC7A6OS | −0.825 | 4.833 | −4.319 | 0.000 | 0.005 | 1.325 |
| LNM Group 3 vs. Group 1 | ENSG00000166974 | MAPRE2 | −0.825 | 5.277 | −3.066 | 0.004 | 0.041 | −2.035 |
| LNM Group 3 vs. Group 1 | ENSG00000155085 | AK9 | −0.825 | 3.484 | −3.438 | 0.001 | 0.021 | −1.055 |
| LNM Group 3 vs. Group 1 | ENSG00000198833 | UBE2J1 | −0.826 | 6.412 | −3.373 | 0.002 | 0.024 | −1.328 |
| LNM Group 3 vs. Group 1 | ENSG00000159110 | IFNAR2 | −0.826 | 5.668 | −4.147 | 0.000 | 0.006 | 0.814 |
| LNM Group 3 vs. Group 1 | ENSG00000177663 | ID7RA | −0.827 | 6.232 | −3.718 | 0.001 | 0.013 | −0.400 |
| LNM Group 3 vs. Group 1 | ENSG00000134109 | EDEM1 | −0.827 | 7.327 | −4.000 | 0.000 | 0.008 | 0.356 |
| LNM Group 3 vs. Group 1 | ENSG00000103150 | MLYCD | −0.828 | 5.555 | −3.494 | 0.001 | 0.019 | −0.972 |
| LNM Group 3 vs. Group 1 | ENSG00000168792 | ABHD15 | −0.828 | 4.603 | −3.723 | 0.001 | 0.013 | −0.322 |
| LNM Group 3 vs. Group 1 | ENSG00000260916 | CCPG1 | −0.829 | 5.924 | −2.928 | 0.006 | 0.050 | −2.419 |
| LNM Group 3 vs. Group 1 | ENSG00000120910 | PPP3CC | −0.838 | 4.548 | −3.377 | 0.002 | 0.024 | −1.227 |
| LNM Group 3 vs. Group 1 | ENSG00000247596 | TWF2 | −0.840 | 4.632 | −3.901 | 0.000 | 0.009 | 0.161 |
| LNM Group 3 vs. Group 1 | ENSG00000213809 | KLRK1 | −0.841 | 4.097 | −3.184 | 0.003 | 0.033 | −1.695 |
| LNM Group 3 vs. Group 1 | ENSG00000196544 | BORCS6 | −0.842 | 2.801 | −3.450 | 0.001 | 0.021 | −0.999 |
| LNM Group 3 vs. Group 1 | ENSG00000134545 | KLRC1 | −0.842 | 2.452 | −2.982 | 0.005 | 0.046 | −2.145 |
| LNM Group 3 vs. Group 1 | ENSG00000103479 | RBL2 | −0.845 | 7.342 | −5.089 | 0.000 | 0.001 | 3.576 |
| LNM Group 3 vs. Group 1 | ENSG00000228570 | NUTM2E | −0.846 | 2.282 | −3.245 | 0.002 | 0.030 | −1.509 |
| LNM Group 3 vs. Group 1 | ENSG00000137486 | ARRB1 | −0.847 | 4.630 | −3.344 | 0.002 | 0.025 | −1.294 |
| LNM Group 3 vs. Group 1 | ENSG00000166971 | AKTIP | −0.848 | 4.686 | −3.324 | 0.002 | 0.026 | −1.348 |
| LNM Group 3 vs. Group 1 | ENSG00000205542 | TMSB4X | −0.849 | 9.240 | −3.655 | 0.001 | 0.014 | −0.598 |
| LNM Group 3 vs. Group 1 | ENSG00000205268 | PDE7A | −0.849 | 7.026 | −3.981 | 0.000 | 0.008 | 0.312 |
| LNM Group 3 vs. Group 1 | ENSG00000138185 | ENTPD1 | −0.849 | 6.268 | −3.252 | 0.002 | 0.029 | −1.612 |
| LNM Group 3 vs. Group 1 | ENSG00000068724 | TTC7A | −0.852 | 6.470 | −3.585 | 0.001 | 0.016 | −0.755 |
| LNM Group 3 vs. Group 1 | ENSG00000152689 | RASGRP3 | −0.854 | 5.719 | −3.899 | 0.000 | 0.009 | 0.113 |
| LNM Group 3 vs. Group 1 | ENSG00000032219 | ARID4A | −0.854 | 5.738 | −4.259 | 0.000 | 0.006 | 1.150 |
| LNM Group 3 vs. Group 1 | ENSG00000162241 | SLC25A45 | −0.856 | 5.265 | −3.420 | 0.001 | 0.022 | −1.168 |
| LNM Group 3 vs. Group 1 | ENSG00000173559 | NABP1 | −0.856 | 8.027 | −3.703 | 0.001 | 0.013 | −0.481 |
| LNM Group 3 vs. Group 1 | ENSG00000173698 | ADGRG2 | −0.859 | 3.974 | −3.444 | 0.001 | 0.021 | −1.058 |
| LNM Group 3 vs. Group 1 | ENSG00000221995 | TIAF1 | −0.859 | 4.552 | −4.345 | 0.000 | 0.005 | 1.401 |
| LNM Group 3 vs. Group 1 | ENSG00000258555 | SPECC1L- | −0.860 | 2.914 | −2.974 | 0.005 | 0.047 | −2.172 |
| | | ADORA2A | | | | | | |
| LNM Group 3 vs. Group 1 | ENSG00000069966 | GNB5 | −0.862 | 5.827 | −4.168 | 0.000 | 0.006 | 0.868 |
| LNM Group 3 vs. Group 1 | ENSG00000047346 | FAM214A | −0.862 | 6.618 | −3.039 | 0.004 | 0.043 | −2.179 |
| LNM Group 3 vs. Group 1 | ENSG00000013364 | MVP | −0.863 | 7.190 | −3.959 | 0.000 | 0.009 | 0.258 |
| LNM Group 3 vs. Group 1 | ENSG00000027075 | PRKCH | −0.864 | 6.399 | −3.440 | 0.001 | 0.021 | −1.142 |
| LNM Group 3 vs. Group 1 | ENSG00000028116 | VRK2 | −0.864 | 5.123 | −3.404 | 0.001 | 0.023 | −1.208 |
| LNM Group 3 vs. Group 1 | ENSG00000197619 | ZNF615 | −0.867 | 3.811 | −3.784 | 0.000 | 0.011 | −0.152 |
| LNM Group 3 vs. Group 1 | ENSG00000165972 | CCDC38 | −0.869 | 2.296 | −3.072 | 0.004 | 0.040 | −1.933 |
| LNM Group 3 vs. Group 1 | ENSG00000101347 | SAMHD1 | −0.870 | 7.445 | −3.422 | 0.001 | 0.022 | −1.206 |
| LNM Group 3 vs. Group 1 | ENSG00000271605 | MILR1 | −0.873 | 3.197 | −3.740 | 0.001 | 0.012 | −0.257 |
| LNM Group 3 vs. Group 1 | ENSG00000101160 | CTSZ | −0.873 | 6.724 | −4.612 | 0.000 | 0.003 | 2.145 |
| LNM Group 3 vs. Group 1 | ENSG00000166436 | TRIM66 | −0.874 | 5.651 | −3.006 | 0.004 | 0.045 | −2.204 |
| LNM Group 3 vs. Group 1 | ENSG00000241399 | CD302 | −0.877 | 4.228 | −2.939 | 0.005 | 0.049 | −2.310 |
| LNM Group 3 vs. Group 1 | ENSG00000137478 | FCHSD2 | −0.879 | 6.271 | −4.119 | 0.000 | 0.007 | 0.722 |
| LNM Group 3 vs. Group 1 | ENSG00000159496 | RGL4 | −0.880 | 3.332 | −3.094 | 0.004 | 0.039 | −1.903 |
| LNM Group 3 vs. Group 1 | ENSG00000188452 | CERKL | −0.880 | 3.996 | −3.555 | 0.001 | 0.017 | −0.763 |
| LNM Group 3 vs. Group 1 | ENSG00000135090 | TAOK3 | −0.881 | 6.128 | −5.609 | 0.000 | 0.001 | 5.167 |
| LNM Group 3 vs. Group 1 | ENSG00000181045 | SLC26A11 | −0.884 | 4.331 | −3.143 | 0.003 | 0.036 | −1.795 |
| LNM Group 3 vs. Group 1 | ENSG00000178695 | KCTD12 | −0.887 | 7.260 | −3.418 | 0.001 | 0.022 | −1.222 |
| LNM Group 3 vs. Group 1 | ENSG00000109323 | MANBA | −0.889 | 5.513 | −3.819 | 0.000 | 0.011 | −0.096 |
| LNM Group 3 vs. Group 1 | ENSG00000109063 | MYH3 | −0.890 | 3.473 | −2.991 | 0.005 | 0.045 | −2.161 |
| LNM Group 3 vs. Group 1 | ENSG00000106560 | GIMAP2 | −0.891 | 4.214 | −3.879 | 0.000 | 0.010 | 0.104 |
| LNM Group 3 vs. Group 1 | ENSG00000163719 | MTMR14 | −0.891 | 5.326 | −5.298 | 0.000 | 0.001 | 4.214 |
| LNM Group 3 vs. Group 1 | ENSG00000184619 | KRBA2 | −0.892 | 2.744 | −3.768 | 0.001 | 0.012 | −0.179 |
| LNM Group 3 vs. Group 1 | ENSG00000154144 | TBRG1 | −0.896 | 5.684 | −6.402 | 0.000 | 0.000 | 7.577 |
| LNM Group 3 vs. Group 1 | ENSG00000178607 | ERN1 | −0.896 | 6.416 | −3.059 | 0.004 | 0.041 | −2.111 |
| LNM Group 3 vs. Group 1 | ENSG00000178927 | C17orf62 | −0.898 | 7.044 | −4.009 | 0.000 | 0.008 | 0.383 |
| LNM Group 3 vs. Group 1 | ENSG00000174718 | KIAA1551 | −0.900 | 7.676 | −3.107 | 0.003 | 0.038 | −2.041 |
| LNM Group 3 vs. Group 1 | ENSG00000173011 | TADA2B | −0.905 | 5.005 | −4.640 | 0.000 | 0.003 | 2.252 |
| LNM Group 3 vs. Group 1 | ENSG00000241839 | PLEKHO2 | −0.905 | 5.640 | −4.107 | 0.000 | 0.007 | 0.706 |
| LNM Group 3 vs. Group 1 | ENSG00000197043 | ANXA6 | −0.906 | 6.430 | −3.243 | 0.002 | 0.030 | −1.642 |
| LNM Group 3 vs. Group 1 | ENSG00000163686 | ABHD6 | −0.906 | 3.150 | −3.542 | 0.001 | 0.018 | −0.776 |
| LNM Group 3 vs. Group 1 | ENSG00000076928 | ARHGEF1 | −0.907 | 7.681 | −4.175 | 0.000 | 0.006 | 0.852 |
| LNM Group 3 vs. Group 1 | ENSG00000196405 | EVL | −0.908 | 7.403 | −3.039 | 0.004 | 0.043 | −2.203 |
| LNM Group 3 vs. Group 1 | ENSG00000026297 | RNASET2 | −0.908 | 6.213 | −3.911 | 0.000 | 0.009 | 0.133 |
| LNM Group 3 vs. Group 1 | ENSG00000114779 | ABHD14B | −0.908 | 4.423 | −3.581 | 0.001 | 0.016 | −0.688 |
| LNM Group 3 vs. Group 1 | ENSG00000145685 | LHFPL2 | −0.909 | 6.653 | −3.518 | 0.001 | 0.018 | −0.924 |
| LNM Group 3 vs. Group 1 | ENSG00000019144 | PHLDB1 | −0.910 | 7.590 | −3.192 | 0.003 | 0.033 | −1.821 |
| LNM Group 3 vs. Group 1 | ENSG00000121281 | ADCY7 | −0.911 | 6.971 | −3.507 | 0.001 | 0.019 | −0.975 |
| LNM Group 3 vs. Group 1 | ENSG00000277531 | LLNLF- | −0.921 | 1.751 | −3.257 | 0.002 | 0.029 | −1.477 |
| | | 187D8.1 | | | | | | |
| LNM Group 3 vs. Group 1 | ENSG00000118596 | SLC16A7 | −0.922 | 5.772 | −2.937 | 0.005 | 0.049 | −2.418 |
| LNM Group 3 vs. Group 1 | ENSG00000145029 | NICN1 | −0.922 | 4.136 | −3.353 | 0.002 | 0.025 | −1.271 |
| LNM Group 3 vs. Group 1 | ENSG00000159140 | SON | −0.923 | 9.249 | −3.684 | 0.001 | 0.013 | −0.508 |
| LNM Group 3 vs. Group 1 | ENSG00000137474 | MYO7A | −0.923 | 4.279 | −3.184 | 0.003 | 0.033 | −1.686 |
| LNM Group 3 vs. Group 1 | ENSG00000213445 | SIPA1 | −0.928 | 6.070 | −3.863 | 0.000 | 0.010 | −0.001 |
| LNM Group 3 vs. Group 1 | ENSG00000128294 | TPST2 | −0.929 | 5.303 | −4.394 | 0.000 | 0.004 | 1.529 |
| LNM Group 3 vs. Group 1 | ENSG00000003402 | CFLAR | −0.929 | 9.324 | −5.232 | 0.000 | 0.001 | 4.014 |
| LNM Group 3 vs. Group 1 | ENSG00000125457 | MIF4GD | −0.930 | 3.375 | −3.730 | 0.001 | 0.013 | −0.286 |
| LNM Group 3 vs. Group 1 | ENSG00000176715 | ACSF3 | −0.932 | 6.493 | −4.466 | 0.000 | 0.004 | 1.720 |
| LNM Group 3 vs. Group 1 | ENSG00000183722 | LHFP | −0.935 | 5.048 | −3.400 | 0.001 | 0.023 | −1.164 |
| LNM Group 3 vs. Group 1 | ENSG00000198879 | SFMBT2 | −0.936 | 5.954 | −3.209 | 0.003 | 0.032 | −1.683 |
| LNM Group 3 vs. Group 1 | ENSG00000171488 | LRRC8C | −0.938 | 6.054 | −3.643 | 0.001 | 0.015 | −0.591 |
| LNM Group 3 vs. Group 1 | ENSG00000139436 | GIT2 | −0.941 | 6.684 | −6.050 | 0.000 | 0.000 | 6.540 |
| LNM Group 3 vs. Group 1 | ENSG00000120860 | CCDC53 | −0.943 | 4.073 | −4.870 | 0.000 | 0.002 | 2.913 |
| LNM Group 3 vs. Group 1 | ENSG00000144711 | IQSEC1 | −0.944 | 6.846 | −4.304 | 0.000 | 0.005 | 1.234 |
| LNM Group 3 vs. Group 1 | ENSG00000091879 | ANGPT2 | −0.945 | 5.375 | −3.875 | 0.000 | 0.010 | 0.046 |
| LNM Group 3 vs. Group 1 | ENSG00000170542 | SERPINB9 | −0.946 | 6.327 | −3.449 | 0.001 | 0.021 | −1.148 |
| LNM Group 3 vs. Group 1 | ENSG00000181007 | ZFP82 | −0.949 | 3.630 | −4.320 | 0.000 | 0.005 | 1.331 |
| LNM Group 3 vs. Group 1 | ENSG00000082512 | TRAF5 | −0.949 | 6.182 | −3.446 | 0.001 | 0.021 | −1.124 |
| LNM Group 3 vs. Group 1 | ENSG00000196535 | MYO18A | −0.949 | 7.449 | −5.101 | 0.000 | 0.001 | 3.617 |
| LNM Group 3 vs. Group 1 | ENSG00000033867 | SLC4A7 | −0.951 | 6.919 | −4.452 | 0.000 | 0.004 | 1.670 |
| LNM Group 3 vs. Group 1 | ENSG00000149577 | SIDT2 | −0.951 | 6.053 | −5.016 | 0.000 | 0.002 | 3.366 |
| LNM Group 3 vs. Group 1 | ENSG00000196954 | CASP4 | −0.952 | 6.337 | −3.162 | 0.003 | 0.034 | −1.801 |
| LNM Group 3 vs. Group 1 | ENSG00000153551 | CMTM7 | −0.952 | 4.175 | −3.273 | 0.002 | 0.029 | −1.500 |
| LNM Group 3 vs. Group 1 | ENSG00000183023 | SLC8A1 | −0.953 | 5.831 | −3.049 | 0.004 | 0.042 | −2.080 |
| LNM Group 3 vs. Group 1 | ENSG00000174306 | ZHX3 | −0.954 | 5.962 | −3.535 | 0.001 | 0.018 | −0.879 |
| LNM Group 3 vs. Group 1 | ENSG00000198624 | CCDC69 | −0.955 | 5.502 | −3.766 | 0.001 | 0.012 | −0.263 |
| LNM Group 3 vs. Group 1 | ENSG00000126860 | EVI2A | −0.956 | 4.368 | −3.277 | 0.002 | 0.028 | −1.469 |
| LNM Group 3 vs. Group 1 | ENSG00000128604 | IRF5 | −0.957 | 4.473 | −3.799 | 0.000 | 0.011 | −0.111 |
| LNM Group 3 vs. Group 1 | ENSG00000172716 | SLFN11 | −0.958 | 5.773 | −3.127 | 0.003 | 0.037 | −1.892 |
| LNM Group 3 vs. Group 1 | ENSG00000164483 | SAMD3 | −0.960 | 2.608 | −2.927 | 0.006 | 0.050 | −2.269 |
| LNM Group 3 vs. Group 1 | ENSG00000069974 | RAB27A | −0.962 | 5.335 | −4.918 | 0.000 | 0.002 | 3.071 |
| LNM Group 3 vs. Group 1 | ENSG00000143891 | GALM | −0.964 | 4.026 | −3.514 | 0.001 | 0.019 | −0.852 |
| LNM Group 3 vs. Group 1 | ENSG00000244274 | DBNDD2 | −0.965 | 4.028 | −3.572 | 0.001 | 0.016 | −0.694 |
| LNM Group 3 vs. Group 1 | ENSG00000186469 | GNG2 | −0.967 | 5.157 | −3.055 | 0.004 | 0.041 | −2.029 |
| LNM Group 3 vs. Group 1 | ENSG00000105483 | CARD8 | −0.968 | 6.511 | −4.251 | 0.000 | 0.006 | 1.093 |
| LNM Group 3 vs. Group 1 | ENSG00000145246 | ATP10D | −0.968 | 6.334 | −3.312 | 0.002 | 0.026 | −1.435 |
| LNM Group 3 vs. Group 1 | ENSG00000111885 | MAN1A1 | −0.970 | 6.377 | −3.324 | 0.002 | 0.026 | −1.453 |
| LNM Group 3 vs. Group 1 | ENSG00000115993 | TRAK2 | −0.970 | 6.070 | −4.284 | 0.000 | 0.005 | 1.209 |
| LNM Group 3 vs. Group 1 | ENSG00000141968 | VAV1 | −0.971 | 4.844 | −3.936 | 0.000 | 0.009 | 0.233 |
| LNM Group 3 vs. Group 1 | ENSG00000087589 | CASS4 | −0.973 | 3.162 | −3.452 | 0.001 | 0.021 | −1.000 |
| LNM Group 3 vs. Group 1 | ENSG00000169499 | PLEKHA2 | −0.977 | 7.164 | −3.895 | 0.000 | 0.009 | 0.058 |
| LNM Group 3 vs. Group 1 | ENSG00000145416 | MARCH1 | −0.978 | 6.187 | −3.446 | 0.001 | 0.021 | −1.128 |
| LNM Group 3 vs. Group 1 | ENSG00000198673 | FAM19A2 | −0.979 | 2.686 | −3.372 | 0.002 | 0.024 | −1.200 |
| LNM Group 3 vs. Group 1 | ENSG00000182578 | CSF1R | −0.980 | 6.325 | −3.321 | 0.002 | 0.026 | −1.435 |
| LNM Group 3 vs. Group 1 | ENSG00000031081 | ARHGAP31 | −0.981 | 6.390 | −4.116 | 0.000 | 0.007 | 0.694 |
| LNM Group 3 vs. Group 1 | ENSG00000164308 | ERAP2 | −0.986 | 6.453 | −3.573 | 0.001 | 0.016 | −0.800 |
| LNM Group 3 vs. Group 1 | ENSG00000160613 | PCSK7 | −0.988 | 7.889 | −5.743 | 0.000 | 0.001 | 5.591 |
| LNM Group 3 vs. Group 1 | ENSG00000010810 | FYN | −0.988 | 6.392 | −3.755 | 0.001 | 0.012 | −0.327 |
| LNM Group 3 vs. Group 1 | ENSG00000198668 | CALM1 | −0.990 | 8.268 | −4.944 | 0.000 | 0.002 | 3.134 |
| LNM Group 3 vs. Group 1 | ENSG00000166946 | CCNDBP1 | −0.995 | 5.713 | −4.851 | 0.000 | 0.002 | 2.872 |
| LNM Group 3 vs. Group 1 | ENSG00000123146 | ADGRE5 | −0.995 | 6.191 | −4.135 | 0.000 | 0.007 | 0.754 |
| LNM Group 3 vs. Group 1 | ENSG00000161791 | FMNL3 | −0.997 | 7.528 | −4.876 | 0.000 | 0.002 | 2.926 |
| LNM Group 3 vs. Group 1 | ENSG00000142185 | TRPM2 | −0.997 | 4.995 | −2.966 | 0.005 | 0.047 | −2.266 |
| LNM Group 3 vs. Group 1 | ENSG00000070190 | DAPP1 | −0.997 | 5.574 | −3.020 | 0.004 | 0.044 | −2.185 |
| LNM Group 3 vs. Group 1 | ENSG00000254087 | LYN | −1.002 | 6.334 | −3.214 | 0.003 | 0.032 | −1.705 |
| LNM Group 3 vs. Group 1 | ENSG00000110514 | MADD | −1.002 | 6.497 | −4.379 | 0.000 | 0.005 | 1.460 |
| LNM Group 3 vs. Group 1 | ENSG00000172037 | LAMB2 | −1.006 | 6.653 | −3.271 | 0.002 | 0.029 | −1.578 |
| LNM Group 3 vs. Group 1 | ENSG00000020577 | SAMD4A | −1.007 | 6.187 | −3.020 | 0.004 | 0.044 | −2.218 |
| LNM Group 3 vs. Group 1 | ENSG00000129675 | ARHGEF6 | −1.008 | 5.572 | −3.032 | 0.004 | 0.043 | −2.097 |
| LNM Group 3 vs. Group 1 | ENSG00000142347 | MYO1F | −1.011 | 5.629 | −3.413 | 0.001 | 0.022 | −1.165 |
| LNM Group 3 vs. Group 1 | ENSG00000069493 | CLEC2D | −1.014 | 6.220 | −3.082 | 0.004 | 0.040 | −2.057 |
| LNM Group 3 vs. Group 1 | ENSG00000157350 | ST3GAL2 | −1.014 | 6.304 | −4.288 | 0.000 | 0.005 | 1.194 |
| LNM Group 3 vs. Group 1 | ENSG00000130038 | CRACR2A | −1.021 | 4.941 | −3.769 | 0.001 | 0.012 | −0.223 |
| LNM Group 3 vs. Group 1 | ENSG00000188677 | PARVB | −1.024 | 5.176 | −3.359 | 0.002 | 0.024 | −1.300 |
| LNM Group 3 vs. Group 1 | ENSG00000170949 | ZNF160 | −1.025 | 5.448 | −3.183 | 0.003 | 0.033 | −1.808 |
| LNM Group 3 vs. Group 1 | ENSG00000128340 | RAC2 | −1.027 | 5.883 | −3.025 | 0.004 | 0.043 | −2.180 |
| LNM Group 3 vs. Group 1 | ENSG00000087495 | PHACTR3 | −1.027 | 2.594 | −3.066 | 0.004 | 0.041 | −1.961 |
| LNM Group 3 vs. Group 1 | ENSG00000099308 | MAST3 | −1.029 | 5.307 | −3.750 | 0.001 | 0.012 | −0.280 |
| LNM Group 3 vs. Group 1 | ENSG00000162777 | DENND2D | −1.035 | 5.905 | −3.168 | 0.003 | 0.034 | −1.823 |
| LNM Group 3 vs. Group 1 | ENSG00000048471 | SNX29 | −1.036 | 6.604 | −4.789 | 0.000 | 0.002 | 2.679 |
| LNM Group 3 vs. Group 1 | ENSG00000158985 | CDC42SE2 | −1.042 | 6.699 | −3.934 | 0.000 | 0.009 | 0.189 |
| LNM Group 3 vs. Group 1 | ENSG00000101109 | STK4 | −1.043 | 7.643 | −5.129 | 0.000 | 0.001 | 3.700 |
| LNM Group 3 vs. Group 1 | ENSG00000105339 | DENND3 | −1.044 | 7.060 | −3.804 | 0.000 | 0.011 | −0.189 |
| LNM Group 3 vs. Group 1 | ENSG00000068028 | RASSF1 | −1.050 | 4.918 | −5.050 | 0.000 | 0.002 | 3.458 |
| LNM Group 3 vs. Group 1 | ENSG00000166128 | RAB8B | −1.051 | 6.318 | −3.806 | 0.000 | 0.011 | −0.148 |
| LNM Group 3 vs. Group 1 | ENSG00000125637 | PSD4 | −1.053 | 7.444 | −4.227 | 0.000 | 0.006 | 1.007 |
| LNM Group 3 vs. Group 1 | ENSG00000168404 | MLKL | −1.053 | 5.151 | −3.726 | 0.001 | 0.013 | −0.318 |
| LNM Group 3 vs. Group 1 | ENSG00000012779 | ALOX5 | −1.057 | 4.999 | −2.956 | 0.005 | 0.048 | −2.266 |
| LNM Group 3 vs. Group 1 | ENSG00000116852 | KIF21B | −1.058 | 5.481 | −3.609 | 0.001 | 0.015 | −0.683 |
| LNM Group 3 vs. Group 1 | ENSG00000138641 | HERC3 | −1.059 | 6.303 | −4.207 | 0.000 | 0.006 | 0.982 |
| LNM Group 3 vs. Group 1 | ENSG00000066056 | TIE1 | −1.062 | 4.404 | −3.395 | 0.002 | 0.023 | −1.168 |
| LNM Group 3 vs. Group 1 | ENSG00000129566 | TEP1 | −1.064 | 7.063 | −5.355 | 0.000 | 0.001 | 4.394 |
| LNM Group 3 vs. Group 1 | ENSG00000100600 | LGMN | −1.069 | 6.142 | −4.247 | 0.000 | 0.006 | 1.108 |
| LNM Group 3 vs. Group 1 | ENSG00000065809 | FAM107B | −1.070 | 6.305 | −3.018 | 0.004 | 0.044 | −2.210 |
| LNM Group 3 vs. Group 1 | ENSG00000182158 | CREB3L2 | −1.074 | 7.465 | −3.300 | 0.002 | 0.027 | −1.556 |
| LNM Group 3 vs. Group 1 | ENSG00000059377 | TBXAS1 | −1.075 | 4.305 | −4.134 | 0.000 | 0.007 | 0.812 |
| LNM Group 3 vs. Group 1 | ENSG00000163297 | ANTXR2 | −1.076 | 5.846 | −3.086 | 0.004 | 0.039 | −2.003 |
| LNM Group 3 vs. Group 1 | ENSG00000146376 | ARHGAP18 | −1.078 | 5.226 | −4.124 | 0.000 | 0.007 | 0.774 |
| LNM Group 3 vs. Group 1 | ENSG00000115935 | WIPF1 | −1.080 | 7.390 | −3.760 | 0.001 | 0.012 | −0.321 |
| LNM Group 3 vs. Group 1 | ENSG00000071073 | MGAT4A | −1.081 | 6.301 | −3.088 | 0.004 | 0.039 | −2.016 |
| LNM Group 3 vs. Group 1 | ENSG00000103047 | TANGO6 | −1.086 | 5.126 | −4.426 | 0.000 | 0.004 | 1.628 |
| LNM Group 3 vs. Group 1 | ENSG00000092929 | UNC13D | −1.086 | 5.772 | −3.623 | 0.001 | 0.015 | −0.621 |
| LNM Group 3 vs. Group 1 | ENSG00000150995 | ITPR1 | −1.086 | 6.573 | −3.033 | 0.004 | 0.043 | −2.173 |
| LNM Group 3 vs. Group 1 | ENSG00000197892 | KIF13B | −1.087 | 6.143 | −4.615 | 0.000 | 0.003 | 2.168 |
| LNM Group 3 vs. Group 1 | ENSG00000161955 | TNFSF13 | −1.093 | 4.314 | −3.977 | 0.000 | 0.008 | 0.368 |
| LNM Group 3 vs. Group 1 | ENSG00000206538 | VGLL3 | −1.094 | 6.158 | −3.350 | 0.002 | 0.025 | −1.382 |
| LNM Group 3 vs. Group 1 | ENSG00000106991 | ENG | −1.097 | 6.195 | −5.131 | 0.000 | 0.001 | 3.710 |
| LNM Group 3 vs. Group 1 | ENSG00000116191 | RALGPS2 | −1.098 | 6.321 | −3.450 | 0.001 | 0.021 | −1.107 |
| LNM Group 3 vs. Group 1 | ENSG00000168461 | RAB31 | −1.098 | 7.199 | −3.419 | 0.001 | 0.022 | −1.213 |
| LNM Group 3 vs. Group 1 | ENSG00000134242 | PTPN22 | −1.099 | 4.137 | −3.277 | 0.002 | 0.028 | −1.453 |
| LNM Group 3 vs. Group 1 | ENSG00000137502 | RAB30 | −1.103 | 5.630 | −3.186 | 0.003 | 0.033 | −1.766 |
| LNM Group 3 vs. Group 1 | ENSG00000155629 | PIK3AP1 | −1.103 | 5.975 | −3.337 | 0.002 | 0.026 | −1.428 |
| LNM Group 3 vs. Group 1 | ENSG00000093072 | CECR1 | −1.110 | 6.003 | −3.008 | 0.004 | 0.045 | −2.223 |
| LNM Group 3 vs. Group 1 | ENSG00000171310 | CHST11 | −1.112 | 6.371 | −4.828 | 0.000 | 0.002 | 2.801 |
| LNM Group 3 vs. Group 1 | ENSG00000163820 | FYCO1 | −1.113 | 6.072 | −3.865 | 0.000 | 0.010 | 0.026 |
| LNM Group 3 vs. Group 1 | ENSG00000213648 | SULT1A4 | −1.114 | 2.810 | −3.098 | 0.003 | 0.039 | −1.872 |
| LNM Group 3 vs. Group 1 | ENSG00000145020 | AMT | −1.117 | 4.533 | −3.312 | 0.002 | 0.026 | −1.396 |
| LNM Group 3 vs. Group 1 | ENSG00000096996 | IL12RB1 | −1.117 | 4.014 | −3.661 | 0.001 | 0.014 | −0.480 |
| LNM Group 3 vs. Group 1 | ENSG00000039523 | FAM65A | −1.120 | 6.034 | −5.351 | 0.000 | 0.001 | 4.381 |
| LNM Group 3 vs. Group 1 | ENSG00000175155 | YPEL2 | −1.121 | 5.977 | −4.427 | 0.000 | 0.004 | 1.621 |
| LNM Group 3 vs. Group 1 | ENSG00000198805 | PNP | −1.125 | 5.417 | −3.776 | 0.000 | 0.012 | −0.166 |
| LNM Group 3 vs. Group 1 | ENSG00000157734 | SNX22 | −1.127 | 4.420 | −3.071 | 0.004 | 0.040 | −2.021 |
| LNM Group 3 vs. Group 1 | ENSG00000187800 | PEAR1 | −1.128 | 4.040 | −3.167 | 0.003 | 0.034 | −1.727 |
| LNM Group 3 vs. Group 1 | ENSG00000176435 | CLEC14A | −1.130 | 2.626 | −3.664 | 0.001 | 0.014 | −0.447 |
| LNM Group 3 vs. Group 1 | ENSG00000149311 | ATM | −1.131 | 8.318 | −4.414 | 0.000 | 0.004 | 1.543 |
| LNM Group 3 vs. Group 1 | ENSG00000188641 | DPYD | −1.134 | 6.028 | −3.456 | 0.001 | 0.021 | −1.037 |
| LNM Group 3 vs. Group 1 | ENSG00000105366 | SIGLEC8 | −1.135 | 2.659 | −3.270 | 0.002 | 0.029 | −1.451 |
| LNM Group 3 vs. Group 1 | ENSG00000100385 | IL2RB | −1.138 | 5.071 | −3.778 | 0.000 | 0.012 | −0.187 |
| LNM Group 3 vs. Group 1 | ENSG00000106948 | AKNA | −1.139 | 7.642 | −2.973 | 0.005 | 0.047 | −2.371 |
| LNM Group 3 vs. Group 1 | ENSG00000135074 | ADAM19 | −1.143 | 6.949 | −4.202 | 0.000 | 0.006 | 0.931 |
| LNM Group 3 vs. Group 1 | ENSG00000047365 | ARAP2 | −1.144 | 7.054 | −3.674 | 0.001 | 0.014 | −0.539 |
| LNM Group 3 vs. Group 1 | ENSG00000172243 | CLEC7A | −1.145 | 5.358 | −3.865 | 0.000 | 0.010 | 0.056 |
| LNM Group 3 vs. Group 1 | ENSG00000163703 | CRELD1 | −1.145 | 3.910 | −4.256 | 0.000 | 0.006 | 1.152 |
| LNM Group 3 vs. Group 1 | ENSG00000197134 | ZNF257 | −1.147 | 2.637 | −4.418 | 0.000 | 0.004 | 1.602 |
| LNM Group 3 vs. Group 1 | ENSG00000176927 | EFCAB5 | −1.157 | 2.790 | −4.749 | 0.000 | 0.002 | 2.551 |
| LNM Group 3 vs. Group 1 | ENSG00000179344 | HLA-DQB1 | −1.160 | 6.280 | −3.352 | 0.002 | 0.025 | −1.339 |
| LNM Group 3 vs. Group 1 | ENSG00000109861 | CTSC | −1.161 | 8.068 | −3.166 | 0.003 | 0.034 | −1.882 |
| LNM Group 3 vs. Group 1 | ENSG00000138613 | APH1B | −1.162 | 4.311 | −4.190 | 0.000 | 0.006 | 0.968 |
| LNM Group 3 vs. Group 1 | ENSG00000216490 | IFI30 | −1.168 | 6.374 | −3.997 | 0.000 | 0.008 | 0.383 |
| LNM Group 3 vs. Group 1 | ENSG00000122986 | HVCN1 | −1.169 | 3.889 | −3.400 | 0.001 | 0.023 | −1.166 |
| LNM Group 3 vs. Group 1 | ENSG00000184922 | FMNL1 | −1.177 | 6.217 | −4.636 | 0.000 | 0.003 | 2.229 |
| LNM Group 3 vs. Group 1 | ENSG00000136286 | MYO1G | −1.180 | 5.435 | −3.689 | 0.001 | 0.013 | −0.440 |
| LNM Group 3 vs. Group 1 | ENSG00000140368 | PSTPIP1 | −1.180 | 4.421 | −2.955 | 0.005 | 0.048 | −2.265 |
| LNM Group 3 vs. Group 1 | ENSG00000107738 | C100rf54 | −1.184 | 6.132 | −3.734 | 0.001 | 0.012 | −0.323 |
| LNM Group 3 vs. Group 1 | ENSG00000074266 | EED | −1.185 | 4.950 | −3.675 | 0.001 | 0.014 | −0.446 |
| LNM Group 3 vs. Group 1 | ENSG00000129467 | ADCY4 | −1.187 | 4.374 | −3.258 | 0.002 | 0.029 | −1.502 |
| LNM Group 3 vs. Group 1 | ENSG00000090104 | RGS1 | −1.191 | 7.501 | −3.232 | 0.002 | 0.031 | −1.708 |
| LNM Group 3 vs. Group 1 | ENSG00000141480 | ARRB2 | −1.193 | 5.844 | −5.017 | 0.000 | 0.002 | 3.368 |
| LNM Group 3 vs. Group 1 | ENSG00000086730 | LAT2 | −1.194 | 5.132 | −3.052 | 0.004 | 0.042 | −2.066 |
| LNM Group 3 vs. Group 1 | ENSG00000103187 | COTL1 | −1.194 | 6.738 | −4.410 | 0.000 | 0.004 | 1.556 |
| LNM Group 3 vs. Group 1 | ENSG00000175567 | UCP2 | −1.195 | 5.557 | −3.289 | 0.002 | 0.028 | −1.487 |
| LNM Group 3 vs. Group 1 | ENSG00000115525 | ST3GAL5 | −1.195 | 4.626 | −3.774 | 0.001 | 0.012 | −0.170 |
| LNM Group 3 vs. Group 1 | ENSG00000172183 | ISG20 | −1.196 | 6.178 | −3.722 | 0.001 | 0.013 | −0.404 |
| LNM Group 3 vs. Group 1 | ENSG00000135678 | CPM | −1.197 | 5.991 | −3.171 | 0.003 | 0.034 | −1.774 |
| LNM Group 3 vs. Group 1 | ENSG00000163453 | IGFBP7 | −1.199 | 7.045 | −4.389 | 0.000 | 0.005 | 1.484 |
| LNM Group 3 vs. Group 1 | ENSG00000113742 | CPEB4 | −1.201 | 6.574 | −3.750 | 0.001 | 0.012 | −0.291 |
| LNM Group 3 vs. Group 1 | ENSG00000185201 | IFITM2 | −1.201 | 4.765 | −3.573 | 0.001 | 0.016 | −0.712 |
| LNM Group 3 vs. Group 1 | ENSG00000168010 | ATG16L2 | −1.204 | 6.296 | −5.262 | 0.000 | 0.001 | 4.109 |
| LNM Group 3 vs. Group 1 | ENSG00000111679 | PTPN6 | −1.204 | 5.799 | −3.611 | 0.001 | 0.015 | −0.666 |
| LNM Group 3 vs. Group 1 | ENSG00000002933 | TMEM176A | −1.207 | 4.062 | −4.146 | 0.000 | 0.006 | 0.846 |
| LNM Group 3 vs. Group 1 | ENSG00000240583 | AQP1 | −1.208 | 5.353 | −3.079 | 0.004 | 0.040 | −1.997 |
| LNM Group 3 vs. Group 1 | ENSG00000105643 | ARRDC2 | −1.209 | 5.605 | −3.884 | 0.000 | 0.010 | 0.069 |
| LNM Group 3 vs. Group 1 | ENSG00000153071 | DAB2 | −1.211 | 6.322 | −3.739 | 0.001 | 0.012 | −0.349 |
| LNM Group 3 vs. Group 1 | ENSG00000134954 | ETS1 | −1.215 | 8.326 | −4.769 | 0.000 | 0.002 | 2.602 |
| LNM Group 3 vs. Group 1 | ENSG00000153208 | MERTK | −1.215 | 5.186 | −3.269 | 0.002 | 0.029 | −1.507 |
| LNM Group 3 vs. Group 1 | ENSG00000143851 | PTPN7 | −1.219 | 5.008 | −3.552 | 0.001 | 0.017 | −0.793 |
| LNM Group 3 vs. Group 1 | ENSG00000171608 | PIK3CD | −1.223 | 6.149 | −4.000 | 0.000 | 0.008 | 0.377 |
| LNM Group 3 vs. Group 1 | ENSG00000198502 | HLA-DRB5 | −1.224 | 5.465 | −3.435 | 0.001 | 0.021 | −1.084 |
| LNM Group 3 vs. Group 1 | ENSG00000167323 | STIM1 | −1.226 | 6.876 | −3.230 | 0.002 | 0.031 | −1.702 |
| LNM Group 3 vs. Group 1 | ENSG00000081320 | STK17B | −1.227 | 7.868 | −4.402 | 0.000 | 0.004 | 1.508 |
| LNM Group 3 vs. Group 1 | ENSG00000100100 | PIK3IP1 | −1.233 | 6.385 | −3.028 | 0.004 | 0.043 | −2.180 |
| LNM Group 3 vs. Group 1 | ENSG00000066336 | SPI1 | −1.234 | 4.610 | −4.256 | 0.000 | 0.006 | 1.148 |
| LNM Group 3 vs. Group 1 | ENSG00000069424 | KCNAB2 | −1.236 | 5.933 | −3.962 | 0.000 | 0.009 | 0.297 |
| LNM Group 3 vs. Group 1 | ENSG00000160219 | GAB3 | −1.237 | 4.005 | −4.908 | 0.000 | 0.002 | 3.023 |
| LNM Group 3 vs. Group 1 | ENSG00000175489 | LRRC25 | −1.239 | 3.420 | −5.032 | 0.000 | 0.002 | 3.370 |
| LNM Group 3 vs. Group 1 | ENSG00000161638 | ITGA5 | −1.240 | 7.393 | −2.973 | 0.005 | 0.047 | −2.347 |
| LNM Group 3 vs. Group 1 | ENSG00000101194 | SLC17A9 | −1.240 | 4.676 | −3.189 | 0.003 | 0.033 | −1.709 |
| LNM Group 3 vs. Group 1 | ENSG00000198909 | MAP3K3 | −1.247 | 5.826 | −5.865 | 0.000 | 0.000 | 5.958 |
| LNM Group 3 vs. Group 1 | ENSG00000120899 | PTK2B | −1.249 | 6.900 | −4.433 | 0.000 | 0.004 | 1.617 |
| LNM Group 3 vs. Group 1 | ENSG00000101265 | RASSF2 | −1.259 | 6.133 | −3.078 | 0.004 | 0.040 | −2.040 |
| LNM Group 3 vs. Group 1 | ENSG00000099998 | GGT5 | −1.261 | 5.447 | −3.004 | 0.005 | 0.045 | −2.157 |
| LNM Group 3 vs. Group 1 | ENSG00000155465 | SLC7A7 | −1.264 | 4.553 | −4.455 | 0.000 | 0.004 | 1.714 |
| LNM Group 3 vs. Group 1 | ENSG00000136205 | TN53 | −1.268 | 7.632 | −5.069 | 0.000 | 0.001 | 3.515 |
| LNM Group 3 vs. Group 1 | ENSG00000241978 | AKAP2 | −1.269 | 6.986 | −3.887 | 0.000 | 0.010 | 0.028 |
| LNM Group 3 vs. Group 1 | ENSG00000140030 | GPR65 | −1.272 | 3.563 | −3.945 | 0.000 | 0.009 | 0.296 |
| LNM Group 3 vs. Group 1 | ENSG00000149564 | ESAM | −1.275 | 3.323 | −3.665 | 0.001 | 0.014 | −0.455 |
| LNM Group 3 vs. Group 1 | ENSG00000257017 | HP | −1.275 | 2.639 | −2.972 | 0.005 | 0.047 | −2.165 |
| LNM Group 3 vs. Group 1 | ENSG00000106565 | TMEM176B | −1.279 | 4.520 | −4.538 | 0.000 | 0.004 | 1.953 |
| LNM Group 3 vs. Group 1 | ENSG00000135047 | CTSL | −1.281 | 5.471 | −4.089 | 0.000 | 0.007 | 0.668 |
| LNM Group 3 vs. Group 1 | ENSG00000158717 | RNF166 | −1.282 | 5.196 | −4.180 | 0.000 | 0.006 | 0.924 |
| LNM Group 3 vs. Group 1 | ENSG00000110031 | LPXN | −1.282 | 4.750 | −3.752 | 0.001 | 0.012 | −0.237 |
| LNM Group 3 vs. Group 1 | ENSG00000105538 | RASIP1 | −1.288 | 3.235 | −3.810 | 0.000 | 0.011 | −0.064 |
| LNM Group 3 vs. Group 1 | ENSG00000173200 | PARP15 | −1.289 | 7.023 | −4.093 | 0.000 | 0.007 | 0.616 |
| LNM Group 3 vs. Group 1 | ENSG00000010610 | CD4 | −1.291 | 5.993 | −3.641 | 0 001 | 0.015 | −0.594 |
| LNM Group 3 vs. Group 1 | ENSG00000149781 | FERMT3 | −1.292 | 4.948 | −4.329 | 0.000 | 0.005 | 1 345 |
| LNM Group 3 vs. Group 1 | ENSG00000135077 | HAVCR2 | −1.303 | 5.005 | −4.712 | 0.000 | 0.003 | 2.460 |
| LNM Group 3 vs. Group 1 | ENSG00000110934 | BIN2 | −1.303 | 4.508 | −3.008 | 0.004 | 0.045 | −2.114 |
| LNM Group 3 vs. Group 1 | ENSG00000150867 | PIP4K2A | −1.303 | 6.207 | −4.553 | 0.000 | 0.003 | 1.991 |
| LNM Group 3 vs. Group 1 | ENSG00000155849 | ELMO1 | −1.304 | 5.964 | −4.039 | 0.000 | 0.008 | 0.493 |
| LNM Group 3 vs. Group 1 | ENSG00000153283 | CD96 | −1.305 | 4.602 | −3.257 | 0.002 | 0.029 | −1.522 |
| LNM Group 3 vs. Group 1 | ENSG00000136541 | ERMN | −1.305 | 2.728 | −3.525 | 0.001 | 0.018 | −0.816 |
| LNM Group 3 vs. Group 1 | ENSG00000117643 | MAN1C1 | −1.308 | 4.603 | −3.904 | 0.000 | 0.009 | 0.159 |
| LNM Group 3 vs. Group 1 | ENSG00000213654 | GPSM3 | −1.309 | 4.580 | −3.642 | 0.001 | 0.015 | −0.534 |
| LNM Group 3 vs. Group 1 | ENSG00000051108 | HERPUD1 | −1.311 | 6.974 | −3.795 | 0.000 | 0.011 | −0.198 |
| LNM Group 3 vs. Group 1 | ENSG00000164330 | EBF1 | −1.319 | 4.908 | −4.246 | 0.000 | 0.006 | 1.123 |
| LNM Group 3 vs. Group 1 | ENSG00000162645 | GBP2 | −1.325 | 5.772 | −3.096 | 0.004 | 0.039 | −1.952 |
| LNM Group 3 vs. Group 1 | ENSG00000104814 | MAP4K1 | −1.325 | 4.835 | −3.127 | 0.003 | 0.037 | −1.865 |
| LNM Group 3 vs. Group 1 | ENSG00000172578 | KLHL6 | −1.325 | 6.365 | −3.865 | 0.000 | 0.010 | 0.007 |
| LNM Group 3 vs. Group 1 | ENSG00000084070 | SMAP2 | −1.333 | 7.092 | −3.903 | 0.000 | 0.009 | 0.092 |
| LNM Group 3 vs. Group 1 | ENSG00000204257 | HLA-DMA | −1.335 | 5.557 | −4.029 | 0.000 | 0.008 | 0.504 |
| LNM Group 3 vs. Group 1 | ENSG00000145703 | IQGAP2 | −1.335 | 5.472 | −3.639 | 0.001 | 0.015 | −0.557 |
| LNM Group 3 vs. Group 1 | ENSG00000051523 | CYBA | −1.338 | 6.100 | −4.997 | 0.000 | 0.002 | 3.308 |
| LNM Group 3 vs. Group 1 | ENSG00000114166 | KAT2B | −1.339 | 5.467 | −4.341 | 0.000 | 0.005 | 1.387 |
| LNM Group 3 vs. Group 1 | ENSG00000000938 | FGR | −1.341 | 4.403 | −3.119 | 0.003 | 0.037 | −1.860 |
| LNM Group 3 vs. Group 1 | ENSG00000185862 | EVI2B | −1.343 | 5.092 | −3.898 | 0.000 | 0.009 | 0.142 |
| LNM Group 3 vs. Group 1 | ENSG00000111348 | ARHGDIB | −1.345 | 6.685 | −3.988 | 0.000 | 0.008 | 0.342 |
| LNM Group 3 vs. Group 1 | ENSG00000130775 | THEMIS2 | −1.346 | 6.274 | −3.741 | 0.001 | 0.012 | −0.316 |
| LNM Group 3 vs. Group 1 | ENSG00000140678 | ITGAX | −1.348 | 6.568 | −3.385 | 0.002 | 0.023 | −1.293 |
| LNM Group 3 vs. Group 1 | ENSG00000231389 | HLA-DPA1 | −1.352 | 7.688 | −4.204 | 0.000 | 0.006 | 0.934 |
| LNM Group 3 vs. Group 1 | ENSG00000163219 | ARHGAP25 | −1.353 | 5.145 | −4.223 | 0.000 | 0.006 | 1.040 |
| LNM Group 3 vs. Group 1 | ENSG00000196735 | HLA-DQA1 | −1.353 | 6.399 | −3.267 | 0.002 | 0.029 | −1.541 |
| LNM Group 3 vs. Group 1 | ENSG00000163359 | COL6A3 | −1.354 | 10.354 | −3.159 | 0.003 | 0.035 | −1.888 |
| LNM Group 3 vs. Group 1 | ENSG00000010327 | STAB1 | −1.356 | 7.455 | −3.445 | 0.001 | 0.021 | −1.157 |
| LNM Group 3 vs. Group 1 | ENSG00000174130 | TLR6 | −1.357 | 5.014 | −4.422 | 0.000 | 0.004 | 1.616 |
| LNM Group 3 vs. Group 1 | ENSG00000128052 | KDR | −1.363 | 4.276 | −3.648 | 0.001 | 0.014 | −0.533 |
| LNM Group 3 vs. Group 1 | ENSG00000168071 | CCDC88B | −1.364 | 6.283 | −3.838 | 0.000 | 0.010 | −0.072 |
| LNM Group 3 vs. Group 1 | ENSG00000126785 | RHOJ | −1.365 | 3.907 | −3.191 | 0.003 | 0.033 | −1.674 |
| LNM Group 3 vs. Group 1 | ENSG00000154217 | PITPNC1 | −1.368 | 5.134 | −6.029 | 0.000 | 0.000 | 6.430 |
| LNM Group 3 vs. Group 1 | ENSG00000010295 | IFFO1 | −1.369 | 4.508 | −4.462 | 0.000 | 0.004 | 1.737 |
| LNM Group 3 vs. Group 1 | ENSG00000159733 | ZFYVE28 | −1.373 | 3.826 | −4.085 | 0.000 | 0.007 | 0.677 |
| LNM Group 3 vs. Group 1 | ENSG00000011600 | TYROBP | −1.377 | 3.824 | −4.188 | 0.000 | 0.006 | 0.961 |
| LNM Group 3 vs. Group 1 | ENSG00000100219 | XBP1 | −1.377 | 7.426 | −3.081 | 0.004 | 0.040 | −2.085 |
| LNM Group 3 vs. Group 1 | ENSG00000181896 | ZNF101 | −1.378 | 6.042 | −3.185 | 0.003 | 0.033 | −1.788 |
| LNM Group 3 vs. Group 1 | ENSG00000131378 | RFTN1 | −1.379 | 6.038 | −4.490 | 0.000 | 0.004 | 1.800 |
| LNM Group 3 vs. Group 1 | ENSG00000127329 | PTPRB | −1.384 | 5.478 | −4.395 | 0.000 | 0.004 | 1.523 |
| LNM Group 3 vs. Group 1 | ENSG00000123338 | NCKAP1L | −1.386 | 6.793 | −4.626 | 0.000 | 0.003 | 2.187 |
| LNM Group 3 vs. Group 1 | ENSG00000148516 | ZEB1 | −1.386 | 5.941 | −3.889 | 0.000 | 0.010 | 0.086 |
| LNM Group 3 vs. Group 1 | ENSG00000149054 | ZNF215 | −1.389 | 2.944 | −3.183 | 0.003 | 0.033 | −1.664 |
| LNM Group 3 vs. Group 1 | ENSG00000182568 | SATB1 | −1.390 | 6.057 | −3.520 | 0.001 | 0.018 | −0.929 |
| LNM Group 3 vs. Group 1 | ENSG00000154814 | OXNAD1 | −1.390 | 5.352 | −4.715 | 0.000 | 0.003 | 2.467 |
| LNM Group 3 vs. Group 1 | ENSG00000101082 | SLA2 | −1.390 | 3.573 | −3.760 | 0.001 | 0.012 | −0.213 |
| LNM Group 3 vs. Group 1 | ENSG00000196126 | HLA-DRB1 | −1.390 | 7.074 | −4.043 | 0.000 | 0.008 | 0.505 |
| LNM Group 3 vs. Group 1 | ENSG00000184060 | ADAP2 | −1.400 | 4.480 | −3.954 | 0.000 | 0.009 | 0.314 |
| LNM Group 3 vs. Group 1 | ENSG00000164674 | SYTL3 | −1.400 | 4.341 | −3.037 | 0.004 | 0.043 | −2.058 |
| LNM Group 3 vs. Group 1 | ENSG00000158869 | FCER1G | −1.402 | 4.294 | −4.045 | 0.000 | 0.008 | 0.565 |
| LNM Group 3 vs. Group 1 | ENSG00000091490 | SEL1L3 | −1.409 | 6.742 | −3.615 | 0.001 | 0.015 | −0.698 |
| LNM Group 3 vs. Group 1 | ENSG00000196209 | SIRPB2 | −1.411 | 3.345 | −3.478 | 0.001 | 0.020 | −0.940 |
| LNM Group 3 vs. Group 1 | ENSG00000184497 | TMEM255B | −1.411 | 3.912 | −3.612 | 0.001 | 0.015 | −0.602 |
| LNM Group 3 vs. Group 1 | ENSG00000179776 | CDH5 | −1.411 | 4.453 | −3.000 | 0.005 | 0.045 | −2.151 |
| LNM Group 3 vs. Group 1 | ENSG00000159640 | ACE | −1.411 | 4.406 | −3.322 | 0.002 | 0.026 | −1.356 |
| LNM Group 3 vs. Group 1 | ENSG00000178199 | ZC3H12D | −1.412 | 5.381 | −3.574 | 0.001 | 0.016 | −0.748 |
| LNM Group 3 vs. Group 1 | ENSG00000101096 | NFATC2 | −1.413 | 6.151 | −3.205 | 0.003 | 0.032 | −1.740 |
| LNM Group 3 vs. Group 1 | ENSG00000105609 | LILRB5 | −1.415 | 3.526 | −2.942 | 0.005 | 0.049 | −2.255 |
| LNM Group 3 vs. Group 1 | ENSG00000130830 | MPP1 | −1.416 | 4.241 | −4.456 | 0.000 | 0.004 | 1.718 |
| LNM Group 3 vs. Group 1 | ENSG00000130653 | PNPLA7 | −1.420 | 4.295 | −3.631 | 0.001 | 0.015 | −0.559 |
| LNM Group 3 vs. Group 1 | ENSG00000133392 | MYH11 | −1.423 | 6.163 | −3.087 | 0.004 | 0.039 | −2.048 |
| LNM Group 3 vs. Group 1 | ENSG00000146826 | C7orf43 | −1.423 | 5.137 | −3.172 | 0.003 | 0.034 | −1.819 |
| LNM Group 3 vs. Group 1 | ENSG00000223865 | HLA-DPB1 | −1.427 | 6.980 | −4.237 | 0.000 | 0.006 | 1.046 |
| LNM Group 3 vs. Group 1 | ENSG00000078237 | TIGAR | −1.435 | 4.361 | −2.978 | 0.005 | 0.047 | −2.210 |
| LNM Group 3 vs. Group 1 | ENSG00000185477 | GPRIN3 | −1.436 | 6.767 | −4.515 | 0.000 | 0.004 | 1.848 |
| LNM Group 3 vs. Group 1 | ENSG00000072786 | STK10 | −1.442 | 6.579 | −6.890 | 0.000 | 0.000 | 9.126 |
| LNM Group 3 vs. Group 1 | ENSG00000007237 | GAS7 | −1.443 | 5.701 | −3.849 | 0.000 | 0.010 | −0.023 |
| LNM Group 3 vs. Group 1 | ENSG00000104714 | ERICH1 | −1.445 | 5.889 | −4.960 | 0.000 | 0.002 | 3.185 |
| LNM Group 3 vs. Group 1 | ENSG00000111860 | CEP85L | −1.445 | 5.181 | −5.119 | 0.000 | 0.001 | 3.672 |
| LNM Group 3 vs. Group 1 | ENSG00000176928 | GCNT4 | −1.451 | 3.522 | −3.230 | 0.002 | 0.031 | −1.572 |
| LNM Group 3 vs. Group 1 | ENSG00000134830 | C5AR2 | −1.451 | 4.874 | −3.968 | 0.000 | 0.009 | 0.329 |
| LNM Group 3 vs. Group 1 | ENSG00000126882 | FAM78A | −1.454 | 4.841 | −3.584 | 0.001 | 0.016 | −0.392 |
| LNM Group 3 vs. Group 1 | ENSG00000163568 | AIM2 | −1.458 | 3.740 | −3.710 | 0.001 | 0.013 | −0.334 |
| LNM Group 3 vs. Group 1 | ENSG00000076641 | PAG1 | −1.465 | 6.732 | −4.691 | 0.000 | 0.003 | 2.380 |
| LNM Group 3 vs. Group 1 | ENSG00000115956 | PLEK | −1.466 | 5.488 | −3.401 | 0.002 | 0.023 | −1.193 |
| LNM Group 3 vs. Group 1 | ENSG00000180448 | ARHGAP45 | −1.477 | 6.594 | −3.961 | 0.000 | 0.009 | 0.260 |
| LNM Group 3 vs. Group 1 | ENSG00000138134 | STAMBPL1 | −1.482 | 4.427 | −4.076 | 0.000 | 0.007 | 0.637 |
| LNM Group 3 vs. Group 1 | ENSG00000155307 | SAMSN1 | −1.486 | 4.594 | −3.929 | 0.000 | 0.009 | 0.231 |
| LNM Group 3 vs. Group 1 | ENSG00000170571 | EMB | −1.486 | 6.398 | −4.312 | 0.000 | 0.005 | 1.281 |
| LNM Group 3 vs. Group 1 | ENSG00000110876 | SELPLG | −1.491 | 4.647 | −4.097 | 0.000 | 0.007 | 0.699 |
| LNM Group 3 vs. Group 1 | ENSG00000162618 | ADGRL4 | −1.494 | 3.411 | −3.851 | 0.000 | 0.010 | 0.043 |
| LNM Group 3 vs. Group 1 | ENSG00000198440 | ZNF583 | −1.496 | 3.358 | −4.123 | 0.000 | 0.007 | 0.761 |
| LNM Group 3 vs. Group 1 | ENSG00000107968 | MAP3K8 | −1.498 | 4.960 | −3.687 | 0.001 | 0.013 | −0.423 |
| LNM Group 3 vs. Group 1 | ENSG00000115232 | ITGA4 | −1.500 | 5.797 | −3.770 | 0.001 | 0.012 | −0.228 |
| LNM Group 3 vs. Group 1 | ENSG00000180353 | HCLS1 | −1.503 | 6.474 | −4.215 | 0.000 | 0.006 | 0.991 |
| LNM Group 3 vs. Group 1 | ENSG00000141338 | ABCA8 | −1.504 | 3.217 | −2.972 | 0.005 | 0.047 | −2.170 |
| LNM Group 3 vs. Group 1 | ENSG00000146070 | PLA2G7 | −1.510 | 3.797 | −4.125 | 0.000 | 0.007 | 0.784 |
| LNM Group 3 vs. Group 1 | ENSG00000271503 | CCL5 | −1.511 | 4.877 | −4.271 | 0.000 | 0.006 | 1.183 |
| LNM Group 3 vs. Group 1 | ENSG00000169744 | LDB2 | −1.513 | 3.759 | −4.115 | 0.000 | 0.007 | 0.757 |
| LNM Group 3 vs. Group 1 | ENSG00000151067 | CACNA1C | −1.519 | 4.975 | −3.172 | 0.003 | 0.034 | −1.765 |
| LNM Group 3 vs. Group 1 | ENSG00000135709 | KIAA0513 | −1.520 | 5.999 | −4.113 | 0.000 | 0.007 | 0.738 |
| LNM Group 3 vs. Group 1 | ENSG00000079215 | SLC1A3 | −1.521 | 5.467 | −3.458 | 0.001 | 0.021 | −1.033 |
| LNM Group 3 vs. Group 1 | ENSG00000160255 | ITGB2 | −1.524 | 6.600 | −4.370 | 0.000 | 0.005 | 1.437 |
| LNM Group 3 vs. Group 1 | ENSG00000167895 | TMC8 | −1.524 | 6.024 | −3.383 | 0.002 | 0.024 | −1.283 |
| LNM Group 3 vs. Group 1 | ENSG00000124126 | PREX1 | −1.524 | 6.531 | −4.974 | 0.000 | 0.002 | 3.226 |
| LNM Group 3 vs. Group 1 | ENSG00000104894 | CD37 | −1.529 | 5.981 | −3.191 | 0.003 | 0.033 | −1.770 |
| LNM Group 3 vs. Group 1 | ENSG00000204287 | HLA-DRA | −1.534 | 8.392 | −4.823 | 0.000 | 0.002 | 2.758 |
| LNM Group 3 vs. Group 1 | ENSG00000174059 | CD34 | −1.534 | 4.740 | −3.107 | 0.003 | 0.038 | −1.874 |
| LNM Group 3 vs. Group 1 | ENSG00000113532 | ST8SIA4 | −1.537 | 5.509 | −4.469 | 0.000 | 0.004 | 1.743 |
| LNM Group 3 vs. Group 1 | ENSG00000043462 | LCP2 | −1.538 | 5.996 | −4.946 | 0.000 | 0.002 | 3.149 |
| LNM Group 3 vs. Group 1 | ENSG00000107551 | RASSF4 | −1.538 | 6.337 | −4.225 | 0.000 | 0.006 | 1.034 |
| LNM Group 3 vs. Group 1 | ENSG00000028137 | TNFRSF1B | −1.540 | 6.371 | −3.577 | 0.001 | 0.016 | −0.787 |
| LNM Group 3 vs. Group 1 | ENSG00000166250 | CLMP | −1.548 | 5.567 | −3.491 | 0.001 | 0.019 | −0.934 |
| LNM Group 3 vs. Group 1 | ENSG00000163513 | TGFBR2 | −1.551 | 7.259 | −5.361 | 0.000 | 0.001 | 4.404 |
| LNM Group 3 vs. Group 1 | ENSG00000136250 | AOAH | −1.552 | 4.430 | −4.132 | 0.000 | 0.007 | 0.796 |
| LNM Group 3 vs. Group 1 | ENSG00000165168 | CYBB | −1.554 | 6.904 | −4.468 | 0.000 | 0.004 | 1.732 |
| LNM Group 3 vs. Group 1 | ENSG00000188906 | LRRK2 | −1.555 | 5.285 | −3.713 | 0.001 | 0.013 | −0.366 |
| LNM Group 3 vs. Group 1 | ENSG00000023171 | GRAMD1B | −1.556 | 4.403 | −3.863 | 0.000 | 0.010 | 0.055 |
| LNM Group 3 vs. Group 1 | ENSG00000125354 | SEPT6 | −1.561 | 6.122 | −4.316 | 0.000 | 0.005 | 1.287 |
| LNM Group 3 vs. Group 1 | ENSG00000163131 | CTSS | −1.563 | 6.658 | −4.294 | 0.000 | 0.005 | 1.228 |
| LNM Group 3 vs. Group 1 | ENSG00000123329 | ARHGAP9 | −1.569 | 5.244 | −2.951 | 0.005 | 0.048 | −2.287 |
| LNM Group 3 vs. Group 1 | ENSG00000157514 | TSC22D3 | −1.571 | 7.754 | −4.063 | 0.000 | 0.007 | 0.550 |
| LNM Group 3 vs. Group 1 | ENSG00000072818 | ACAP1 | −1.574 | 6.160 | −4.513 | 0.000 | 0.004 | 1.858 |
| LNM Group 3 vs. Group 1 | ENSG00000013725 | CD6 | −1.574 | 5.043 | −3.327 | 0.002 | 0.026 | −1.373 |
| LNM Group 3 vs. Group 1 | ENSG00000154127 | UBASH3B | −1.575 | 5.263 | −4.906 | 0.000 | 0.002 | 3.029 |
| LNM Group 3 vs. Group 1 | ENSG00000107099 | DOCK8 | −1.576 | 7.251 | −4.449 | 0.000 | 0.004 | 1.657 |
| LNM Group 3 vs. Group 1 | ENSG00000122122 | SASH3 | −1.578 | 4.957 | −3.371 | 0.002 | 0.024 | −1.247 |
| LNM Group 3 vs. Group 1 | ENSG00000197249 | SERPINA1 | −1.582 | 4.599 | −3.260 | 0.002 | 0.029 | −1.508 |
| LNM Group 3 vs. Group 1 | ENSG00000115165 | CYTIP | −1.583 | 5.726 | −3.506 | 0.001 | 0.019 | −0.922 |
| LNM Group 3 vs. Group 1 | ENSG00000158517 | NCF1 | −1.584 | 4.067 | −3.573 | 0.001 | 0.017 | −0.710 |
| LNM Group 3 vs. Group 1 | ENSG00000055163 | CYFIP2 | −1.584 | 5.674 | −3.225 | 0.002 | 0.031 | −1.655 |
| LNM Group 3 vs. Group 1 | ENSG00000159189 | C1QC | −1.590 | 5.953 | −3.923 | 0.000 | 0.009 | 0.199 |
| LNM Group 3 vs. Group 1 | ENSG00000168685 | IL7R | −1.592 | 7.463 | −4.384 | 0.000 | 0.005 | 1.461 |
| LNM Group 3 vs. Group 1 | ENSG00000204252 | HLA-DOA | −1.595 | 5.489 | −3.005 | 0.005 | 0.045 | −2.186 |
| LNM Group 3 vs. Group 1 | ENSG00000198771 | RCSD1 | −1.595 | 5.765 | −4.288 | 0.000 | 0.005 | 1.204 |
| LNM Group 3 vs. Group 1 | ENSG00000102575 | ACP5 | −1.605 | 5.001 | −4.330 | 0.000 | 0.005 | 1.351 |
| LNM Group 3 vs. Group 1 | ENSG00000166278 | C2 | −1.610 | 5.364 | −4.026 | 0.000 | 0.008 | 0.498 |
| LNM Group 3 vs. Group 1 | ENSG00000033327 | GAB2 | −1.611 | 5.449 | −4.676 | 0.000 | 0.003 | 2.345 |
| LNM Group 3 vs. Group 1 | ENSG00000172572 | PDE3A | −1.612 | 4.914 | −3.906 | 0.000 | 0.009 | 0.154 |
| LNM Group 3 vs. Group 1 | ENSG00000172349 | IL16 | −1.614 | 6.119 | −3.723 | 0.001 | 0.013 | −0.382 |
| LNM Group 3 vs. Group 1 | ENSG00000009790 | TRAF3IP3 | −1.617 | 5.648 | −3.401 | 0.002 | 0.023 | −1.203 |
| LNM Group 3 vs. Group 1 | ENSG00000130203 | APOE | −1.618 | 6.301 | −3.830 | 0.000 | 0.011 | −0.071 |
| LNM Group 3 vs. Group 1 | ENSG00000104903 | LYL1 | −1.619 | 3.187 | −4.479 | 0.000 | 0.004 | 1.779 |
| LNM Group 3 vs. Group 1 | ENSG00000170458 | CD14 | −1.619 | 4.771 | −3.944 | 0.000 | 0.009 | 0.273 |
| LNM Group 3 vs. Group 1 | ENSG00000242574 | HLA-DMB | −1.620 | 6.042 | −4.700 | 0.000 | 0.003 | 2.414 |
| LNM Group 3 vs. Group 1 | ENSG00000019582 | CD74 | −1.627 | 10.000 | −5.365 | 0.000 | 0.001 | 4.404 |
| LNM Group 3 vs. Group 1 | ENSG00000129226 | CD68 | −1.628 | 6.067 | −4.523 | 0.000 | 0.004 | 1.901 |
| LNM Group 3 vs. Group 1 | ENSG00000172215 | CXCR6 | −1.629 | 3.773 | −5.709 | 0.000 | 0.001 | 5.381 |
| LNM Group 3 vs. Group 1 | ENSG00000127528 | KLF2 | −1.634 | 5.222 | −2.936 | 0.005 | 0.049 | −2.329 |
| LNM Group 3 vs. Group 1 | ENSG00000147168 | IL2RG | −1.638 | 4.862 | −4.182 | 0.000 | 0.006 | 0.930 |
| LNM Group 3 vs. Group 1 | ENSG00000130475 | FCHO1 | −1.643 | 3.866 | −5.198 | 0.000 | 0.001 | 3.886 |
| LNM Group 3 vs. Group 1 | ENSG00000261371 | PECAM1 | −1.644 | 6.787 | −5.983 | 0.000 | 0.000 | 6.316 |
| LNM Group 3 vs. Group 1 | ENSG00000151702 | FLI1 | −1.653 | 5.747 | −5.204 | 0.000 | 0.001 | 3.922 |
| LNM Group 3 vs. Group 1 | ENSG00000125810 | CD93 | −1.653 | 6.515 | −5.218 | 0.000 | 0.001 | 3.961 |
| LNM Group 3 vs. Group 1 | ENSG00000122862 | SRGN | −1.653 | 6.383 | −5.341 | 0.000 | 0.001 | 4.336 |
| LNM Group 3 vs. Group 1 | ENSG00000075884 | ARHGAP15 | −1.654 | 5.701 | −3.609 | 0.001 | 0.015 | −0.656 |
| LNM Group 3 vs. Group 1 | ENSG00000131018 | SYNE1 | −1.663 | 7.671 | −6.146 | 0.000 | 0.000 | 6.832 |
| LNM Group 3 vs. Group 1 | ENSG00000173369 | C1QB | −1.667 | 5.963 | −3.670 | 0.001 | 0.014 | −0.483 |
| LNM Group 3 vs. Group 1 | ENSG00000182183 | FAM159A | −1.668 | 3.510 | −3.887 | 0.000 | 0.010 | 0.125 |
| LNM Group 3 vs. Group 1 | ENSG00000149131 | SERPING1 | −1.668 | 6.271 | −3.763 | 0.001 | 0.012 | −0.228 |
| LNM Group 3 vs. Group 1 | ENSG00000074966 | TXK | −1.669 | 3.750 | −3.084 | 0.004 | 0.040 | −1.925 |
| LNM Group 3 vs. Group 1 | ENSG00000113263 | ITK | −1.670 | 6.134 | −3.261 | 0.002 | 0.029 | −1.594 |
| LNM Group 3 vs. Group 1 | ENSG00000135905 | DOCK10 | −1.672 | 6.688 | −4.819 | 0.000 | 0.002 | 2.758 |
| LNM Group 3 vs. Group 1 | ENSG00000174600 | CMKLR1 | −1.673 | 4.770 | −3.895 | 0.000 | 0.010 | 0.146 |
| LNM Group 3 vs. Group 1 | ENSG00000149591 | TAGLN | −1.676 | 6.751 | −3.317 | 0.002 | 0.026 | −1.491 |
| LNM Group 3 vs. Group 1 | ENSG00000102879 | CORO1A | −1.691 | 6.289 | −3.264 | 0.002 | 0.029 | −1.580 |
| LNM Group 3 vs. Group 1 | ENSG00000177575 | CD163 | −1.697 | 6.905 | −3.298 | 0.002 | 0.027 | −1.471 |
| LNM Group 3 vs. Group 1 | ENSG00000250722 | SEPP1 | −1.709 | 6.223 | −2.962 | 0.005 | 0.047 | −2.332 |
| LNM Group 3 vs. Group 1 | ENSG00000175899 | A2M | −1.709 | 7.554 | −5.300 | 0.000 | 0.001 | 4.206 |
| LNM Group 3 vs. Group 1 | ENSG00000166741 | NNMT | −1.709 | 5.332 | −3.083 | 0.004 | 0.040 | −1.963 |
| LNM Group 3 vs. Group 1 | ENSG00000137801 | THBS1 | −1.715 | 9.562 | −3.468 | 0.001 | 0.020 | −1.103 |
| LNM Group 3 vs. Group 1 | ENSG00000161405 | IKZF3 | −1.717 | 7.038 | −3.707 | 0.001 | 0.013 | −0.440 |
| LNM Group 3 vs. Group 1 | ENSG00000077238 | IL4R | −1.719 | 7.293 | −4.881 | 0.000 | 0.002 | 2.934 |
| LNM Group 3 vs. Group 1 | ENSG00000185989 | RASA3 | −1.726 | 5.993 | −5.793 | 0.000 | 0.000 | 5.717 |
| LNM Group 3 vs. Group 1 | ENSG00000179583 | CIITA | −1.740 | 7.843 | −4.243 | 0.000 | 0.006 | 1.043 |
| LNM Group 3 vs. Group 1 | ENSG00000100055 | CYTH4 | −1.747 | 5.662 | −5.902 | 0.000 | 0.000 | 6.045 |
| LNM Group 3 vs. Group 1 | ENSG00000169554 | ZEB2 | −1.751 | 7.836 | −4.719 | 0.000 | 0.003 | 2.440 |
| LNM Group 3 vs. Group 1 | ENSG00000116016 | EPAS1 | −1.751 | 7.912 | −8.279 | 0.000 | 0.000 | 13.305 |
| LNM Group 3 vs. Group 1 | ENSG00000128815 | WDFY4 | −1.755 | 4.967 | −3.297 | 0.002 | 0.027 | −1.432 |
| LNM Group 3 vs. Group 1 | ENSG00000171860 | C3AR1 | −1.759 | 3.620 | −4.083 | 0.000 | 0.007 | 0.664 |
| LNM Group 3 vs. Group 1 | ENSG00000104972 | LILRB1 | −1.767 | 4.493 | −4.401 | 0.000 | 0.004 | 1.546 |
| LNM Group 3 vs. Group 1 | ENSG00000171115 | GIMAP8 | −1.774 | 4.344 | −5.332 | 0.000 | 0.001 | 4.265 |
| LNM Group 3 vs. Group 1 | ENSG00000081237 | PTPRC | −1.777 | 7.711 | −4.243 | 0.000 | 0.006 | 1.050 |
| LNM Group 3 vs. Group 1 | ENSG00000135636 | DYSF | −1.795 | 5.415 | −3.816 | 0.000 | 0.011 | −0.074 |
| LNM Group 3 vs. Group 1 | ENSG00000155926 | SLA | −1.799 | 6.359 | −4.873 | 0.000 | 0.002 | 2.919 |
| LNM Group 3 vs. Group 1 | ENSG00000117009 | KMO | −1.800 | 3.867 | −3.269 | 0.002 | 0.029 | −1.467 |
| LNM Group 3 vs. Group 1 | ENSG00000113749 | HRH2 | −1.801 | 3.722 | −3.494 | 0.001 | 0.019 | −0.900 |
| LNM Group 3 vs. Group 1 | ENSG00000197943 | PLCG2 | −1.807 | 5.984 | −3.790 | 0.000 | 0.011 | −0.171 |
| LNM Group 3 vs. Group 1 | ENSG00000146094 | DOK3 | −1.813 | 4.438 | −4.657 | 0.000 | 0.003 | 2.285 |
| LNM Group 3 vs. Group 1 | ENSG00000139567 | ACVRL1 | −1.814 | 4.570 | −5.678 | 0.000 | 0.001 | 5.301 |
| LNM Group 3 vs. Group 1 | ENSG00000158714 | SLAMF8 | −1.820 | 4.665 | −4.737 | 0.000 | 0.003 | 2.512 |
| LNM Group 3 vs. Group 1 | ENSG00000137033 | IL33 | −1.826 | 4.084 | −3.132 | 0.003 | 0.037 | −1.829 |
| LNM Group 3 vs. Group 1 | ENSG00000072694 | FCGR2B | −1.835 | 4.904 | −4.037 | 0.000 | 0.008 | 0.529 |
| LNM Group 3 vs. Group 1 | ENSG00000026751 | SLAMF7 | −1.835 | 4.997 | −3.091 | 0.004 | 0.039 | −1.927 |
| LNM Group 3 vs. Group 1 | ENSG00000121904 | CSMD2 | −1.837 | 3.400 | −3.130 | 0.003 | 0.037 | −1.815 |
| LNM Group 3 vs. Group 1 | ENSG00000157110 | RBPMS | −1.838 | 5.558 | −3.877 | 0.000 | 0.010 | 0.053 |
| LNM Group 3 vs. Group 1 | ENSG00000090382 | LYZ | −1.844 | 7.279 | −4.074 | 0.000 | 0.007 | 0.575 |
| LNM Group 3 vs. Group 1 | ENSG00000167613 | LAIR1 | −1.850 | 5.707 | −5.346 | 0.000 | 0.001 | 4.337 |
| LNM Group 3 vs. Group 1 | ENSG00000186818 | LILRB4 | −1.851 | 4.655 | −4.506 | 0.000 | 0.004 | 1.849 |
| LNM Group 3 vs. Group 1 | ENSG00000082074 | FYB | −1.855 | 6.661 | −4.249 | 0.000 | 0.006 | 1.098 |
| LNM Group 3 vs. Group 1 | ENSG00000115523 | GNLY | −1.858 | 3.611 | −3.996 | 0.000 | 0.008 | 0.425 |
| LNM Group 3 vs. Group 1 | ENSG00000168918 | INPP5D | −1.860 | 6.562 | −3.895 | 0.000 | 0.010 | 0.105 |
| LNM Group 3 vs. Group 1 | ENSG00000005844 | ITGAL | −1.870 | 5.812 | −3.713 | 0.001 | 0.013 | −0.370 |
| LNM Group 3 vs. Group 1 | ENSG00000198959 | TGM2 | −1.880 | 7.010 | −4.403 | 0.000 | 0.004 | 1.525 |
| LNM Group 3 vs. Group 1 | ENSG00000131042 | LILRB2 | −1.881 | 3.751 | −4.142 | 0.000 | 0.007 | 0.825 |
| LNM Group 3 vs. Group 1 | ENSG00000146192 | FGD2 | −1.894 | 6.172 | −4.822 | 0.000 | 0.002 | 2.770 |
| LNM Group 3 vs. Group 1 | ENSG00000197629 | MPEG1 | −1.900 | 6.038 | −5.043 | 0.000 | 0.002 | 3.428 |
| LNM Group 3 vs. Group 1 | ENSG00000138964 | PARVG | −1.901 | 5.810 | −4.043 | 0.000 | 0.008 | 0.523 |
| LNM Group 3 vs. Group 1 | ENSG00000130755 | GMFG | −1.910 | 4.241 | −4.101 | 0.000 | 0.007 | 0.711 |
| LNM Group 3 vs. Group 1 | ENSG00000140968 | IRF8 | −1.913 | 5.673 | −3.382 | 0.002 | 0.024 | −1.240 |
| LNM Group 3 vs. Group 1 | ENSG00000134516 | DOCK2 | −1.915 | 6.540 | −4.254 | 0.000 | 0.006 | 1.119 |
| LNM Group 3 vs. Group 1 | ENSG00000110077 | MS4A6A | −1.919 | 5.824 | −4.836 | 0.000 | 0.002 | 2.810 |
| LNM Group 3 vs. Group 1 | ENSG00000137491 | SLCO2B1 | −1.926 | 7.133 | −5.577 | 0.000 | 0.001 | 5.042 |
| LNM Group 3 vs. Group 1 | ENSG00000162511 | LAPTM5 | −1.932 | 7.971 | −5.776 | 0.000 | 0.001 | 5.664 |
| LNM Group 3 vs. Group 1 | ENSG00000133574 | GIMAP4 | −1.951 | 4.506 | −4.377 | 0.000 | 0.005 | 1.482 |
| LNM Group 3 vs. Group 1 | ENSG00000259207 | ITGB3 | −1.958 | 3.076 | −3.515 | 0.001 | 0.019 | −0.842 |
| LNM Group 3 vs. Group 1 | ENSG00000141506 | PIK3R5 | −1.963 | 5.404 | −4.198 | 0.000 | 0.006 | 0.968 |
| LNM Group 3 vs. Group 1 | ENSG00000143226 | FCGR2A | −1.966 | 5.987 | −4.109 | 0.000 | 0.007 | 0.724 |
| LNM Group 3 vs. Group 1 | ENSG00000166927 | MS4A7 | −1.968 | 5.592 | −4.062 | 0.000 | 0.007 | 0.588 |
| LNM Group 3 vs. Group 1 | ENSG00000173372 | C1QA | −1.969 | 5.769 | −4.221 | 0.000 | 0.006 | 1.037 |
| LNM Group 3 vs. Group 1 | ENSG00000137841 | PLCB2 | −2.006 | 5.407 | −4.278 | 0.000 | 0.006 | 1.201 |
| LNM Group 3 vs. Group 1 | ENSG00000283652 | WDFY4 | −2.016 | 5.915 | −3.567 | 0.001 | 0.017 | −0.772 |
| LNM Group 3 vs. Group 1 | ENSG00000143119 | CD53 | −2.023 | 6.129 | −4.705 | 0.000 | 0.003 | 2.428 |
| LNM Group 3 vs. Group 1 | ENSG00000066294 | CD84 | −2.023 | 6.667 | −4.910 | 0.000 | 0.002 | 3.031 |
| LNM Group 3 vs. Group 1 | ENSG00000170989 | S1PR1 | −2.035 | 4.546 | −4.386 | 0.000 | 0.005 | 1.509 |
| LNM Group 3 vs. Group 1 | ENSG00000127084 | FGD3 | −2.055 | 5.444 | −3.655 | 0.001 | 0.014 | −0.529 |
| LNM Group 3 vs. Group 1 | ENSG00000124406 | ATP8A1 | −2.068 | 5.412 | −3.523 | 0.001 | 0.018 | −0.848 |
| LNM Group 3 vs. Group 1 | ENSG00000110324 | IL10RA | −2.089 | 6.457 | −4.922 | 0.000 | 0.002 | 3.068 |
| LNM Group 3 vs. Group 1 | ENSG00000129538 | RNASE1 | −2.103 | 4.986 | −2.961 | 0.005 | 0.047 | −2.208 |
| LNM Group 3 vs. Group 1 | ENSG00000111913 | FAM65B | −2.119 | 6.221 | −3.604 | 0.001 | 0.016 | −0.690 |
| LNM Group 3 vs. Group 1 | ENSG00000136960 | ENPP2 | −2.173 | 4.829 | −5.046 | 0.000 | 0.002 | 3.419 |
| LNM Group 3 vs. Group 1 | ENSG00000103522 | IL21R | −2.192 | 5.182 | −3.932 | 0.000 | 0.009 | 0.226 |
| LNM Group 3 vs. Group 1 | ENSG00000144218 | AFF3 | −2.216 | 4.918 | −3.384 | 0.002 | 0.023 | −1.245 |
| LNM Group 3 vs. Group 1 | ENSG00000117215 | PLA2G2D | −2.287 | 3.360 | −3.213 | 0.003 | 0.032 | −1.621 |
| LNM Group 3 vs. Group 1 | ENSG00000131016 | AKAP12 | −2.289 | 5.954 | −3.272 | 0.002 | 0.029 | −1.544 |
| LNM Group 3 vs. Group 1 | ENSG00000196329 | GIMAP5 | −2.515 | 5.025 | −5.204 | 0.000 | 0.001 | 3.895 |
| LNM Group 3 vs. Group 1 | ENSG00000203710 | CR1 | −2.632 | 4.585 | −3.975 | 0.000 | 0.009 | 0.359 |
| LNM Group 3 vs. Group 1 | ENSG00000090659 | CD209 | −2.731 | 4.128 | −3.788 | 0.000 | 0.011 | −0.144 |
| LNM Group 3 vs. Group 1 | ENSG00000136869 | TLR4 | −2.859 | 5.010 | −6.218 | 0.000 | 0.000 | 6.883 |
| LNM Group 3 vs. Group 1 | ENSG00000260314 | MRC1 | −3.453 | 5.521 | −5.875 | 0.000 | 0.000 | 5.868 |
| LNM Group 1 vs. Group 2 | miRNA | hsa-miR-205-5p | −0.990 | 15.614 | −3.953 | 0.000 | 0.083 | 0.246 |
| LNM Group 1 vs. Group 2 | miRNA | hsa-miR-106a-5p | 1.150 | 3.816 | 3.753 | 0.001 | 0.083 | −0.197 |
| LNM Group 2 vs. Group 3 | miRNA | hsa-miR-183-5p | −2.744 | 10.364 | −4.558 | 0.000 | 0.014 | 2.046 |
| LNM Group 2 vs. Group 3 | miRNA | hsa-miR-182-5p | −1.961 | 13.618 | −4.194 | 0.000 | 0.018 | 0.999 |
| LNM Group 2 vs. Group 3 | miRNA | hsa-miR-30a-3p | −1.753 | 8.004 | −4.094 | 0.000 | 0.018 | 0.727 |
| LNM Group 2 vs. Group 3 | miRNA | hsa-miR-877*5p | −1.906 | 6.713 | −3.980 | 0.000 | 0.018 | 0.406 |
| LNM Group 2 vs. Group 3 | miRNA | hsa-miR-30a-5p | −1.507 | 13.008 | −3.962 | 0.000 | 0.018 | 0.351 |
| LNM Group 2 vs. Group 3 | miRNA | hsa-miR-17-5p | −1.488 | 8.659 | −3.806 | 0.000 | 0.024 | −0.054 |
| LNM Group 2 vs. Group 3 | miRNA | hsa-miR-96-5p | −1.832 | 5.054 | −3.640 | 0.001 | 0.031 | −0.500 |
| LNM Group 2 vs. Group 3 | miRNA | hsa-miR-30d-5p | −1.099 | 13.929 | −3.615 | 0.001 | 0.031 | −0.594 |
| LNM Group 2 vs. Group 3 | miRNA | hsa-miR-324-5p | −1.287 | 7.058 | −3.541 | 0.001 | 0.035 | −0.738 |
| LNM Group 2 vs. Group 3 | miRNA | hsa-miR-92a-3p | −1.157 | 15.337 | −3.359 | 0.002 | 0.053 | −1.289 |
| LNM Group 2 vs. Group 3 | miRNA | hsa-miR-7706 | −1.801 | 6.906 | −3.320 | 0.002 | 0.053 | −1.284 |
| LNM Group 2 vs. Group 3 | miRNA | hsa-miR-9-5p | −2.206 | 5.610 | −3.175 | 0.003 | 0.066 | −1.633 |
| LNM Group 2 vs. Group 3 | miRNA | hsa-miR-30b-5p | −0.959 | 10.547 | −3.161 | 0.003 | 0.066 | −1.713 |
| LNM Group 2 vs. Group 3 | miRNA | hsa-miR-17-3p | −1.492 | 5.656 | −3.160 | 0.003 | 0.066 | −1.657 |
| LNM Group 2 vs. Group 3 | miRNA | hsa-miR-320a | −1.569 | 12.671 | −3.009 | 0.004 | 0.093 | −2.110 |
| LNM Group 2 vs. Group 3 | miRNA | hsa-miR-320d | −1.599 | 5.172 | −2.898 | 0.006 | 0.099 | −2.260 |
| LNM Group 2 vs. Group 3 | miRNA | hsa-miR-421 | 0.912 | 7.619 | 2.890 | 0.006 | 0.099 | −2.315 |
| LNM Group 2 vs. Group 3 | miRNA | hsa-miR-484 | −1.091 | 9.439 | −2.880 | 0.006 | 0.099 | −2.369 |
| LNM Group 2 vs. Group 3 | miRNA | hsa-miR-324-3p | −0.945 | 6.581 | −2.878 | 0.006 | 0.099 | −2.330 |
| LNM Group 2 vs. Group 3 | miRNA | hsa-miR-320c | −1.428 | 6.848 | −2.863 | 0.006 | 0.099 | −2.364 |
| LNM Group 2 vs. Group 3 | miRNA | hsa-miR-125a-5p | −1.108 | 12.943 | −2.860 | 0.007 | 0.099 | −2.461 |
| LNM Group 3 vs. Group 1 | miRNA | hsa-miR-182-5p | 2.673 | 13.618 | 5.986 | 0.000 | 0.000 | 6.429 |
| LNM Group 3 vs. Group 1 | miRNA | hsa-miR-183-5p | 3.181 | 10.364 | 5.527 | 0.000 | 0.000 | 4.986 |
| LNM Group 3 vs. Group 1 | miRNA | hsa-miR-96-5p | 2.412 | 5.054 | 5.020 | 0.000 | 0.001 | 3.420 |
| LNM Group 3 vs. Group 1 | miRNA | hsa-miR-877-5p | 2.080 | 6.713 | 4.582 | 0.000 | 0.003 | 2.108 |
| LNM Group 3 vs. Group 1 | miRNA | hsa-miR-30a-3p | 1.670 | 8.004 | 4.089 | 0.000 | 0.012 | 0.632 |
| LNM Group 3 vs. Group 1 | miRNA | hsa-miR-30a-5p | 1.437 | 13.008 | 3.952 | 0.000 | 0.015 | 0.163 |
| LNM Group 3 vs. Group 1 | miRNA | hsa-miR-324-5p | 1.226 | 7.058 | 3.551 | 0.001 | 0.036 | −0.826 |
| LNM Group 3 vs. Group 1 | miRNA | hsa-miR-324-3p | 1.106 | 6.581 | 3.549 | 0.001 | 0.036 | −0.825 |
| LNM Group 3 vs. Group 1 | miRNA | hsa-miR-17-5p | 1.321 | 8.659 | 3.531 | 0.001 | 0.036 | −0.925 |
| LNM Group 3 vs. Group 1 | miRNA | hsa-miR-205-5p | 1.378 | 15.614 | 3.354 | 0.002 | 0.053 | −1.544 |
| LNM Group 3 vs. Group 1 | miRNA | hsa-miR-30d-5p | 0.962 | 13.929 | 3.297 | 0.002 | 0.055 | −1.635 |
| LNM Group 3 vs. Group 1 | miRNA | hsa-miR-143-5p | −1.903 | 5.030 | −3.256 | 0.002 | 0.055 | −1.499 |
| LNM Group 3 vs. Group 1 | miRNA | hsa-miR-9-5p | 2.134 | 5.610 | 3.252 | 0.002 | 0.055 | −1.584 |
| LNM Group 3 vs. Group 1 | miRNA | hsa-miR-7706 | 1.652 | 6.906 | 3.190 | 0.003 | 0.061 | −1.733 |
| LNM Group 3 vs. Group 1 | miRNA | hsa-miR-320c | 1.473 | 6.848 | 3.105 | 0.003 | 0.072 | −1.971 |
| LNM Group 3 vs. Group 1 | miRNA | hsa-miR-320d | 1.604 | 5.172 | 3.056 | 0.004 | 0.077 | −2.040 |

TABLE 5
Accuracy of existing classifiers to predict Group 2 LNM subtype.

| | 172 gene | | 30 gene | | 10 gene RSi | | 13 gene OSCC | |
|---|---|---|---|---|---|---|---|---|
| | signature | | PI score | | (Eschrich | | risk score | |
| | (De Cecco | | (Enokida | | et al. 2000) | | (Lohavanichbutr | |
| | et al. 2014) | | et al. 2017) | | Non- | | et al. 2012) | |
| | High risk | Low risk | PI-high | PI-low | Responder | Responder | High | Low |
| iCluster grouping | | | | | | | | |
| Group 2 | 18 | 11 | 26 | 3 | 28 | 1 | 25 | 4 |
| Group 1&3 | 17 | 26 | 11 | 32 | 27 | 16 | 11 | 32 |
| Sensitivity | 0.621 | (0.440-0.773) | 0.897 | (0.736-0.964) | 0.966 | (0.828-0.994) | 0.862 | (0.694-0.945) |
| Specificity | 0.605 | (0.456-0.786) | 0.744 | (0.598-0.851) | 0.372 | (0.244-0.521) | 0.744 | (0.598-0.851) |
| Positive | 0.514 | (0.356-0.670) | 0.703 | (0.542-0.825) | 0.509 | (0.381-0.636) | 0.694 | (0.531-0.820) |
| predictive | | | | | | | | |
| value | | | | | | | | |
| Negative | 0.703 | (0.542-0.825) | 0.914 | (0.776-0.970) | 0.941 | (0.730-0.990) | 0.889 | (0.747-0.956) |
| predictive | | | | | | | | |
| value | | | | | | | | |
| Accuracy | 0.611 | (0.488-0.724)) | 0.806 | (0.695-0.889) | 0.611 | (6.489-0.724) | 0.791 | (0.679-0.878) |

TABLE 6
73 gene classifier

| | | | | | prop- |
|---|---|---|---|---|---|
| | | | | | se- |
| | | | | av- | lect- |
| | | | | rank- | ed- |
| | Gene | C13- | C2- | in- | in- |
| Ensemble ID | Name | score | score | CV | CV |
| ENSG00000106799 | TGFBR1 | −0.1064 | 0.1596 | 1.9 | 1 |
| ENSG00000173391 | OLR1 | −0.1062 | 0.1593 | 1.7 | 1 |
| ENSG00000145431 | PDGFC | −0.0974 | 0.1461 | 2.9 | 1 |
| ENSG00000159640 | ACE | −0.0664 | 0.0995 | 8.8 | 1 |
| ENSG00000174197 | MGA | 0.066 | −0.099 | 8 | 1 |
| ENSG00000182752 | PAPPA | −0.0656 | 0.0984 | 9.3 | 1 |
| ENSG00000163697 | APBB2 | −0.0591 | 0.0886 | 12.5 | 1 |
| ENSG00000165233 | CARD19 | −0.058 | 0.087 | 11.2 | 1 |
| ENSG00000214300 | SPDYE3 | 0.0546 | −0.0819 | 11.7 | 1 |
| ENSG00000123130 | ACOT9 | −0.0543 | 0.0815 | 13.9 | 1 |
| ENSG00000133466 | C1QTNF6 | −0.0474 | 0.0711 | 17.9 | 1 |
| ENSG00000182230 | FAM153B | 0.0449 | −0.0673 | 18.4 | 1 |
| ENSG00000222047 | C10orf55 | −0.0446 | 0.0669 | 17.9 | 1 |
| ENSG00000171612 | SLC25A33 | 0.0442 | −0.0663 | 21.2 | 1 |
| ENSG00000136504 | KAT7 | 0.0442 | −0.0633 | 18.3 | 1 |
| ENSG00000176945 | MUC20 | 0.0429 | −0.0644 | 18.8 | 1 |
| ENSG00000198018 | ENTPD7 | −0.0428 | 0.0642 | 20.4 | 1 |
| ENSG00000111711 | GOLT1B | −0.0415 | 0.0623 | 21.9 | 1 |
| ENSG00000148840 | PPRC1 | −0.0366 | 0.0549 | 26.8 | 1 |
| ENSG00000205730 | ITPRIPL2 | −0.0359 | 0.0539 | 26.6 | 1 |
| ENSG00000149289 | ZC3H12C | −0.0352 | 0.0529 | 32.7 | 0.9 |
| ENSG00000168374 | ARF4 | −0.0344 | 0.0516 | 28.9 | 1 |
| ENSG00000196739 | COL27A1 | −0.0335 | 0.0502 | 30.2 | 1 |
| ENSG00000267041 | ZNF850 | 0.0285 | −0.0428 | 34.2 | 1 |
| ENSG00000167397 | VKORC1 | −0.0276 | 0.0415 | 34.6 | 1 |
| ENSG00000100461 | RBM23 | 0.0257 | −0.0386 | 38.6 | 0.9 |
| ENSG00000042493 | CAPG | −0.0236 | 0.0354 | 44.3 | 1 |
| ENSG00000198113 | TOR4A | −0.0224 | 0.0335 | 44.6 | 0.9 |
| ENSG00000123427 | METTL21B | −0.0222 | 0.0333 | 44.1 | 1 |
| ENSG00000188917 | TRMT2B | 0.0218 | −0.0327 | 44.2 | 0.9 |
| ENSG00000072840 | EVC | −0.0205 | 0.0308 | 47.4 | 0.9 |
| ENSG00000147364 | ZBTB14 | 0.0204 | −0.0306 | 48.5 | 0.9 |
| ENSG00000113068 | PFDN1 | −0.0199 | 0.0299 | 49.3 | 0.9 |
| ENSG00000147364 | FBXO25 | 0.0194 | −0.0291 | 57.6 | 0.9 |
| ENSG00000086062 | B4GALT1 | −0.0191 | 0.0286 | 51.7 | 0.9 |
| ENSG00000148335 | NTMT1 | −0.019 | 0.0284 | 51.4 | 0.9 |
| ENSG00000166262 | FAM227B | 0.0187 | −0.028 | 49.4 | 0.9 |
| ENSG00000177674 | AGTRAP | −0.0186 | 0.0278 | 48.3 | 0.9 |
| ENSG00000126458 | RRAS | −0.0172 | 0.0258 | 51.2 | 0.9 |
| ENSG00000128578 | STRIP2 | −0.0166 | 0.0248 | 52.7 | 0.8 |
| ENSG00000126107 | HECTD3 | −0.0155 | 0.0232 | 55.8 | 1 |
| ENSG00000198478 | SH3BGRL2 | 0.0152 | −0.0229 | 58.7 | 0.8 |
| ENSG00000145685 | LHFPL2 | −0.0147 | 0.022 | 66.4 | 0.8 |
| ENSG00000147548 | WHSC1L1 | 0.0142 | −0.0212 | 55.8 | 0.8 |
| ENSG00000164465 | DCBLD1 | −0.0139 | 0.0208 | 56.8 | 0.9 |
| ENSG00000166068 | SPRED1 | −0.0132 | 0.0198 | 59.3 | 0.7 |
| ENSG00000233436 | BTBD18 | 0.0121 | −0.0181 | 67 | 0.7 |
| ENSG00000068438 | FTSJ1 | −0.0116 | 0.0174 | 69.9 | 0.7 |
| ENSG00000148834 | GSTO1 | −0.0115 | 0.0173 | 63.3 | 0.7 |
| ENSG00000167106 | FAM102A | 0.0105 | −0.0157 | 68.9 | 0.9 |
| ENSG00000099904 | ZDHHC8 | −0.0102 | 0.0154 | 66.3 | 0.7 |
| ENSG00000100311 | PDGFB | −0.0097 | 0.0146 | 67.2 | 0.8 |
| ENSG00000154079 | SDHAF4 | 0.0095 | −0.0142 | 79.3 | 0.8 |
| ENSG00000090097 | PCBP4 | −0.0093 | 0.0139 | 70.2 | 0.6 |
| ENSG00000196639 | HRH1 | −0.0092 | 0.0139 | 73.3 | 0.7 |
| ENSG00000164898 | FMC1 | 0.009 | −0.0136 | 79.7 | 0.8 |
| ENSG00000136068 | FLNB | −0.0084 | 0.0126 | 70.1 | 0.8 |
| ENSG00000129197 | RPAIN | 0.0083 | −0.0124 | 72.3 | 0.6 |
| ENSG00000127364 | TAS2R4 | 0.008 | −0.012 | 70.4 | 0.6 |
| ENSG00000108423 | TUBD1 | 0.0077 | −0.0116 | 73.6 | 0.6 |
| ENSG00000106080 | FKBP14 | −0.0075 | 0.0112 | 70.8 | 0.6 |
| ENSG00000144401 | METTL21A | −0.0067 | −0.01 | 82.3 | 0.7 |
| ENSG00000170921 | TANC2 | −0.0055 | 0.0082 | 82.9 | 0.6 |
| ENSG00000070501 | POLB | 0.005 | −0.0075 | 79.8 | 0.8 |
| ENSG00000206579 | XKR4 | 0.0043 | −0.0065 | 76.9 | 0.7 |
| ENSG00000157429 | ZNF19 | 0.0042 | −0.0062 | 77.4 | 0.6 |
| ENSG00000255346 | NOX5 | −0.0041 | 0.0061 | 90.7 | 0.7 |
| ENSG00000105856 | HBP1 | 0.0038 | −0.0057 | 86.6 | 0.6 |
| ENSG00000143443 | C1orf56 | 0.0038 | −0.0057 | 78.7 | 0.6 |
| ENSG00000157551 | KCNJ15 | −0.0037 | 0.0055 | 95.1 | 0.6 |
| ENSG00000293590 | N0OR1 | −0.0027 | 0.004 | 86.4 | 0.6 |
| ENSG00000164402 | 43351 | −0.001 | 0.0016 | 90.5 | 0.5 |
| ENSG00000087495 | PHACTR3 | 0.001 | −0.0015 | 87.7 | 0.4 |

TABLE 7
Reference Sequences for normalization.

| SEQ | | |
|---|---|---|
| ID | | |
| NO: | Name | Target Sequence (5′-3′) |
| 1 | ACE | GAAGTACGGCACCCAGGCCAGGAAGTTTGATGTGAACCAGTTGCAGAACACCAC |
| | | TATCAAGCGGATCATAAAGAAGGTTCAGGACCTAGAACGGGCAGCA |
| 2 | ACIN1 | CCTGCAGAGCATGAAGTAAAGAAAGTGACTTTAGGAGATACCTTAACTCGACGT |
| | | TCCATTAGCCAGCAGAAGTCCGGAGTTTCCATTACCATTGATGACC |
| 3 | ACOT9 | AAATTTGCCACCCTCAGGAGCGGAACATTTTCAATCGGATCTTTGGTGGTTTCC |
| | | TTATGAGGAAGGCATATGAACTTGCGTGGGCTACTGCTTGTAGCTT |
| 4 | AGTRAP | TGGCTCAGCGGGACTCCATCGACGCCATAAGCATGTTTCTGGGTGGCTTGCTGG |
| | | CCACCATCTTCCTGGACATCGTGCACATCAGCATCTTCTACCCGCG |
| 5 | ANAPC5 | GGTTACAGTACTTGGCCTCTGAAACAGTGCTGAACTTGGCTTTTGCGCAGCTCA |
| | | TTCTTGGAATCCCAGAACAGGCCTTAAGTCTTCTCCACATGGCCAT |
| 6 | APBB2 | AAATTCCTTACGCAGTGGTATTCATGATGGTGCCCTATCTAAGTCCAGGACTGT |
| | | TTTCCTACAGCGTGCCTCAAAAGTGTTGTAGAGGGCAGGATTCTAC |
| 7 | ARF4 | GTCTCTTCGTAACAGAACATGGTATGTTCAAGCCACTTGTGCAACACAAGGAAC |
| | | TGGTCTGTATGAAGGACTTGACTGGCTGTCAAATGAGCTTTCAAAA |
| 8 | ATF2 | GTGGGAGCCCGTGGCCTTTAAAGTGCCGTTCAGCCTTTTCCTCCAGGGGTGCTT |
| | | TGTAAACACGGCTGTGCTCAGGGCTCGCGGGTGACCGAAAGGATCA |
| 9 | ATXN2L | TTCAATGAGGAGAACTACGGTGTGAAGACTACCTATGATAGCAGTCTTTCTTCT |
| | | TATACGGTGCCCTTAGAAAAGGACAACTCAGAAGAGTTTCGTCAGC |
| 10 | B4GALT1 | ACACAAAGGAGACAATGCTCTCTGATGGTTTGAACTCACTCACCTACCAGGTGC |
| | | TGGATGTACAGAGATACCCATTGTATACCCAAATCACAGTGGACAT |
| 11 | BTBD18 | CTTGAAGATTATACCTGTGGAGCTGGGATGTGGTGCTCAGGAAGAATATAAGTA |
| | | GTCAGCATTGTAGCAGTTGAGTCTCAGTGGTAATGGAAATGAGGAA |
| 12 | C10orf55 | TTATTGCTTTCGGCAGTAAGGGGGTTTGTTTGATGGTGCTATCAGAGGGGGAAA |
| | | GGCAAGGCCAGATTACTGAAAATTTGCAGCTTGGTTTAAAGTCCGT |
| 13 | C10orf56 | GCTTTTTGGAAACGGGTCAGGATTGGCCTGGAGGATATTTGGAATAGCCTCTCT |
| | | TCAGTGTTCACAGAGATGCAACCAATAGACAGAAACCAGAGGTAAT |
| 14 | C1QTNF6 | CCCTGCACAGCGGCGAGGACTTCCAGACGCTGCTCTTCGAAAGGGTCTTTGTGA |
| | | ACCTTGATGGGTGCTTTGACATGGCGACCGGCCAGTTTGCTGCTCC |
| 15 | CAPG | TGACTCCAGCCCATTTGCCCTTGAACTGCTGATATCTGATGACTGCTTTGTGCT |
| | | GGACAACGGGCTCTGTGGCAAGATCTATATCTGGAAGGGGCGAAAA |
| 16 | CARD19 | CCTGGTGCAGGACACGCCTTTCCTGACAGGCCATGGGCGCTTGAGTGAGCAGCA |
| | | GGTGGACAGGATCATCCTCCAGCTGAACCGTTACTACCCACAGATC |
| 17 | COL27A1 | CACCGCCCAGCACCCGGCCCCTACCTCCTACCACCAGCTCCTCTAAAAAACCCA |
| | | TTCCCACACTAGCTCGGACTGAGGCCAAGATAACCAGCCATGCCAG |
| 18 | DCBLD1 | CTCTTGTTGAACACAAGTGAAGTAACCGTCCGCTTTGAGAGTGGATCCCACATT |
| | | TCTGGCCGGGGTTTTTTGCTGACCTATGCGAGCAGCGACCATCCAG |
| 19 | DCTN4 | CTTCCAGCCAGTCTGTGCTTCACAGCTCTATCCTCGCCACAAACATCTTCTGAT |
| | | CAAACGGTCCCTGCGCTGCCGTAAATGTGAACATAATTTGAGCAAG |
| 20 | METTL21B | CTGTTCTCAGAGAAGGAGGATGGTAAGGTATCCAGGATTTTTAGGGGGTAAGGG |
| | | AGATATATGGGATGTGAGAGCAGTGATTGTAGACAGACTGTCACTG |
| 21 | ENTPD7 | GGGAGCCATTCTATATAAAACACGATTCTTACCACTCAGGGATCTTCGGCAGGA |
| | | AGGTGTCCGACAAGCCCATGGTAGCTGGTTCCGTCTCTCCTTTGTA |
| 22 | EVC | TTTCTTGTGGACCAGTTTAAGTGTTCCAGCTCCAAAGCCCGACAGCTGATGATG |
| | | ACTCTGACGGAAAGAATGATTGCAGCCGAAGGGCTATTGTGCGATT |
| 23 | EWSR1 | GAACCTATGGACAGCCCACTGATGTCAGCTATACCCAGGCTCAGACCACTGCAA |
| | | CCTATGGGCAGACCGCCTATGCAACTTCTTATGGACAGCCTCCCAC |
| 24 | FAM102A | CTGGAGAAGAGTCCTGCTTTCTCTGGAGTCCAGACCTGTATCATTCCATGAGGA |
| | | ACTTTCCCCTTCAGATCACCTCTGCGCCACATCTCATCCATGCCTC |
| 25 | FAM153B | CAGGGGTCCTTCAGCCTCTGTGGGGGCCTGGGGTGGCCTCATGGCCCCATTTTT |
| | | CAAGATGAGGTTGGAGGCTTCTGTATGTAGAGGGCACTGGCTTGGG |
| 26 | FAM227B | CCGCCTCAAGCCTCGCAGCCTTTCACCCAGTTTCCTCCCGATTCTTGCTGGGAC |
| | | AACAAAATTGGGAAGCGGTTCTCGAAACATACTGTCAAGGCGTAGT |
| 27 | FBXO25 | TCTTCAGATGACTAAGCAAGTGAACAATGGCCTCACCCTCAGTGACCTTCCTCT |
| | | GCACATGCTGAACAACATCCTATACCGGTTCTCAGACGGATGGGAC |
| 28 | FKBP14 | TTATGTGTAACTGGCTCTGAGTCTCTGCTTGAGGACCAGAGGAAAATGGTTGTT |
| | | GGACCTGACTTGTTAATGGCTACTGCTTTACTAAGGAGATGTGCAA |
| 29 | FLNB | GGTTTGGAGAAATCTGGATGCATTGTCAACAACCTGGCCGAGTTCACTGTGGAT |
| | | CCTAAGGATGCTGGAAAAGCTCCCTTAAAGATATTTGCTCAGGATG |
| 30 | FMC1 | GAGTCTGCTGGCTTGGTGGGTCTCAAGTTGCCCCATCAGCCTGGAGGGAAGGGC |
| | | TGGGAGCCATGAACATGGAGAATATCCTTGGATGCTGCATTCATAG |
| 31 | FTSJ1 | GATGACGGGGCTCACGTTGTACGTTCACATCAGGTCCCGGCCCGCCGGAACCTG |
| | | GGCGATCCACGATGCCGAGTTTGCCACGCTGCGACAGCCCATAGGC |
| 32 | GOLT1B | TCCGGAAGACGTGGCGGCTCTCGCCTGGGCTGTTTCCCGGCTTCATTTCTCCCG |
| | | ACTCAGCTTCCCACCCTGGGCTTTCCGAGGTGCTGTCGCCGCTGTC |
| 33 | GSTO1 | TCTGACTAATAAGAAGACGACCTTCTTTGGTGGCAATTCTATCTCTATGATTGA |
| | | TTACCTCATCTGGCCCTGGTTTGAACGGCTGGAAGCAATGAAGTTA |
| 34 | HBP1 | CTTGTGGAGGACCTGGTGGTCAAGACTTTGCAAGATCTGGATTCAGTAAAAACT |
| | | GTGGCTCACCTGGATCATCACAGCTCTCTTCCAATTCTTTGTATGC |
| 35 | HECTD3 | CCGTCCACCTCCCGATCATCGAGATCCGCATCGTGGAGTGCCGAGATGATGGGA |
| | | TTGATGTTCGTCTCCGAGGGGTCAAGATCAAGTCATCTAGACAGCG |
| 36 | HRH1 | GTGGCAGCTCAAAATGATATGTTTGAGTAGACGAACAGCTGACATGGAGTTCCC |
| | | GTGCACCTACGGAAGGGGACGCTTTGAAGGAACCAAGTGCATTTTT |
| 37 | ITPRIPL2 | TTCTGACTCAGCAGCTCCACCAAAATTGACATCCTAGCAAACACTGTGAAGGAA |
| | | TTAACCTAAGTGCTTCCAGAGCATCTCATGTAACCTCTATGGAGTA |
| 38 | KAT7 | TCCAGTTACTCCTTATATGTGTGTTCTATGGAGGGGCAGGGAAAAGGTGGCACT |
| | | TGTGAGTGTGTGTGGATTGGCAGGGGGTCCATTCACTTTGGGTTCC |
| 39 | KCNJ15 | AGACCCGCCACATCAGCACCATCCGAGAGCTTATTAGTAATGTGTAGATTCTCA |
| | | TGTCCCAGCCAGACTTACAGAATTGGGGTCTGTATCTTAACAAGAA |
| 40 | KIAA0100 | ATGGTTCTCAAGGTGGATACCTCTGAGTCCTTATGGCATATTCAGATCAGTAGA |
| | | AGCAGATTTCTTTTGGATAGTGATGGGAAAAGGCTAATCTGTGAGG |
| 41 | LHFPL2 | TGATTAACTCCGTGGACTCCTGACTCTTTCTTCGCCCGGAACATCAATATGTGT |
| | | CATGTCATTGTCACCTGTCGCTCGATGCTCTGGACCTTGCTGAGTA |
| 42 | METTL21A | TCGTGCCCTATGAGGAGACCACGGAATTTGGGTTGCAGAAATTCCACAAGCCTC |
| | | TTGCAACTTTTTCCTTTGCAAACCACACGATCCAGATCCGGCAGGA |
| 43 | MGA | TCCAAGTGGAATGAACTTATTCAGGCACCCTAATGGGCAGATTGTCCAGCTTCT |
| | | ACCTTTGCATCAGCTTCGAGGCTCTAATACCCAGCCCAACTTACAG |
| 44 | MUC20 | TTGAGACACCAAGTTACGTCAAAGTCTCAGGAGCAGCTCCGGTCTCCATAGAGG |
| | | CTGGGTCAGCAGTGGGCAAAACAACTTCCTTTGCTGGGAGCTCTGC |
| 45 | NCOR1 | TAGGAGTGAGCATGAGATTTCTGAAATTATTGATGGGCTCTCTGAGCAGGAGAA |
| | | TAATGAGAAACAAATGCGGCAGCTCTCTGTGATTCCACCTATGATG |
| 46 | NDOR1 | ACCTGCCCTCCACTGCCCTCTGTCAGATGGACTTTGCCGTCCTGGGCCTCGGGG |
| | | ACTCCTCATACGCCAAGTTCAACTTCGTGGCCAAGAAGCTGCACCG |
| 47 | NOX5 | AGCTCCATAAGGTGGACTTTATCTGGATCAACAGAGACCAGCGGTCTTTCGAGT |
| | | GGTTTGTGAGCCTGCTGACTAAACTGGAGATGGACCAGGCCGAGGA |
| 48 | WHSC1L1 | GAAAGGGAAAGTTAAGGATGCTGGAGCAGAACAATGGATTTCTCTTTCTCTTTC |
| | | ATGCAAGGGATCATGGGAAACACAATTCAGCAACCACCTCAACTCA |
| 49 | NTMT1 | AGGATCACCAAGCGGCTGCTCCTGCCGCTGTTCAGAGAGGTGGATATGGTCGAC |
| | | ATAACGGAGGACTTCCTGGTTCAAGCCAAGACCTACCTGGGGGAGG |
| 50 | OLR1 | CCCAGGTGTCTGACCTCCTAACACAAGAGCAAGCAAACCTAACTCACCAGAAAA |
| | | AGAAACTGGAGGGACAGATCTCAGCCCGGCAACAAGCAGAAGAAGC |
| 51 | PAPPA | CAATGCGACGACATGAATAAGATCAATGGTGATGGCTGCTCCCTTTTCTGCCGA |
| | | CAAGAAGTCTCCTTCAATTGTATTGATGAACCCAGCCGGTGCTATT |
| 52 | PCBP4 | CATCACCGGGTCTACAGCAGCTGTCTTCCATGCAGTCTCCATGATTGCTTTCAA |
| | | ACTGGATGAGGACCTTTGTGCTGCTCCTGCAAATGGTGGAAATGTC |
| 53 | PDGFB | GATTCACCTCTTCCTCTGGTTCCTTTCATCTCTCTACCTCCACCCTGCATTTTC |
| | | CTCTTGTCCTGGCCCTTCAGTCTGCTCCACCAAGGGGCTCTTGAAC |
| 54 | PDGFC | GCAACTTATGAAAGTAGACATTCAGATCCAGCCATTACTAACCTATTCCTTTTT |
| | | TGGGGAAATCTGAGCCTAGCTCAGAAAAACATAAAGCACCTTGAAA |
| 55 | PFDN1 | TGAGAGAGGCAGTGGCCGTCTCCACCTCAGCTCCTGCTCCCTCTGCATCAGAGC |
| | | CCTTCCTTTCTTGGGGGATGGGCCTTGCCCTCTTCTCTTTTCCCTT |
| 56 | PHACTR3 | GGAGAACAAGGAGAACCTGATCATAAATTCTGAACTCAAAGACGACTTGCTTTT |
| | | GTATCAGGACGAGGAGGCGCTGAACGACTCCATTATTTCTGGAACA |
| 57 | POLB | TCTGAATACATTGCTACAGTCTGTGGCAGTTTCAGAAGAGGTGCAGAGTCCAGT |
| | | GGTGACATGGATGTTCTCCTGACCCATCCCAGCTTCACTTCAGAAT |
| 58 | PPRC1 | AGAGAAGTGCTGGACAAAGTAGTCCTGCTAAAGAAGGCCCTCTAGACCTCTACC |
| | | CAAAGCTGGCTGACACTATCCAAACCAATCCTATACCAACCCATCT |
| 59 | RAB5B | TGTGGATCTCCATGAACAGTCCCAGCAGAACAAGAGCCAGTGTTGTAGCAACTG |
| | | AGGGGGTGGCTAGCAGCAAACAAGTATGGAGCTAGCACAAGAGCTA |
| 60 | RBM23 | TCAATATCACTGAAGACATGCTCCGGGGCATCTTTGAGCCCTTTGGTAAAATTG |
| | | ATAATATTGTCCTGATGAAGGACTCAGATACAGGCCGCTCTAAAGG |
| 61 | RPAIN | CTGAGACTTGTGTATCTAATTGCCTATTGGACACCTCCATCTGGGTAAACCCAG |
| | | GCATCCAGAACTTTCCCTCAAGACTTACCTCCCTCAGTGCAGGTCG |
| 62 | RRAS | CAGTACATGCGTGCTGGCCACGGCTTCCTGCTGGTGTTCGCCATTAACGACCGG |
| | | CAGAGTTTCAACGAGGTGGGCAAGCTCTTCACGCAGATTCTGCGGG |
| 63 | SDHAF4 | AGTCTGAACTTGTCAAACAGTCCCTTAAGAAGCCGAAGTTACCAGAAGGTCGTT |
| | | TTGATGCACCAGAGGATTCCCATTTAGAGAAAGAACCACTGGAAAA |
| 64 | 8-Sep | TCGATGCGCAGTTTGAAAATTATCTGCAGGAGGAGCTGAAGATCCGCCGCTCGC |
| | | TCTTCGACTACCATGACACAAGGATCCACGTTTGCCTCTACTTCAT |
| 65 | SH3BGRL2 | CAGGTGTACTTTCTTAATGACTGAAAGATAAGTGGGTAGCACCGTCAGAGAGAA |
| | | AATGTTGGATCTGCCCTAGGTTATAGTAGATGCCGGAATTGCGTAA |
| 66 | SLC25A33 | CGCCTGGTGTTCCGGGAAGAAGGCTACCTTGCCTTTTATAGAGGACTGTTTGCC |
| | | CAGCTTATCCGGCAGATCCCAAATACTGCCATTGTGTTGTCTACTT |
| 67 | SPDYE3 | AACAAAAGATCTTCTACTTCCTGTACGGGAAGACCCACTCTCACATACCCTTGC |
| | | GCCCTAAGCATTGGTTCCAGTTATGCCGTCCCATGAACCCGAGGGC |
| 68 | SPEN | CTATCCAGCTCGAGGGAGAGAGTTTTATTCAGAATGGGAAACTTACCAAGGAGA |
| | | CTACTATGAATCACGATACTACGATGATCCTCGGGAATACAGGGAT |
| 69 | SPRED1 | TTCAGAGCAGAAGTATGGAATACGTACAGCGGCAAATATCCAAGGAATGTGGAA |
| | | GCCTAAAGTCCCAAAATAGGGTCCCTTTGAAATCAATCAGACATGT |
| 70 | SRRM1 | CCCGACCACGATCTCGGTCACGCTCCAAATCAAGATCCCGGACGCGGTCCCGCT |
| | | CTCCTTCTCACACTCGACCTAGACGGCGCCATAGATCCCGATCAAG |
| 71 | STRIP2 | AGCCGAGTTGTCAGAATTGTATAGTTACACTGAGAACCTGGAATTCACCAATAA |
| | | CAGGAGGTGCTTTGAAGAAGATTTCAAGACTCAAGTGCAGGGCAAG |
| 72 | TANC2 | AGGGTTTGAGTAAAAAAGTTGGTGTATCATCCTCCATCCTCCAAGGTCTCTGGA |
| | | TCTCTTATAGCACAGAAGGTCTTTCCATGGCACTGGCGTCTTTACG |
| 73 | TAS2R4 | GGACTCGAGCAGTGTCTGGTTTGTGACCTTGCTCAATATCTTGTACTGTGTGAA |
| | | GATTACTAACTTCCAACACTCAGTGTTTCTCCTGCTGAAGCGGAAT |
| 74 | TGFBR1 | GAATCCTTCAAACGTGCTGACATCTATGCAATGGGCTTAGTATTCTGGGAAATT |
| | | GCTCGACGATGTTCCATTGGTGGAATTCATGAAGATTACCAACTGC |
| 75 | TOR4A | TCTGGTCTGGCCTTCGTGTTGGCTTCAAGGTGGTAACAGAGAGAGCTGCAGAGT |
| | | GGGGGTGTGTTTGCCAGTCCCTGCATGGGTGAATGTTGGGGTGCCC |
| 76 | TRIP12 | ATGTCCAACCGGCCTAATAACAATCCAGGGGGGTCACTGCGACGTTCACAGAGG |
| | | AACACTGCCGGGGCCCAACCACAAGACGACTCAATAGGAGGAAGAA |
| 77 | TRMT2B | AGTACCATGACCCGTTGCAGCCATCAGCAGTCTCCCTATCAGCTTCTGTTTGGG |
| | | GAACCCTACATCTTTGAAGAACTTCTGAGCTTGAAGATCCGCATCT |
| 78 | TUBD1 | GCCATCCATAAGATCTGTGCAAAACTGATGAATATCAAGCAGATCTCCTTTAGT |
| | | GATATCAATCAAGTCCTCGCACATCAGCTGGGAAGTGTGTTCCAGC |
| 79 | VKORC1 | AAGAGGCACTGAGCCCTCAACCCAAGCCAGGCTGACCTCATCTGCTTTGCTTTG |
| | | GCATGTGAGCCTTGCCTAAGGGGGCATATCTGGGTCCCTAGAAGGC |
| 80 | WDR26 | GGAGGCACTTCAAGTTCTACGCTGTGAATTGACGCCGCTGAAATACAATACAGA |
| | | GCGCATTCATGTTCTTAGTGGGTATCTGATGTGTAGCCATGCAGAA |
| 81 | XKR4 | CCACATCTTGCAGCTCGGGCAAATCTGGAGATATTTCCACACAATATACTTAGG |
| | | TATTCGAAGCCGACAGAGTGGGGAGAATGACAGATGGAGGTTTTAC |
| 82 | ZBTB14 | GTTTGAGTATTTGCTTTATGGTCACCATCGGGAGCAGATTGCCTGCCAGGCGTG |
| | | TGGGAAGACGTTTTCTGATGAAGGCAGATTGAGGAAGCATGAGAAA |
| 83 | ZC3H12C | CAAATCTGAGTCTCTCAGGCCCACGAAGCCCTGAAAGGCGTTTCTCCTTAGACA |
| | | CAGATTATAGAATAAGTTCCGTAGCTTCTGACTGCAGCAGTGAAGG |
| 84 | ZDHHC8 | ACCACTGCCCCTGGGTCAACAACTGCATCGGGCGTCGAAACTATCGCTACTTCT |
| | | TCCTGTTCCTGCTGTCACTCAGTGCACACATGGTGGGCGTCGTGGC |
| 85 | ZNF19 | GGGAAAGTCTTTAGTTATAGTCATGGCAGCTCCTCAACCCCATGATTGTGATGA |
| | | GATTGGGAAGCCCACTGTAACACTGGAGAGCCCAGGTTATATCAGA |
| 86 | ZNF207 | AAGCCTACATTCCCTGCTTATACACAGTCTACAGCTTCAACAACTAGTACAACA |
| | | AATAGTACTGCAGCTAAACCAGCGGCTTCAATAACAAGTAAGCCTG |
| 87 | ZNF850 | GGTGAATAAATGGAGCCAATAGACTTGTTCACTGCATGGTTACGTCAGAGGCGT |
| | | GTAAACCAGAGCAACTTCTTCTTGAATAGGAGCTGTGTAAAGTAAC |

### REFERENCES

1. Pereira E R, Kedrin D, Seano G, Gautier O, Meijer E F J, Jones D, et al. Lymph node metastases can invade local blood vessels, exit the node, and colonize distant organs in mice. Science 2018; 359(6382): 1403-7 doi 10.1126/science.aal3622.
2. Sleeman J, Schmid A, Thiele W. Tumor lymphatics. Seminars in cancer biology 2009; 19(5):285-97 doi 10.1016/j.semcancer.2009.05.005.
3. Weinberg R A. Mechanisms of malignant progression. Carcinogenesis 2008; 29(6):1092-5 doi 10.1093/carcin/bgn104.
4. Gervasoni J E, Jr., Sbayi S, Cady B. Role of lymphadenectomy in surgical treatment of solid tumors: an update on the clinical data. Annals of surgical oncology 2007; 14(9):2443-62 doi 10.1245/s10434-007-9360-5.
5. Leemans C R, Tiwari R, Nauta J J, van der Waal I, Snow G B. Recurrence at the primary site in head and neck cancer and the significance of neck lymph node metastases as a prognostic factor. Cancer 1994; 73(1):187-90.
6. Ranck M C, Abundo R, Jefferson G, Kolokythas A, Wenig B L, Weichselbaum R R, et al. Effect of postradiotherapy neck dissection on nonregional disease sites. JAMA otolaryngology—head & neck surgery 2014; 140(1):12-21 doi 10.1001/jamaoto.2013.5754.
7. Carter C L, Allen C, Henson D E. Relation of tumor size, lymph node status, and survival in 24,740 breast cancer cases. Cancer 1989; 63(1):181-7.
8. Chen Z L, Perez S, Holmes E C, Wang H J, Coulson W F, Wen D R, et al. Frequency and distribution of occult micrometastases in lymph nodes of patients with non-small-cell lung carcinoma. Journal of the National Cancer Institute 1993; 85(6):493-8.
9. Kim D Y, Seo K W, Joo J K, Park Y K, Ryu S Y, Kim H R, et al. Prognostic factors in patients with node-negative gastric carcinoma: a comparison with node-positive gastric carcinoma. World journal of gastroenterology 2006; 12(8):1182-6.
10. Noguti J, De Moura C F, De Jesus G P, Da Silva V H, Hossaka T A, Oshima C T, et al. Metastasis from oral cancer: an overview. Cancer genomics & proteomics 2012; 9(5):329-35.
11. Cerezo L, Millan I, Torre A, Aragon G, Otero J. Prognostic factors for survival and tumor control in cervical lymph node metastases from head and neck cancer. A multivariate study of 492 cases. Cancer 1992; 69(5):1224-34.
12. Ho A S, Kim S, Tighiouart M, Gudino C, Mita A, Scher K S, et al. Metastatic Lymph Node Burden and Survival in Oral Cavity Cancer. Journal of clinical oncology: official journal of the American Society of Clinical Oncology 2017; 35(31):3601-9 doi 10.1200/JC0.2016.71.1176.
13. Rudoltz M S, Benammar A, Mohiuddin M. Does pathologic node status affect local control in patients with carcinoma of the head and neck treated with radical surgery and postoperative radiotherapy? International journal of radiation oncology, biology, physics 1995; 31(3):503-8 doi 10.1016/0360-3016(94)00394-Z.
14. Gil Z, Carlson D L, Boyle J O, Kraus D H, Shah J P, Shaha A R, et al. Lymph node density is a significant predictor of outcome in patients with oral cancer. Cancer 2009; 115(24):5700-10 doi 10.1002/cncr.24631.
15. Mamelle G, Pampurik J, Luboinski B, Lancar R, Lusinchi A, Bosq J. Lymph node prognostic factors in head and neck squamous cell carcinomas. American journal of surgery 1994; 168(5):494-8.
16. Colella S, Richards K L, Bachinski L L, Baggerly K A, Tsavachidis S, Lang J C, et al. Molecular signatures of metastasis in head and neck cancer. Head & neck 2008; 30(10):1273-83 doi 10.1002/hed.20871.
17. Roepman P, de Jager A, Groot Koerkamp M J, Kummer J A, Slootweg P J, Holstege F C. Maintenance of head and neck tumor gene expression profiles upon lymph node metastasis. Cancer research 2006; 66(23):11110-4 doi 10.1158/0008-5472.CAN-06-3161.
18. Shah J P, Candela F C, Poddar A K. The patterns of cervical lymph node metastases from squamous carcinoma of the oral cavity. Cancer 1990; 66(1):109-13.
19. Shen R, Olshen A B, Ladanyi M. Integrative clustering of multiple genomic data types using a joint latent variable model with application to breast and lung cancer subtype analysis. Bioinformatics 2009; 25(22):2906-12 doi 10.1093/bioinformatics/btp543.
20. Subramanian A, Tamayo P, Mootha V K, Mukherjee S, Ebert B L, Gillette M A, et al. Gene set enrichment analysis: a knowledge-based approach for interpreting genome-wide expression profiles. Proceedings of the National Academy of Sciences of the United States of America 2005; 102(43):15545-50 doi 10.1073/pnas.0506580102.
21. Alhamdoosh M, Ng M, Wilson N J, Sheridan J M, Huynh H, Wilson M J, et al. Combining multiple tools outperforms individual methods in gene set enrichment analyses. Bioinformatics 2017; 33(3):414-24 doi 10.1093/bioinformatics/btw623.
22. Tobin N P, Harrell J C, Lovrot J, Egyhazi Brage S, Frostvik Stolt M, Carlsson L, et al. Molecular subtype and tumor characteristics of breast cancer metastases as assessed by gene expression significantly influence patient post-relapse survival. Annals of oncology: official journal of the European Society for Medical Oncology 2015; 26(1):81-8 doi 10.1093/annonc/mdu498.
23. Cirenajwis H, Ekedahl H, Lauss M, Harbst K, Carneiro A, Enoksson J, et al. Molecular stratification of metastatic melanoma using gene expression profiling: Prediction of survival outcome and benefit from molecular targeted therapy. Oncotarget 2015; 6(14):12297-309 doi 10.18632/oncotarget.3655.
24. Tibshirani R, Hastie T, Narasimhan B, Chu G. Diagnosis of multiple cancer types by shrunken centroids of gene expression. Proceedings of the National Academy of Sciences of the United States of America 2002; 99(10):6567-72 doi 10.1073/pnas.082099299.
25. De Cecco L, Bossi P, Locati L, Canevari S, Licitra L. Comprehensive gene expression meta-analysis of head and neck squamous cell carcinoma microarray data defines a robust survival predictor. Annals of oncology: official journal of the European Society for Medical Oncology 2014; 25(8):1628-35 doi 10.1093/annonc/mdu173.
26. Enokida T, Fujii S, Takahashi M, Higuchi Y, Nomura S, Wakasugi T, et al. Gene expression profiling to predict recurrence of advanced squamous cell carcinoma of the tongue: discovery and external validation. Oncotarget 2017; 8(37):61786-99 doi 10.18632/oncotarget.18692.
27. Eschrich S A, Pramana J, Zhang H, Zhao H, Boulware D, Lee J H, et al. A gene expression model of intrinsic tumor radiosensitivity: prediction of response and prognosis after chemoradiation. International journal of radiation oncology, biology, physics 2009; 75(2):489-96 doi 10.1016/j.ijrobp.2009.06.014.
28. Lohavanichbutr P, Mendez E, Holsinger F C, Rue T C, Zhang Y, Houck J, et al. A 13-gene signature prognostic of HPV-negative OSCC: discovery and external validation. Clinical cancer research: an official journal of the American Association for Cancer Research 2013; 19(5):1197-203 doi 10.1158/1078-0432.CCR-12-2647.
29. Weigelt B, Glas A M, Wessels L F, Witteveen A T, Peterse J L, van't Veer L J. Gene expression profiles of primary breast tumors maintained in distant metastases. Proceedings of the National Academy of Sciences of the United States of America 2003; 100(26):15901-5 doi 10.1073/pnas.2634067100.
30. Hensen E F, De Herdt M J, Goeman J J, Oosting J, Smit V T, Cornelisse C J, et al. Gene-expression of metastasized versus non-metastasized primary head and neck squamous cell carcinomas: a pathway-based analysis. BMC cancer 2008; 8:168 doi 10.1186/1471-2407-8-168.
31. Ginos M A, Page G P, Michalowicz B S, Patel K J, Volker S E, Pambuccian S E, et al. Identification of a gene expression signature associated with recurrent disease in squamous cell carcinoma of the head and neck. Cancer research 2004; 64(1):55-63.
32. Mes S W, Te Beest D, Poli T, Rossi S, Scheckenbach K, van Wieringen W N, et al. Prognostic modeling of oral cancer by gene profiles and clinicopathological co-variables. Oncotarget 2017; 8(35):59312-23 doi 10.18632/oncotarget.19576.
33. Tonella L, Giannoccaro M, Alfieri S, Canevari S, De Cecco L. Gene Expression Signatures for Head and Neck Cancer Patient Stratification: Are Results Ready for Clinical Application? Current treatment options in oncology 2017; 18(5):32 doi 10.1007/s11864-017-0472-2.
34. Chung C H, Parker J S, Karaca G, Wu J, Funkhouser W K, Moore D, et al. Molecular classification of head and neck squamous cell carcinomas using patterns of gene expression. Cancer cell 2004; 5(5):489-500.
35. Keck M K, Zuo Z, Khattri A, Stricker T P, Brown C D, Imanguli M, et al. Integrative analysis of head and neck cancer identifies two biologically distinct HPV and three non-HPV subtypes. Clinical cancer research: an official journal of the American Association for Cancer Research 2015; 21(4):870-81 doi 10.1158/1078-0432.CCR-14-2481.
36. Roepman P, Wessels L F, Kettelarij N, Kemmeren P, Miles A J, Lijnzaad P, et al. An expression profile for diagnosis of lymph node metastases from primary head and neck squamous cell carcinomas. Nature genetics 2005; 37(2):182-6 doi 10.1038/ng1502.
37. Chung C H, Parker J S, Ely K, Carter J, Yi Y, Murphy B A, et al. Gene expression profiles identify epithelial-to-mesenchymal transition and activation of nuclear factor-kappaB signaling as characteristics of a high-risk head and neck squamous cell carcinoma. Cancer research 2006; 66(16):8210-8 doi 10.1158/0008-5472.CAN-06-1213.
38. De Cecco L, Nicolau M, Giannoccaro M, Daidone M G, Bossi P, Locati L, et al. Head and neck cancer subtypes with biological and clinical relevance: Meta-analysis of gene-expression data. Oncotarget 2015; 6(11):9627-42 doi 10.18632/oncotarget.3301.
39. Gerlinger M, Rowan A J, Horswell S, Math M, Larkin J, Endesfelder D, et al. Intratumor heterogeneity and branched evolution revealed by multiregion sequencing. The New England journal of medicine 2012; 366(10):883-92 doi 10.1056/NEJMoa1113205.
40. Vecchi M, Confalonieri S, Nuciforo P, Vigano M A, Capra M, Bianchi M, et al. Breast cancer metastases are molecularly distinct from their primary tumors. Oncogene 2008; 27(15):2148-58 doi 10.1038/sj.onc.1210858.
41. Suzuki M, Tarin D. Gene expression profiling of human lymph node metastases and matched primary breast carcinomas: clinical implications. Molecular oncology 2007; 1(2):172-80 doi 10.1016/j.molonc.2007.03.005.
42. Cerutti J M, Oler G, Michaluart P, Jr., Delcelo R, Beaty R M, Shoemaker J, et al. Molecular profiling of matched samples identifies biomarkers of papillary thyroid carcinoma lymph node metastasis. Cancer research 2007; 67(16):7885-92 doi 10.1158/0008-5472.CAN-06-4771.
43. Hedberg M L, Goh G, Chiosea S I, Bauman J E, Freilino M L, Zeng Y, et al. Genetic landscape of metastatic and recurrent head and neck squamous cell carcinoma. The Journal of clinical investigation 2016; 126(1):169-80 doi 10.1172/JCI82066.
44. Walter V, Yin X, Wilkerson M D, Cabanski C R, Zhao N, Du Y, et al. Molecular subtypes in head and neck cancer exhibit distinct patterns of chromosomal gain and loss of canonical cancer genes. PloS one 2013; 8(2):e56823 doi 10.1371/journal.pone.0056823.
45. Cancer Genome Atlas N. Comprehensive genomic characterization of head and neck squamous cell carcinomas. Nature 2015; 517(7536):576-82 doi 10.1038/nature14129.
46. Kimbung S, Johansson I, Danielsson A, Veerla S, Egyhazi Brage S, Frostvik Stolt M, et al. Transcriptional Profiling of Breast Cancer Metastases Identifies Liver Metastasis-Selective Genes Associated with Adverse Outcome in Luminal A Primary Breast Cancer. Clinical cancer research: an official journal of the American Association for Cancer Research 2016; 22(1):146-57 doi 10.1158/1078-0432.CCR-15-0487.

### SUPPLEMENTARY REFERENCES

1. Charlson M E, Pompei P, Ales K L, MacKenzie CR. A new method of classifying prognostic comorbidity in longitudinal studies: development and validation. J Chronic Dis. 1987; 40: 373-383.
2. Karnofsky D A, Burchenal J. The Clinical Evaluation of Chemotherapeutic Agents in Cancer. Columbia Univ Press, 1949.
3. Patro R, Duggal G, Love M I, Irizarry R A, Kingsford C. Salmon provides fast and bias-aware quantification of transcript expression. Nat Methods. 2017; 14: 417-419.
4. Soneson C, Love M I, Robinson M D. Differential analyses for RNA-seq: transcript-level estimates improve gene-level inferences. F1000Res. 2015; 4: 1521.
5. Friedlander M R, Chen W, Adamidi C, et al. Discovering microRNAs from deep sequencing data using miRDeep. Nat Biotechnol. 2008; 26: 407-415.
6. Robinson M D, McCarthy D J, Smyth G K. edgeR: a Bioconductor package for differential expression analysis of digital gene expression data. Bioinformatics. 2010; 26: 139-140.
7. Ritchie M E, Phipson B, Wu D, et al. limma powers differential expression analyses for RNA-sequencing and microarray studies. Nucleic Acids Res. 2015; 43: e47.
8. Wilkerson M D, Hayes D N. ConsensusClusterPlus: a class discovery tool with confidence assessments and item tracking. Bioinformatics. 2010; 26: 1572-1573.
9. Senbabaoglu Y, Michailidis G, Li J Z. Critical limitations of consensus clustering in class discovery. Sci Rep. 2014; 4: 6207.

1: A method of treating cancer in a subject, the method comprising:
(a) obtaining a sample comprising lymph node metastasis (LNM) cells from the subject;
(b) measuring gene expression levels in the sample of at least ten genes selected from the group consisting of: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3;
(c) determining a LNM-specific molecular subtype comprising the gene expression levels of the at least ten genes;
(d) providing an indication as to the outcome of locoregional control (LRC), relapse-free survival (RFS), and/or overall survival (OS) when the LNM-specific molecular subtype indicates that the expression levels of the at least ten genes are altered in a predictive manner; and
(e) administering an aggressive cancer treatment regimen to the subject when a determination is made that the subject has LNM cells with decreased LRC, decreased RFS, and/or decreased OS.

2: The method of claim 1, wherein measuring gene expression levels of the at least ten genes comprises measurement by RNAseq, miRNAseq, RT-PCR, microRNA, nanostring, northern blot, qRT-PCR, in situ hybridization, immunohistochemistry, or Western blotting.

3: The method of claim 1, wherein the LNM cells from the subject are obtained from a primary cancer selected from the group consisting of: human papillomavirus-negative head and neck cancer, head and neck squamous cell cancer, breast cancer, melanoma, acute lymphoblastic leukemia (ALL), acute myeloid leukemia (AML), adrenocortical carcinoma, kaposi sarcoma, aids-related lymphoma, primary CNS lymphoma, anal cancer, appendix cancer, astrocytomas, brain cancer, atypical teratoid/rhabdoid tumor, basal cell carcinoma of the skin, bile duct cancer, bladder cancer, bone cancer, bronchial tumors, Burkitt lymphoma, non-Hodgkin lymphoma, carcinoid tumor (gastrointestinal), cardiac tumors, cervical cancer, cholangiocarcinoma, chordoma, chronic lymphocytic leukemia (CLL), chronic myelogenous leukemia (CML), colorectal cancer, craniopharyngioma, cutaneous T-cell lymphoma, ductal carcinoma in situ (DCIS), endometrial cancer, esophageal cancer, esthesioneuroblastoma, extragonadal germ cell tumor, eye cancer, intraocular melanoma, retinoblastoma, fallopian tube cancer, gallbladder cancer, gastric cancer, gastrointestinal carcinoid tumor, gastrointestinal stromal tumors (GIST), germ cell tumors, gestational trophoblastic disease, hairy cell leukemia, head and neck cancer, liver cancer, Hodgkin lymphoma, hypopharyngeal cancer, islet cell tumors, pancreatic neuroendocrine tumors, Langerhans cell histiocytosis, laryngeal cancer, leukemia, lip and oral cavity cancer, lung cancer, lymphoma, Merkel cell carcinoma, mesothelioma, plasma cell neoplasms, mycosis fungoides, myelodysplastic syndromes, myelodysplastic/myeloproliferative neoplasms, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paraganglioma, parathyroid cancer, penile cancer, multiple myeloma, prostate cancer, renal cell cancer, retinoblastoma, rhabdomyosarcoma, salivary gland cancer, sarcoma, uterine sarcoma, small cell lung cancer, small intestine cancer, soft tissue sarcoma, T-cell lymphoma, testicular cancer, throat cancer, thymoma, thyroid cancer, urethral cancer, uterine cancer, vaginal cancer, and Wilms tumor.

4: The method of claim 3, wherein the primary cancer is a head and neck squamous cell cancer.

5: The method of claim 4, wherein the head and neck squamous cell cancer is human papillomavirus-negative.

6: The method of claim 3, wherein the primary cancer is breast cancer.

7: The method of claim 3, wherein the primary cancer is melanoma.

8: The method of claim 1, wherein the aggressive cancer treatment regimen comprises surgical resection of the primary tumor and lymph node dissection followed by radiotherapy with or without chemotherapy.

9: The method of claim 1, wherein the lymph node metastasis is taken from a formalin-fixed, paraffin-embedded sample, fine needle aspirate, or fresh frozen sample.

10: The method of claim 1, wherein the subject is a human.

11: A method for predicting outcome of locoregional control (LRC), relapse-free survival (RFS), and/or overall survival (OS) of a lymph node metastasis (LNM) in a subject, the method comprising:
(a) obtaining a sample comprising LNM cells from the subject;
(b) measuring gene expression levels in the sample of at least ten genes selected from the group consisting of: TGFBR1, OLR1, PDGFC, ACE, MGA, PAPPA, APBB2, CARD19, SPDYE3, ACOT9, C1QTNF6, FAM153B, C10orf55, SLC25A33, KAT7, MUC20, ENTPD7, GOLT1B, PPRC1, ITPRIPL2, ZC3H12C, ARF4, COL27A1, ZNF850, VKORC1, RBM23, CAPG, TOR4A, METTL21B, TRMT2B, EVC, ZBTB14, PFDN1, FBXO25, B4GALT1, NTMT1, FAM227B, AGTRAP, RRAS, STRIP2, HECTD3, SH3BGRL2, LHFPL2, WHSC1L1, DCBLD1, SPRED1, BTBD18, FTSJ1, GSTO1, FAM102A, ZDHHC8, PDGFB, SDHAF4, PCBP4, HRH1, FMC1, FLNB, RPAIN, TAS2R4, TUBD1, FKBP14, METTL21A, TANC2, POLB, XKR4, ZNF19, NOX5, HBP1, C1orf56, KCNJ15, NDOR1, 43351, and PHACTR3;
(c) determining a LNM-specific molecular subtype comprising the gene expression levels of the at least ten genes; and
(d) providing an indication as to the outcome of LRC, RFS, and/or OS when the LNM-specific molecular subtype indicates that the expression levels of the at least ten genes are altered in a predictive manner.

12: The method of claim 11, wherein the LNM-specific molecular subtype for the LNM is classified as Group 1, Group 2, or Group 3.

13: The method of claim 12, wherein Group 1 indicates an immune subtype, Group 2 indicates a significantly worse LRC, RFS, and/or OS, and Group 3 indicates a metabolic/proliferative subtype.

14: The method of claim 11, wherein measuring gene expression levels of the at least ten genes comprises measurement by RNAseq, miRNAseq, or RT-PCR, microRNA, nanostring, northern blot, qRT-PCR, in situ hybridization, immunohistochemistry, or Western blotting.

15: The method of claim 11, wherein the LNM cells from the subject are obtained from a primary cancer selected from the group consisting of: human papillomavirus-negative head and neck cancer, head and neck squamous cell cancer, breast cancer, melanoma, acute lymphoblastic leukemia (ALL), acute myeloid leukemia (AML), adrenocortical carcinoma, kaposi sarcoma, aids-related lymphoma, primary CNS lymphoma, anal cancer, appendix cancer, astrocytomas, brain cancer, atypical teratoid/rhabdoid tumor, basal cell carcinoma of the skin, bile duct cancer, bladder cancer, bone cancer, bronchial tumors, Burkitt lymphoma, non-Hodgkin lymphoma, carcinoid tumor (gastrointestinal), cardiac tumors, cervical cancer, cholangiocarcinoma, chordoma, chronic lymphocytic leukemia (CLL), chronic myelogenous leukemia (CML), colorectal cancer, craniopharyngioma, cutaneous T-cell lymphoma, ductal carcinoma in situ (DCIS), endometrial cancer, esophageal cancer, esthesioneuroblastoma, extragonadal germ cell tumor, eye cancer, intraocular melanoma, retinoblastoma, fallopian tube cancer, gallbladder cancer, gastric cancer, gastrointestinal carcinoid tumor, gastrointestinal stromal tumors (GIST), germ cell tumors, gestational trophoblastic disease, hairy cell leukemia, head and neck cancer, liver cancer, Hodgkin lymphoma, hypopharyngeal cancer, islet cell tumors, pancreatic neuroendocrine tumors, Langerhans cell histiocytosis, laryngeal cancer, leukemia, lip and oral cavity cancer, lung cancer, lymphoma, Merkel cell carcinoma, mesothelioma, plasma cell neoplasms, mycosis fungoides, myelodysplastic syndromes, myelodysplastic/myeloproliferative neoplasms, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paraganglioma, parathyroid cancer, penile cancer, multiple myeloma, prostate cancer, renal cell cancer, retinoblastoma, rhabdomyosarcoma, salivary gland cancer, sarcoma, uterine sarcoma, small cell lung cancer, small intestine cancer, soft tissue sarcoma, T-cell lymphoma, testicular cancer, throat cancer, thymoma, thyroid cancer, urethral cancer, uterine cancer, vaginal cancer, and Wilms tumor.

16: The method of claim 15, wherein the primary cancer is a head and neck squamous cell cancer.

17: The method of claim 16, wherein the head and neck squamous cell cancer is human papillomavirus-negative.

18: The method of claim 15, wherein the primary cancer is breast cancer.

19: The method of claim 15, wherein the primary cancer is melanoma.

20: The method of claim 11, wherein the lymph node metastasis is taken from a formalin-fixed, paraffin-embedded sample, fine needle aspirate, or fresh frozen sample.

21: The method of claim 11, wherein the subject is a human.


SEQUENCE LISTING (88 sequences)

SEQ ID NO: 1 - DNA, 100 bp, Artificial Sequence
  Synthetic oligonucleotide
gaagtacggcacccaggccaggaagtttgatgtgaaccagttgcagaacaccactatcaagcggatcataaagaaggttcaggacctagaacgggcagca

SEQ ID NO: 2 - DNA, 100 bp, Artificial Sequence
  Synthetic oligonucleotide
cctgcagagcatgaagtaaagaaagtgactttaggagataccttaactcgacgttccattagccagcagaagtccggagtttccattaccattgatgacc

SEQ ID NO: 3 - DNA, 100 bp, Artificial Sequence
  Synthetic oligonucleotide
aaatttgccaccctcaggagcggaacattttcaatcggatctttggtggtttccttatgaggaaggcatatgaacttgcgtgggctactgcttgtagctt

SEQ ID NO: 4 - DNA, 100 bp, Artificial Sequence
  Synthetic oligonucleotide
tggctcagcgggactccatcgacgccataagcatgtttctgggtggcttgctggccaccatcttcctggacatcgtgcacatcagcatcttctacccgcg

SEQ ID NO: 5 - DNA, 100 bp, Artificial Sequence
  Synthetic oligonucleotide
ggttacagtacttggcctctgaaacagtgctgaacttggcttttgcgcagctcattcttggaatcccagaacaggccttaagtcttctccacatggccat

SEQ ID NO: 6 - DNA, 100 bp, Artificial Sequence
  Synthetic oligonucleotide
aaattccttacgcagtggtattcatgatggtgccctatctaagtccaggactgttttcctacagcgtgcctcaaaagtgttgtagagggcaggattctac

SEQ ID NO: 7 - DNA, 100 bp, Artificial Sequence
  Synthetic oligonucleotide
gtctcttcgtaacagaacatggtatgttcaagccacttgtgcaacacaaggaactggtctgtatgaaggacttgactggctgtcaaatgagctttcaaaa

SEQ ID NO: 8 - DNA, 100 bp, Artificial Sequence
  Synthetic oligonucleotide
gtgggagcccgtggcctttaaagtgccgttcagccttttcctccaggggtgctttgtaaacacggctgtgctcagggctcgcgggtgaccgaaaggatca

SEQ ID NO: 9 - DNA, 100 bp, Artificial Sequence
  Synthetic oligonucleotide
ttcaatgaggagaactacggtgtgaagactacctatgatagcagtctttcttcttatacggtgcccttagaaaaggacaactcagaagagtttcgtcagc

SEQ ID NO: 10 - DNA, 100 bp, Artificial Sequence
  Synthetic oligonucleotide
acacaaaggagacaatgctctctgatggtttgaactcactcacctaccaggtgctggatgtacagagatacccattgtatacccaaatcacagtggacat

SEQ ID NO: 11 - DNA, 100 bp, Artificial Sequence
  Synthetic oligonucleotide
cttgaagattatacctgtggagctgggatgtggtgctcaggaagaatataagtagtcagcattgtagcagttgagtctcagtggtaatggaaatgaggaa

SEQ ID NO: 12 - DNA, 100 bp, Artificial Sequence
  Synthetic oligonucleotide
ttattgctttcggcagtaagggggtttgtttgatggtgctatcagagggggaaaggcaaggccagattactgaaaatttgcagcttggtttaaagtccgt

SEQ ID NO: 13 - DNA, 100 bp, Artificial Sequence
  Synthetic oligonucleotide
gctttttggaaacgggtcaggattggcctggaggatatttggaatagcctctcttcagtgttcacagagatgcaaccaatagacagaaaccagaggtaat

SEQ ID NO: 14 - DNA, 100 bp, Artificial Sequence
  Synthetic oligonucleotide
ccctgcacagcggcgaggacttccagacgctgctcttcgaaagggtctttgtgaaccttgatgggtgctttgacatggcgaccggccagtttgctgctcc

SEQ ID NO: 15 - DNA, 100 bp, Artificial Sequence
  Synthetic oligonucleotide
tgactccagcccatttgcccttgaactgctgatatctgatgactgctttgtgctggacaacgggctctgtggcaagatctatatctggaaggggcgaaaa

SEQ ID NO: 16 - DNA, 100 bp, Artificial Sequence
  Synthetic oligonucleotide
cctggtgcaggacacgcctttcctgacaggccatgggcgcttgagtgagcagcaggtggacaggatcatcctccagctgaaccgttactacccacagatc

SEQ ID NO: 17 - DNA, 100 bp, Artificial Sequence
  Synthetic oligonucleotide
caccgcccagcacccggcccctacctcctaccaccagctcctctaaaaaacccattcccacactagctcggactgaggccaagataaccagccatgccag

SEQ ID NO: 18 - DNA, 100 bp, Artificial Sequence
  Synthetic oligonucleotide
ctcttgttgaacacaagtgaagtaaccgtccgctttgagagtggatcccacatttctggccggggttttttgctgacctatgcgagcagcgaccatccag

SEQ ID NO: 19 - DNA, 100 bp, Artificial Sequence
  Synthetic oligonucleotide
cttccagccagtctgtgcttcacagctctatcctcgccacaaacatcttctgatcaaacggtccctgcgctgccgtaaatgtgaacataatttgagcaag

SEQ ID NO: 20 - DNA, 100 bp, Artificial Sequence
  Synthetic oligonucleotide
ctgttctcagagaaggaggatggtaaggtatccaggatttttagggggtaagggagatatatgggatgtgagagcagtgattgtagacagactgtcactg

SEQ ID NO: 21 - DNA, 100 bp, Artificial Sequence
  Synthetic oligonucleotide
gggagccattctatataaaacacgattcttaccactcagggatcttcggcaggaaggtgtccgacaagcccatggtagctggttccgtctctcctttgta

SEQ ID NO: 22 - DNA, 100 bp, Artificial Sequence
  Synthetic oligonucleotide
tttcttgtggaccagtttaagtgttccagctccaaagcccgacagctgatgatgactctgacggaaagaatgattgcagccgaagggctattgtgcgatt

SEQ ID NO: 23 - DNA, 100 bp, Artificial Sequence
  Synthetic oligonucleotide
gaacctatggacagcccactgatgtcagctatacccaggctcagaccactgcaacctatgggcagaccgcctatgcaacttcttatggacagcctcccac

SEQ ID NO: 24 - DNA, 100 bp, Artificial Sequence
  Synthetic oligonucleotide
ctggagaagagtcctgctttctctggagtccagacctgtatcattccatgaggaactttccccttcagatcacctctgcgccacatctcatccatgcctc

SEQ ID NO: 25 - DNA, 100 bp, Artificial Sequence
  Synthetic oligonucleotide
caggggtccttcagcctctgtgggggcctggggtggcctcatggccccatttttcaagatgaggttggaggcttctgtatgtagagggcactggcttggg

SEQ ID NO: 26 - DNA, 100 bp, Artificial Sequence
  Synthetic oligonucleotide
ccgcctcaagcctcgcagcctttcacccagtttcctcccgattcttgctgggacaacaaaattgggaagcggttctcgaaacatactgtcaaggcgtagt

SEQ ID NO: 27 - DNA, 100 bp, Artificial Sequence
  Synthetic oligonucleotide
tcttcagatgactaagcaagtgaacaatggcctcaccctcagtgaccttcctctgcacatgctgaacaacatcctataccggttctcagacggatgggac

SEQ ID NO: 28 - DNA, 100 bp, Artificial Sequence
  Synthetic oligonucleotide
ttatgtgtaactggctctgagtctctgcttgaggaccagaggaaaatggttgttggacctgacttgttaatggctactgctttactaaggagatgtgcaa

SEQ ID NO: 29 - DNA, 100 bp, Artificial Sequence
  Synthetic oligonucleotide
ggtttggagaaatctggatgcattgtcaacaacctggccgagttcactgtggatcctaaggatgctggaaaagctcccttaaagatatttgctcaggatg

SEQ ID NO: 30 - DNA, 100 bp, Artificial Sequence
  Synthetic oligonucleotide
gagtctgctggcttggtgggtctcaagttgccccatcagcctggagggaagggctgggagccatgaacatggagaatatccttggatgctgcattcatag

SEQ ID NO: 31 - DNA, 100 bp, Artificial Sequence
  Synthetic oligonucleotide
gatgacggggctcacgttgtacgttcacatcaggtcccggcccgccggaacctgggcgatccacgatgccgagtttgccacgctgcgacagcccataggc

SEQ ID NO: 32 - DNA, 100 bp, Artificial Sequence
  Synthetic oligonucleotide
tccggaagacgtggcggctctcgcctgggctgtttcccggcttcatttctcccgactcagcttcccaccctgggctttccgaggtgctgtcgccgctgtc

SEQ ID NO: 33 - DNA, 100 bp, Artificial Sequence
  Synthetic oligonucleotide
tctgactaataagaagacgaccttctttggtggcaattctatctctatgattgattacctcatctggccctggtttgaacggctggaagcaatgaagtta

SEQ ID NO: 34 - DNA, 100 bp, Artificial Sequence
  Synthetic oligonucleotide
cttgtggaggacctggtggtcaagactttgcaagatctggattcagtaaaaactgtggctcacctggatcatcacagctctcttccaattctttgtatgc

SEQ ID NO: 35 - DNA, 100 bp, Artificial Sequence
  Synthetic oligonucleotide
ccgtccacctcccgatcatcgagatccgcatcgtggagtgccgagatgatgggattgatgttcgtctccgaggggtcaagatcaagtcatctagacagcg

SEQ ID NO: 36 - DNA, 100 bp, Artificial Sequence
  Synthetic oligonucleotide
gtggcagctcaaaatgatatgtttgagtagacgaacagctgacatggagttcccgtgcacctacggaaggggacgctttgaaggaaccaagtgcattttt

SEQ ID NO: 37 - DNA, 100 bp, Artificial Sequence
  Synthetic oligonucleotide
ttctgactcagcagctccaccaaaattgacatcctagcaaacactgtgaaggaattaacctaagtgcttccagagcatctcatgtaacctctatggagta

SEQ ID NO: 38 - DNA, 100 bp, Artificial Sequence
  Synthetic oligonucleotide
tccagttactccttatatgtgtgttctatggaggggcagggaaaaggtggcacttgtgagtgtgtgtggattggcagggggtccattcactttgggttcc

SEQ ID NO: 39 - DNA, 100 bp, Artificial Sequence
  Synthetic oligonucleotide
agacccgccacatcagcaccatccgagagcttattagtaatgtgtagattctcatgtcccagccagacttacagaattggggtctgtatcttaacaagaa

SEQ ID NO: 40 - DNA, 100 bp, Artificial Sequence
  Synthetic oligonucleotide
atggttctcaaggtggatacctctgagtccttatggcatattcagatcagtagaagcagatttcttttggatagtgatgggaaaaggctaatctgtgagg

SEQ ID NO: 41 - DNA, 100 bp, Artificial Sequence
  Synthetic oligonucleotide
tgattaactccgtggactcctgactctttcttcgcccggaacatcaatatgtgtcatgtcattgtcacctgtcgctcgatgctctggaccttgctgagta

SEQ ID NO: 42 - DNA, 100 bp, Artificial Sequence
  Synthetic oligonucleotide
tcgtgccctatgaggagaccacggaatttgggttgcagaaattccacaagcctcttgcaactttttcctttgcaaaccacacgatccagatccggcagga

SEQ ID NO: 43 - DNA, 100 bp, Artificial Sequence
  Synthetic oligonucleotide
tccaagtggaatgaacttattcaggcaccctaatgggcagattgtccagcttctacctttgcatcagcttcgaggctctaatacccagcccaacttacag

SEQ ID NO: 44 - DNA, 100 bp, Artificial Sequence
  Synthetic oligonucleotide
ttgagacaccaagttacgtcaaagtctcaggagcagctccggtctccatagaggctgggtcagcagtgggcaaaacaacttcctttgctgggagctctgc

SEQ ID NO: 45 - DNA, 100 bp, Artificial Sequence
  Synthetic oligonucleotide
taggagtgagcatgagatttctgaaattattgatgggctctctgagcaggagaataatgagaaacaaatgcggcagctctctgtgattccacctatgatg

SEQ ID NO: 46 - DNA, 100 bp, Artificial Sequence
  Synthetic oligonucleotide
acctgccctccactgccctctgtcagatggactttgccgtcctgggcctcggggactcctcatacgccaagttcaacttcgtggccaagaagctgcaccg

SEQ ID NO: 47 - DNA, 100 bp, Artificial Sequence
  Synthetic oligonucleotide
agctccataaggtggactttatctggatcaacagagaccagcggtctttcgagtggtttgtgagcctgctgactaaactggagatggaccaggccgagga

SEQ ID NO: 48 - DNA, 100 bp, Artificial Sequence
  Synthetic oligonucleotide
gaaagggaaagttaaggatgctggagcagaacaatggatttctctttctctttcatgcaagggatcatgggaaacacaattcagcaaccacctcaactca

SEQ ID NO: 49 - DNA, 100 bp, Artificial Sequence
  Synthetic oligonucleotide
aggatcaccaagcggctgctcctgccgctgttcagagaggtggatatggtcgacataacggaggacttcctggttcaagccaagacctacctgggggagg

SEQ ID NO: 50 - DNA, 100 bp, Artificial Sequence
  Synthetic oligonucleotide
cccaggtgtctgacctcctaacacaagagcaagcaaacctaactcaccagaaaaagaaactggagggacagatctcagcccggcaacaagcagaagaagc

SEQ ID NO: 51 - DNA, 100 bp, Artificial Sequence
  Synthetic oligonucleotide
caatgcgacgacatgaataagatcaatggtgatggctgctcccttttctgccgacaagaagtctccttcaattgtattgatgaacccagccggtgctatt

SEQ ID NO: 52 - DNA, 100 bp, Artificial Sequence
  Synthetic oligonucleotide
catcaccgggtctacagcagctgtcttccatgcagtctccatgattgctttcaaactggatgaggacctttgtgctgctcctgcaaatggtggaaatgtc

SEQ ID NO: 53 - DNA, 100 bp, Artificial Sequence
  Synthetic oligonucleotide
gattcacctcttcctctggttcctttcatctctctacctccaccctgcattttcctcttgtcctggcccttcagtctgctccaccaaggggctcttgaac

SEQ ID NO: 54 - DNA, 100 bp, Artificial Sequence
  Synthetic oligonucleotide
gcaacttatgaaagtagacattcagatccagccattactaacctattccttttttggggaaatctgagcctagctcagaaaaacataaagcaccttgaaa

SEQ ID NO: 55 - DNA, 100 bp, Artificial Sequence
  Synthetic oligonucleotide
tgagagaggcagtggccgtctccacctcagctcctgctccctctgcatcagagcccttcctttcttgggggatgggccttgccctcttctcttttccctt

SEQ ID NO: 56 - DNA, 100 bp, Artificial Sequence
  Synthetic oligonucleotide
ggagaacaaggagaacctgatcataaattctgaactcaaagacgacttgcttttgtatcaggacgaggaggcgctgaacgactccattatttctggaaca

SEQ ID NO: 57 - DNA, 100 bp, Artificial Sequence
  Synthetic oligonucleotide
tctgaatacattgctacagtctgtggcagtttcagaagaggtgcagagtccagtggtgacatggatgttctcctgacccatcccagcttcacttcagaat

SEQ ID NO: 58 - DNA, 100 bp, Artificial Sequence
  Synthetic oligonucleotide
agagaagtgctggacaaagtagtcctgctaaagaaggccctctagacctctacccaaagctggctgacactatccaaaccaatcctataccaacccatct

SEQ ID NO: 59 - DNA, 100 bp, Artificial Sequence
  Synthetic oligonucleotide
tgtggatctccatgaacagtcccagcagaacaagagccagtgttgtagcaactgagggggtggctagcagcaaacaagtatggagctagcacaagagcta

SEQ ID NO: 60 - DNA, 100 bp, Artificial Sequence
  Synthetic oligonucleotide
tcaatatcactgaagacatgctccggggcatctttgagccctttggtaaaattgataatattgtcctgatgaaggactcagatacaggccgctctaaagg

SEQ ID NO: 61 - DNA, 100 bp, Artificial Sequence
  Synthetic oligonucleotide
ctgagacttgtgtatctaattgcctattggacacctccatctgggtaaacccaggcatccagaactttccctcaagacttacctccctcagtgcaggtcg

SEQ ID NO: 62 - DNA, 100 bp, Artificial Sequence
  Synthetic oligonucleotide
cagtacatgcgtgctggccacggcttcctgctggtgttcgccattaacgaccggcagagtttcaacgaggtgggcaagctcttcacgcagattctgcggg

SEQ ID NO: 63 - DNA, 100 bp, Artificial Sequence
  Synthetic oligonucleotide
agtctgaacttgtcaaacagtcccttaagaagccgaagttaccagaaggtcgttttgatgcaccagaggattcccatttagagaaagaaccactggaaaa

SEQ ID NO: 64 - DNA, 100 bp, Artificial Sequence
  Synthetic oligonucleotide
tcgatgcgcagtttgaaaattatctgcaggaggagctgaagatccgccgctcgctcttcgactaccatgacacaaggatccacgtttgcctctacttcat

SEQ ID NO: 65 - DNA, 100 bp, Artificial Sequence
  Synthetic oligonucleotide
caggtgtactttcttaatgactgaaagataagtgggtagcaccgtcagagagaaaatgttggatctgccctaggttatagtagatgccggaattgcgtaa

SEQ ID NO: 66 - DNA, 100 bp, Artificial Sequence
  Synthetic oligonucleotide
cgcctggtgttccgggaagaaggctaccttgccttttatagaggactgtttgcccagcttatccggcagatcccaaatactgccattgtgttgtctactt

SEQ ID NO: 67 - DNA, 100 bp, Artificial Sequence
  Synthetic oligonucleotide
aacaaaagatcttctacttcctgtacgggaagacccactctcacatacccttgcgccctaagcattggttccagttatgccgtcccatgaacccgagggc

SEQ ID NO: 68 - DNA, 100 bp, Artificial Sequence
  Synthetic oligonucleotide
ctatccagctcgagggagagagttttattcagaatgggaaacttaccaaggagactactatgaatcacgatactacgatgatcctcgggaatacagggat

SEQ ID NO: 69 - DNA, 100 bp, Artificial Sequence
  Synthetic oligonucleotide
ttcagagcagaagtatggaatacgtacagcggcaaatatccaaggaatgtggaagcctaaagtcccaaaatagggtccctttgaaatcaatcagacatgt

SEQ ID NO: 70 - DNA, 100 bp, Artificial Sequence
  Synthetic oligonucleotide
cccgaccacgatctcggtcacgctccaaatcaagatcccggacgcggtcccgctctccttctcacactcgacctagacggcgccatagatcccgatcaag

SEQ ID NO: 71 - DNA, 100 bp, Artificial Sequence
  Synthetic oligonucleotide
agccgagttgtcagaattgtatagttacactgagaacctggaattcaccaataacaggaggtgctttgaagaagatttcaagactcaagtgcagggcaag

SEQ ID NO: 72 - DNA, 100 bp, Artificial Sequence
  Synthetic oligonucleotide
agggtttgagtaaaaaagttggtgtatcatcctccatcctccaaggtctctggatctcttatagcacagaaggtctttccatggcactggcgtctttacg

SEQ ID NO: 73 - DNA, 100 bp, Artificial Sequence
  Synthetic oligonucleotide
ggactcgagcagtgtctggtttgtgaccttgctcaatatcttgtactgtgtgaagattactaacttccaacactcagtgtttctcctgctgaagcggaat

SEQ ID NO: 74 - DNA, 100 bp, Artificial Sequence
  Synthetic oligonucleotide
gaatccttcaaacgtgctgacatctatgcaatgggcttagtattctgggaaattgctcgacgatgttccattggtggaattcatgaagattaccaactgc

SEQ ID NO: 75 - DNA, 100 bp, Artificial Sequence
  Synthetic oligonucleotide
tctggtctggccttcgtgttggcttcaaggtggtaacagagagagctgcagagtgggggtgtgtttgccagtccctgcatgggtgaatgttggggtgccc

SEQ ID NO: 76 - DNA, 100 bp, Artificial Sequence
  Synthetic oligonucleotide
atgtccaaccggcctaataacaatccaggggggtcactgcgacgttcacagaggaacactgccggggcccaaccacaagacgactcaataggaggaagaa

SEQ ID NO: 77 - DNA, 100 bp, Artificial Sequence
  Synthetic oligonucleotide
agtaccatgacccgttgcagccatcagcagtctccctatcagcttctgtttggggaaccctacatctttgaagaacttctgagcttgaagatccgcatct

SEQ ID NO: 78 - DNA, 100 bp, Artificial Sequence
  Synthetic oligonucleotide
gccatccataagatctgtgcaaaactgatgaatatcaagcagatctcctttagtgatatcaatcaagtcctcgcacatcagctgggaagtgtgttccagc

SEQ ID NO: 79 - DNA, 100 bp, Artificial Sequence
  Synthetic oligonucleotide
aagaggcactgagccctcaacccaagccaggctgacctcatctgctttgctttggcatgtgagccttgcctaagggggcatatctgggtccctagaaggc

SEQ ID NO: 80 - DNA, 100 bp, Artificial Sequence
  Synthetic oligonucleotide
ggaggcacttcaagttctacgctgtgaattgacgccgctgaaatacaatacagagcgcattcatgttcttagtgggtatctgatgtgtagccatgcagaa

SEQ ID NO: 81 - DNA, 100 bp, Artificial Sequence
  Synthetic oligonucleotide
ccacatcttgcagctcgggcaaatctggagatatttccacacaatatacttaggtattcgaagccgacagagtggggagaatgacagatggaggttttac

SEQ ID NO: 82 - DNA, 100 bp, Artificial Sequence
  Synthetic oligonucleotide
gtttgagtatttgctttatggtcaccatcgggagcagattgcctgccaggcgtgtgggaagacgttttctgatgaaggcagattgaggaagcatgagaaa

SEQ ID NO: 83 - DNA, 100 bp, Artificial Sequence
  Synthetic oligonucleotide
caaatctgagtctctcaggcccacgaagccctgaaaggcgtttctccttagacacagattatagaataagttccgtagcttctgactgcagcagtgaagg

SEQ ID NO: 84 - DNA, 100 bp, Artificial Sequence
  Synthetic oligonucleotide
accactgcccctgggtcaacaactgcatcgggcgtcgaaactatcgctacttcttcctgttcctgctgtcactcagtgcacacatggtgggcgtcgtggc

SEQ ID NO: 85 - DNA, 100 bp, Artificial Sequence
  Synthetic oligonucleotide
gggaaagtctttagttatagtcatggcagctcctcaaccccatgattgtgatgagattgggaagcccactgtaacactggagagcccaggttatatcaga

SEQ ID NO: 86 - DNA, 100 bp, Artificial Sequence
  Synthetic oligonucleotide
aagcctacattccctgcttatacacagtctacagcttcaacaactagtacaacaaatagtactgcagctaaaccagcggcttcaataacaagtaagcctg

SEQ ID NO: 87 - DNA, 100 bp, Artificial Sequence
  Synthetic oligonucleotide
ggtgaataaatggagccaatagacttgttcactgcatggttacgtcagaggcgtgtaaaccagagcaacttcttcttgaataggagctgtgtaaagtaac

SEQ ID NO: 88 - DNA, 12 bp, Artificial Sequence
  Synthetic oligonucleotide
tggaattctcgg